(19) United States
(12) Patent Application Publication
(10) Pub. No.: US 2004/0248256 A1
(43) Pub. Date: Dec. 9, 2004

(54) Secreted proteins and polynucleotides encoding them

(76) Inventors: Kenneth Jacobs, Newton, MA (US); John M. McCoy, Reading, MA (US); Edward R. LaVallie, Harvard, MA (US); Lisa Collins-Racie, Acton, MA (US); David Merberg, Acton, MA (US); Cheryl Evans, Germantown, MD (US); Maurice Treacy, Dublin (IE); Michael J. Agostino, Andover, MA (US); Robert J. Steininger II, Cambridge, MA (US); Michael R. Bowman, Canton, MA (US); Elizabeth DiBlasio-Smith, Tyngsboro, MA (US); Angela Widom, Acton, MA (US)

Correspondence Address: FITZPATRICK CELLA HARPER & SCINTO, 30 ROCKEFELLER PLAZA, NEW YORK, NY, 10112, US

(21) Appl. No.: 10/821,273

(22) Filed: Apr. 9, 2004

Related U.S. Application Data
(63) Continuation of application No. 09/306,111, filed on May 6, 1999, now abandoned.
(60) Provisional application No. 60/084,564, filed on May 7, 1998.
(60) Provisional application No. 60/087,645, filed on Jun. 2, 1998.
(60) Provisional application No. 60/093,712, filed on Jul. 22, 1998.
(60) Provisional application No. 60/094,935, filed on Jul. 31, 1998.
(60) Provisional application No. 60/095,880, filed on Aug. 10, 1998.
(60) Provisional application No. 60/096,068, filed on Aug. 11, 1998.

(51) Int. Cl. C07H 21/04; C07K 14/705

(52) U.S. Cl. 435/69.1; 435/320.1; 435/325; 530/350; 536/23.5

(57) ABSTRACT

Novel polynucleotides and the proteins encoded thereby are disclosed.

This application is a continuation-in-part of the following applications:

(1) provisional application Ser. No. 60/084,564, filed May 7, 1998;

(2) provisional application Ser. No. 60/087,645, filed Jun. 2, 1998;

(3) provisional application Ser. No. 60/093,712, filed Jul. 22, 1998;

(4) provisional application Ser. No. 60/094,935, filed Jul. 31, 1998;

(5) provisional application Ser. No. 60/095,880, filed Aug. 10, 1998;

(6) provisional application Ser. No. 60/096,068, filed Aug. 11, 1998;

all of which are incorporated by reference herein.

### FIELD OF THE INVENTION

The present invention provides novel polynucleotides and proteins encoded by such polynucleotides, along with therapeutic, diagnostic and research utilities for these polynucleotides and proteins.

### BACKGROUND OF THE INVENTION

Technology aimed at the discovery of protein factors (including e.g., cytokines, such as lymphokines, interferons, CSFs and interleukins) has matured rapidly over the past decade. The now routine hybridization cloning and expression cloning techniques clone novel polynucleotides “directly” in the sense that they rely on information directly related to the discovered protein (i.e., partial DNA/amino acid sequence of the protein in the case of hybridization cloning; activity of the protein in the case of expression cloning). More recent “indirect cloning techniques such as signal sequence cloning, which isolates DNA sequences based on the presence of a now well-recognized secretory leader sequence motif, as well as various PCR-based or low stringency hybridization cloning techniques, have advanced the state of the art by making available large numbers of DNA/amino acid sequences for proteins that are known to have biological activity by virtue of their secreted nature in the case of leader sequence cloning, or by virtue of the cell or tissue source in the case of PCR-based techniques. It is to these proteins and the polynucleotides encoding them that the present invention is directed.

### SUMMARY OF THE INVENTION

In one embodiment, the present invention provides a composition comprising an isolated polynucleotide selected from the group consisting of:

(a) a polynucleotide comprising the nucleotide sequence of SEQ ID NO:1;

(b) a polynucleotide comprising the nucleotide sequence of SEQ ID NO:1 from nucleotide 61 to nucleotide 366;

(c) a polynucleotide comprising the nucleotide sequence of the full-length protein coding sequence of clone bn365_{—}53 deposited under accession number ATCC 98752;

(d) a polynucleotide encoding the full-length protein encoded by the cDNA insert of clone bn365_{—}53 deposited under accession number ATCC 98752;

(e) a polynucleotide comprising the nucleotide sequence of a mature protein coding sequence of clone bn365_{—}53 deposited under accession number ATCC 98752;

(f) a polynucleotide encoding a mature protein encoded by the cDNA insert of clone bn365_{—}53 deposited under accession number ATCC 98752;

(g) a polynucleotide encoding a protein comprising the amino acid sequence of SEQ ID NO:2;

(h) a polynucleotide encoding a protein comprising a fragment of the amino acid sequence of SEQ ID NO:2 having biological activity, the fragment comprising eight contiguous amino adds of SEQ ID NO:2;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:1.

Preferably, such polynucleotide comprises the nucleotide sequence of SEQ ID NO:1 from nucleotide 61 to nucleotide 366; the nucleotide sequence of the full-length protein coding sequence of clone bn365_{—}53 deposited under accession number ATCC 98752; or the nucleotide sequence of a mature protein coding sequence of clone bn365_{—}53 deposited under accession number ATCC 98752. In other preferred embodiments, the polynucleotide encodes the full-length or a mature protein encoded by the cDNA insert of clone bn365_{—}53 deposited under accession number ATCC 98752. In further preferred embodiments, the present invention provides a polynucleotide encoding a protein comprising a fragment of the amino acid sequence of SEQ ID NO:2 having biological activity, the fragment preferably comprising eight (more preferably twenty, most preferably thirty) contiguous amino adds of SEQ ID NO:2, or a polynucleotide encoding a protein comprising a fragment of the amino acid sequence of SEQ ID NO:2 having biological activity, the fragment comprising the amino acid sequence from amino acid 46 to amino acid 55 of SEQ ID NO:2.

Other embodiments provide the gene corresponding to the cDNA sequence of SEQ ID NO:1.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:1, but excluding the poly(A) tail at the 3′ end of SEQ ID NO:1; and

(ab) the nucleotide sequence of the cDNA insert of clone bn365_{—}53 deposited under accession number ATCC 98752;

(ii) hybridizing said probe(s) to human genomic DNA in conditions at least as stringent as 4× SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:1, but excluding the poly(A) tail at the 3′ end of SEQ ID NO:1; and

(bb) the nucleotide sequence of the cDNA inset of clone bn365_{—}53 deposited under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:1, and extending contiguously from a nucleotide sequence corresponding to the 5′ end of SEQ ID NO:1 to a nucleotide sequence corresponding to the 3′ end of SEQ ID NO:1, but excluding the poly(A) tail at the 3′ end of SEQ ID NO:1. Also preferably the polynucleotide isolated according to the above process comprises a nucleotide sequence corresponding to the cDNA sequence of SEQ ID NO:1 from nucleotide 61 to nucleotide 366, and extending contiguously from a nucleotide sequence corresponding to the 5′ end of said sequence of SEQ ID NO:1 from nucleotide 61 to nucleotide 366, to a nucleotide sequence corresponding to the 3′ end of said sequence of SEQ ID NO:1 from nucleotide 61 to nucleotide 366.

In other embodiments, the present invention provides a composition comprising a protein, wherein said protein comprises an amino acid sequence selected from the group consisting of:

(a) the amino add sequence of SEQ ID NO:2;

(b) a fragment of the amino add sequence of SEQ ID NO:2, the fragment comprising eight contiguous amino acids of SEQ ID NO:2; and

(c) the amino add sequence encoded by the cDNA insert of clone bn365_{—}53 deposited under accession number ATCC 98752;

the protein being substantially free from other mammalian proteins. Preferably such protein comprises the amino add sequence of SEQ ID NO:2. In further preferred embodiments, the present invention provides a protein comprising a fragment of the amino add sequence of SEQ ID NO:2 having biological activity, the fragment preferably comprising eight (more preferably twenty, most preferably thirty) contiguous amino adds of SEQ ID NO:2, or a protein comprising a fragment of the amino acid sequence of SEQ ID NO:2 having biological activity, the fragment comprising the amino acid sequence from amino acid 46 to amino acid 55 of SEQ ID NO:2.

In one embodiment, the present invention provides a composition comprising an isolated polynucleotide selected from the group consisting of:

(a) a polynucleotide comprising the nucleotide sequence of SEQ ID NO:3;

(b) a polynucleotide comprising the nucleotide sequence of SEQ ID NO:3 from nucleotide 206 to nucleotide 1915;

(c) a polynucleotide comprising the nucleotide sequence of SEQ ID NO:3 from nucleotide 1358 to nucleotide 1915;

(d) a polynucleotide comprising the nucleotide sequence of the full-length protein coding sequence of clone bo342_{—}2 deposited under accession number ATCC 98752;

(e) a polynucleotide encoding the full-length protein encoded by the cDNA insert of clone bo342_{—}2 deposited under accession number ATCC 98752;

(f) a polynucleotide comprising the nucleotide sequence of a mature protein coding sequence of clone bo342_{—}2 deposited under accession number ATCC 98752;

(g) a polynucleotide encoding a mature protein encoded by the cDNA insert of clone bo342_{—}2 deposited under accession number ATCC 98752;

(h) a polynucleotide encoding a protein comprising the amino acid sequence of SEQ ID NO:4;

(i) a polynucleotide encoding a protein comprising a fragment of the amino acid sequence of SEQ ID NO:4 having biological activity, the fragment comprising eight contiguous amino acids of SEQ ID NO:4;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:3.

Preferably, such polynucleotide comprises the nucleotide sequence of. SEQ ID NO:3 from nucleotide 206 to nucleotide 1915; the nucleotide sequence of SEQ ID NO:3 from nucleotide 1358 to nucleotide 1915; the nucleotide sequence of the full-length protein coding sequence of clone bo342_{—}2 deposited under accession number ATCC 98752; or the nucleotide sequence of a mature protein coding sequence of clone bo342_{—}2 deposited under accession number ATCC 98752. In other preferred embodiments, the polynucleotide encodes the full-length or a mature protein encoded by the cDNA insert of clone bo342_{—}2 deposited under accession number ATCC 98752. In further preferred embodiments, the present invention provides a polynucleotide encoding a protein comprising a fragment of the amino add sequence of SEQ ID NO:4 having biological activity, the fragment preferably comprising eight (more preferably twenty, most preferably thirty) contiguous amino acids of SEQ ID NO:4, or a polynucleotide encoding a protein comprising a fragment of the amino acid sequence of SEQ ID NO:4 having biological activity, the fragment comprising the amino acid sequence from amino acid 280 to amino add 289 of SEQ ID NO:4.

Other embodiments provide the gene corresponding to the cDNA sequence of SEQ ID NO:3.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:3, but excluding the poly(A) tail at the 3′ end of SEQ ID NO:3; and

(ab) the nucleotide sequence of the cDNA insert of clone bo342_{—}2 deposited under accession number ATCC 98752;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:3, but excluding the poly(A) tail at the 3′ end of SEQ ID NO:3; and

(bb) the nucleotide sequence of the cDNA insert of clone bo342_{—}2 deposited under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:3, and extending contiguously from a nucleotide sequence corresponding to the 5′ end of SEQ ID NO:3 to a nucleotide sequence corresponding to the 3′ end of SEQ ID NO:3, but excluding the poly(A) tail at the 3′ end of SEQ ID NO:3. Also preferably the polynucleotide isolated according to the above process comprises a nucleotide sequence corresponding to the cDNA sequence of SEQ ID NO:3 from nucleotide 206 to nucleotide 1915, and extending contiguously from a nucleotide sequence corresponding to the 5′ end of said sequence of SEQ ID NO:3 from nucleotide 206 to nucleotide 1915, to a nucleotide sequence corresponding to the 3′ end of said sequence of SEQ ID NO:3 from nucleotide 206 to nucleotide 1915. Also preferably the polynucleotide isolated according to the above process comprises a nucleotide sequence corresponding to the cDNA sequence of SEQ ID NO:3 from nucleotide 1358 to nucleotide 1915, and extending contiguously from a nucleotide sequence corresponding to the 5′ end of said sequence of SEQ ID NO:3 from nucleotide 1358 to nucleotide 1915, to a nucleotide sequence corresponding to the 3′ end of said sequence of SEQ ID NO:3 from nucleotide 1358 to nucleotide 1915.

In other embodiments, the present invention provides a composition comprising a protein, wherein said protein comprises an amino acid sequence selected from the group consisting of:

(a) the amino acid sequence of SEQ ID NO:4;

(b) a fragment of the amino add sequence of SEQ ID NO:4, the fragment comprising eight contiguous amino adds of SEQ ID NO:4; and

(c) the amino add sequence encoded by the cDNA insert of clone bo342_{—}2 deposited under accession number ATCC 98752;

the protein being substantially free from other mammalian proteins. Preferably such protein comprises the amino add sequence of SEQ ID NO:4. In further preferred embodiments, the present invention provides a protein comprising a fragment of the amino acid sequence of SEQ ID NO:4 having biological activity, the fragment preferably comprising eight (more preferably twenty, most preferably thirty) contiguous amino acids of SEQ ID NO:4, or a protein comprising a fragment of the amino acid sequence of SEQ ID NO:4 having biological activity, the fragment comprising the amino acid sequence from amino acid 280 to amino acid 289 of SEQ ID NO:4.

In one embodiment, the present invention provides a composition comprising an isolated polynucleotide selected from the group consisting of:

(a) a polynucleotide comprising the nucleotide sequence of SEQ ID NO:5;

(b) a polynucleotide comprising the nucleotide sequence of SEQ ID NO:5 from nucleotide 749 to nucleotide 2689;

(c) a polynucleotide comprising the nucleotide sequence of the full-length protein coding sequence of clone dn721_{—}8 deposited under accession number ATCC 98752;

(d) a polynucleotide encoding the full-length protein encoded by the cDNA insert of clone dn721_{—}8 deposited under accession number ATCC 98752;

(e) a polynucleotide comprising the nucleotide sequence of a mature protein coding sequence of clone dn721_{—}8 deposited under accession number ATCC 98752;

(f) a polynucleotide encoding a mature protein encoded by the cDNA insert of clone dn721_{—}8 deposited under accession number ATCC 98752;

(g) a polynucleotide encoding a protein comprising the amino add sequence of SEQ ID NO:6;

(h) a polynucleotide encoding a protein comprising a fragment of the amino acid sequence of SEQ ID NO:6 having biological activity, the fragment comprising eight contiguous amino acids of SEQ ID NO:6;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:5.

Preferably, such polynucleotide comprises the nucleotide sequence of SEQ ID NO:5 from nucleotide 749 to nucleotide 2689; the nucleotide sequence of the full-length protein coding sequence of clone dn721_{—}8 deposited under accession number ATCC 98752; or the nucleotide sequence of a mature protein coding sequence of clone dn721_{—}8 deposited under accession number ATCC 98752. In other preferred embodiments, the polynucleotide encodes the full-length or a mature protein encoded by the cDNA insert of clone dn721_{—}8 deposited under accession number ATCC 98752. In further preferred embodiments, the present invention provides a polynucleotide encoding a protein comprising a fragment of the amino acid sequence of SEQ ID NO:6 having biological activity, the fragment preferably comprising eight (more preferably twenty, most preferably thirty) contiguous amino acids of SEQ ID NO:6, or a polynucleotide encoding a protein comprising a fragment of the amino acid sequence of SEQ ID NO:6 having biological activity, the fragment comprising the amino acid sequence from amino acid 318 to amino acid 327 of SEQ ID NO:6.

Other embodiments provide the gene corresponding to the cDNA sequence of SEQ ID NO:5.

Further embodiments of the invention provide isolated polynucleotides produced according to a process selected from the group consisting of:

(a) a process comprising the steps of.

(i) preparing one or more polynucleotide probes that hybridize in 6×SSC at 65 degrees C. to a nucleotide sequence selected from the group consisting of:

(aa) SEQ ID NO:5, but excluding the poly(A) tail at the 3′ end of SEQ ID NO:5; and

(ab) the nucleotide sequence of the cDNA insert of clone dn721_{—}8 deposited under accession number ATCC 98752;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of

(i) preparing one or more polynucleotide primers that hybridize in 6×SSC at 65 degrees C. to a nucleotide sequence selected from the group consisting of:

(ba) SEQ ID NO:5, but excluding the poly(A) tail at the 3′ end of SEQ ID NO:5; and

(bb) the nucleotide sequence of the cDNA insert of clone dn721_{—}8 deposited under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:5, and extending contiguously from a nucleotide sequence corresponding to the 5′ end of SEQ ID NO:5 to a nucleotide sequence corresponding to the 3′ end of SEQ ID NO:5, but excluding the poly(A) tail at the 3′ end of SEQ ID NO:5. Also preferably the polynucleotide isolated according to the above process comprises a nucleotide sequence corresponding to the cDNA sequence of SEQ ID NO:5 from nucleotide 749 to nucleotide 2689, and extending contiguously from a nucleotide sequence corresponding to the 5′ end of said sequence of SEQ ID NO:5 from nucleotide 749 to nucleotide 2689, to a nucleotide sequence corresponding to the 3′ end of said sequence of SEQ ID NO:5 from nucleotide 749 to nucleotide 2689.

In other embodiments, the present invention provides a composition comprising a protein, wherein said protein comprises an amino acid sequence selected from the group consisting of:

(a) the amino add sequence of SEQ ID NO:6;

(b) a fragment of the amino acid sequence of SEQ ID NO:6, the fragment comprising eight contiguous amino acids of SEQ ID NO:6; and

(c) the amino acid sequence encoded by the cDNA insert of clone dn721_{—}8 deposited under accession number ATCC 98752;

the protein being substantially free from other mammalian proteins. Preferably such protein comprises the amino acid sequence of SEQ ID NO:6. In further preferred embodiments, the present invention provides a protein comprising a fragment of the amino add sequence of SEQ ID NO:6 having biological activity, the fragment preferably comprising eight (more preferably twenty, most preferably thirty) contiguous amino acids of SEQ ID NO:6, or a protein comprising a fragment of the amino acid sequence of SEQ ID NO:6 having biological activity, the fragment comprising the amino acid sequence from amino acid 318 to amino acid 327 of SEQ ID NO:6.

In one embodiment, the present invention provides a composition comprising an isolated polynucleotide selected from the group consisting of:

(a) a polynucleotide comprising the nucleotide sequence of SEQ ID NO:7;

(b) a polynucleotide comprising the nucleotide sequence of SEQ ID NO:7 from nucleotide 20 to nucleotide 484;

(c) a polynucleotide comprising the nucleotide sequence of SEQ ID NO:7 from nucleotide 18 to nucleotide 892;

(d) a polynucleotide comprising the nucleotide sequence of the full-length protein coding sequence of clone dn834_{—}1 deposited under accession number ATCC 98752;

(e) a polynucleotide encoding the full-length protein encoded by the cDNA insert of clone dn834_{—}1 deposited under accession number ATCC 98752;

(f) a polynucleotide comprising the nucleotide sequence of a mature protein coding sequence of clone dn834_{—}1 deposited under accession number ATCC 98752;

(g) a polynucleotide encoding a mature protein encoded by the cDNA insert of clone dn834_{—}1 deposited under accession number ATCC 98752;

(h) a polynucleotide encoding a protein comprising the amino acid sequence of SEQ ID NO:8;

(i) a polynucleotide encoding a protein comprising a fragment of the amino acid sequence of SEQ ID NO:8 having biological activity, the fragment comprising eight contiguous amino acids of SEQ ID NO:8;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:7.

Preferably, such polynucleotide comprises the nucleotide sequence of SEQ ID NO:7 from nucleotide 20 to nucleotide 484; the nucleotide sequence of SEQ ID NO:7 from nucleotide 18 to nucleotide 892; the nucleotide sequence of the full-length protein coding sequence of clone dn834_{—}1 deposited under accession number ATCC 98752; or the nucleotide sequence of a mature protein coding sequence of clone dn834_{—}1 deposited under accession number ATCC 98752. In other preferred embodiments, the polynucleotide encodes the full-length or a mature protein encoded by the cDNA insert of clone dn834_{—}1 deposited under accession number ATCC 98752. In further preferred embodiments, the present invention provides a polynucleotide encoding a protein comprising a fragment of the amino add sequence of SEQ ID NO:8 having biological activity, the fragment preferably comprising eight (more preferably twenty, most preferably thirty) contiguous amino adds of SEQ ID NO:8, or a polynucleotide encoding a protein comprising a fragment of the amino acid sequence of SEQ ID NO:8 having biological activity, the fragment comprising the ammo acid sequence from amino acid 72 to amino acid 81 of SEQ ID NO:8.

Other embodiments provide the gene corresponding to the cDNA sequence of SEQ ID NO:7.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:7, but excluding the poly(A) tail at the 3′ end of SEQ ID NO:7; and

(ab) the nucleotide sequence of the cDNA insert of clone dn834_{—}1 deposited under accession number ATCC 98752;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:7, but excluding the poly(A) tail at the 3′ end of SEQ ID NO:7; and

(bb) the nucleotide sequence of the cDNA insert of clone dn834_{—}1 deposited under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:7, and extending contiguously from a nucleotide sequence corresponding to the 5′ end of SEQ ID NO:7 to a nucleotide sequence corresponding to the 3′ end of SEQ ID NO:7, but excluding the poly(A) tail at the 3′ end of SEQ ID NO:7. Also preferably the polynucleotide isolated according to the above process comprises a nucleotide sequence corresponding to the cDNA sequence of SEQ ID NO:7 from nucleotide 20 to nucleotide 484, and extending contiguously from a nucleotide sequence corresponding to the 5′ end of said sequence of SEQ ID NO:7 from nucleotide 20 to nucleotide 484, to a nucleotide sequence corresponding to the 3′ end of said sequence of SEQ ID NO:7 from nucleotide 20 to nucleotide 484. Also preferably the polynucleotide isolated according to the above process comprises a nucleotide sequence corresponding to the cDNA sequence of SEQ ID NO:7 from nucleotide 18 to nucleotide 892, and extending contiguously from a nucleotide sequence corresponding to the 5′ end of said sequence of SEQ ID NO:7 from nucleotide 18 to nucleotide 892, to a nucleotide sequence corresponding to the 3′ end of said sequence of SEQ ID NO:7 from nucleotide 18 to nucleotide 892.

In other embodiments, the present invention provides a composition comprising a protein, wherein said protein comprises an amino add sequence selected from the group consisting of

(a) the amino acid sequence of SEQ ID NO:8;

(b) a fragment of the amino acid sequence of SEQ ID NO:8, the fragment comprising eight contiguous amino acids of SEQ ID NO:8; and

(c) the amino acid sequence encoded by the cDNA insert of clone dn834_{—}1 deposited under accession number ATCC 98752;

the protein being substantially free from other mammalian proteins. Preferably such protein comprises the amino acid sequence of SEQ ID NO:8. In further preferred embodiments, the present invention provides a protein comprising a fragment of the amino acid sequence of SEQ ID NO:8 having biological activity, the fragment preferably comprising eight (more preferably twenty, most preferably thirty) contiguous amino acids of SEQ ID NO:8, or a protein comprising a fragment of the amino acid sequence of SEQ ID NO:8 having biological activity, the fragment comprising the amino acid sequence from amino acid 72 to amino acid 81 of SEQ ID NO:8.

In one embodiment, the present invention provides a composition comprising an isolated polynucleotide selected from the group consisting of:

(a) a polynucleotide comprising the nucleotide sequence of SEQ ID NO:9;

(b) a polynucleotide comprising the nucleotide sequence of SEQ ID NO:9 from nucleotide 803 to nucleotide 1420;

(c) a polynucleotide comprising the nucleotide sequence of SEQ ID NO:9 from nucleotide 1022 to nucleotide 1420;

(d) a polynucleotide comprising the nucleotide sequence of the full-length protein coding sequence of clone pd278_{—}5 deposited under accession number ATCC 98752;

(e) a polynucleotide encoding the full-length protein encoded by the cDNA insert of clone pd278_{—}5 deposited under accession number ATCC 98752;

(f) a polynucleotide comprising the nucleotide sequence of a mature protein coding sequence of clone pd2785 deposited under accession number ATCC 98752;

(g) a polynucleotide encoding a mature protein encoded by the cDNA insert of clone pd278_{—}5 deposited under accession number ATCC 98752;

(h) a polynucleotide encoding a protein comprising the amino acid sequence of SEQ ID NO:10;

(i) a polynucleotide encoding a protein comprising a fragment of the amino acid sequence of SEQ ID NO:10 having biological activity, the fragment comprising eight contiguous amino acids of SEQ ID NO:10;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:9.

Preferably, such polynucleotide comprises the nucleotide sequence of SEQ ID NO:9 from nucleotide 803 to nucleotide 1420; the nucleotide sequence of SEQ ID NO:9 from nucleotide 1022 to nucleotide 1420; the nucleotide sequence of the full-length protein coding sequence of clone pd278_{—}5 deposited under accession number ATCC 98752; or the nucleotide sequence of a mature protein coding sequence of clone pd278_{—}5 deposited under accession number ATCC 98752. In other preferred embodiments, the polynucleotide encodes the full-length or a mature protein encoded by the cDNA insert of clone pd278_{—}5 deposited under accession number ATCC 98752. In further preferred embodiments, the present invention provides a polynucleotide encoding a protein comprising a fragment of the amino acid sequence of SEQ ID NO:10 having biological activity, the fragment preferably comprising eight (more preferably twenty, most preferably thirty) contiguous amino adds of SEQ ID NO:10, or a polynucleotide encoding a protein comprising a fragment of the amino acid sequence of SEQ ID NO:10 having biological activity, the fragment comprising the amino acid sequence from amino acid 98 to amino acid 107 of SEQ ID NO:10.

Other embodiments provide the gene corresponding to the cDNA sequence of SEQ ID NO:9.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:9, but excluding the poly(A) tail at the 3′ end of SEQ ID NO:9; and

(ab) the nucleotide sequence of the cDNA insert of clone pd278_{—}5 deposited under accession number ATCC 98752;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:9, but excluding the poly(A) tail at the 3′ end of SEQ ID NO:9; and

(bb) the nucleotide sequence of the cDNA insert of clone pd278_{—}5 deposited-under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:9, and extending contiguously from a nucleotide sequence corresponding to the 5′ end of SEQ ID NO:9 to a nucleotide sequence corresponding to the 3′ end of SEQ ID NO:9, but excluding the poly(A) tail at the 3′ end of SEQ ID NO:9. Also preferably the polynucleotide isolated according to the above process comprises a nucleotide sequence corresponding to the cDNA sequence of SEQ ID NO:9 from nucleotide 803 to nucleotide 1420, and extending contiguously from a nucleotide sequence corresponding to the 5′ end of said sequence of SEQ ID NO:9 from nucleotide 803 to nucleotide 1420, to a nucleotide sequence corresponding to the 3′ end of said sequence of SEQ ID NO:9 from nucleotide 803 to nucleotide 1420. Also preferably the polynucleotide isolated according to the above process comprises a nucleotide sequence corresponding to the cDNA sequence of SEQ ID NO:9 from nucleotide 1022 to nucleotide 1420, and extending contiguously from a nucleotide sequence corresponding to the 5′ end of said sequence of SEQ ID NO:9 from nucleotide 1022 to nucleotide 1420, to a nucleotide sequence corresponding to the 3′ end of said sequence of SEQ ID NO:9 from nucleotide 1022 to nucleotide 1420.

In other embodiments, the present invention provides a composition comprising a protein, wherein said protein comprises an amino add sequence selected from the group consisting of:

(a) the amino acid sequence of SEQ ID NO: 10;

(b) a fragment of the amino add sequence of SEQ ID NO:10, the fragment comprising eight contiguous amino acids of SEQ ID NO:10; and

(c) the amino acid sequence encoded by the cDNA insert of clone pd278_{—}5 deposited under accession number ATCC 98752;

the protein being substantially free from other mammalian proteins. Preferably such protein comprises the amino acid sequence of SEQ ID NO:10. In further preferred embodiments, the present invention provides a protein comprising a fragment of the amino acid sequence of SEQ ID NO:10 having biological activity, the fragment preferably comprising eight (more preferably twenty, most preferably thirty) contiguous amino acids of SEQ ID NO:10, or a protein comprising a fragment of the amino acid sequence of SEQ ID NO:10 having biological activity, the fragment comprising the amino acid sequence from amino acid 98 to amino add 107 of SEQ ID NO:10.

In one embodiment, the present invention provides a composition comprising an isolated polynucleotide selected from the group consisting of

(a) a polynucleotide comprising the nucleotide sequence of SEQ ID NO:11;

(b) a polynucleotide comprising the nucleotide sequence of SEQ ID NO:11 from nucleotide 918 to nucleotide 1295;

(c) a polynucleotide comprising the nucleotide sequence of the full-length protein coding sequence of clone pe80_{—}1 deposited under accession number ATCC 98752;

(d) a polynucleotide encoding the full-length protein encoded by the cDNA insert of clone pe80_{—}1 deposited under accession number ATCC 98752;

(e) a polynucleotide comprising the nucleotide sequence of a mature protein coding sequence of clone pe80_{—}1 deposited under accession number ATCC 98752;

(f) a polynucleotide encoding a mature protein encoded by the cDNA insert of clone pe80_deposited under accession number ATCC 98752;

(g) a polynucleotide encoding a protein comprising the amino acid sequence of SEQ ID NO:12;

(h) a polynucleotide encoding a protein comprising a fragment of the amino acid sequence of SEQ ID NO:12 having biological activity, the fragment comprising eight contiguous amino acids of SEQ ID NO:12;

(i) a polynucleotide which is an allelic variant of a polynucleotide of (a)-f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:11.

Preferably, such polynucleotide comprises the nucleotide sequence of SEQ ID NO:11 from nucleotide 918 to nucleotide 1295; the nucleotide sequence of the full-length protein coding sequence of clone pe80_{—}1 deposited under accession number ATCC 98752; or the nucleotide sequence of a mature protein coding sequence of clone pe80_{—}1 deposited under accession number ATCC 98752. In other preferred embodiments, the polynucleotide encodes the full-length or a mature protein encoded by the cDNA insert of clone pe80_{—}1 deposited under accession number ATCC 98752. In further preferred embodiments, the present invention provides a polynucleotide encoding a proteins comprising a fragment of the amino add sequence of SEQ ID NO:12 having biological activity, the fragment preferably comprising eight (more preferably twenty, most preferably thirty) contiguous amino acids of SEQ ID NO:12, or a polynucleotide encoding a protein comprising a fragment of the amino acid sequence of SEQ ID NO:12 having biological activity, the fragment comprising the amino add sequence from amino acid 58 to amino acid 67 of SEQ ID NO:12.

Other embodiments provide the gene corresponding to the cDNA sequence of SEQ ID NO:11.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:11, but excluding the poly(A) tail at the 3′ end of SEQ ID NO:11; and

(ab) the nucleotide sequence of the cDNA insert of clone pe80_{—}1 deposited under accession number ATCC 98752;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:11, but excluding the poly(A) tail at the 3′ end of SEQ ID NO:11; and

(bb) the nucleotide sequence of the cDNA insert of clone pe80_{—}1 deposited under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:11, and extending contiguously from a nucleotide sequence corresponding to the 5′ end of SEQ ID NO-11 to a nucleotide sequence corresponding to the 3′ end of SEQ ID NO:11, but excluding the poly(A) tail at the 3′ end of SEQ ID NO:11. Also preferably the polynucleotide isolated according to the above process comprises a nucleotide sequence corresponding to the cDNA sequence of SEQ ID NO:11 from nucleotide 918 to nucleotide 1295, and extending contiguously from a nucleotide sequence corresponding to the 5′ end of said sequence of SEQ ID NO:11 from nucleotide 918 to nucleotide 1295, to a nucleotide sequence corresponding to the 3′ end of said sequence of SEQ ID NO:11 from nucleotide 918 to nucleotide 1295.

In other embodiments, the present invention provides a composition comprising a protein, wherein said protein comprises an amino acid sequence selected from the group consisting of:

(a) the amino acid sequence of SEQ ID NO:12;

(b) a fragment of the amino add sequence of SEQ ID NO:12, the fragment comprising eight contiguous amino adds of SEQ ID NO:12; and

(c) the amino acid sequence encoded by the cDNA insert of clone pe80_{—}1 deposited under accession number ATCC 98752;

the protein being substantially free from other mammalian proteins. Preferably such protein comprises the amino add sequence of SEQ ID NO:12. In further preferred embodiments, the present invention provides a protein comprising a fragment of the amino acid sequence of SEQ ID NO:12 having biological activity, the fragment preferably comprising eight (more preferably twenty, most preferably thirty) contiguous amino acids of SEQ ID NO:12, or a protein comprising a fragment of the amino acid sequence of SEQ ID NO:12 having biological activity, the fragment comprising the amino acid sequence from amino acid 58 to amino add 67 of SEQ ID NO:12.

In one embodiment, the present invention provides a composition comprising an isolated polynucleotide selected from the group consisting of:

(a) a polynucleotide comprising the nucleotide sequence of SEQ ID NO:13;

(b) a polynucleotide comprising the nucleotide sequence of SEQ ID NO:13 from nucleotide 189 to nucleotide 428;

(c) a polynucleotide comprising the nucleotide sequence of SEQ ID NO:13 from nucleotide 348 to nucleotide 428;

(d) a polynucleotide comprising the nucleotide sequence of the full-length protein coding sequence of clone pm113_{—}1 deposited under accession number ATCC 98752;

(e) a polynucleotide encoding the full-length protein encoded by the cDNA insert of clone pm113_{—}1 deposited under accession number ATCC 98752;

(f) a polynucleotide comprising the nucleotide sequence of a mature protein coding sequence of clone pm113 _{—}1 deposited under accession number ATCC 98752;

(g) a polynucleotide encoding a mature protein encoded by the cDNA insert of clone pm113_{—}1 deposited under accession number ATCC 98752;

(h) a polynucleotide encoding a protein comprising the amino acid sequence of SEQ ID NO:14;

(i) a polynucleotide encoding a protein comprising a fragment of the amino add sequence of SEQ ID NO:14 having biological activity, the fragment comprising eight contiguous amino acids of SEQ ID NO:14;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:13.

Preferably, such polynucleotide comprises the nucleotide sequence of SEQ ID NO:13 from nucleotide 189 to nucleotide 428; the nucleotide sequence of SEQ ID NO:13 from nucleotide 348 to nucleotide 428; the nucleotide sequence of the full-length protein coding sequence of clone pm113_{—}1 deposited under accession number ATCC 98752; or the nucleotide sequence of a mature protein coding sequence of clone pm113_{—}1 deposited under accession number ATCC 98752. In other preferred embodiments, the polynucleotide encodes the full-length or a mature protein encoded by the cDNA insert of clone pm113_{—}1 deposited under accession number ATCC 98752. In further preferred embodiments, the present invention provides a polynucleotide encoding a protein comprising a fragment of the amino add sequence of SEQ ID NO:14 having biological activity, the fragment preferably comprising eight (more preferably twenty, most preferably thirty) contiguous amino adds of SEQ ID NO:14, or a polynucleotide encoding a protein comprising a fragment of the amino add sequence of SEQ ID NO:14 having biological activity, the fragment comprising the amino acid sequence from amino acid 35 to amino acid 44 of SEQ ID NO:14.

Other embodiments provide the gene corresponding to the cDNA sequence of SEQ ID NO:13.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:13, but excluding the poly(A) tail at the 3′ end of SEQ ID NO:13; and

(ab) the nucleotide sequence of the cDNA insert of clone pm1131 deposited under accession number ATCC 98752;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:13, but excluding the poly(A) tail at the 3′ end of SEQ ID NO:13; and

(bb) the nucleotide sequence of the cDNA insert of clone pm113 μl deposited under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:13, and extending contiguously from a nucleotide sequence corresponding to the 5′ end of SEQ ID NO:13 to a nucleotide sequence corresponding to the 3′ end of SEQ ID NO:13, but excluding the poly(A) tail at the 3′ end of SEQ ID NO:13. Also preferably the polynucleotide isolated according to the above process comprises a nucleotide sequence corresponding to the cDNA sequence of SEQ ID NO:13 from nucleotide 189 to nucleotide 428, and extending contiguously from a nucleotide sequence corresponding to the 5′ end of said sequence of SEQ ID NO:13 from nucleotide 189 to nucleotide 428, to a nucleotide sequence corresponding to the 3′ end of said sequence of SEQ ID NO:13 from nucleotide 189 to nucleotide 428. Also preferably the polynucleotide isolated according to the above process comprises a nucleotide sequence corresponding to the cDNA sequence of SEQ ID NO:13 from nucleotide 348 to nucleotide 428, and extending contiguously from a nucleotide sequence corresponding to the 5′ end of said sequence of SEQ ID NO:13 from nucleotide 348 to nucleotide 428, to a nucleotide sequence corresponding to the 3′ end of said sequence of SEQ ID NO:13 from nucleotide 348 to nucleotide 428.

In other embodiments, the present invention provides a composition comprising a protein, wherein said protein comprises an amino acid sequence selected from the group consisting of:

(a) the amino acid sequence of SEQ ID NO:14;

(b) a fragment of the amino add sequence of SEQ ID NO:14, the fragment comprising eight contiguous amino acids of SEQ ID NO:14; and

(c) the amino acid sequence encoded by the cDNA insert of clone pm113_{—}1 deposited under accession number ATCC 98752;

the protein being substantially free from other mammalian proteins. Preferably such protein comprises the amino add sequence of SEQ ID NO:14. In further preferred embodiments, the present invention provides a protein comprising a fragment of the amino acid sequence of SEQ ID NO:14 having biological activity, the fragment preferably comprising eight (more preferably twenty, most preferably thirty) contiguous amino adds of SEQ ID NO:14, or a protein comprising a fragment of the amino add sequence of SEQ ID NO:14 having biological activity, the fragment comprising the amino acid sequence from amino acid 35 to amino acid 44 of SEQ ID NO: 14.

In one embodiment, the present invention provides a composition comprising an isolated polynucleotide selected from the group consisting of:

(a) a polynucleotide comprising the nucleotide sequence of SEQ ID NO:15;

(b) a polynucleotide comprising the nucleotide sequence of SEQ ID NO:15 from nucleotide 108 to nucleotide 1496;

(c) a polynucleotide comprising the nucleotide sequence of the full-length protein coding sequence of clone pm749_{—}8 deposited under accession number ATCC 98752;

(d) a polynucleotide encoding the full-length protein encoded by the cDNA insert of clone pm749_{—}8 deposited under accession number ATCC98752;

(e) a polynucleotide comprising the nucleotide sequence of a mature protein coding sequence of clone pm749_{—}8 deposited under accession number ATCC98752;

(f) a polynucleotide encoding a mature protein encoded by the cDNA insert of clone pm749_{—}8 deposited under accession number ATCC 98752;

(g) a polynucleotide encoding a protein comprising the amino add sequence of SEQ ID NO:16;

(h) a polynucleotide encoding a protein comprising a fragment of the amino acid sequence of SEQ ID NO:16 having biological activity, the fragment comprising eight contiguous amino acids of SEQ ID NO:16;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:15.

Preferably, such polynucleotide comprises the nucleotide sequence of SEQ ID NO:15 from nucleotide 108 to nucleotide 1496; the nucleotide sequence of the full-length protein coding sequence of clone pm749_{—}8 deposited under accession number ATCC 98752; or the nucleotide sequence of a mature protein coding sequence of clone pm749_{—}8 deposited under accession number ATCC 98752. In other preferred embodiments, the polynucleotide encodes the full-length or a mature protein encoded by the cDNA insert of clone pm749_{—}8 deposited under accession number ATCC 98752. In further preferred embodiments, the present invention provides a polynucleotide encoding a protein comprising a fragment of the amino add sequence of SEQ ID NO:16 having biological activity, the fragment preferably comprising eight (more preferably twenty, most preferably thirty) contiguous amino acids of SEQ ID NO:16, or a polynucleotide encoding a protein comprising a fragment of the amino add sequence of SEQ ID NO:16 having biological activity, the fragment comprising the amino add sequence from amino acid 226 to amino add 235 of SEQ ID NO:16.

Other embodiments provide the gene corresponding to the cDNA sequence of SEQ ID NO:15.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:15, but excluding the poly(A) tail at the 3′ end of SEQ ID NO:15; and

(ab) the nucleotide sequence of the cDNA insert of clone pm749_{—}8 deposited under accession number ATCC 98752;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:15, but excluding the poly(A) tail at the 3′ end of SEQ ID NO:15; and

(bb) the nucleotide sequence of the cDNA insert of clone pm749_{—}8 deposited under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(ii).

Preferably the polynucleotide isolated according to the above process comprises a nucleotide sequence corresponding to the cDNA sequence of SEQ ID NO:15, and extending contiguously from a nucleotide sequence corresponding to the 5′ end of SEQ ID NO:15 to a nucleotide sequence corresponding to the 3′ end of SEQ ID NO:15, but excluding the poly(A) tail at the 3′ end of SEQ ID NO:15. Also preferably the polynucleotide isolated according to the above process comprises a nucleotide sequence corresponding to the cDNA sequence of SEQ ID NO:15 from nucleotide 108 to nucleotide 1496, and extending contiguously from a nucleotide sequence corresponding to the 5′ end of said sequence of SEQ ID NO: 15 from nucleotide 108 to nucleotide 1496, to a nucleotide sequence corresponding to the 3′ end of said sequence of SEQ ID NO:15 from nucleotide 108 to nucleotide 1496.

In other embodiments, the present invention provides a composition comprising a protein, wherein said protein comprises an ammo acid sequence selected from the group consisting of:

(a) the amino acid sequence of SEQ ID NO:16;

(b) a fragment of the amino acid sequence of SEQ ID NO:16, the fragment comprising eight contiguous amino acids of SEQ ID NO: 16; and

(c) the amino add sequence encoded by the cDNA insert of clone pm749_{—}8 deposited under accession number ATCC 98752;

the protein being substantially free from other mammalian proteins. Preferably such protein comprises the amino add sequence of SEQ ID NO:16. In further preferred embodiments, the present invention provides a protein comprising a fragment of the amino acid sequence of SEQ ID NO:16 having biological activity, the fragment preferably comprising eight (more preferably twenty, most preferably thirty) contiguous amino acids of SEQ ID NO:16, or a protein comprising a fragment of the amino add sequence of SEQ ID NO:16 having biological activity, the fragment comprising the amino acid sequence from amino acid 226 to amino add 235 of SEQ ID NO:16.

In one embodiment, the present invention provides a composition comprising an isolated polynucleotide selected from the group consisting of:

(a) a polynucleotide comprising the nucleotide sequence of SEQ ID NO:17;

(b) a polynucleotide comprising the nucleotide sequence of SEQ ID NO:17 from nucleotide 44 to nucleotide 2023;

(c) a polynucleotide comprising the nucleotide sequence of SEQ ID NO:17 from nucleotide 137 to nucleotide 2023;

(d) a polynucleotide comprising the nucleotide sequence of the full-length protein coding sequence of clone pt31_{—}4 deposited under accession number ATCC 98752;

(e) a polynucleotide encoding the full-length protein encoded by the cDNA insert of clone pt31_{—}4 deposited under accession number ATCC 98752;

(f) a polynucleotide comprising the nucleotide sequence of a mature protein coding sequence of clone pt31_{—}4 deposited under accession number ATCC 98752;

(g) a polynucleotide encoding a mature protein encoded by the cDNA insert of clone pt31_{—}4 deposited under accession number ATCC 98752;

(h) a polynucleotide encoding a protein comprising the amino acid sequence of SEQ ID NO:0.18;

(i) a polynucleotide encoding a protein comprising a fragment of the amino acid sequence of SEQ ID NO:18 having biological activity, the fragment comprising eight contiguous amino acids of SEQ ID NO:18;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:17.

Preferably, such polynucleotide comprises the nucleotide sequence of SEQ ID NO:17 from nucleotide 44 to nucleotide 2023; the nucleotide sequence of SEQ ID NO:17 from nucleotide 137 to nucleotide 2023; the nucleotide sequence of the full-length protein coding sequence of clone pt31_A deposited: under accession number ATCC 98752; or the nucleotide sequence of a mature protein coding sequence of clone pt31_{—}4 deposited under accession number ATCC 98752. In other preferred embodiments, the polynucleotide encodes the full-length or a mature protein encoded by the cDNA insert of clone pt31_{—}4 deposited under accession number ATCC 98752. In further preferred embodiments, the present invention provides a polynucleotide encoding a protein comprising a fragment of the amino acid sequence of SEQ ID NO:18 having biological activity, the fragment preferably comprising eight (more preferably twenty, most preferably thirty) contiguous amino acids of SEQ ID NO:18, or a polynucleotide encoding a protein comprising a fragment of the amino acid sequence of SEQ ID NO:18 having biological activity, the fragment comprising the amino acid sequence from amino acid 325 to amino acid 334 of SEQ ID NO:18.

Other embodiments provide the gene corresponding to the cDNA sequence of SEQ ID NO:17.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:17, but excluding the poly(A) tail at the 3′ end of SEQ ID NO:17; and

(ab) the nucleotide sequence of the cDNA insert of clone pt31_{—}4 deposited under accession number ATCC 98752;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:17, but excluding the poly(A) tail at the 3′ end of SEQ ID NO:17; and

(bb) the nucleotide sequence of the cDNA insert of clone pt31_{—}4 deposited under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:17, and extending contiguously from a nucleotide sequence corresponding to the 5′ end of SEQ ID NO:17 to a nucleotide sequence corresponding to the 3′ end of SEQ ID NO:17, but excluding the poly(A) tail at the 3′ end of SEQ ID NO:17. Also preferably the polynucleotide isolated according to the above process comprises a nucleotide sequence corresponding to the cDNA sequence of SEQ ID NO:17 from nucleotide 44 to nucleotide 2023, and extending contiguously from a nucleotide sequence corresponding to the 5′ end of said sequence of SEQ ID NO:17 from nucleotide 44 to nucleotide 2023, to a nucleotide sequence corresponding to the 3′ end of said sequence of SEQ ID NO:17 from nucleotide 44 to nucleotide 2023. Also preferably the polynucleotide isolated according to the above process comprises a nucleotide sequence corresponding to the cDNA sequence of SEQ ID NO:17 from nucleotide 137 to nucleotide 2023, and extending contiguously from a nucleotide sequence corresponding to the 5′ end of said sequence of SEQ ID NO:17 from nucleotide 137 to nucleotide 2023, to a nucleotide sequence corresponding to the 3′ end of said sequence of SEQ ID NO:17 from nucleotide 137 to nucleotide 2023.

In other embodiments, the present invention provides a composition comprising a protein, wherein said protein comprises an amino acid sequence selected from the group consisting of:

(a) the amino acid sequence of SEQ ID NO: 18;

(b) a fragment of the amino acid sequence of SEQ ID NO:18, the fragment comprising eight contiguous amino adds of SEQ ID NO:18; and

(c) the amino acid sequence encoded by the cDNA insert of clone pt31_{—}4 deposited under accession number ATCC 98752;

the protein being substantially free from other mammalian proteins. Preferably such protein comprises the amino acid sequence of SEQ ID NO:18. In further preferred embodiments, the present invention provides a protein comprising a fragment of the amino acid sequence of SEQ ID NO:18 having biological activity, the fragment preferably comprising eight (more preferably twenty, most preferably thirty) contiguous amino adds of SEQ ID NO:18, or a protein comprising a fragment of the amino acid sequence of SEQ ID NO:18 having biological activity, the fragment comprising the amino acid sequence from amino add 325 to amino add 334 of SEQ ID NO:18.

In one embodiment, the present invention provides a composition comprising an isolated polynucleotide selected from the group consisting of:

(a) a polynucleotide comprising the nucleotide sequence of SEQ ID NO:19;

(b) a polynucleotide comprising the nucleotide sequence of SEQ ID NO:19 from nucleotide 24 to nucleotide 299;

(c) a polynucleotide comprising the nucleotide sequence of the full-length protein coding sequence of clone pv296_{—}5 deposited under accession number ATCC 98752;

(d) a polynucleotide encoding the full-length protein encoded by the cDNA insert of clone pv296_{—}5 deposited under accession number ATCC 98752;

(e) a polynucleotide comprising the nucleotide sequence of a mature protein coding sequence of clone pv296_{—}5 deposited under accession number ATCC 98752;

(f) a polynucleotide encoding a mature protein encoded by the cDNA insert of clone pv296_{—}5 deposited under accession number ATCC 98752;

(g) a polynucleotide encoding a protein comprising the amino acid sequence of SEQ ID NO:20;

(h) a polynucleotide encoding a protein comprising a fragment of the amino acid sequence of SEQ ID NO:20 having biological activity, the fragment comprising eight contiguous amino acids of SEQ ID NO:20;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:19.

Preferably, such polynucleotide comprises the nucleotide sequence of SEQ ID NO:19 from nucleotide 24 to nucleotide 299; the nucleotide sequence of the full-length protein coding sequence of clone pv296_{—}5 deposited under accession number ATCC 98752; or the nucleotide sequence of a mature protein coding sequence of clone pv296_{—}5

In one embodiment, the present invention provides a composition comprising an isolated polynucleotide selected from the group consisting of:

(a) a polynucleotide comprising the nucleotide sequence of SEQ ID NO:19;

(b) a polynucleotide comprising the nucleotide sequence of SEQ ID NO:19 from nucleotide 24 to nucleotide 299;

(c) a polynucleotide comprising the nucleotide sequence of the full-length protein coding sequence of clone pv296_{—}5 deposited under accession number ATCC 98752;

(d) a polynucleotide encoding the full-length protein encoded by the cDNA insert of clone pv296_{—}5 deposited under accession number ATCC 98752;

(e) a polynucleotide comprising the nucleotide sequence of a mature protein coding sequence of clone pv296_{—}5 deposited under accession number ATCC 98752;

(f) a polynucleotide encoding a mature protein encoded by the cDNA insert of clone pv296_{—}5 deposited under accession number ATCC 98752;

(g) a polynucleotide encoding a protein comprising the amino acid sequence of SEQ ID NO:20;

(h) a polynucleotide encoding a protein comprising a fragment of the amino acid sequence of SEQ ID NO:20 having biological activity, the fragment comprising eight contiguous amino acids of SEQ ID NO:20;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:19.

Preferably, such polynucleotide comprises the nucleotide sequence of SEQ ID NO:19 from nucleotide 24 to nucleotide 299; the nucleotide sequence of the full-length protein coding sequence of clone pv296_{—}5 deposited under accession number ATCC 98752; or the nucleotide sequence of a mature protein coding sequence of clone pV296_{—}5 deposited under accession number ATCC 98752. In other preferred embodiments, the polynucleotide encodes the full-length or a mature protein encoded by the cDNA insert of clone pv296_{—}5 deposited under accession number ATCC 98752. In further preferred embodiments, the present invention provides a polynucleotide encoding a protein comprising a fragment of the amino acid sequence of SEQ ID NO:20 having biological activity, the fragment preferably comprising eight (more preferably twenty, most preferably thirty) contiguous amino acids of SEQ ID NO:20, or a polynucleotide encoding a protein comprising a fragment of the amino acid sequence of SEQ ID NO:20 having biological activity, the fragment comprising the amino acid sequence from amino acid 41 to amino acid 50 of SEQ ID NO:20.

Other embodiments provide the gene corresponding to the cDNA sequence of SEQ ID NO:19.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:19, but excluding the poly(A) tail at the 3′ end of SEQ ID NO:19; and

(ab) the nucleotide sequence of the cDNA insert of clone pv296_{—}5 deposited under accession number ATCC 98752;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:19, but excluding the poly(A) tail at the 3′ end of SEQ ID NO:19; and

(bb) the nucleotide sequence of the cDNA insert of clone pv296_{—}5 deposited under accession number ATCC 98752;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:19, and extending contiguously from a nucleotide sequence corresponding to the 5′ end of SEQ ID NO:19 to a nucleotide sequence corresponding to the 3′ end of SEQ ID NO:19, but excluding the poly(A) tail at the 3′ end of SEQ ID NO:19. Also preferably the polynucleotide isolated according to the above process comprises a nucleotide sequence corresponding to the cDNA sequence of SEQ ID NO:19 from nucleotide 24 to nucleotide 299, and extending contiguously from a nucleotide sequence corresponding to the 5′ end of said sequence of SEQ ID NO:19 from nucleotide 24 to nucleotide 299, to a nucleotide sequence corresponding to the 3′ end of said sequence of SEQ ID NO:19 from nucleotide 24 to nucleotide 299.

In other embodiments, the present invention provides a composition comprising a protein, wherein said protein comprises an amino acid sequence selected from the group consisting of:

(a) the amino acid sequence of SEQ ID NO:20;

(b) a fragment of the amino acid sequence of SEQ ID NO:20, the fragment comprising eight contiguous amino acids of SEQ ID NO:20; and

(c) the amino acid sequence encoded by the cDNA insert of clone pv296_{—}5 deposited under accession number ATCC 98752;

the protein being substantially free from other mammalian proteins. Preferably such protein comprises the amino acid sequence of SEQ ID NO:20. In further preferred embodiments, the present invention provides a protein comprising a fragment of the amino add sequence of SEQ ID NO:20 having biological activity, the fragment preferably comprising eight (more preferably twenty, most preferably thirty) contiguous amino acids of SEQ ID NO:20, or a protein comprising a fragment of the amino acid sequence of SEQ ID NO:20 having biological activity, the fragment comprising the amino acid sequence from amino acid 41 to amino acid 50 of SEQ ID NO:20.

In one embodiment, the present invention provides a composition comprising an isolated polynucleotide selected from the group consisting of:

(a) a polynucleotide comprising the nucleotide sequence of SEQ ID NO:21;

(b) a polynucleotide comprising the nucleotide sequence of SEQ ID NO:21 from nucleotide 8 to nucleotide 2008;

(c) a polynucleotide comprising the nucleotide sequence of the full-length protein coding sequence of clone er311_{—}20 deposited under accession number ATCC 98781;

(d) a polynucleotide encoding the full-length protein encoded by the cDNA insert of clone er311_{—}20 deposited under accession number ATCC 98781;

(e) a polynucleotide comprising the nucleotide sequence of a mature protein coding sequence of clone er3111_{—}20 deposited under accession number ATCC 98781;

(f) a polynucleotide encoding a mature protein encoded by the cDNA insert of clone er311120 deposited under accession number ATCC 98781;

(g) a polynucleotide encoding a protein comprising the amino acid sequence of SEQ ID NO:22;

(h) a polynucleotide encoding a protein comprising a fragment of the amino acid sequence of SEQ ID NO:22 having biological activity, the fragment comprising eight contiguous amino acids of SEQ ID NO:22;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:21.

Preferably, such polynucleotide comprises the nucleotide sequence of SEQ ID NO:21 from nucleotide 8 to nucleotide 2008; the nucleotide sequence of the full-length protein coding sequence of clone er311_{—}20 deposited under accession number ATCC 98781; or the nucleotide sequence of a mature protein coding sequence of clone er311_{—}20 deposited under accession number ATCC 98781. In other preferred embodiments, the polynucleotide encodes the full-length or a mature protein encoded by the cDNA insert of clone er311_{—}20 deposited under accession number ATCC 98781. In further preferred embodiments, the present invention provides a polynucleotide encoding a protein comprising a fragment of the amino acid sequence of SEQ ID NO:22 having biological activity, the fragment preferably comprising eight (more preferably twenty, most preferably thirty) contiguous amino acids of SEQ ID NO:22, or a polynucleotide encoding a protein comprising a fragment of the amino acid sequence of SEQ ID NO:22 having biological activity, the fragment comprising the amino acid sequence from amino acid 328 to amino acid 337 of SEQ ID NO:22.

Other embodiments provide the gene corresponding to the cDNA sequence of SEQ ID NO:21.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:21, but excluding the poly(A) tail at the 3′ end of SEQ ID NO:21; and

(ab) the nucleotide sequence of the cDNA insert of clone er311120 deposited under accession number ATCC 98781;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:21, but excluding the poly(A) tail at the 3′ end of SEQ ID NO:21; and

(bb) the nucleotide sequence of the cDNA insert of clone er311_{—}20 deposited under accession number ATCC 98781;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:21, and extending contiguously from a nucleotide sequence corresponding to the 5′ end of SEQ ID NO:21 to a nucleotide sequence corresponding to the 3′ end of SEQ ID NO:21, but excluding the poly(A) tail at the 3′ end of SEQ ID NO:21. Also preferably the polynucleotide isolated according to the above process comprises a nucleotide sequence corresponding to the cDNA sequence of SEQ ID NO:21 from nucleotide 8 to nucleotide 2008, and extending contiguously from a nucleotide sequence corresponding to the 5′ end of said sequence of SEQ ID NO:21 from nucleotide 8 to nucleotide 2008, to a nucleotide sequence corresponding to the 3′ end of said sequence of SEQ ID NO:21 from nucleotide 8 to nucleotide 2008.

In other embodiments, the present invention provides a composition comprising a protein, wherein said protein comprises an amino acid sequence selected from the group consisting of:

(a) the amino acid sequence of SEQ ID NO:22;

(b) a fragment of the amino acid sequence of SEQ ID NO:22, the fragment comprising eight contiguous amino acids of SEQ ID NO:22; and

(c) the amino acid sequence encoded by the cDNA insert of clone er311_{—}20 deposited under accession number ATCC 98781;

the protein being substantially free from other mammalian proteins. Preferably such protein comprises the amino acid sequence of SEQ ID NO:22. In further preferred embodiments, the present invention provides a protein comprising a fragment of the amino add sequence of SEQ ID NO:22 having biological activity, the fragment preferably comprising eight (more preferably twenty, most preferably thirty) contiguous amino acids of SEQ ID NO:22, or a protein comprising a fragment of the amino acid sequence of SEQ ID NO:22 having biological activity, the fragment comprising the amino acid sequence from amino acid 328 to amino acid 337 of SEQ ID NO:22.

In one embodiment, the present invention provides a composition comprising an isolated polynucleotide selected from the group consisting of:

(a) a polynucleotide comprising the nucleotide sequence of SEQ ID NO:23;

(b) a polynucleotide comprising the nucleotide sequence of SEQ ID NO:23 from nucleotide 484 to nucleotide 2043;

(c) a polynucleotide comprising the nucleotide sequence of SEQ ID NO:23 from nucleotide 919 to nucleotide 2043;

(d) a polynucleotide comprising the nucleotide sequence of the full-length protein coding sequence of clone fh149_{—}12 deposited under accession number ATCC 98781;

(e) a polynucleotide encoding the full-length protein encoded by the cDNA insert of clone fh149_{—}12 deposited under accession number ATCC 98781;

(f) a polynucleotide comprising the nucleotide sequence of a mature protein coding sequence of clone fh149_{—}12 deposited under accession number ATCC 98781;

(g) a polynucleotide encoding a mature protein encoded by the cDNA insert of clone fh149_{—}12 deposited under accession number ATCC 98781;

(h) a polynucleotide encoding a protein comprising the amino acid sequence of SEQ ID NO:24;

(i) a polynucleotide encoding a protein comprising a fragment of the amino acid sequence of SEQ ID NO:24 having biological activity, the fragment comprising eight contiguous amino acids of SEQ ID NO:24;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:23.

Preferably, such polynucleotide comprises the nucleotide sequence of SEQ ID NO:23 from nucleotide 484 to nucleotide 2043; the nucleotide sequence of SEQ ID NO:23 from nucleotide 919 to nucleotide 2043; the nucleotide sequence of the full-length protein coding sequence of clone fh149_{—}12 deposited under accession number ATCC 98781; or the nucleotide sequence of a mature protein coding sequence of clone fh149_{—}12 deposited under accession number ATCC 98781. In other preferred embodiments, the polynucleotide encodes the full-length or a mature protein encoded by the cDNA insert of clone fh149_{—}12 deposited under accession number ATCC 98781. In further preferred embodiments, the present invention provides a polynucleotide encoding a protein comprising a fragment of the amino acid sequence of SEQ ID NO:24 having biological activity, the fragment preferably comprising eight (more preferably twenty, most preferably thirty) contiguous amino acids of SEQ ID NO:24, or a polynucleotide encoding a protein comprising a fragment of the amino acid sequence of SEQ ID NO:24 having biological activity, the fragment comprising the amino acid sequence from amino acid 255 to amino acid 264 of SEQ ID NO:24.

Other embodiments provide the gene corresponding to the cDNA sequence of SEQ ID NO:23.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:23, but excluding the poly(A) tail at the 3′ end of SEQ ID NO:23; and

(ab) the nucleotide sequence of the cDNA insert of clone fh149_{—}12 deposited under accession number ATCC 98781;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO-23, but excluding the poly(A) tail at the 3′ end of SEQ ID NO:23; and

(bb) the nucleotide sequence of the cDNA insert of clone fh149_{—}12 deposited under accession number ATCC 98781;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:23, and extending contiguously from a nucleotide sequence corresponding to the 5′ end of SEQ ID NO:23 to a nucleotide sequence corresponding to the 3′ end of SEQ ID NO:23, but excluding the poly(A) tail at the 3′ end of SEQ ID NO:23. Also preferably the polynucleotide isolated according to the above process comprises a nucleotide sequence corresponding to the cDNA sequence of SEQ ID NO:23 from nucleotide 484 to nucleotide 2043, and extending contiguously from a nucleotide sequence corresponding to the 5′ end of said sequence of SEQ ID NO:23 from nucleotide 484 to nucleotide 2043, to a nucleotide sequence corresponding to the 3′ end of said sequence of SEQ ID NO:23 from nucleotide 484 to nucleotide 2043. Also preferably the polynucleotide isolated according to the above process comprises a nucleotide sequence corresponding to the cDNA sequence of SEQ ID NO:23 from nucleotide 919 to nucleotide 2043, and extending contiguously from a nucleotide sequence corresponding to the 5′ end of said sequence of SEQ ID NO:23 from nucleotide 919 to nucleotide 2043, to a nucleotide sequence corresponding to the 3′ end of said sequence of SEQ ID NO:23 from nucleotide 919 to nucleotide 2043.

In other embodiments, the present invention provides a composition comprising a protein, wherein said protein comprises an amino acid sequence selected from the group consisting of:

(a) the amino acid sequence of SEQ ID NO:24;

(b) a fragment of the amino acid sequence of SEQ ID NO:24, the fragment comprising eight contiguous amino adds of SEQ ID NO:24; and

(c) the amino acid sequence encoded by the cDNA insert of clone fh149_{—}12 deposited under accession number ATCC 98781;

the protein being substantially free from other mammalian proteins. Preferably such protein comprises the amino add sequence of SEQ ID NO:24. In further preferred embodiments, the present invention provides a protein comprising a fragment of the amino acid sequence of SEQ ID NO:24 having biological activity, the fragment preferably comprising eight (more preferably twenty, most preferably thirty) contiguous amino acids of SEQ ID NO:24, or a protein comprising a fragment of the amino acid sequence of SEQ ID NO:24 having biological activity, the fragment comprising the amino acid sequence from amino add 255 to amino acid 264 of SEQ ID NO:24.

In one embodiment, the present invention provides a composition comprising an isolated polynucleotide selected from the group consisting of:

(a) a polynucleotide comprising the nucleotide sequence of SEQ ID NO:25;

(b) a polynucleotide comprising the nucleotide sequence of SEQ ID NO:25 from nucleotide 47 to nucleotide 1099;

(c) a polynucleotide comprising the nucleotide sequence of SEQ ID NO:25 from nucleotide 143 to nucleotide 1099;

(d) a polynucleotide comprising the nucleotide sequence of the full-length protein coding sequence of clone pc2016 deposited under accession number ATCC 98781;

(e) a polynucleotide encoding the full-length protein encoded by the cDNA insert of clone pc201_{—}6 deposited under accession number ATCC 98781;

(f) a polynucleotide comprising the nucleotide sequence of a mature protein coding sequence of clone pc201_{—}6 deposited under accession number ATCC 98781;

(g) a polynucleotide encoding a mature protein encoded by the cDNA insert of clone pc201_{—}6 deposited under accession number ATCC 98781;

(h) a polynucleotide encoding a protein comprising the amino acid sequence of SEQ ID NO:26;

(i) a polynucleotide encoding a protein comprising a fragment of the amino acid sequence of SEQ ID NO:26 having biological activity, the fragment comprising eight contiguous amino acids of SEQ ID NO:26;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:25.

Preferably, such polynucleotide comprises the nucleotide sequence of SEQ ID NO:25 from nucleotide 47 to nucleotide 1099; the nucleotide sequence of SEQ ID NO:25 from nucleotide 143 to nucleotide 1099; the nucleotide sequence of the full-length protein coding sequence of clone pc201_{—}6 deposited under accession number ATCC 98781; or the nucleotide sequence of a mature protein coding sequence of clone pc2016 deposited under accession number ATCC 98781. In other preferred embodiments, the polynucleotide encodes the full-length or a mature protein encoded by the cDNA insert of clone pc201_{—}6 deposited under accession number ATCC 98781. In further preferred embodiments, the present invention provides a polynucleotide encoding a protein comprising a fragment of the amino acid sequence of SEQ ID NO:26 having biological activity, the fragment preferably comprising eight (more preferably twenty, most preferably thirty) contiguous amino acids of SEQ ID NO:26, or a polynucleotide encoding a protein comprising a fragment of the amino acid sequence of SEQ ID NO:26 having biological activity, the fragment comprising the amino acid sequence from amino acid 170 to amino acid 179 of SEQ ID NO:26.

Other embodiments provide the gene corresponding to the cDNA sequence of SEQ ID NO:25.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:25, but excluding the poly(A) tail at the 3′ end of SEQ ID NO:25; and

(ab) the nucleotide sequence of the cDNA insert of clone pc201_deposited under accession number ATCC 98781;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:25, but excluding the poly(A) tail at the 3′ end of SEQ ID NO:25; and

(bb) the nucleotide sequence of the cDNA insert of clone pc201_{—}6 deposited under accession number ATCC 98781;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:25, and extending contiguously from a nucleotide sequence corresponding to the 5′ end of SEQ ID NO:25 to a nucleotide sequence corresponding to the 3′ end of SEQ ID NO:25, but excluding the poly(A) tail at the 3′ end of SEQ ID NO:25. Also preferably the polynucleotide isolated according to the above process comprises a nucleotide sequence corresponding to the cDNA sequence of SEQ ID NO:25 from nucleotide 47 to nucleotide 1099, and extending contiguously from a nucleotide sequence corresponding to the 5′ end of said sequence of SEQ ID NO:25 from nucleotide 47 to nucleotide 1099, to a nucleotide sequence corresponding to the 3′ end of said sequence of SEQ ID NO:25 from nucleotide 47 to nucleotide 1099. Also preferably the polynucleotide isolated according to the above process comprises a nucleotide sequence corresponding to the cDNA sequence of SEQ ID NO:25 from nucleotide 143 to nucleotide 1099, and extending contiguously from a nucleotide sequence corresponding to the 5′ end of said sequence of SEQ ID NO:25 from nucleotide 143 to nucleotide 1099, to a nucleotide sequence corresponding to the 3′ end of said sequence of SEQ ID NO:25 from nucleotide 143 to nucleotide 1099.

In other embodiments, the present invention provides a composition comprising a protein, wherein said protein comprises an amino acid sequence selected from the group consisting of:

(a) the amino acid sequence of SEQ ID NO:26;

(b) a fragment of the amino acid sequence of SEQ ID NO:26, the fragment comprising eight contiguous amino acids of SEQ ID NO:26; and

(c) the amino acid sequence encoded by the cDNA insert of clone pc201_{—}6 deposited under accession number ATCC 98781;

the protein being substantially free from other mammalian proteins. Preferably such protein comprises the amino acid sequence of SEQ ID NO:26. In further preferred embodiments, the present invention provides a protein comprising a fragment of the amino acid sequence of SEQ ID NO:26 having biological activity, the fragment preferably comprising eight (more preferably twenty, most preferably thirty) contiguous amino acids of SEQ ID NO:26, or a protein comprising a fragment of the amino acid sequence of SEQ ID NO:26 having biological activity, the fragment comprising the amino acid sequence from amino acid 170 to amino acid 179 of SEQ ID NO:26.

In one embodiment, the present invention provides a composition comprising an isolated polynucleotide selected from the group consisting of:

(a) a polynucleotide comprising the nucleotide sequence of SEQ ID NO:27;

(b) a polynucleotide comprising the nucleotide sequence of SEQ ID NO:27 from nucleotide 5 to nucleotide 259;

(c) a polynucleotide comprising the nucleotide sequence of the full-length protein coding sequence of clone p187_{—}1 deposited under accession number ATCC 98781;

(d) a polynucleotide encoding the full-length protein encoded by the cDNA insert of clone p187_{—}1 deposited under accession number ATCC 98781;

(e) a polynucleotide comprising the nucleotide sequence of a mature protein coding sequence of clone p187_deposited under accession number ATCC 98781;

(f) a polynucleotide encoding a mature protein encoded by the cDNA insert of clone p187_{—}1 deposited under accession number ATCC 98781;

(g) a polynucleotide encoding a protein comprising the amino acid sequence of SEQ ID NO:28;

(h) a polynucleotide encoding a protein comprising a fragment of the amino acid sequence of SEQ ID NO:28 having biological activity, the fragment comprising eight contiguous amino acids of SEQ ID NO:28;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:27.

Preferably, such polynucleotide comprises the nucleotide sequence of SEQ ID NO:27 from nucleotide 5 to nucleotide 259; the nucleotide sequence of the full-length protein coding sequence of clone p187_{—}1 deposited under accession number ATCC 98781; or the nucleotide sequence of a mature protein coding sequence of clone p187_{—}1 deposited under accession number ATCC 98781. In other preferred embodiments, the polynucleotide encodes the full-length or a mature protein encoded by the cDNA insert of clone p187_{—}1 deposited under accession number ATCC 98781. In further preferred embodiments, the present invention provides a polynucleotide encoding a protein comprising a fragment of the amino acid sequence of SEQ ID NO:28 having biological activity, the fragment preferably comprising eight (more preferably twenty, most preferably thirty) contiguous amino acids of SEQ ID NO:28, or a polynucleotide encoding a protein comprising a fragment of the amino acid sequence of SEQ ID NO:28 having biological activity, the fragment comprising the amino acid sequence from amino acid 37 to amino acid 46 of SEQ ID NO:28.

Other embodiments provide the gene corresponding to the cDNA sequence of SEQ ID NO:27.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:27, but excluding the poly(A) tail at the 3′ end of SEQ ID NO:27; and

(ab) the nucleotide sequence of the cDNA insert of clone p187_{—}1 deposited under accession number ATCC 98781;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:27, but excluding the poly(A) tail at the 3′ end of SEQ ID NO:27; and

(bb) the nucleotide sequence of the cDNA insert of clone p187_{—}1 deposited under accession number ATCC 98781;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:27, and extending contiguously from a nucleotide sequence corresponding to the 5′ end of SEQ ID NO:27 to a nucleotide sequence corresponding to the 3′ end of SEQ ID NO:27, but excluding the poly(A) tail at the 3′ end of SEQ ID NO:27. Also preferably the polynucleotide isolated according to the above process comprises a nucleotide sequence corresponding to the cDNA sequence of SEQ ID NO:27 from nucleotide 5 to nucleotide 259, and extending contiguously from a nucleotide sequence corresponding to the 5′ end of said sequence of SEQ ID NO:27 from nucleotide 5 to nucleotide 259, to a nucleotide sequence corresponding to the 3′ end of said sequence of SEQ ID NO:27 from nucleotide 5 to nucleotide 259.

In other embodiments, the present invention provides a composition comprising a protein, wherein said protein comprises an amino acid sequence selected from the group consisting of:

(a) the amino acid sequence of SEQ ID NO:28;

(b) a fragment of the amino acid sequence of SEQ ID NO:28, the fragment comprising eight contiguous amino acids of SEQ ID NO:28; and

(c) the amino acid sequence encoded by the cDNA insert of clone p187_{—}1 deposited under accession number ATCC 98781;

the protein being substantially free from other mammalian proteins. Preferably such protein comprises the amino acid sequence of SEQ ID NO:28. In further preferred embodiments, the present invention provides a protein comprising a fragment of the amino acid sequence of SEQ ID NO:28 having biological activity, the fragment preferably comprising eight (more preferably twenty, most preferably thirty) contiguous amino acids of SEQ ID NO:28, or a protein comprising a fragment of the amino acid sequence of SEQ ID NO:28 having biological activity, the fragment comprising the amino acid sequence from amino acid 37 to amino acid 46 of SEQ ID NO:28.

In one embodiment, the present invention provides a composition comprising an isolated polynucleotide selected from the group consisting of:

(a) a polynucleotide comprising the nucleotide sequence of SEQ ID NO:29;

(b) a polynucleotide comprising the nucleotide sequence of SEQ ID NO:29 from nucleotide 62 to nucleotide 2284;

(c) a polynucleotide comprising the nucleotide sequence of the full-length protein coding sequence of clone pm514_{—}4 deposited under accession number ATCC 98781;

(d) a polynucleotide encoding the full-length protein encoded by the cDNA insert of clone pm514_{—}4 deposited under accession number ATCC 98781;

(e) a polynucleotide comprising the nucleotide sequence of a mature protein coding sequence of clone pm51144 deposited under accession number ATCC 98781;

(f) a polynucleotide encoding a mature protein encoded by the cDNA insert of clone pm514_{—}4 deposited under accession number ATCC 98781;

(g) a polynucleotide encoding a protein comprising the amino acid sequence of SEQ ID NO:30;

(h) a polynucleotide encoding a protein comprising a fragment of the amino acid sequence of SEQ ID NO:30 having biological activity, the fragment comprising eight contiguous amino acids of SEQ ID NO:30;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-h) and that has a length that is at least 25% of the length of SEQ ID NO:29.

Preferably, such polynucleotide comprises the nucleotide sequence of SEQ ID NO:29 from nucleotide 62 to nucleotide 2284; the nucleotide sequence of the full-length protein coding sequence of clone pm514_{—}4 deposited under accession number ATCC 98781; or the nucleotide sequence of a mature protein coding sequence of clone pm514_{—}4 deposited under accession number ATCC 98781. In other preferred embodiments, the polynucleotide encodes the full-length or a mature protein encoded by the cDNA insert of clone pm514_{—}4 deposited under accession number ATCC 98781. In further preferred embodiments, the present invention provides a polynucleotide encoding a protein comprising a fragment of the amino acid sequence of SEQ ID NO:30 having biological activity, the fragment preferably comprising eight (more preferably twenty, most preferably thirty) contiguous amino acids of SEQ ID NO:30, or a polynucleotide encoding a protein comprising a fragment of the amino acid sequence of SEQ ID NO:30 having biological activity, the fragment comprising the amino acid sequence from amino acid 365 to amino acid 374 of SEQ ID NO:30.

Other embodiments provide the gene corresponding to the cDNA sequence of SEQ ID NO:29.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:29, but excluding the poly(A) tail at the 3′ end of SEQ ID NO:29; and

(ab) the nucleotide sequence of the cDNA insert of clone pm514_{—}4 deposited under accession number ATCC 98781;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:29, but excluding the poly(A) tail at the 3′ end of SEQ ID NO:29; and

(bb) the nucleotide sequence of the cDNA insert of clone pm5144 deposited under accession number ATCC 98781;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:29, and extending contiguously from a nucleotide sequence corresponding to the 5′ end of SEQ ID NO:29 to a nucleotide sequence corresponding to the 3′ end of SEQ ID NO:29, but excluding the poly(A) tail at the 3′ end of SEQ ID NO:29. Also preferably the polynucleotide isolated according to the above process comprises a nucleotide sequence corresponding to the cDNA sequence of SEQ ID NO:29 from nucleotide 62 to nucleotide 2284, and extending contiguously from a nucleotide sequence corresponding to the 5′ end of said sequence of SEQ ID NO:29 from nucleotide 62 to nucleotide 2284, to a nucleotide sequence corresponding to the 3′ end of said sequence of SEQ ID NO:29 from nucleotide 62 to nucleotide 2284.

In other embodiments, the present invention provides a composition comprising a protein, wherein said protein comprises an amino acid sequence selected from the group consisting of:

(a) the amino acid sequence of SEQ ID NO:30;

(b) a fragment of the amino acid sequence of SEQ ID NO:30, the fragment comprising eight contiguous amino adds of SEQ ID NO:30; and

(c) the amino acid sequence encoded by the cDNA insert of clone pm514_{—}4 deposited under accession number ATCC 98781;

the protein being substantially free from other mammalian proteins. Preferably such protein comprises the amino add sequence of SEQ ID NO:30. In further preferred embodiments, the present invention provides a protein comprising a fragment of the amino add sequence of SEQ ID NO:30 having biological activity, the fragment preferably comprising eight (more preferably twenty, most preferably thirty) contiguous amino adds of SEQ ID NO-0.30, or a protein comprising a fragment of the amino acid sequence of SEQ ID NO:30 having biological activity, the fragment comprising the amino acid sequence from amino add 365 to amino add 374 of SEQ ID NO:30.

In one embodiment, the present invention provides a composition comprising an isolated polynucleotide selected from the group consisting of:

(a) a polynucleotide comprising the nucleotide sequence of SEQ ID NO:31;

(b) a polynucleotide comprising the nucleotide sequence of SEQ ID NO:31 from nucleotide 36 to nucleotide 1997;

(c) a polynucleotide comprising the nucleotide sequence of SEQ ID NO:31 from nucleotide 135 to nucleotide 1997;

(d) a polynucleotide comprising the nucleotide sequence of the full-length protein coding sequence of clone co155_{—}12 deposited under accession number ATCC 98808;

(e) a polynucleotide encoding the full-length protein encoded by the cDNA insert of clone co155_{—}12 deposited under accession number ATCC 98808;

(f) a polynucleotide comprising the nucleotide sequence of a mature protein coding sequence of clone co155_{—}12 deposited under accession number ATCC 98808;

(g) a polynucleotide encoding a mature protein encoded by the cDNA insert of clone co155_{—}12 deposited under accession number ATCC 98808;

(h) a polynucleotide encoding a protein comprising the amino acid sequence of SEQ ID NO:32;

(i) a polynucleotide encoding a protein comprising a fragment of the amino acid sequence of SEQ ID NO:32 having biological activity, the fragment comprising eight contiguous amino acids of SEQ ID NO:32;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:31.

Preferably, such polynucleotide comprises the nucleotide sequence of SEQ ID NO:31 from nucleotide 36 to nucleotide 1997; the nucleotide sequence of SEQ ID NO:31 from nucleotide 135 to nucleotide 1997; the nucleotide sequence of the full-length protein coding sequence of clone co155_{—}12 deposited under accession number ATCC 98808; or the nucleotide sequence of a mature protein coding sequence of clone co155_{—}12 deposited under accession number ATCC 98808. In other preferred embodiments, the polynucleotide encodes the full-length or a mature protein encoded by the cDNA insert of clone co155_{—}12 deposited under accession number ATCC 98808. In further preferred embodiments, the present invention provides a polynucleotide encoding a protein comprising a fragment of the amino acid sequence of SEQ ID NO:32 having biological activity, the fragment preferably comprising eight (more preferably twenty, most preferably thirty) contiguous amino acids of SEQ ID NO:32, or a polynucleotide encoding a protein comprising a fragment of the amino acid sequence of SEQ ID NO:32 having biological activity, the fragment comprising the amino acid sequence from amino acid 322 to amino acid 331 of SEQ ID NO:32.

Other embodiments provide the gene corresponding to the cDNA sequence of SEQ ID NO:31.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:31, but excluding the poly(A) tail at the 3′ end of SEQ ID NO:31; and

(ab) the nucleotide sequence of the cDNA insert of clone co155_{—}12 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:31, but excluding the poly(A) tail at the 3′ end of SEQ ID NO:31; and

(bb) the nucleotide sequence of the cDNA insert of clone co155_{—}12 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:31, and extending contiguously from a nucleotide sequence corresponding to the 5′ end of SEQ ID NO:31 to a nucleotide sequence corresponding to the 3′ end of SEQ ID NO:31, but excluding the poly(A) tail at the 3′ end of SEQ ID NO:31. Also preferably the polynucleotide isolated according to the above process comprises a nucleotide sequence corresponding to the cDNA sequence of SEQ ID NO:31 from nucleotide 36 to nucleotide 1997, and extending contiguously from a nucleotide sequence corresponding to the 5′ end of said sequence of SEQ ID NO:31 from nucleotide 36 to nucleotide 1997, to a nucleotide sequence corresponding to the 3′ end of said sequence of SEQ ID NO:31 from nucleotide 36 to nucleotide 1997. Also preferably the polynucleotide isolated according to the above process comprises a nucleotide sequence corresponding to the cDNA sequence of SEQ ID NO:31 from nucleotide 135 to nucleotide 1997, and extending contiguously from a nucleotide sequence corresponding to the 5′ end of said sequence of SEQ ID NO:31 from nucleotide 135 to nucleotide 1997, to a nucleotide sequence corresponding to the 3′ end of said sequence of SEQ ID NO:31 from nucleotide 135 to nucleotide 1997.

In other embodiments, the present invention provides a composition comprising a protein, wherein said protein comprises an amino acid sequence selected from the group consisting of:

(a) the amino acid sequence of SEQ ID NO:32;

(b) a fragment of the amino acid sequence of SEQ ID NO:32, the fragment comprising eight contiguous amino acids of SEQ ID NO:32; and

(c) the amino acid sequence encoded by the cDNA insert of clone co155_{—}12 deposited under accession number ATCC 98808;

the protein being substantially free from other mammalian proteins. Preferably such protein comprises the amino acid sequence of SEQ ID NO:32. In further preferred embodiments, the present invention provides a protein comprising a fragment of the amino acid sequence of SEQ ID NO:32 having biological activity, the fragment preferably comprising eight (more preferably twenty, most preferably thirty) contiguous amino acids of SEQ ID NO:32, or a protein comprising a fragment of the amino acid sequence of SEQ ID NO:32 having biological activity, the fragment comprising the amino acid sequence from amino acid 322 to amino acid 331 of SEQ ID NO:32.

In one embodiment, the present invention provides a composition comprising an isolated polynucleotide selected from the group consisting of:

(a) a polynucleotide comprising the nucleotide sequence of SEQ ID NO:33;

(b) a polynucleotide comprising the nucleotide sequence of SEQ ID NO:33 from nucleotide 21 to nucleotide 1343;

(c) a polynucleotide comprising the nucleotide sequence of SEQ ID NO:33 from nucleotide 84 to nucleotide 1343;

(d) a polynucleotide comprising the nucleotide sequence of the full-length protein coding sequence of clone fn189_{—}13 deposited under accession number ATCC 98808;

(e) a polynucleotide encoding the full-length protein encoded by the cDNA insert of clone fn189_{—}13 deposited under accession number ATCC 98808;

(f) a polynucleotide comprising the nucleotide sequence of a mature protein coding sequence of clone fn189_{—}13 deposited under accession number ATCC 98808;

(g) a polynucleotide encoding a mature protein encoded by the cDNA insert of clone fn189_{—}13 deposited under accession number ATCC 98808;

(h) a polynucleotide encoding a protein comprising the amino acid sequence of SEQ ID NO:34;

(i) a polynucleotide encoding a protein comprising a fragment of the amino acid sequence of SEQ ID NO:34 having biological activity, the fragment comprising eight contiguous amino adds of SEQ ID NO:34;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:33.

Preferably, such polynucleotide comprises the nucleotide sequence of SEQ ID NO:33 from nucleotide 21 to nucleotide 1343; the nucleotide sequence of SEQ ID NO:33 from nucleotide 84 to nucleotide 1343; the nucleotide sequence of the full-length protein coding sequence of clone fn189_{—}13 deposited under accession number ATCC 98808; or the nucleotide sequence of a mature protein coding sequence of clone fn189_{—}13 deposited under accession number ATCC 98808. In other preferred embodiments, the polynucleotide encodes the full-length or a mature protein encoded by the cDNA insert of clone fn189_{—}13 deposited under accession number ATCC 98808. In further preferred embodiments, the present invention provides a polynucleotide encoding a protein comprising a fragment of the amino acid sequence of SEQ ID NO:34 having biological activity, the fragment preferably comprising eight (more preferably twenty, most preferably thirty) contiguous amino adds of SEQ ID NO:34, or a polynucleotide encoding a protein comprising a fragment of the amino acid sequence of SEQ ID NO-34 having biological activity, the fragment comprising the amino acid sequence from amino acid 215 to amino acid 224 of SEQ ID NO:34.

Other embodiments provide the gene corresponding to the cDNA sequence of SEQ ID NO:33.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:33, but excluding the poly(A) tail at the 3′ end of SEQ ID NO:33; and

(ab) the nucleotide sequence of the cDNA insert of clone fn189_{—}13 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:33, but excluding the poly(A) tail at the 3′end of SEQ ID NO:33; and

(bb) the nucleotide sequence of the cDNA insert of clone fn18913 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:33, and extending contiguously from a nucleotide sequence corresponding to the 5′ end of SEQ ID NO:33 to a nucleotide sequence corresponding to the 3′ end of SEQ ID NO:33, but excluding the poly(A) tail at the 3′ end of SEQ ID NO:33. Also preferably the polynucleotide isolated according to the above process comprises a nucleotide sequence corresponding to the cDNA sequence of SEQ ID NO:33 from nucleotide 21 to nucleotide 1343, and extending contiguously from a nucleotide sequence corresponding to the 5′ end of said sequence of SEQ ID NO:33 from nucleotide 21 to nucleotide 1343, to a nucleotide sequence corresponding to the 3′ end of said sequence of SEQ ID NO:33 from nucleotide 21 to nucleotide 1343. Also preferably the polynucleotide isolated according to the above process comprises a nucleotide sequence corresponding to the cDNA sequence of SEQ ID NO:33 from nucleotide 84 to nucleotide 1343, and extending contiguously from a nucleotide sequence corresponding to the 5′ end of said sequence of SEQ ID NO:33 from nucleotide 84 to nucleotide 1343, to a nucleotide sequence corresponding to the 3′ end of said sequence of SEQ ID NO:33 from nucleotide 84 to nucleotide 1343.

In other embodiments, the present invention provides a composition comprising a protein, wherein said protein comprises an amino add sequence selected from the group consisting of:

(a) the amino acid sequence of SEQ ID NO:34;

(b) a fragment of the amino acid sequence of SEQ ID NO:34, the fragment comprising eight contiguous amino acids of SEQ ID NO:34; and

(c) the amino acid sequence encoded by the cDNA insert of clone fn189_{—}13 deposited under accession number ATCC 98808;

the protein being substantially free from other mammalian proteins. Preferably such protein comprises the amino acid sequence of SEQ ID NO:34. In further preferred embodiments, the present invention provides a protein comprising a fragment of the amino add sequence of SEQ ID NO:34 having biological activity, the fragment preferably comprising eight (more preferably twenty, most preferably thirty) contiguous amino adds of SEQ ID NO:34, or a protein comprising a fragment of the amino acid sequence of SEQ ID NO:34 having biological activity, the fragment comprising the amino acid sequence from amino add 215 to amino acid 224 of SEQ ID NO:34.

In one embodiment, the present invention provides a composition comprising an isolated polynucleotide selected from the group consisting of:

(a) a polynucleotide comprising the nucleotide sequence of SEQ ID NO:35;

(b) a polynucleotide comprising the nucleotide sequence of SEQ ID NO:35 from nucleotide 66 to nucleotide 557;

(c) a polynucleotide comprising the nucleotide sequence of SEQ ID NO:35 from nucleotide 235 to nucleotide 899;

(d) a polynucleotide comprising the nucleotide sequence of the full-length protein coding sequence of clone lv2_{—}47 deposited under accession number ATCC 98808;

(e) a polynucleotide encoding the full-length protein encoded by the cDNA insert of clone lv2_{—}47 deposited under accession number ATCC 98808;

(f) a polynucleotide comprising the nucleotide sequence of a mature protein coding sequence of clone lv2_{—}47 deposited under accession number ATCC 98808;

(g) a polynucleotide encoding a mature protein encoded by the cDNA insert of clone lv2_{—}47 deposited under accession number ATCC 98808;

(h) a polynucleotide encoding a protein comprising the amino acid sequence of SEQ ID NO:36;

(i) a polynucleotide encoding a protein comprising a fragment of the amino acid sequence of SEQ ID NO:36 having biological activity, the fragment comprising eight contiguous amino acids of SEQ ID NO:36;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:35.

Preferably, such polynucleotide comprises the nucleotide sequence of SEQ ID NO:35 from nucleotide 66 to nucleotide 557; the nucleotide sequence of SEQ ID NO:35 from nucleotide 235 to nucleotide 899; the nucleotide sequence of the full-length protein coding sequence of clone lv2_{—}47 deposited under accession number ATCC 98808; or the nucleotide sequence of a mature protein coding sequence of clone lv2_{—}47 deposited under accession number ATCC 98808. In other preferred embodiments, the polynucleotide encodes the full-length or a mature protein encoded by the cDNA insert of clone lv2_{—}47 deposited under accession number ATCC 98808. In yet other preferred embodiments, the present invention provides a polynucleotide encoding a protein comprising the amino acid sequence of SEQ ID NO:36 from amino acid 58 to amino acid 164. In further preferred embodiments, the present invention provides a polynucleotide encoding a protein comprising a fragment of the amino acid sequence of SEQ ID NO:36 having biological activity, the fragment preferably comprising eight (more preferably twenty, most preferably thirty) contiguous amino acids of SEQ ID NO:36, or a polynucleotide encoding a protein comprising a fragment of the amino acid sequence of SEQ ID NO:36 having biological activity, the fragment comprising the amino acid sequence from amino acid 77 to amino acid 86 of SEQ ID NO:36.

Other embodiments provide the gene corresponding to the cDNA sequence of SEQ ID NO:35.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:35, but excluding the poly(A) tail at the 3′ end of SEQ ID NO:35; and

(ab) the nucleotide sequence of the cDNA insert of clone lv2_{—}47 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:35, but excluding the poly(A) tail at the 3′ end of SEQ ID NO:35; and

(bb) the nucleotide sequence of the cDNA insert of clone lv2_{—}47 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:35, and extending contiguously from a nucleotide sequence corresponding to the 5′ end of SEQ ID NO:35 to a nucleotide sequence corresponding to the 3′ end of SEQ ID NO:35, but excluding the poly(A) tail at the 3′ end of SEQ ID NO:35. Also preferably the polynucleotide isolated according to the above process comprises a nucleotide sequence corresponding to the cDNA sequence of SEQ ID NO:35 from nucleotide 66 to nucleotide 557, and extending contiguously from a nucleotide sequence corresponding to the 5′ end of said sequence of SEQ ID NO:35 from nucleotide 66 to nucleotide 557, to a nucleotide sequence corresponding to the 3′ end of said sequence of SEQ ID NO:35 from nucleotide 66 to nucleotide 557. Also preferably the polynucleotide isolated according to the above process comprises a nucleotide sequence corresponding to the cDNA sequence of SEQ ID NO:35 from nucleotide 235 to nucleotide 899, and extending contiguously from a nucleotide sequence corresponding to the 5′ end of said sequence of SEQ ID NO:35 from nucleotide 235 to nucleotide 899, to a nucleotide sequence corresponding to the 3′ end of said sequence of SEQ ID NO:35 from nucleotide 235 to nucleotide 899.

In other embodiments, the present invention provides a composition comprising a protein, wherein said protein comprises an amino add sequence selected from the group consisting of:

(a) the amino acid sequence of SEQ ID NO:36;

(b) the amino acid sequence of SEQ ID NO:36 from amino acid 58 to amino acid 164;

(c) a fragment of the amino add sequence of SEQ ID NO:36, the fragment comprising eight contiguous amino acids of SEQ ID NO:36; and

(d) the amino acid sequence encoded by the cDNA insert of clone lv2_{—}47 deposited under accession number ATCC 98808;

the protein being substantially free from other mammalian proteins. Preferably such protein comprises the amino add sequence of SEQ ID NO:36 or the amino acid sequence of SEQ ID NO:36 from amino acid 58 to amino acid 164. In further preferred embodiments, the present invention provides a protein comprising a fragment of the amino acid sequence of SEQ ID NO:36 having biological activity, the fragment preferably comprising eight (more preferably twenty, most preferably thirty) contiguous amino acids of SEQ ID NO:36, or a protein comprising a fragment of the amino acid sequence of SEQ ID NO:36 having biological activity, the fragment comprising the amino acid sequence from amino add 77 to amino acid 86 of SEQ ID NO:36.

In one embodiment, the present invention provides a composition comprising an isolated polynucleotide selected from the group consisting of:

(a) a polynucleotide comprising the nucleotide sequence of SEQ ID NO:37;

(b) a polynucleotide comprising the nucleotide sequence of SEQ ID NO:37 from nucleotide 104 to nucleotide 499;

(c) a polynucleotide comprising the nucleotide sequence of SEQ ID NO:37 from nucleotide 215 to nucleotide 499;

(d) a polynucleotide comprising the nucleotide sequence of the full-length protein coding sequence of clone m1243_{—}1 deposited under accession number ATCC 98808;

(e) a polynucleotide encoding the full-length protein encoded by the cDNA insert of clone m1243_{—}1 deposited under accession number ATCC 98808;

(f) a polynucleotide comprising the nucleotide sequence of a mature protein coding sequence of clone m1243_{—}1 deposited under accession number ATCC 98808;

(g) a polynucleotide encoding a mature protein encoded by the cDNA insert of clone m1243_deposited under accession number ATCC 98808;

(h) a polynucleotide encoding a protein comprising the amino acid sequence of SEQ ID NO:38;

(i) a polynucleotide encoding a protein comprising a fragment of the amino acid sequence of SEQ ID NO:38 having biological activity, the fragment comprising eight contiguous amino acids of SEQ ID NO:38;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:37.

Preferably, such polynucleotide comprises the nucleotide sequence of SEQ ID NO:37 from nucleotide 104 to nucleotide 499; the nucleotide sequence of SEQ ID NO:37 from nucleotide 215 to nucleotide 499; the nucleotide sequence of the full-length protein coding sequence of clone ml243_{—}1 deposited under accession number ATCC 98808; or the nucleotide sequence of a mature protein coding sequence of clone ml243_{—}1 deposited under accession number ATCC 98808. In other preferred embodiments, the polynucleotide encodes the full-length or a mature protein encoded by the cDNA insert of clone ml243_{—}1 deposited under accession number ATCC 98808. In further preferred embodiments, the present invention provides a polynucleotide encoding a protein comprising a fragment of the amino acid sequence of SEQ ID NO:38 having biological activity, the fragment preferably comprising eight (more preferably twenty, most preferably thirty) contiguous amino adds of SEQ ID NO:38, or a polynucleotide encoding a protein comprising a fragment of the amino acid sequence of SEQ ID NO:38 having biological activity, the fragment comprising the amino acid sequence from amino add 61 to amino acid 70 of SEQ ID NO:38.

Other embodiments provide the gene corresponding to the cDNA sequence of SEQ ID NO:37.

Further embodiments of the invention provide isolated polynucleotides produced according to a process selected from the group consisting of:

(a) a process comprising the steps of.

(i) preparing one or more polynucleotide probes that hybridize in 6×SSC at 65 degrees C. to a nucleotide sequence selected from the group consisting of:

(aa) SEQ ID NO:37, but excluding the poly(A) tail at the 3′ end of SEQ ID NO:37; and

(ab) the nucleotide sequence of the cDNA insert of clone ml243_deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:37, but excluding the poly(A) tail at the 3′ end of SEQ ID NO:37; and

(bb) the nucleotide sequence of the cDNA insert of clone ml243_{—}1 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:37, and extending contiguously from a nucleotide sequence corresponding to the 5′ end of SEQ ID NO:37 to a nucleotide sequence corresponding to the 3′ end of SEQ ID NO:37, but excluding the poly(A) tail at the 3′ end of SEQ ID NO:37. Also preferably the polynucleotide isolated according to the above process comprises a nucleotide sequence corresponding to the cDNA sequence of SEQ ID NO:37 from nucleotide 104 to nucleotide 499, and extending contiguously from a nucleotide sequence corresponding to the 5′ end of said sequence of SEQ ID NO:37 from nucleotide 104 to nucleotide 499, to a nucleotide sequence corresponding to the 3′ end of said sequence of SEQ ID NO:37 from nucleotide 104 to nucleotide 499. Also preferably the polynucleotide isolated according to the above process comprises a nucleotide sequence corresponding to the cDNA sequence of SEQ ID NO:37 from nucleotide 215 to nucleotide 499, and extending contiguously from a nucleotide sequence corresponding to the 5′ end of said sequence of SEQ ID NO:37 from nucleotide 215 to nucleotide 499, to a nucleotide sequence corresponding to the 3′ end of said sequence of SEQ ID NO:37 from nucleotide 215 to nucleotide 499.

In other embodiments, the present invention provides a composition comprising a protein, wherein said protein comprises an amino acid sequence selected from the group consisting of:

(a) the amino acid sequence of SEQ ID NO:38;

(b) a fragment of the amino acid sequence of SEQ ID NO:38, the fragment comprising eight contiguous amino acids of SEQ ID NO:38; and

(c) the amino acid sequence encoded by the cDNA insert of clone ml243_{—}1 deposited under accession number ATCC 98808;

the protein being substantially free from other mammalian proteins. Preferably such protein comprises the amino acid sequence of SEQ ID NO:38. In further preferred embodiments, the present invention provides a protein comprising a fragment of the amino acid sequence of SEQ ID NO:38 having biological activity, the fragment preferably comprising eight (more preferably twenty, most preferably thirty) contiguous amino acids of SEQ ID NO:38, or a protein comprising a fragment of the amino acid sequence of SEQ ID NO:38 having biological activity, the fragment comprising the amino acid sequence from amino acid 61 to amino acid 70 of SEQ ID NO:38.

In one embodiment, the present invention provides a composition comprising an isolated polynucleotide selected from the group consisting of:

(a) a polynucleotide comprising the nucleotide sequence of SEQ ID NO:39;

(b) a polynucleotide comprising the nucleotide sequence of SEQ ID NO:39 from nucleotide 2172 to nucleotide 2861;

(c) a polynucleotide comprising the nucleotide sequence of the full-length protein coding sequence of clone pm96_{—}9 deposited under accession number ATCC 98808;

(d) a polynucleotide encoding the full-length protein encoded by the cDNA insert of clone pm96_{—}9 deposited under accession number ATCC 98808;

(e) a polynucleotide comprising the nucleotide sequence of a mature protein coding sequence of clone pm96_{—}9 deposited under accession number ATCC 98808;

(f) a polynucleotide encoding a mature protein encoded by the cDNA insert of clone pm96_{—}9 deposited under accession number ATCC 98808;

(g) a polynucleotide encoding a protein comprising the amino acid sequence of SEQ ID NO:40;

(h) a polynucleotide encoding a protein comprising a fragment of the amino acid sequence of SEQ ID NO:40 having biological activity, the fragment comprising eight contiguous amino acids of SEQ ID NO:40;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:39.

Preferably, such polynucleotide comprises the nucleotide sequence of SEQ ID NO:39 from nucleotide 2172 to nucleotide 2861; the nucleotide sequence of the full-length protein coding sequence of clone pm96_{—}9 deposited under accession number ATCC 98808; or the nucleotide sequence of a mature protein coding sequence of clone pm96_{—}9 deposited under accession number ATCC 98808. In other preferred embodiments, the polynucleotide encodes the full-length or a mature protein encoded by the cDNA insert of clone pm96_{—}9 deposited under accession number ATCC 98808. In further preferred embodiments, the present invention provides a polynucleotide encoding a protein comprising a fragment of the amino acid sequence of SEQ ID NO:40 having biological activity, the fragment preferably comprising eight (more preferably twenty, most preferably thirty) contiguous amino acids of SEQ ID NO:40, or a polynucleotide encoding a protein comprising a fragment of the amino acid sequence of SEQ ID NO:40 having biological activity, the fragment comprising the amino acid sequence from amino acid 110 to amino acid 119 of SEQ ID NO:40.

Other embodiments provide the gene corresponding to the cDNA sequence of SEQ ID NO:39.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:39, but excluding the poly(A) tail at the 3′ end of SEQ ID NO:39; and

(ab) the nucleotide sequence of the cDNA insert of clone pm96_{—}9 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of,

(i) preparing one or more polynucleotide primers that hybridize in 6×SSC at 65 degrees C. to a nucleotide sequence selected from the group consisting of:

(ba) SEQ ID NO:39, but excluding the poly(A) tail at the 3′ end of SEQ ID NO:39; and

(bb) the nucleotide sequence of the cDNA insert of clone pm96_{—}9 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:39, and extending contiguously from a nucleotide sequence corresponding to the 5′ end of SEQ ID NO:39 to a nucleotide sequence corresponding to the 3′ end of SEQ ID NO:39, but excluding the poly(A) tail at the 3′ end of SEQ ID NO:39. Also preferably the polynucleotide isolated according to the above process comprises a nucleotide sequence corresponding to the cDNA sequence of SEQ ID NO:39 from nucleotide 2172 to nucleotide 2861, and extending contiguously from a nucleotide sequence corresponding to the 5′ end of said sequence of SEQ ID NO:39 from nucleotide 2172 to nucleotide 2861, to a nucleotide sequence corresponding to the 3′ end of said sequence of SEQ ID NO:39 from nucleotide 2172 to nucleotide 2861.

In other embodiments, the present invention provides a composition comprising a protein, wherein said protein comprises an amino add sequence selected from the group consisting of:

(a) the amino acid sequence of SEQ ID NO:40;

(b) a fragment of the amino acid sequence of SEQ ID NO:40, the fragment comprising eight contiguous amino acids of SEQ ID NO:40; and

(c) the amino acid sequence encoded by the cDNA insert of clone pm96_{—}9 deposited under accession number ATCC 98808;

the protein being substantially free from other mammalian proteins. Preferably such protein comprises the amino acid sequence of SEQ ID NO:40. In further preferred embodiments, the present invention provides a protein comprising a fragment of the amino acid sequence of SEQ ID NO:40 having biological activity, the fragment preferably comprising eight (more preferably twenty, most preferably thirty) contiguous amino acids of SEQ ID NO:40, or a protein comprising a fragment of the amino acid sequence of SEQ ID NO:40 having biological activity, the fragment comprising the amino acid sequence from amino acid 110 to amino acid 119 of SEQ ID NO:40.

In one embodiment, the present invention provides a composition comprising an isolated polynucleotide selected from the group consisting of:

(a) a polynucleotide comprising the nucleotide sequence of SEQ ID NO:41;

(b) a polynucleotide comprising the nucleotide sequence of SEQ ID NO:41 from nucleotide 43 to nucleotide 762;

(c) a polynucleotide comprising the nucleotide sequence of SEQ ID NO:41 from nucleotide 427 to nucleotide 762;

(d) a polynucleotide comprising the nucleotide sequence of the full-length protein coding sequence of clone pu261_{—}1 deposited under accession number ATCC 98808;

(e) a polynucleotide encoding the full-length protein encoded by the cDNA insert of clone pu261_{—}1 deposited under accession number ATCC 98808;

(f) a polynucleotide comprising the nucleotide sequence of a mature protein coding sequence of clone pu261_{—}1 deposited under accession number ATCC 98808;

(g) a polynucleotide encoding a mature protein encoded by the cDNA insert of clone pu261_{—}1 deposited under accession number ATCC 98808;

(h) a polynucleotide encoding a protein comprising the amino acid sequence of SEQ ID NO:42;

(i) a polynucleotide encoding a protein comprising a fragment of the amino add sequence of SEQ ID NO:42 having biological activity, the fragment comprising eight contiguous amino acids of SEQ ID NO:42;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:41.

Preferably, such polynucleotide comprises the nucleotide sequence of SEQ ID NO:41 from nucleotide 43 to nucleotide 762; the nucleotide sequence of SEQ ID NO:41 from nucleotide 427 to nucleotide 762; the nucleotide sequence of the full-length protein coding sequence of clone pu261_{—}1 deposited under accession number ATCC 98808; or the nucleotide sequence of a mature protein coding sequence of clone pu261_{—}1 deposited under accession number ATCC 98808. In other preferred embodiments, the polynucleotide encodes the full-length or a mature protein encoded by the cDNA insert of clone pu261_{—}1 deposited under accession number ATCC 98808. In further preferred embodiments, the present invention provides a polynucleotide encoding a protein comprising a fragment of the amino add sequence of SEQ ID NO:42 having biological activity, the fragment preferably comprising eight (more preferably twenty, most preferably this) contiguous amino acids of SEQ ID NO:42, or a polynucleotide encoding a protein comprising a fragment of the amino acid sequence of SEQ ID NO:42 having biological activity, the fragment comprising the amino acid sequence from amino acid 115 to amino acid 124 of SEQ ID NO:42.

Other embodiments provide the gene corresponding to the cDNA sequence of SEQ ID NO:41.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:41, but excluding the poly(A) tail at the 3′ end of SEQ ID NO:41; and

(ab) the nucleotide sequence of the cDNA insert of clone pu261_{—}1 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:41, but excluding the poly(A) tail at the 3′ end of SEQ ID NO:41; and

(bb) the nucleotide sequence of the cDNA insert of clone pu261_{—}1 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:41, and extending contiguously from a nucleotide sequence corresponding to the 5′ end of SEQ ID NO:41 to a nucleotide sequence corresponding to the 3′ end of SEQ ID NO:41, but excluding the poly(A) tail at the 3′ end of SEQ ID NO:41. Also preferably the polynucleotide isolated according to the above process comprises a nucleotide sequence corresponding to the cDNA sequence of SEQ ID NO:41 from nucleotide 43 to nucleotide 762, and extending contiguously from a nucleotide sequence corresponding to the 5′ end of said sequence of SEQ ID NO:41 from nucleotide 43 to nucleotide 762, to a nucleotide sequence corresponding to the 3′ end of said sequence of SEQ ID NO:41 from nucleotide 43 to nucleotide 762. Also preferably the polynucleotide isolated according to the above process comprises a nucleotide sequence corresponding to the cDNA sequence of SEQ ID NO:41 from nucleotide 427 to nucleotide 762, and extending contiguously from a nucleotide sequence corresponding to the 5′ end of said sequence of SEQ ID NO:41 from nucleotide 427 to nucleotide 762, to a nucleotide sequence corresponding to the 3′ end of said sequence of SEQ ID NO:41 from nucleotide 427 to nucleotide 762.

In other embodiments, the present invention provides a composition comprising a protein, wherein said protein comprises an amino acid sequence selected from the group consisting of:

(a) the amino acid sequence of SEQ ID NO:42;

(b) a fragment of the amino add sequence of SEQ ID NO:42, the fragment comprising eight contiguous amino acids of SEQ ID NO:42; and

(c) the amino acid sequence encoded by the cDNA insert of clone pu261_{—}1 deposited under accession number ATCC 98808;

the protein being substantially free from other mammalian proteins. Preferably such protein comprises the amino acid sequence of SEQ ID NO-0.42. In further preferred embodiments, the present invention provides a protein comprising a fragment of the amino acid sequence of SEQ ID NO:42 having biological activity, the fragment preferably comprising eight (more preferably twenty, most preferably thirty) contiguous amino adds of SEQ ID NO:42, or a protein comprising a fragment of the amino acid sequence of SEQ ID NO:42 having biological activity, the fragment comprising the amino acid sequence from amino acid 115 to amino acid 124 of SEQ ID NO:42.

In one embodiment, the present invention provides a composition comprising an isolated polynucleotide selected from the group consisting of:

(a) a polynucleotide comprising the nucleotide sequence of SEQ ID NO:43;

(b) a polynucleotide comprising the nucleotide sequence of SEQ ID NO:43 from nucleotide 579 to nucleotide 824;

(c) a polynucleotide comprising the nucleotide sequence of the full-length protein coding sequence of clone pw214_{—}15 deposited under accession number ATCC 98808;

(d) a polynucleotide encoding the full-length protein encoded by the cDNA insert of clone pw214_{—}15 deposited under accession number ATCC 98808;

(e) a polynucleotide comprising the nucleotide sequence of a mature protein coding sequence of clone pw214_{—}15 deposited under accession number ATCC 98808;

(f) a polynucleotide encoding a mature protein encoded by the cDNA insert of clone pw214_{—}15 deposited under accession number ATCC 98808;

(g) a polynucleotide encoding a protein comprising the amino acid sequence of SEQ ID NO:44;

(h) a polynucleotide encoding a protein comprising a fragment of the amino acid sequence of SEQ ID NO:44 having biological activity, the fragment comprising eight contiguous amino acids of SEQ ID NO:44;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:43.

Preferably, such polynucleotide comprises the nucleotide sequence of SEQ ID NO:43 from nucleotide 579 to nucleotide 824; the nucleotide sequence of the full-length protein coding sequence of clone pw214_{—}15 deposited under accession number ATCC 98808; or the nucleotide sequence of a mature protein coding sequence of clone pw214_{—}15 deposited under accession number ATCC 98808. In other preferred embodiments, the polynucleotide encodes the full-length or a mature protein encoded by the cDNA insert of clone pw214_{—}15 deposited under accession number ATCC 98808. In further preferred embodiments, the present invention provides a polynucleotide encoding a protein comprising a fragment of the amino acid sequence of SEQ ID NO:44 having biological activity, the fragment preferably comprising eight (more preferably twenty, most preferably thirty) contiguous amino acids of SEQ ID NO:44, or a polynucleotide encoding a protein comprising a fragment of the amino acid sequence of SEQ ID NO:44 having biological activity, the fragment comprising the amino acid sequence from amino acid 36 to amino acid 45 of SEQ ID NO:44.

Other embodiments provide the gene corresponding to the cDNA sequence of SEQ ID NO:43.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:43, but excluding the poly(A) tail at the 3′ end of SEQ ID NO:43; and

(ab) the nucleotide sequence of the cDNA insert of clone pw214_{—}15 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:43, but excluding the poly(A) tail at the 3′ end of SEQ ID NO:43; and

(bb) the nucleotide sequence of the cDNA insert of clone pw214_{—}15 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:43, and extending contiguously from a nucleotide sequence corresponding to the 5′ end of SEQ ID NO:43 to a nucleotide sequence corresponding to the 3′ end of SEQ ID NO:43, but excluding the poly(A) tail at the 3′ end of SEQ ID NO:43. Also preferably the polynucleotide isolated according to the above process comprises a nucleotide sequence corresponding to the cDNA sequence of SEQ ID NO:43 from nucleotide 579 to nucleotide 824, and extending contiguously from a nucleotide sequence corresponding to the 5′ end of said sequence of SEQ ID NO:43 from nucleotide 579 to nucleotide 824, to a nucleotide sequence corresponding to the 3′ end of said sequence of SEQ ID NO:43 from nucleotide 579 to nucleotide 824.

In other embodiments, the present invention provides a composition comprising a protein, wherein said protein comprises an amino acid sequence selected from the group consisting of:

(a) the amino acid sequence of SEQ ID NO:44;

(b) a fragment of the amino acid sequence of SEQ ID NO:44, the fragment comprising eight contiguous amino acids of SEQ ID NO:44; and

(c) the amino acid sequence encoded by the cDNA insert of clone pw214_{—}15 deposited under accession number ATCC 98808;

the protein being substantially free from other mammalian proteins. Preferably such protein comprises the amino acid sequence of SEQ ID NO:44. In further preferred embodiments, the present invention provides a protein comprising a fragment of the amino acid sequence of SEQ ID NO:44 having biological activity, the fragment preferably comprising eight (more preferably twenty, most preferably thirty) contiguous amino acids of SEQ ID NO:44, or a protein comprising a fragment of the amino acid sequence of SEQ ID NO:44 having biological activity, the fragment comprising the amino acid sequence from amino acid 36 to amino acid 45 of SEQ ID NO:44.

In one embodiment, the present invention provides a composition comprising an isolated polynucleotide selected from the group consisting of:

(a) a polynucleotide comprising the nucleotide sequence of SEQ ID NO:45;

(b) a polynucleotide comprising the nucleotide sequence of SEQ ID NO:45 from nucleotide 6 to nucleotide 383;

(c) a polynucleotide comprising the nucleotide sequence of the full-length protein coding sequence of clone qb56_{—}19 deposited under accession number ATCC 98808;

(d) a polynucleotide encoding the full-length protein encoded by the cDNA insert of clone qb56_{—}19 deposited under accession number ATCC 98808;

(e) a polynucleotide comprising the nucleotide sequence of a mature protein coding sequence of clone qb56_{—}19 deposited under accession number ATCC 98808;

(f) a polynucleotide encoding a mature protein encoded by the cDNA insert of clone qb5619 deposited under accession number ATCC 98808;

(g) a polynucleotide encoding a protein comprising the amino acid sequence of SEQ ID NO:46;

(h) a polynucleotide encoding a protein comprising a fragment of the amino acid sequence of SEQ ID NO:46 having biological activity, the fragment comprising eight contiguous amino acids of SEQ ID NO:46;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:45.

Preferably, such polynucleotide comprises the nucleotide sequence of SEQ ID NO:45 from nucleotide 6 to nucleotide 383; the nucleotide sequence of the full-length protein coding sequence of clone qb56_{—}19 deposited under accession number ATCC 98808; or the nucleotide sequence of a mature protein coding sequence of clone qb56_{—}19 deposited under accession number ATCC 98808. In other preferred embodiments, the polynucleotide encodes the full-length or a mature protein encoded by the cDNA insert of clone qb56_{—}19 deposited under accession number ATCC 98808. In further preferred embodiments, the present invention provides a polynucleotide encoding a protein comprising a fragment of the amino acid sequence of SEQ ID NO:46 having biological activity, the fragment preferably comprising eight (more preferably twenty, most preferably thirty) contiguous amino acids of SEQ ID NO:46, or a polynucleotide encoding a protein comprising a fragment of the amino acid sequence of SEQ ID NO:46 having biological activity, the fragment comprising the amino acid sequence from amino acid 58 to amino acid 67 of SEQ ID NO:46.

Other embodiments provide the gene corresponding to the cDNA sequence of SEQ ID NO:45.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:45, but excluding the poly(A) tail at the 3′ end of SEQ ID NO:45; and

(ab) the nucleotide sequence of the cDNA insert of clone qb56_{—}19 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of

(ba) SEQ ID NO:45, but excluding the poly(A) tail at the 3′ end of SEQ ID NO:45; and

(bb) the nucleotide sequence of the cDNA insert of clone qb56_{—}19 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:45, and extending contiguously from a nucleotide sequence corresponding to the 5′ end of SEQ ID NO:45 to a nucleotide sequence corresponding to the 3′ end of SEQ ID NO:45, but excluding the poly(A) tail at the 3′ end of SEQ ID NO:45. Also preferably the polynucleotide isolated according to the above process comprises a nucleotide sequence corresponding to the cDNA sequence of SEQ ID NO:45 from nucleotide 6 to nucleotide 383, and extending contiguously from a nucleotide sequence corresponding to the 5′ end of said sequence of SEQ ID NO:45 from nucleotide 6 to nucleotide 383, to a nucleotide sequence corresponding to the 3′ end of said sequence of SEQ ID NO:45 from nucleotide 6 to nucleotide 383.

In other embodiments, the present invention provides a composition comprising a protein, wherein said protein comprises an amino acid sequence selected from the group consisting of:

(a) the amino acid sequence of SEQ ID NO:46;

(b) a fragment of the amino acid sequence of SEQ ID NO:46, the fragment comprising eight contiguous amino acids of SEQ ID NO:46; and

(c) the amino acid sequence encoded by the cDNA insert of clone qb56_{—}19 deposited under accession number ATCC 98808;

the protein being substantially free from other mammalian proteins. Preferably such protein comprises the amino acid sequence of SEQ ID NO:46. In further preferred embodiments, the present invention provides a protein comprising a fragment of the amino acid sequence of SEQ ID NO:46 having biological activity, the fragment preferably comprising eight (more preferably twenty, most preferably thirty) contiguous amino acids of SEQ ID NO:46, or a protein comprising a fragment of the amino acid sequence of SEQ ID NO:46 having biological activity, the fragment comprising the amino acid sequence from amino acid 58 to amino acid 67 of SEQ ID NO:46.

In one embodiment, the present invention provides a composition comprising an isolated polynucleotide selected from the group consisting of:

(a) a polynucleotide comprising the nucleotide sequence of SEQ ID NO:47;

(b) a polynucleotide comprising the nucleotide sequence of SEQ ID NO:47 from nucleotide 170 to nucleotide 1273;

(c) a polynucleotide comprising the nucleotide sequence of SEQ ID NO:47 from nucleotide 242 to nucleotide 1273;

(d) a polynucleotide comprising the nucleotide sequence of the full-length protein coding sequence of clone qc646_{—}1 deposited under accession number ATCC 98808;

(e) a polynucleotide encoding the full-length protein encoded by the cDNA insert of clone qc646_{—}1 deposited under accession number ATCC 98808;

(f) a polynucleotide comprising the nucleotide sequence of a mature protein coding sequence of clone qc646_{—}1 deposited under accession number ATCC 98808;

(g) a polynucleotide encoding a mature protein encoded by the cDNA insert of clone qc646_{—}1 deposited under accession number ATCC 98808;

(h) a polynucleotide encoding a protein comprising the amino acid sequence of SEQ ID NO:48;

(i) a polynucleotide encoding a protein comprising a fragment of the amino acid sequence of SEQ ID NO:48 having biological activity, the fragment comprising eight contiguous amino acids of SEQ ID NO:48;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:47.

Preferably, such polynucleotide comprises the nucleotide sequence of SEQ ID NO:47 from nucleotide 170 to nucleotide 1273; the nucleotide sequence of SEQ ID NO:47 from nucleotide 242 to nucleotide 1273; the nucleotide sequence of the full-length protein coding sequence of clone qc646_{—}1 deposited under accession number ATCC 98808; or the nucleotide sequence of a mature protein coding sequence of clone qc646_{—}1 deposited under accession number ATCC 98808. In other preferred embodiments, the polynucleotide encodes the full-length or a mature protein encoded by the cDNA insert of clone qc646_{—}1 deposited under accession number ATCC 98808. In further preferred embodiments, the present invention provides a polynucleotide encoding a protein comprising a fragment of the amino acid sequence of SEQ ID NO:48 having biological activity, the fragment preferably comprising eight (more preferably twenty, most preferably thirty) contiguous amino acids of SEQ ID NO:48, or a polynucleotide encoding a protein comprising a fragment of the amino acid sequence of SEQ ID NO:48 having biological activity, the fragment comprising the amino acid sequence from amino acid 179 to amino acid 188 of SEQ ID NO:48.

Other embodiments provide the gene corresponding to the cDNA sequence of SEQ ID NO:47.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:47, but excluding the poly(A) tail at the 3′ end of SEQ ID NO:47; and

(ab) the nucleotide sequence of the cDNA insert of clone qc646_{—}1 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:47, but excluding the poly(A) tail at the 3′ end of SEQ ID NO:47; and

(bb) the nucleotide sequence of the cDNA insert of clone qc646_{—}1 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:47, and extending contiguously from a nucleotide sequence corresponding to the 5′ end of SEQ ID NO:47 to a nucleotide sequence corresponding to the 3′ end of SEQ ID NO:47, but excluding the poly(A) tail at the 3′ end of SEQ ID NO:47. Also preferably the polynucleotide isolated according to the above process comprises a nucleotide sequence corresponding to the cDNA sequence of SEQ ID NO:47 from nucleotide 170 to nucleotide 1273, and extending contiguously from a nucleotide sequence corresponding to the 5′ end of said sequence of SEQ ID NO:47 from nucleotide 170 to nucleotide 1273, to a nucleotide sequence corresponding to the 3′ end of said sequence of SEQ ID NO:47 from nucleotide 170 to nucleotide 1273. Also preferably the polynucleotide isolated according to the above process comprises a nucleotide sequence corresponding to the cDNA sequence of SEQ ID NO:47 from nucleotide 242 to nucleotide 1273, and extending contiguously from a nucleotide sequence corresponding to the 5′ end of said sequence of SEQ ID NO:47 from nucleotide 242 to nucleotide 1273, to a nucleotide sequence corresponding to the 3′ end of said sequence of SEQ ID NO:47 from nucleotide 242 to nucleotide 1273.

In other embodiments, the present invention provides a composition comprising a protein, wherein said protein comprises an amino acid sequence selected from the group consisting of:

(a) the amino acid sequence of SEQ ID NO:48;

(b) a fragment of the amino acid sequence of SEQ ID NO:48, the fragment comprising eight contiguous amino acids of SEQ ID NO:48; and

(c) the amino acid sequence encoded by the cDNA insert of clone qc646_{—}1 deposited under accession number ATCC 98808;

the protein being substantially free from other mammalian proteins. Preferably such protein comprises the amino acid sequence of SEQ ID NO:48. In further preferred embodiments, the present invention provides a protein comprising a fragment of the amino acid sequence of SEQ ID. NO:48 having biological activity, the fragment preferably comprising eight (more preferably twenty, most preferably thirty) contiguous amino acids of SEQ ID NO:48, or a protein comprising a fragment of the amino acid sequence of SEQ ID NO:48 having biological activity, the fragment comprising the amino acid sequence from amino acid 179 to amino acid 188 of SEQ ID NO:48.

In one embodiment, the present invention provides a composition comprising an isolated polynucleotide selected from the group consisting of:

(a) a polynucleotide comprising the nucleotide sequence of SEQ ID NO:49;

(b) a polynucleotide comprising the nucleotide sequence of SEQ ID NO:49 from nucleotide 183 to nucleotide 1097;

(c) a polynucleotide comprising the nucleotide sequence of the full-length protein coding sequence of clone qf116_{—}2 deposited under accession number ATCC 98808;

(d) a polynucleotide encoding the full-length protein encoded by the cDNA insert of clone qf116_{—}2 deposited under accession number ATCC 98808;

(e) a polynucleotide comprising the nucleotide sequence of a mature protein coding sequence of clone qf116_{—}2 deposited under accession number ATCC 98808;

(f) a polynucleotide encoding a mature protein encoded by the cDNA insert of clone qf116_{—}2 deposited under accession number ATCC 98808;

(g) a polynucleotide encoding a protein comprising the amino acid sequence of SEQ ID NO:50;

(h) a polynucleotide encoding a protein comprising a fragment of the amino acid sequence of SEQ ID NO:50 having biological activity, the fragment comprising eight contiguous amino acids of SEQ ID NO:50;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:49.

Preferably, such polynucleotide comprises the nucleotide sequence of SEQ ID NO:49 from nucleotide 183 to nucleotide 1097; the nucleotide sequence of the full-length protein coding sequence of clone qf116_{—}2 deposited under accession number ATCC 98808; or the nucleotide sequence of a mature protein coding sequence of clone qf116_{—}2 deposited under accession number ATCC 98808. In other preferred embodiments, the polynucleotide encodes the full-length or a mature protein encoded by the cDNA insert of clone qf116_{—}2 deposited under accession number ATCC 98808. In further preferred embodiments, the present invention provides a polynucleotide encoding a protein comprising a fragment of the amino add sequence of SEQ ID NO:50 having biological activity, the fragment preferably comprising eight (more preferably twenty, most preferably thirty) contiguous amino adds of SEQ ID NO:50, or a polynucleotide encoding a protein comprising a fragment of the amino acid sequence of SEQ ID NO:50 having biological activity, the fragment comprising the amino add sequence from amino add 147 to amino acid 156 of SEQ ID NO:50.

Other embodiments provide the gene corresponding to the cDNA sequence of SEQ ID NO:49.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:49, but excluding the poly(A) tail at the 3′ end of SEQ ID NO:49; and

(ab) the nucleotide sequence of the cDNA insert of clone qf116_{—}2 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:49, but excluding the poly(A) tail at the 3′ end of SEQ ID NO:49; and

(bb) the nucleotide sequence of the cDNA insert of clone qf116_{—}2 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:49, and extending contiguously from a nucleotide sequence corresponding to the 5′ end of SEQ ID NO:49 to a nucleotide sequence corresponding to the 3′ end of SEQ ID NO:49, but excluding the poly(A) tail at the 3′ end of SEQ ID NO:49. Also preferably the polynucleotide isolated according to the above process comprises a nucleotide sequence corresponding to the cDNA sequence of SEQ ID NO:49 from nucleotide 183 to nucleotide 1097, and extending contiguously from a nucleotide sequence corresponding to the 5′ end of said sequence of SEQ ID NO:49 from nucleotide 183 to nucleotide 1097, to a nucleotide sequence corresponding to the 3′ end of said sequence of SEQ ID NO:49 from nucleotide 183 to nucleotide 1097.

In other embodiments, the present invention provides a composition comprising a protein, wherein said protein comprises an amino acid sequence selected from the group consisting of:

(a) the amino acid sequence of SEQ ID NO:50;

(b) a fragment of the amino acid sequence of SEQ ID NO:50, the fragment comprising eight contiguous amino acids of SEQ ID NO:50; and

(c) the amino acid sequence encoded by the cDNA insert of clone qf116_{—}2 deposited under accession number ATCC 98808;

the protein being substantially free from other mammalian proteins. Preferably such protein comprises the amino acid sequence of SEQ ID NO:50. In further preferred embodiments, the present invention provides a protein comprising a fragment of the amino acid sequence of SEQ ID NO:50 having biological activity, the fragment preferably comprising eight (more preferably twenty, most preferably thirty) contiguous amino acids of SEQ ID NO:50, or a protein comprising a fragment of the amino add sequence of SEQ ID NO:50 having biological activity, the fragment comprising the amino acid sequence from amino acid 147 to amino acid 156 of SEQ ID NO:50.

In one embodiment, the present invention provides a composition comprising an isolated polynucleotide selected from the group consisting of:

(a) a polynucleotide comprising the nucleotide sequence of SEQ ID NO:51;

(b) a polynucleotide comprising the nucleotide sequence of SEQ ID NO:51 from nucleotide 160 to nucleotide 741;

(c) a polynucleotide comprising the nucleotide sequence of SEQ ID NO:51 from nucleotide 595 to nucleotide 741;

(d) a polynucleotide comprising the nucleotide sequence of the full-length protein coding sequence of clone qf662_{—}3 deposited under accession number ATCC 98808;

(e) a polynucleotide encoding the full-length protein encoded by the cDNA insert of clone qf662_{—}3 deposited under accession number ATCC 98808;

(f) a polynucleotide comprising the nucleotide sequence of a mature protein coding sequence of clone qf662_{—}3 deposited under accession number ATCC 98808;

(g) a polynucleotide encoding a mature protein encoded by the cDNA insert of clone qf662_{—}3 deposited under accession number ATCC 98808;

(h) a polynucleotide encoding a protein comprising the amino acid sequence of SEQ ID NO:52;

(i) a polynucleotide encoding a protein comprising a fragment of the amino acid sequence of SEQ ID NO:52 having biological activity, the fragment comprising eight contiguous amino acids of SEQ ID NO:52;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:51.

Preferably, such polynucleotide comprises the nucleotide sequence of SEQ ID NO:51 from nucleotide 160 to nucleotide 741; the nucleotide sequence of SEQ ID NO:51 from nucleotide 595 to nucleotide 741; the nucleotide sequence of the full-length protein coding sequence of clone qf662_{—}3 deposited under accession number ATCC 98808; or the nucleotide sequence of a mature protein coding sequence of clone qf662_{—}3 deposited under accession number ATCC 98808. In other preferred embodiments, the polynucleotide encodes the full-length or a mature protein encoded by the cDNA insert of clone qf62_{—}3 deposited under accession number ATCC 98808. In further preferred embodiments, the present invention provides a polynucleotide encoding a protein comprising a fragment of the amino acid sequence of SEQ ID NO:52 having biological activity, the fragment preferably comprising eight (more preferably twenty, most preferably thirty) contiguous amino acids of SEQ ID NO:52, or a polynucleotide encoding a protein comprising a fragment of the amino acid sequence of SEQ ID NO:52 having biological activity, the fragment comprising the amino acid sequence from amino acid 92 to amino acid 101 of SEQ ID NO:52.

Other embodiments provide the gene corresponding to the cDNA sequence of SEQ ID NO:51.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:51, but excluding the poly(A) tail at the 3′ end of SEQ ID NO:51; and

(ab) the nucleotide sequence of the cDNA insert of clone qf662_{—}3 deposited under accession number ATCC 98808;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of.

(ba) SEQ ID NO:51, but excluding the poly(A) tail at the 3′ end of SEQ ID NO:51; and

(bb) the nucleotide sequence of the cDNA insert of clone qf662_{—}3 deposited under accession number ATCC 98808;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:51, and extending contiguously from a nucleotide sequence corresponding to the 5′ end of SEQ ID NO:51 to a nucleotide sequence corresponding to the 3′ end of SEQ ID NO:51, but excluding the poly(A) tail at the 3′ end of SEQ ID NO:51. Also preferably the polynucleotide isolated according to the above process comprises a nucleotide sequence corresponding to the cDNA sequence of SEQ ID NO:51 from nucleotide 160 to nucleotide 741, and extending contiguously from a nucleotide sequence corresponding to the 5′ end of said sequence of SEQ ID NO:51 from nucleotide 160 to nucleotide 741, to a nucleotide sequence corresponding to the 3′ end of said sequence of SEQ ID NO:51 from nucleotide 160 to nucleotide 741. Also preferably the polynucleotide isolated according to the above process comprises a nucleotide sequence corresponding to the cDNA sequence of SEQ ID NO:51 from nucleotide 595 to nucleotide 741, and extending contiguously from a nucleotide sequence corresponding to the 5′ end of said sequence of SEQ ID NO:51 from nucleotide 595 to nucleotide 741, to a nucleotide sequence corresponding to the 3′ end of said sequence of SEQ ID NO:51 from nucleotide 595 to nucleotide 741.

In other embodiments, the present invention provides a composition comprising a protein, wherein said protein comprises an amino add sequence selected from the group consisting of:

(a) the amino acid sequence of SEQ ID NO:52;

(b) a fragment of the amino add sequence of SEQ ID NO:52, the fragment comprising eight contiguous amino adds of SEQ ID NO:52; and

(c) the amino acid sequence encoded by the cDNA insert of clone qf662_{—}3 deposited under accession number ATCC 98808;

the protein being substantially free from other mammalian proteins. Preferably such protein comprises the amino acid sequence of SEQ ID NO:52. In further preferred embodiments, the present invention provides a protein comprising a fragment of the amino acid sequence of SEQ ID NO:52 having biological activity, the fragment preferably comprising eight (more preferably twenty, most preferably thirty) contiguous amino adds of SEQ ID NO:52, or a protein comprising a fragment of the amino acid sequence of SEQ ID NO:52 having biological activity, the fragment comprising the amino add sequence from amino add 92 to amino acid 101 of SEQ ID NO:52.

In one embodiment, the present invention provides a composition comprising an isolated polynucleotide selected from the group consisting of:

(a) a polynucleotide comprising the nucleotide sequence of SEQ ID NO:53;

(b) a polynucleotide comprising the nucleotide sequence of SEQ ID NO:53 from nucleotide 924 to nucleotide 1196;

(c) a polynucleotide comprising the nucleotide sequence of SEQ ID NO:53 from nucleotide 1002 to nucleotide 1196;

(d) a polynucleotide comprising the nucleotide sequence of the full-length protein coding sequence of clone am748_{—}5 deposited under accession number ATCC 98817;

(e) a polynucleotide encoding the full-length protein encoded by the cDNA insert of clone am748_{—}5 deposited under accession number ATCC 98817;

(f) a polynucleotide comprising the nucleotide sequence of a mature protein coding sequence of clone am748_{—}5 deposited under accession number ATCC 98817;

(g) a polynucleotide encoding a mature protein encoded by the cDNA insert of clone am748_{—}5 deposited under accession number ATCC 98817;

(h) a polynucleotide encoding a protein comprising the amino acid sequence of SEQ ID NO:54;

(i) a polynucleotide encoding a protein comprising a fragment of the amino add sequence of SEQ ID NO:54 having biological activity, the fragment comprising eight contiguous amino acids of SEQ ID NO:54;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:53.

Preferably, such polynucleotide comprises the nucleotide sequence of SEQ ID NO:53 from nucleotide 924 to nucleotide 1196; the nucleotide sequence of SEQ ID NO:53 from nucleotide 1002 to nucleotide 1196; the nucleotide sequence of the full-length protein coding sequence of clone am748_{—}5 deposited under accession number ATCC 98817; or the nucleotide sequence of a mature protein coding sequence of clone am748_{—}5 deposited under accession number ATCC 98817. In other preferred embodiments, the polynucleotide encodes the full-length or a mature protein encoded by the cDNA insert of clone am748_{—}5 deposited under accession number ATCC 98817. In further preferred embodiments, the present invention provides a polynucleotide encoding a protein comprising a fragment of the amino acid sequence of SEQ ID NO:54 having biological activity, the fragment preferably comprising eight (more preferably twenty, most preferably thirty) contiguous amino adds of SEQ ID NO:54, or a polynucleotide encoding a protein comprising a fragment of the amino acid sequence of SEQ ID NO:54 having biological activity, the fragment comprising the amino acid sequence from amino acid 40 to amino acid 49 of SEQ ID NO:54.

Other embodiments provide the gene corresponding to the cDNA sequence of SEQ ID NO:53.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO-53, but excluding the poly(A) tail at the 3′ end of SEQ ID NO:53; and

(ab) the nucleotide sequence of the cDNA insert of clone am748_{—}5 deposited under accession number ATCC 98817;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:53, but excluding the poly(A) tail at the 3′ end of SEQ ID NO:53; and

(bb) the nucleotide sequence of the cDNA insert of clone am748_{—}5 deposited under accession number ATCC 98817;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)-(iii).

Preferably the polynucleotide isolated according to the above process comprises a nucleotide sequence corresponding to the cDNA sequence of SEQ ID NO:53, and extending contiguously from a nucleotide sequence corresponding to the 5′ end of SEQ ID NO:53 to a nucleotide sequence corresponding to the 3′ end of SEQ ID NO:53, but excluding the poly(A) tail at the 3′ end of SEQ ID NO:53. Also preferably the polynucleotide isolated according to the above process comprises a nucleotide sequence corresponding to the cDNA sequence of SEQ ID NO:53 from nucleotide 924 to nucleotide 1196, and extending contiguously from a nucleotide sequence corresponding to the 5′ end of said sequence of SEQ ID NO:53 from nucleotide 924 to nucleotide 1196, to a nucleotide sequence corresponding to the 3′ end of said sequence of SEQ ID NO:53 from nucleotide 924 to nucleotide 1196. Also preferably the polynucleotide isolated according to the above process comprises a nucleotide sequence corresponding to the cDNA sequence of SEQ ID NO:53 from nucleotide 1002 to nucleotide 1196, and extending contiguously from a nucleotide sequence corresponding to the 5′ end of said sequence of SEQ ID NO:53 from nucleotide 1002 to nucleotide 1196, to a nucleotide sequence corresponding to the 3′ end of said sequence of SEQ ID NO:53 from nucleotide 1002 to nucleotide 1196.

In other embodiments, the present invention provides a composition comprising a protein, wherein said protein comprises an amino add sequence selected from the group consisting of:

(a) the amino acid sequence of SEQ ID NO:54;

(b) a fragment of the amino acid sequence of SEQ ID NO:54, the fragment comprising eight contiguous amino acids of SEQ ID NO:54; and

(c) the amino add sequence encoded by the cDNA insert of clone am748_{—}5 deposited under accession number ATCC 98817;

the protein being substantially free from other mammalian proteins. Preferably such protein comprises the amino add sequence of SEQ ID NO:54. In further preferred embodiments, the present invention provides a protein comprising a fragment of the amino acid sequence of SEQ ID NO:54 having biological activity, the fragment preferably comprising eight (more preferably twenty, most preferably thirty) contiguous amino acids of SEQ ID NO:54, or a protein comprising a fragment of the amino acid sequence of SEQ ID NO:54 having biological activity, the fragment comprising the amino acid sequence from amino acid 40 to amino acid 49 of SEQ ID NO:54.

In one embodiment, the present invention provides a composition comprising an isolated polynucleotide selected from the group consisting of:

(a) a polynucleotide comprising the nucleotide sequence of SEQ ID NO:55;

(b) a polynucleotide comprising the nucleotide sequence of SEQ ID NO:55 from nucleotide 51 to nucleotide 1310;

(c) a polynucleotide comprising the nucleotide sequence of the full-length protein coding sequence of clone cj507_{—}1 deposited under accession number ATCC 98817;

(d) a polynucleotide encoding the full-length protein encoded by the cDNA insert of clone cj5071 deposited under accession number ATCC 98817;

(e) a polynucleotide comprising the nucleotide sequence of a mature protein coding sequence of clone cj507_{—}1 deposited under accession number ATCC 98817;

(f) a polynucleotide encoding a mature protein encoded by the cDNA insert of clone cj507_{—}1 deposited under accession number ATCC 98817;

(g) a polynucleotide encoding a protein comprising the amino acid sequence of SEQ ID NO:56;

(h) a polynucleotide encoding a protein comprising a fragment of the amino acid sequence of SEQ ID NO:56 having biological activity, the fragment comprising eight contiguous amino acids of SEQ ID NO:56;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:55.

Preferably, such polynucleotide comprises the nucleotide sequence of SEQ ID NO:55 from nucleotide 51 to nucleotide 1310; the nucleotide sequence of the full-length protein coding sequence of clone cj507_{—}1 deposited under accession number ATCC 98817; or the nucleotide sequence of a mature protein-coding sequence of clone cj507_deposited under accession number ATCC 98817. In other preferred embodiments, the polynucleotide encodes the full-length or a mature protein encoded by the cDNA insert of clone cj507_{—}1 deposited under accession number ATCC 98817. In further preferred embodiments, the present invention provides a polynucleotide encoding a protein comprising a fragment of the amino acid sequence of SEQ ID NO:56 having biological activity, the fragment preferably comprising eight (more preferably twenty, most preferably thirty) contiguous amino acids of SEQ ID NO:56, or a polynucleotide encoding a protein comprising a fragment of the amino acid sequence of SEQ ID NO:56 having biological activity, the fragment comprising the amino acid sequence from amino acid 205 to amino acid 214 of SEQ ID NO:56.

Other embodiments provide the gene corresponding to the cDNA sequence of SEQ ID NO:55.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:55, but excluding the poly(A) tail at the 3′ end of SEQ ID NO:55; and

(ab) the nucleotide sequence of the cDNA insert of clone cj507_{—}1 deposited under accession number ATCC 98817;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:55, but excluding the poly(A) tail at the 3′ end of SEQ ID NO 55; and

(bb) the nucleotide sequence of the cDNA insert of clone cj507_{—}1 deposited under accession number ATCC 98817;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:55, and extending contiguously from a nucleotide sequence corresponding to the 5′ end of SEQ ID NO:55 to a nucleotide sequence corresponding to the 3′ end of SEQ ID NO:55, but excluding the poly(A) tail at the 3′ end of SEQ ID NO:55. Also preferably the polynucleotide isolated according to the above process comprises a nucleotide sequence corresponding to the cDNA sequence of SEQ ID NO:55 from nucleotide 51 to nucleotide 1310, and extending contiguously from a nucleotide sequence corresponding to the 5′ end of said sequence of SEQ ID NO:55 from nucleotide 51 to nucleotide 1310, to a nucleotide sequence corresponding to the 3′ end of said sequence of SEQ ID NO:55 from nucleotide 51 to nucleotide 1310.

In other embodiments, the present invention provides a composition comprising a protein, wherein said protein comprises an amino acid sequence selected from the group consisting of:

(a) the amino acid sequence of SEQ ID NO:56;

(b) a fragment of the amino acid sequence of SEQ ID NO:56, the fragment comprising eight contiguous amino acids of SEQ ID NO:56; and

(c) the amino acid sequence encoded by the cDNA insert of clone cj507_{—}1 deposited under accession number ATCC 98817;

the protein being substantially free from other mammalian proteins. Preferably such protein comprises the amino acid sequence of SEQ ID NO:56. In further preferred embodiments, the present invention provides a protein comprising a fragment of the amino acid sequence of SEQ ID NO:56 having biological activity, the fragment preferably comprising eight (more preferably twenty, most preferably thirty) contiguous amino acids of SEQ ID NO:56, or a protein comprising a fragment of the amino acid sequence of SEQ ID NO:56 having biological activity, the fragment comprising the amino acid sequence from amino acid 205 to amino acid 214 of SEQ ID NO:56.

In one embodiment, the present invention provides a composition comprising an isolated polynucleotide selected from the group consisting of:

(a) a polynucleotide comprising the nucleotide sequence of SEQ ID NO:57;

(b) a polynucleotide comprising the nucleotide sequence of SEQ ID NO:57 from nucleotide 195 to nucleotide 1328;

(c) a polynucleotide comprising the nucleotide sequence of the full-length protein coding sequence of clone cn922_{—}5 deposited under accession number ATCC 98817;

(d) a polynucleotide encoding the full-length protein encoded by the cDNA insert of clone cn922_{—}5 deposited under accession number ATCC 98817;

(e) a polynucleotide comprising the nucleotide sequence of a mature protein coding sequence of clone cn922_{—}5 deposited under accession number ATCC 98817;

(f) a polynucleotide encoding a mature protein encoded by the cDNA insert of clone cn922_{—}5 deposited under accession number ATCC 98817;

(g) a polynucleotide encoding a protein comprising the amino acid sequence of SEQ ID NO:58;

(h) a polynucleotide encoding a protein comprising a fragment of the amino acid sequence of SEQ ID NO:58 having biological activity, the fragment comprising eight contiguous amino acids of SEQ ID NO:58;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:57.

Preferably, such polynucleotide comprises the nucleotide sequence of SEQ ID NO:57 from nucleotide 195 to nucleotide 1328; the nucleotide sequence of the full-length protein coding sequence of clone cn922_{—}5 deposited under accession number ATCC 98817; or the nucleotide sequence of a mature protein coding sequence of clone cn922_{—}5 deposited under accession number ATCC 98817. In other preferred embodiments, the polynucleotide encodes the full-length or a mature protein encoded by the cDNA insert of clone cn922_{—}5 deposited under accession number ATCC 98817. In further preferred embodiments, the present invention provides a polynucleotide encoding a protein comprising a fragment of the amino acid sequence of SEQ ID NO:58 having biological activity, the fragment preferably comprising eight (more preferably twenty, most preferably thirty) contiguous amino acids of SEQ ID NO:58, or a polynucleotide encoding a protein comprising a fragment of the amino add sequence of SEQ ID NO:58 having biological activity, the fragment comprising the amino add sequence from amino acid 184 to amino acid 193 of SEQ ID NO:58.

Other embodiments provide the gene corresponding to the cDNA sequence of SEQ ID NO:57.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:57, but excluding the poly(A) tail at the 3′ end of SEQ ID NO:57; and

(ab) the nucleotide sequence of the cDNA insert of clone cn922_{—}5 deposited under accession number ATCC 98817;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:57, but excluding the poly(A) tail at the 3′ end of SEQ ID NO:57; and

(bb) the nucleotide sequence of the cDNA insert of clone cn922_{—}5 deposited under accession number ATCC 98817;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:57, and extending contiguously from a nucleotide sequence corresponding to the 5′ end of SEQ ID NO:57 to a nucleotide sequence corresponding to the 3′ end of SEQ ID NO:57, but excluding the poly(A) tail at the 3′ end of SEQ ID NO:57. Also preferably the polynucleotide isolated according to the above process comprises a nucleotide sequence corresponding to the cDNA sequence of SEQ ID NO:57 from nucleotide 195 to nucleotide 1328, and extending contiguously from a nucleotide sequence corresponding to the 5′ end of said sequence of SEQ ID NO:57 from nucleotide 195 to nucleotide 1328, to a nucleotide sequence corresponding to the 3′ end of said sequence of SEQ ID NO:57 from nucleotide 195 to nucleotide 1328.

In other embodiments, the present invention provides a composition comprising a protein, wherein said protein comprises an amino add sequence selected from the group consisting of:

(a) the amino acid sequence of SEQ ID NO:58;

(b) a fragment of the amino acid sequence of SEQ ID NO:58, the fragment comprising eight contiguous amino acids of SEQ ID NO:58; and

(c) the amino acid sequence encoded by the cDNA insert of clone cn922_{—}5 deposited under accession number ATCC 98817;

the protein being substantially free from other mammalian proteins. Preferably such protein comprises the amino acid sequence of SEQ ID NO:58. In further preferred embodiments, the present invention provides a protein comprising a fragment of the amino acid sequence of SEQ ID NO-58 having biological activity, the fragment preferably comprising eight (more preferably twenty, most preferably thirty) contiguous amino adds of SEQ ID NO:58, or a protein comprising a fragment of the amino acid sequence of SEQ ID NO:58 having biological activity, the fragment comprising the amino add sequence from amino acid 184 to amino acid 193 of SEQ ID NO:58.

In one embodiment, the present invention provides a composition comprising an isolated polynucleotide selected from the group consisting of:

(a) a polynucleotide comprising the nucleotide sequence of SEQ ID NO:59;

(b) a polynucleotide comprising the nucleotide sequence of SEQ ID NO:59 from nucleotide 76 to nucleotide 942;

(c) a polynucleotide comprising the nucleotide sequence of the full-length protein coding sequence of clone cw691_{—}11 deposited under accession number ATCC 98817;

(d) a polynucleotide encoding the full-length protein encoded by the cDNA insert of clone cw691_{—}11 deposited under accession number ATCC 98817;

(e) a polynucleotide comprising the nucleotide sequence of a mature protein coding sequence of clone cw691_{—}11 deposited under accession number ATCC 98817;

(f) a polynucleotide encoding a mature protein encoded by the cDNA insert of clone cw691_{—}11 deposited under accession number ATCC 98817;

(g) a polynucleotide encoding a protein comprising the amino acid sequence of SEQ ID NO:60;

(h) a polynucleotide encoding a protein comprising a fragment of the amino acid sequence of SEQ ID NO:60 having biological activity, the fragment comprising eight contiguous amino acids of SEQ ID NO:60;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:59.

Preferably, such polynucleotide comprises the nucleotide sequence of SEQ ID NO:59 from nucleotide 76 to nucleotide 942; the nucleotide sequence of the full-length protein coding sequence of clone cw691_{—}11 deposited under accession number ATCC 98817; or the nucleotide sequence of a mature protein coding sequence of clone cw691_{—}11 deposited under accession number ATCC 98817. In other preferred embodiments, the polynucleotide encodes the full-length or a mature protein encoded by the cDNA insert of clone cw691_{—}11 deposited under accession number ATCC 98817. In further preferred embodiments, the present invention provides a polynucleotide encoding a protein comprising a fragment of the amino acid sequence of SEQ ID NO:60 having biological activity, the fragment preferably comprising eight (more preferably twenty, most preferably thirty) contiguous amino acids of SEQ ID NO:60, or a polynucleotide encoding a protein comprising a fragment of the amino add sequence of SEQ ID NO:60 having biological activity, the fragment comprising the amino add sequence from amino add 139 to amino acid 148 of SEQ ID NO:60.

Other embodiments provide the gene corresponding to the cDNA sequence of SEQ ID NO:59.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:59, but excluding the poly(A) tail at the 3′ end of SEQ ID NO:59; and

(ab) the nucleotide sequence of the cDNA insert of clone cw691_{—}11 deposited under accession number ATCC 98817;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of

(ba) SEQ ID NO:59, but excluding the poly(A) tail at the 3′ end of SEQ ID NO:59; and

(bb) the nucleotide sequence of the cDNA insert of clone cw691_{—}11 deposited under accession number ATCC 98817;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:59, and extending contiguously from a nucleotide sequence corresponding to the 5′ end of SEQ ID NO:59 to a nucleotide sequence corresponding to the 3′ end of SEQ ID NO:59, but excluding the poly(A) tail at the 3′ end of SEQ ID NO:59. Also preferably the polynucleotide isolated according to the above process comprises a nucleotide sequence corresponding to the cDNA sequence of SEQ ID NO:59 from nucleotide 76 to nucleotide 942, and extending contiguously from a nucleotide sequence corresponding to the 5′ end of said sequence of SEQ ID NO:59 from nucleotide 76 to nucleotide 942, to a nucleotide sequence corresponding to the 3′ end of said sequence of SEQ ID NO:59 from nucleotide 76 to nucleotide 942.

In other embodiments, the present invention provides a composition comprising a protein, wherein said protein comprises an amino acid sequence selected from the group consisting of:

(a) the amino acid sequence of SEQ ID NO:60;

(b) a fragment of the amino acid sequence of SEQ ID NO:60, the fragment comprising eight contiguous amino acids of SEQ ID NO:0.60; and

(c) the amino acid sequence encoded by the cDNA insert of clone cw691_{—}11 deposited under accession number ATCC 98817;

the protein being substantially free from other mammalian proteins. Preferably such protein comprises the amino acid sequence of SEQ ID NO:60. In further preferred embodiments, the present invention provides a protein comprising a fragment of the amino acid sequence of SEQ ID NO:60 having biological activity, the fragment preferably comprising eight (more preferably twenty, most preferably thirty) contiguous amino acids of SEQ ID NO:60, or a protein comprising a fragment of the amino acid sequence of SEQ ID NO:60 having biological activity, the fragment comprising the amino acid sequence from amino acid 139 to amino acid 148 of SEQ ID NO:60.

In one embodiment, the present invention provides a composition comprising an isolated polynucleotide selected from the group consisting of:

(a) a polynucleotide comprising the nucleotide sequence of SEQ ID NO:61;

(b) a polynucleotide comprising the nucleotide sequence of SEQ ID NO:61 from nucleotide 11 to nucleotide 1252;

(c) a polynucleotide comprising the nucleotide sequence of SEQ ID NO:61 from nucleotide 119 to nucleotide 1252;

(d) a polynucleotide comprising the nucleotide sequence of the full-length protein coding sequence of clone cw1000_{—}2 deposited under accession number ATCC 98817;

(e) a polynucleotide encoding the full-length protein encoded by the cDNA insert of clone cw1000_{—}2 deposited under accession number ATCC 98817;

(f) a polynucleotide comprising the nucleotide sequence of a mature protein coding sequence of clone cw1000_{—2} deposited under accession number ATCC 98817;

(g) a polynucleotide encoding a mature protein encoded by the cDNA insert of clone cw1000_{—}2 deposited under accession number ATCC 98817;

(h) a polynucleotide encoding a protein comprising the amino acid sequence of SEQ ID NO:62;

(i) a polynucleotide encoding a protein comprising a fragment of the amino acid sequence of SEQ ID NO:62 having biological activity, the fragment comprising eight contiguous amino acids of SEQ ID NO:62;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:61.

Preferably, such polynucleotide comprises the nucleotide sequence of SEQ ID NO:61 from nucleotide 11 to nucleotide 1252; the nucleotide sequence of SEQ ID NO:61 from nucleotide 119 to nucleotide 1252; the nucleotide sequence of the full-length protein coding sequence of clone cw1000_{—}2 deposited under accession number ATCC 98817; or the nucleotide sequence of a mature protein coding sequence of clone cw1000_{—}2 deposited under accession number ATCC 98817. In other preferred embodiments, the polynucleotide encodes the full-length or a mature protein encoded by the cDNA insert of clone cw1000_{—}2 deposited under accession number ATCC 98817. In further preferred embodiments, the present invention provides a polynucleotide encoding a protein comprising a fragment of the amino acid sequence of SEQ ID NO:62 having biological activity, the fragment preferably comprising eight (more preferably twenty, most preferably thirty) contiguous amino acids of SEQ ID NO:62, or a polynucleotide encoding a protein comprising a fragment of the amino acid sequence of SEQ ID NO:62 having biological activity, the fragment comprising the amino acid sequence from amino acid 202 to amino acid 211 of SEQ ID NO:62.

Other embodiments provide the gene corresponding to the cDNA sequence of SEQ ID NO:61.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:61, but excluding the poly(A) tail at the 3′ end of SEQ ID NO:61; and

(ab) the nucleotide sequence of the cDNA insert of clone cw1000_{—}2 deposited under accession number ATCC 98817;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:61, but excluding the poly(A) tail at the 3′ end of SEQ ID NO:61; and

(bb) the nucleotide sequence of the cDNA insert of clone cw1000_{—}2 deposited under accession number ATCC 98817;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:61, and extending contiguously from a nucleotide sequence corresponding to the 5′ end of SEQ ID NO:61 to a nucleotide sequence corresponding to the 3′ end of SEQ ID NO:61, but excluding the poly(A) tail at the 3′ end of SEQ ID NO:61. Also preferably the polynucleotide isolated according to the above process comprises a nucleotide sequence corresponding to the cDNA sequence of SEQ ID NO:61 from nucleotide 11 to nucleotide 1252, and extending contiguously from a nucleotide sequence corresponding to the 5′ end of said sequence of SEQ ID NO:61 from nucleotide 11 to nucleotide 1252, to a nucleotide sequence corresponding to the 3′ end of said sequence of SEQ ID NO:61 from nucleotide 11 to nucleotide 1252. Also preferably the polynucleotide isolated according to the above process comprises a nucleotide sequence corresponding to the cDNA sequence of SEQ ID NO:61 from nucleotide 119 to nucleotide 1252, and extending contiguously from a nucleotide sequence corresponding to the 5′ end of said sequence of SEQ ID NO:61 from nucleotide 119 to nucleotide 1252, to a nucleotide sequence corresponding to the 3′ end of said sequence of SEQ ID NO:61 from nucleotide 119 to nucleotide 1252.

In other embodiments, the present invention provides a composition comprising a protein, wherein said protein comprises an amino acid sequence selected from the group consisting of:

(a) the amino acid sequence of SEQ ID NO:62;

(b) a fragment of the amino acid sequence of SEQ ID NO:62, the fragment comprising eight contiguous amino acids of SEQ ID NO: 62; and

(c) the amino acid sequence encoded by the cDNA insert of clone cw1000_{—}2 deposited under accession number ATCC 98817;

the protein being substantially free from other mammalian proteins. Preferably such protein comprises the amino add sequence of SEQ ID NO:62. In further preferred embodiments, the present invention provides a protein comprising a fragment of the amino add sequence of SEQ ID NO:62 having biological activity, the fragment preferably comprising eight (more preferably twenty, most preferably thirty) contiguous amino adds of SEQ ID NO:62, or a protein comprising a fragment of the amino add sequence of SEQ ID NO:62 having biological activity, the fragment comprising the amino add sequence from amino add 202 to amino acid 211 of SEQ ID NO:62.

In one embodiment, the present invention provides a composition comprising an isolated polynucleotide selected from the group consisting of:

(a) a polynucleotide comprising the nucleotide sequence of SEQ ID NO:63;

(b) a polynucleotide comprising the nucleotide sequence of SEQ ID NO:63 from nucleotide 46 to nucleotide 1296;

(c) a polynucleotide comprising the nucleotide sequence of SEQ ID NO:63 from nucleotide 451 to nucleotide 1296;

(d) a polynucleotide comprising the nucleotide sequence of the full-length protein coding sequence of clone cw1640_{—}1 deposited under accession number ATCC 98817;

(e) a polynucleotide encoding the full-length protein encoded by the cDNA insert of clone cw1640_{—}1 deposited under accession number ATCC 98817;

(f) a polynucleotide comprising the nucleotide sequence of a mature protein coding sequence of clone cw1640_{—}1 deposited under accession number ATCC 98817;

(g) a polynucleotide encoding a mature protein encoded by the cDNA insert of clone cw1640_{—}1 deposited under accession number ATCC 98817;

(h) a polynucleotide encoding a protein comprising the amino add sequence of SEQ ID NO:64;

(i) a polynucleotide encoding a protein comprising a fragment of the amino acid sequence of SEQ ID NO:64 having biological activity, the fragment comprising eight contiguous amino acids of SEQ ID NO:64;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:63.

Preferably, such polynucleotide comprises the nucleotide sequence of SEQ ID NO:63 from nucleotide 46 to nucleotide 1296; the nucleotide sequence of SEQ ID NO:63 from nucleotide 451 to nucleotide 1296; the nucleotide sequence of the full-length protein coding sequence of clone cw1640_{—}1 deposited under accession number ATCC 98817; or the nucleotide sequence of a mature protein coding sequence of clone cw1640_{—}1 deposited under accession number ATCC 98817. In other preferred embodiments, the polynucleotide encodes the full-length or a mature protein encoded by the cDNA insert of clone cw1640_{—}1 deposited under accession number ATCC 98817. In further preferred embodiments, the present invention provides a polynucleotide encoding a protein comprising a fragment of the amino acid sequence of SEQ ID NO:64 having biological activity, the fragment preferably comprising eight (more preferably twenty, most preferably thirty) contiguous amino acids of SEQ ID NO:64, or a polynucleotide encoding a protein comprising a fragment of the amino add sequence of SEQ ID NO:64 having biological activity, the fragment comprising the amino acid sequence from amino acid 203 to amino acid 212 of SEQ ID NO:64.

Other embodiments provide the gene corresponding to the cDNA sequence of SEQ ID NO:63.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:63, but excluding the poly(A) tail at the 3′ end of SEQ ID NO:63; and

(ab) the nucleotide sequence of the cDNA insert of clone cw1640_{—}1 deposited under accession number ATCC 98817;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:63, but excluding the poly(A) tail at the 3′ end of SEQ ID NO:63; and

(bb) the nucleotide sequence of the cDNA insert of clone cw1640_{—}1 deposited under accession number ATCC 98817;

(ii) hybridizing said primer(s) to human genomic DNA in conditions at least as stringent as 4×SSC at 50 degrees C.;

(ii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:63, and extending contiguously from a nucleotide sequence corresponding to the 5′ end of SEQ ID NO:63 to a nucleotide sequence corresponding to the 3′ end of SEQ ID NO:63, but excluding the poly(A) tail at the 3′ end of SEQ ID NO:63. Also preferably the polynucleotide isolated according to the above process comprises a nucleotide sequence corresponding to the cDNA sequence of SEQ ID NO:63 from nucleotide 46 to nucleotide 1296, and extending contiguously from a nucleotide sequence corresponding to the 5′ end of said sequence of SEQ ID NO:63 from nucleotide 46 to nucleotide 1296, to a nucleotide sequence corresponding to the 3′ end of said sequence of SEQ ID NO:63 from nucleotide 46 to nucleotide 1296. Also preferably the polynucleotide isolated according to the above process comprises a nucleotide sequence corresponding to the cDNA sequence of SEQ ID NO:63 from nucleotide 451 to nucleotide 1296, and extending contiguously from a nucleotide sequence corresponding to the 5′ end of said sequence of SEQ ID NO:63 from nucleotide 451 to nucleotide 1296, to a nucleotide sequence corresponding to the 3′ end of said sequence of SEQ ID NO:63 from nucleotide 451 to nucleotide 1296.

In other embodiments, the present invention provides a composition comprising a protein, wherein said protein comprises an amino add sequence selected from the group consisting of:

(a) the amino acid sequence of SEQ ID NO:64;

(b) a fragment of the amino acid sequence of SEQ ID NO:64, the fragment comprising eight contiguous amino acids of SEQ ID NO:64; and

(c) the amino add sequence encoded by the cDNA insert of clone cw1640_{—}1 deposited under accession number ATCC 98817;

the protein being substantially free from other mammalian proteins. Preferably such protein comprises the amino acid sequence of SEQ ID NO:64. In further preferred embodiments, the present invention provides a protein comprising a fragment of the amino add sequence of SEQ ID NO:64 having biological activity, the fragment preferably comprising eight (more preferably twenty, most preferably thirty) contiguous amino acids of SEQ ID NO:64, or a protein comprising a fragment of the amino acid sequence of SEQ ID NO:64 having biological activity, the fragment comprising the amino acid sequence from amino acid 203 to amino acid 212 of SEQ ID NO:64.

In one embodiment, the present invention provides a composition comprising an isolated polynucleotide selected from the group consisting of:

(a) a polynucleotide comprising the nucleotide sequence of SEQ ID NO:65;

(b) a polynucleotide comprising the nucleotide sequence of SEQ ID NO:65 from nucleotide 66 to nucleotide 827;

(c) a polynucleotide comprising the nucleotide sequence of SEQ ID NO:65 from nucleotide 474 to nucleotide 827;

(d) a polynucleotide comprising the nucleotide sequence of the full-length protein coding sequence of clone d24_{—}1 deposited under accession number ATCC 98817;

(e) a polynucleotide encoding the full-length protein encoded by the cDNA insert of clone d24_{—}1 deposited under accession number ATCC 98817;

(f) a polynucleotide comprising the nucleotide sequence of a mature protein coding sequence of clone d24_{—}1 deposited under accession number ATCC 98817;

(g) a polynucleotide encoding a mature protein encoded by the cDNA insert of clone d24_{—}1 deposited under accession number ATCC 98817;

(h) a polynucleotide encoding a protein comprising the amino acid sequence of SEQ ID NO:66;

(i) a polynucleotide encoding a protein comprising a fragment of the amino acid sequence of SEQ ID NO:66 having biological activity, the fragment comprising eight contiguous amino acids of SEQ ID NO:66;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:65.

Preferably, such polynucleotide comprises the nucleotide sequence of SEQ ID NO:65 from nucleotide 66 to nucleotide 827; the nucleotide sequence of SEQ ID NO:65 from nucleotide 474 to nucleotide 827; the nucleotide sequence of the full-length protein coding sequence of clone d24_{—}1 deposited under accession number ATCC 98817; or the nucleotide sequence of a mature protein coding sequence of clone d24_{—}1 deposited under accession number ATCC 98817. In other preferred embodiments, the polynucleotide encodes the full-length or a mature protein encoded by the cDNA insert of clone d24_{—}1 deposited under accession number ATCC 98817. In further preferred embodiments, the present invention provides a polynucleotide encoding a protein comprising a fragment of the amino acid sequence of SEQ ID NO:66 having biological activity, the fragment preferably comprising eight (more preferably twenty, most preferably thirty) contiguous amino acids of SEQ ID NO:66, or a polynucleotide encoding a protein comprising a fragment of the amino acid sequence of SEQ ID NO:66 having biological activity, the fragment comprising the amino acid sequence from amino add 122 to amino acid 131 of SEQ ID NO:66.

Other embodiments provide the gene corresponding to the cDNA sequence of SEQ ID NO:65.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:65, but excluding the poly(A) tail at the 3′ end of SEQ ID NO:65; and

(ab) the nucleotide sequence of the cDNA insert of clone d24_{—}1 deposited under accession number ATCC 98817;

(ii) hybridizing said probe(s) to human genomic DNA in conditions at least as stringent as 4×SSC at 50 degrees C.; and

(ii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:65, but excluding the poly(A) tail at the 3′ end of SEQ ID NO:65; and

(bb) the nucleotide sequence of the cDNA insert of clone d24_{—}1 deposited under accession number ATCC 98817;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:65, and extending contiguously from a nucleotide sequence corresponding to the 5′ end of SEQ ID NO:65 to a nucleotide sequence corresponding to the 3′ end of SEQ ID NO:65, but excluding the poly(A) tail at the 3′ end of SEQ ID NO:65. Also preferably the polynucleotide isolated according to the above process comprises a nucleotide sequence corresponding to the cDNA sequence of SEQ ID NO:65 from nucleotide 66 to nucleotide 827, and extending contiguously from a nucleotide sequence corresponding to the 5′ end of said sequence of SEQ ID NO:65 from nucleotide 66 to nucleotide 827, to a nucleotide sequence corresponding to the 3′ end of said sequence of SEQ ID N 0:65 from nucleotide 66 to nucleotide 827. Also preferably the polynucleotide isolated according to the above process comprises a nucleotide sequence corresponding to the cDNA sequence of SEQ ID NO:65 from nucleotide 474 to nucleotide 827, and extending contiguously from a nucleotide sequence corresponding to the 5′ end of said sequence of SEQ ID NO:65 from nucleotide 474 to nucleotide 827, to a nucleotide sequence corresponding to the 3′ end of said sequence of SEQ ID NO:65 from nucleotide 474 to nucleotide 827.

In other embodiments, the present invention provides a composition comprising a protein, wherein said protein comprises an amino acid sequence selected from the group consisting of:

(a) the amino acid sequence of SEQ ID NO:66;

(b) a fragment of the amino acid sequence of SEQ ID NO:66, the fragment comprising eight contiguous amino acids of SEQ ID NO:66; and

(c) the amino add sequence encoded by the cDNA insert of clone d24_{—}1 deposited under accession number ATCC 98817;

the protein being substantially free from other mammalian proteins. Preferably such protein comprises the amino acid sequence of SEQ ID NO:66. In further preferred embodiments, the present invention provides a protein comprising a fragment of the amino acid sequence of SEQ ID NO:66 having biological activity, the fragment preferably comprising eight (more preferably twenty, most preferably thirty) contiguous amino acids of SEQ ID NO:66, or a protein comprising a fragment of the amino acid sequence of SEQ ID NO:66 having biological activity, the fragment comprising the amino acid sequence from amino acid 122 to amino acid 131 of SEQ ID NO:66.

In one embodiment, the present invention provides a composition comprising an isolated polynucleotide selected from the group consisting of

(a) a polynucleotide comprising the nucleotide sequence of SEQ ID NO:67;

(b) a polynucleotide comprising the nucleotide sequence of SEQ ID NO:67 from nucleotide 149 to nucleotide 529;

(c) a polynucleotide comprising the nucleotide sequence of SEQ ID NO:67 from nucleotide 413 to nucleotide 529;

(d) a polynucleotide comprising the nucleotide sequence of the full-length protein coding sequence of clone dd426_{—}1 deposited under accession number ATCC 98817;

(e) a polynucleotide encoding the full-length protein encoded by the cDNA insert of clone dd426_{—}1 deposited under accession number ATCC 98817;

(f) a polynucleotide comprising the nucleotide sequence of a mature protein coding sequence of clone dd426_{—}1 deposited under accession number ATCC 98817;

(g) a polynucleotide encoding a mature protein encoded by the cDNA insert of clone dd426_{—}1 deposited under accession number ATCC 98817;

(h) a polynucleotide encoding a protein comprising the amino acid sequence of SEQ ID NO:68;

(i) a polynucleotide encoding a protein comprising a fragment of the amino acid sequence of SEQ ID NO:68 having biological activity, the fragment comprising eight contiguous amino acids of SEQ ID NO:68;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)(i); and

(m) a polynucleotide that hybridizes under stringent conditions to any one of the polynucleotides specified in (a)-(i) and that has a length that is at least 25% of the length of SEQ ID NO:67.

Preferably, such polynucleotide comprises the nucleotide sequence of SEQ ID NO:67 from nucleotide 149 to nucleotide 529; the nucleotide sequence of SEQ ID NO:67 from nucleotide 413 to nucleotide 529; the nucleotide sequence of the full-length protein coding sequence of clone dd426_{—}1 deposited under accession number ATCC 98817; or the nucleotide sequence of a mature protein coding sequence of clone dd426_{—}1 deposited under accession number ATCC 98817. In other preferred embodiments, the polynucleotide encodes the full-length or a mature protein encoded by the cDNA insert of clone dd426_{—}1 deposited under accession number ATCC 98817. In further preferred embodiments, the present invention provides a polynucleotide encoding a protein comprising a fragment of the amino acid sequence of SEQ ID NO:68 having biological activity, the fragment preferably comprising eight (more preferably twenty, most preferably thirty) contiguous amino acids of SEQ ID NO:68, or a polynucleotide encoding a protein comprising a fragment of the amino acid sequence of SEQ ID NO:68 having biological activity, the fragment comprising the amino acid sequence from amino acid 58 to amino acid 67 of SEQ ID NO:68.

Other embodiments provide the gene corresponding to the cDNA sequence of SEQ ID NO:67.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:67, but excluding the poly(A) tail at the 3′ end of SEQ ID NO:67; and

(ab) the nucleotide sequence of the cDNA insert of clone dd426_{—}1 deposited under accession number ATCC 98817;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:67, but excluding the poly(A) tail at the 3′ end of SEQ ID NO:67; and

(bb) the nucleotide sequence of the cDNA insert of clone dd426_{—}1 deposited under accession number ATCC 98817;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:67, and extending contiguously from a nucleotide sequence corresponding to the 5′ end of SEQ ID NO:67 to a nucleotide sequence corresponding to the 3′ end of SEQ ID NO:67, but excluding the poly(A) tail at the 3′ end of SEQ ID NO:67. Also preferably the polynucleotide isolated according to the above process comprises a nucleotide sequence corresponding to the cDNA sequence of SEQ ID NO:67 from nucleotide 149 to nucleotide 529, and extending contiguously from a nucleotide sequence corresponding to the 5′ end of said sequence of SEQ ID NO:67 from nucleotide 149 to nucleotide 529, to a nucleotide sequence corresponding to the 3′ end of said sequence of SEQ ID NO:67 from nucleotide 149 to nucleotide 529. Also preferably the polynucleotide isolated according to the above process comprises a nucleotide sequence corresponding to the cDNA sequence of SEQ ID NO:67 from nucleotide 413 to nucleotide 529, and extending contiguously from a nucleotide sequence corresponding to the 5′ end of said sequence of SEQ ID NO:67 from nucleotide 413 to nucleotide 529, to a nucleotide sequence corresponding to the 3′ end of said sequence of SEQ ID NO:67 from nucleotide 413 to nucleotide 529.

In other embodiments, the present invention provides a composition comprising a protein, wherein said protein comprises an amino acid sequence selected from the group consisting of:

(a) the amino acid sequence of SEQ ID NO:68;

(b) a fragment of the amino acid sequence of SEQ ID NO:68, the fragment comprising eight contiguous amino acids of SEQ ID NO:68; and

(c) the amino acid sequence encoded by the cDNA insert of clone dd426_{—}1 deposited under accession number ATCC 98817;

the protein being substantially free from other mammalian proteins. Preferably such protein comprises the amino acid sequence of SEQ ID NO:68. In further preferred embodiments, the present invention provides a protein comprising a fragment of the amino acid sequence of SEQ ID NO:68 having biological activity, the fragment preferably comprising eight (more preferably twenty, most preferably thirty) contiguous amino acids of SEQ ID NO:68, or a protein comprising a fragment of the amino acid sequence of SEQ ID NO:68 having biological activity, the fragment comprising the amino acid sequence from amino acid 58 to amino acid 67 of SEQ ID NO:68.

In one embodiment, the present invention provides a composition comprising an isolated polynucleotide selected from the group consisting of:

(a) a polynucleotide comprising the nucleotide sequence of SEQ ID NO:69;

(b) a polynucleotide comprising the nucleotide sequence of SEQ ID NO:69 from nucleotide 31 to nucleotide 543;

(c) a polynucleotide comprising the nucleotide sequence of SEQ ID NO:69 from nucleotide 88 to nucleotide 543;

(d) a polynucleotide comprising the nucleotide sequence of the full-length protein coding sequence of clone di393_{—}2 deposited under accession number ATCC 98817;

(e) a polynucleotide encoding the full-length protein encoded by the cDNA insert of clone di393_{—}2 deposited under accession number ATCC 98817;

(f) a polynucleotide comprising the nucleotide sequence of a mature protein coding sequence of clone di393_{—}2 deposited under accession number ATCC 98817;

(g) a polynucleotide encoding a mature protein encoded by the cDNA insert of clone di393_{—}2 deposited under accession number ATCC 98817;

(h) a polynucleotide encoding a protein comprising the amino acid sequence of SEQ ID NO:70;

(i) a polynucleotide encoding a protein comprising a fragment of the amino acid sequence of SEQ ID NO:70 having biological activity, the fragment comprising eight contiguous amino acids of SEQ ID NO:70;

a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:69.

Preferably, such polynucleotide comprises the nucleotide sequence of SEQ ID NO:69 from nucleotide 31 to nucleotide 543; the nucleotide sequence of SEQ ID NO:69 from nucleotide 88 to nucleotide 543; the nucleotide sequence of the full-length protein coding sequence of clone di393_{—}2 deposited under accession number ATCC 98817; or the nucleotide sequence of a mature protein coding sequence of clone di393_{—}2 deposited under accession number ATCC 98817. In other preferred embodiments, the polynucleotide encodes the full-length or a mature protein encoded by the cDNA insert of clone di393_{—}2 deposited under accession number ATCC 98817. In further preferred embodiments, the present invention provides a polynucleotide encoding a protein comprising a fragment of the amino acid sequence of SEQ ID NO:70 having biological activity, the fragment preferably comprising eight (more preferably twenty, most preferably thirty) contiguous amino acids of SEQ ID NO:70, or a polynucleotide encoding a protein comprising a fragment of the amino acid sequence of SEQ ID NO:70 having biological activity, the fragment comprising the amino acid sequence from amino acid 80 to amino acid 89 of SEQ ID NO:70.

Other embodiments provide the gene corresponding to the cDNA sequence of SEQ ID NO:69.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:69, but excluding the poly(A) tail at the 3′ end of SEQ ID NO:69; and

(ab) the nucleotide sequence of the cDNA insert of clone di393_{—}2 deposited under accession number ATCC 98817;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:69, but excluding the poly(A) tail at the 3′ end of SEQ ID NO:69; and

(bb) the nucleotide sequence of the cDNA insert of clone di393_{—}2 deposited under accession number ATCC 98817;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:69, and extending contiguously from a nucleotide sequence corresponding to the 5′ end of SEQ ID NO:69 to a nucleotide sequence corresponding to the 3′ end of SEQ ID NO:69, but excluding the poly(A) tail at the 3′ end of SEQ ID NO:69. Also preferably the polynucleotide isolated according to the above process comprises a nucleotide sequence corresponding to the cDNA sequence of SEQ ID NO:69 from nucleotide 31 to nucleotide 543, and extending contiguously from a nucleotide sequence corresponding to the 5′ end of said sequence of SEQ ID NO:69 from nucleotide 31 to nucleotide 543, to a nucleotide sequence corresponding to the 3′ end of said sequence of SEQ ID NO:69 from nucleotide 31 to nucleotide 543. Also preferably the polynucleotide isolated according to the above process comprises a nucleotide sequence corresponding to the cDNA sequence of SEQ ID NO:69 from nucleotide 88 to nucleotide 543, and extending contiguously from a nucleotide sequence corresponding to the 5′ end of said sequence of SEQ ID NO:69 from nucleotide 88 to nucleotide 543, to a nucleotide sequence corresponding to the 3′ end of said sequence of SEQ ID NO:69 from nucleotide 88 to nucleotide 543.

In other embodiments, the present invention provides a composition comprising a protein, wherein said protein comprises an amino acid sequence selected from the group consisting of:

(a) the amino acid sequence of SEQ ID NO:70;

(b) a fragment of the amino acid sequence of SEQ ID NO:70, the fragment comprising eight contiguous amino adds of SEQ ID NO:70; and

(c) the amino acid sequence encoded by the cDNA insert of clone di393_{—}2 deposited under accession number ATCC 98817;

the protein being substantially free from other mammalian proteins. Preferably such protein comprises the amino acid sequence of SEQ ID NO:70. In further preferred embodiments, the present invention provides a protein comprising a fragment of the amino add sequence of SEQ ID NO:70 having biological activity, the fragment preferably comprising eight (more preferably twenty, most preferably thirty) contiguous amino acids of SEQ ID NO:70, or a protein comprising a fragment of the amino acid sequence of SEQ ID NO:70 having biological activity, the fragment comprising the amino acid sequence from amino acid 80 to amino acid 89 of SEQ ID NO:70.

In one embodiment, the present invention provides a composition comprising an isolated polynucleotide selected from the group consisting of:

(a) a polynucleotide comprising the nucleotide sequence of SEQ ID NO:71;

(b) a polynucleotide comprising the nucleotide sequence of SEQ ID NO:71 from nucleotide 157 to nucleotide 1356;

(c) a polynucleotide comprising the nucleotide sequence of the full-length protein coding sequence of clone dj167_{—}2 deposited under accession number ATCC 98818;

(d) a polynucleotide encoding the full-length protein encoded by the cDNA insert of clone dj167_{—}2 deposited under accession number ATCC 98818;

(e) a polynucleotide comprising the nucleotide sequence of a mature protein coding sequence of clone dj167_{—}2 deposited under accession number ATCC 98818;

(f) a polynucleotide encoding a mature protein encoded by the cDNA insert of clone dj167_{—}2 deposited under accession number ATCC 98818;

(g) a polynucleotide encoding a protein comprising the amino acid sequence of SEQ ID NO:72;

(h) a polynucleotide encoding a protein comprising a fragment of the amino acid sequence of SEQ ID NO:72 having biological activity, the fragment comprising eight contiguous amino adds of SEQ ID NO:72;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:71.

Preferably, such polynucleotide comprises the nucleotide sequence of SEQ ID NO:71 from nucleotide 157 to nucleotide 1356; the nucleotide sequence of the full-length protein coding sequence of clone dj167_{—}2 deposited under accession number ATCC 98818; or the nucleotide sequence of a mature protein coding sequence of clone dj167_{—}2 deposited under accession number ATCC 98818. In other preferred embodiments, the polynucleotide encodes the full-length or a mature protein encoded by the cDNA insert of clone dj167_{—}2 deposited under accession number ATCC 98818. In further preferred embodiments, the present invention provides a polynucleotide encoding a protein comprising a fragment of the amino acid sequence of SEQ ID NO:72 having biological activity, the fragment preferably comprising eight (more preferably twenty, most preferably thirty) contiguous amino acids of SEQ ID NO:72, or a polynucleotide encoding a protein comprising a fragment of the amino acid sequence of SEQ ID NO:72 having biological activity, the fragment comprising the amino acid sequence from amino acid 195 to amino acid 204 of SEQ ID NO:72.

Other embodiments provide the gene corresponding to the cDNA sequence of SEQ ID NO:71.

Further embodiments of the invention provide isolated polynucleotides produced according to a process selected from the group consisting of.

(a) a process comprising the steps of:

(i) preparing one or more polynucleotide probes that hybridize in 6×SSC at 65 degrees C. to a nucleotide sequence selected from the group consisting of:

(aa) SEQ ID NO:71, but excluding the poly(A) tail at the 3′ end of SEQ ID NO:71; and

(ab) the nucleotide sequence of the cDNA insert of clone dj167_{—}2 deposited under accession number ATCC 98818;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:71, but excluding the poly(A) tail at the 3′ end of SEQ ID NO:71; and

(bb) the nucleotide sequence of the cDNA insert of clone dj167_{—}2 deposited under accession number ATCC 98818;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:71, and extending contiguously from a nucleotide sequence corresponding to the 5′ end of SEQ ID NO:71 to a nucleotide sequence corresponding to the 3′ end of SEQ ID NO:71, but excluding the poly(A) tail at the 3′ end of SEQ ID NO:71. Also preferably the polynucleotide isolated according to the above process comprises a nucleotide sequence corresponding to the cDNA sequence of SEQ ID NO:71 from nucleotide 157 to nucleotide 1356, and extending contiguously from a nucleotide sequence corresponding to the 5′ end of said sequence of SEQ ID NO:71 from nucleotide 157 to nucleotide 1356, to a nucleotide sequence corresponding to the 3′ end of said sequence of SEQ ID NO:71 from nucleotide 157 to nucleotide 1356.

In other embodiments, the present invention provides a composition comprising a protein, wherein said protein comprises an amino acid sequence selected from the group consisting of:

(a) the amino acid sequence of SEQ ID NO:72;

(b) a fragment of the amino acid sequence of SEQ ID NO:72, the fragment comprising eight contiguous amino acids of SEQ ID NO:72; and

(c) the amino add sequence encoded by the cDNA insert of clone dj167_{—}2 deposited under accession number ATCC 98818;

the protein being substantially free from other mammalian proteins. Preferably such protein comprises the amino add sequence of SEQ ID NO:72. In further preferred embodiments, the present invention provides a protein comprising a fragment of the amino add sequence of SEQ ID NO:72 having biological activity, the fragment preferably comprising eight (more preferably twenty, most preferably thirty) contiguous amino adds of SEQ ID NO:72, or a protein comprising a fragment of the amino acid sequence of SEQ ID NO:72 having biological activity, the fragment comprising the amino add sequence from amino acid 195 to amino acid 204 of SEQ ID NO:72.

In one embodiment, the present invention provides a composition comprising an isolated polynucleotide selected from the group consisting of:

(a) a polynucleotide comprising the nucleotide sequence of SEQ ID NO:73;

(b) a polynucleotide comprising the nucleotide sequence of SEQ ID NO:73 from nucleotide 1383 to nucleotide 4490;

(c) a polynucleotide comprising the nucleotide sequence of SEQ ID NO:73 from nucleotide 1485 to nucleotide 4490;

(d) a polynucleotide comprising the nucleotide sequence of SEQ ID NO:73 from nucleotide 3645 to nucleotide 4343;

(e) a polynucleotide comprising the nucleotide sequence of the full-length protein coding sequence of clone dj167_{—}19 deposited under accession number ATCC 207090;

(f) a polynucleotide encoding the full-length protein encoded by the cDNA insert of clone dj167_{—}19 deposited under accession number ATCC 207090;

(g) a polynucleotide comprising the nucleotide sequence of a mature protein coding sequence of clone dj167_{—}19 deposited under accession number ATCC 207090;

(h) a polynucleotide encoding a mature protein encoded by the cDNA insert of clone dj167_{—}19 deposited under accession number ATCC 207090;

(i) a polynucleotide encoding a protein comprising the amino add sequence of SEQ ID NO:74;

(j) a polynucleotide encoding a protein comprising a fragment of the amino acid sequence of SEQ ID NO:74 having biological activity, the fragment comprising eight contiguous amino acids of SEQ ID NO:74;

(k) a polynucleotide which is an allelic variant of a polynucleotide of (a)-(h) above;

(l) a polynucleotide which encodes a species homologue of the protein of (i) or (j) above;

(m) a polynucleotide that hybridizes under stringent conditions to any one of the polynucleotides specified in (a)-(j); and

(n) a polynucleotide that hybridizes under stringent conditions to any one of the polynucleotides specified in (a)-(j) and that has a length that is at least 25% of the length of SEQ ID NO:73.

Preferably, such polynucleotide comprises the nucleotide sequence of SEQ ID NO:73 from nucleotide 1383 to nucleotide 4490; the nucleotide sequence of SEQ ID NO:73 from nucleotide 1485 to nucleotide 4490; the nucleotide sequence of SEQ ID NO:73 from nucleotide 3645 to nucleotide 4343; the nucleotide sequence of the full-length protein coding sequence of clone dj167_{—}19 deposited under accession number ATCC 207090; or the nucleotide sequence of a mature protein coding sequence of clone dj167_{—}19 deposited under accession number ATCC 207090. In other preferred embodiments, the polynucleotide encodes the full-length or a mature protein encoded by the cDNA insert of clone dj167_{—}19 deposited under accession number ATCC 207090. In yet other preferred embodiments, the present invention provides a polynucleotide encoding a protein comprising the amino acid sequence of SEQ ID NO:74 from amino acid 637 to amino acid 1036. In further preferred embodiments, the present invention provides a polynucleotide encoding a protein comprising a fragment of the amino acid sequence of SEQ ID NO:74 having biological activity, the fragment preferably comprising eight (more preferably twenty, most preferably thirty) contiguous amino acids of SEQ ID NO:74, or a polynucleotide encoding a protein comprising a fragment of the amino add sequence of SEQ ID NO:74 having biological activity, the fragment comprising the amino add sequence from amino acid 513 to amino acid 522 of SEQ ID NO:74.

Other embodiments provide the gene corresponding to the cDNA sequence of SEQ ID NO:73.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of

(aa) SEQ ID NO:73, but excluding the poly(A) tail at the 3′ end of SEQ ID NO:73; and

(ab) the nucleotide sequence of the cDNA insert of clone dj167_{—}19 deposited under accession number ATCC 207090;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:73, but excluding the poly(A) tail at the 3′ end of SEQ ID NO:73; and

(bb) the nucleotide sequence of the cDNA insert of clone dj167_{—}19 deposited under accession number ATCC 207090;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:73, and extending contiguously from a nucleotide sequence corresponding to the 5′ end of SEQ ID NO:73 to a nucleotide sequence corresponding to the 3′ end of SEQ ID NO:73, but excluding the poly(A) tail at the 3′ end of SEQ ID NO:73. Also preferably the polynucleotide isolated according to the above process comprises a nucleotide sequence corresponding to the cDNA sequence of SEQ ID NO:73 from nucleotide 1383 to nucleotide 4490, and extending contiguously from a nucleotide sequence corresponding to the 5′ end of said sequence of SEQ ID NO:73 from nucleotide 1383 to nucleotide 4490, to a nucleotide sequence corresponding to the 3′ end of said sequence of SEQ ID NO:73 from nucleotide 1383 to nucleotide 4490. Also preferably the polynucleotide isolated according to the above process comprises a nucleotide sequence corresponding to the cDNA sequence of SEQ ID NO:73 from nucleotide 1485 to nucleotide 4490, and extending contiguously from a nucleotide sequence corresponding to the 5′ end of said sequence of SEQ ID NO:73 from nucleotide 1485 to nucleotide 4490, to a nucleotide sequence corresponding to the 3′ end of said sequence of SEQ ID NO:73 from nucleotide 1485 to nucleotide 4490. Also preferably the polynucleotide isolated according to the above process comprises a nucleotide sequence corresponding to the cDNA sequence of SEQ ID NO:73 from nucleotide 3645 to nucleotide 4343, and extending contiguously from a nucleotide sequence corresponding to the 5′ end of said sequence of SEQ ID NO:73 from nucleotide 3645 to nucleotide 4343, to a nucleotide sequence corresponding to the 3′ end of said sequence of SEQ ID NO:73 from nucleotide 3645 to nucleotide 4343.

In other embodiments, the present invention provides a composition comprising a protein, wherein said protein comprises an amino add sequence selected from the group consisting of:

(a) the amino acid sequence of SEQ ID NO:74;

(b) the amino acid sequence of SEQ ID NO:74 from amino acid 637 to amino acid 1036;

(c) a fragment of the amino acid sequence of SEQ ID NO:74, the fragment comprising eight contiguous amino adds of SEQ ID NO:74; and

(d) the amino acid sequence encoded by the cDNA insert of clone dj167_{—}19 deposited under accession number ATCC 207090;

the protein being substantially free from other mammalian proteins. Preferably such protein comprises the amino acid sequence of SEQ ID NO:74 or the amino acid sequence of SEQ ID NO:74 from amino acid 637 to amino acid 1036. In further preferred embodiments, the present invention provides a protein comprising a fragment of the amino add sequence of SEQ ID NO:74 having biological activity, the fragment preferably comprising eight (more preferably twenty, most preferably thirty) contiguous amino adds of SEQ ID NO:74, or a protein comprising a fragment of the amino acid sequence of SEQ ID NO:74 having biological activity, the fragment comprising the amino acid sequence from amino acid 513 to amino acid 522 of SEQ ID NO:74.

In one embodiment, the present invention provides a composition comprising an isolated polynucleotide selected from the group consisting of:

(a) a polynucleotide comprising the nucleotide sequence of SEQ ID NO:75;

(b) a polynucleotide comprising the nucleotide sequence of SEQ ID NO:75 from nucleotide 71 to nucleotide 1441;

(c) a polynucleotide comprising the nucleotide sequence of SEQ ID NO:75 from nucleotide 152 to nucleotide 1441;

(d) a polynucleotide comprising the nucleotide sequence of the full-length protein coding sequence of clone dw665_{—}4 deposited under accession number ATCC 98818;

(e) a polynucleotide encoding the full-length protein encoded by the cDNA insert of clone dw665_{—}4 deposited under accession number ATCC 98818;

(f) a polynucleotide comprising the nucleotide sequence of a mature protein coding sequence of clone dw665_{—}4 deposited under accession number ATCC 98818;

(g) a polynucleotide encoding a mature protein encoded by the cDNA insert of clone dw6654 deposited under accession number ATCC 98818;

(h) a polynucleotide encoding a protein comprising the amino acid sequence of SEQ ID NO:76;

(i) a polynucleotide encoding a protein comprising a fragment of the amino acid sequence of SEQ ID NO:76 having biological activity, the fragment comprising eight contiguous amino acids of SEQ ID NO:76;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:75.

Preferably, such polynucleotide comprises the nucleotide sequence of SEQ ID NO:75 from nucleotide 71 to nucleotide 1441; the nucleotide sequence of SEQ ID NO:75 from nucleotide 152 to nucleotide 1441; the nucleotide sequence of the full-length protein coding sequence of clone dw665_{—}4 deposited under accession number ATCC 98818; or the nucleotide sequence of a mature protein coding sequence of clone dw665_{—}4 deposited under accession number ATCC 98818. In other preferred embodiments, the polynucleotide encodes the full-length or a mature protein encoded by the cDNA insert of clone dw665_{—}4 deposited under accession number ATCC 98818. In further preferred embodiments, the present invention provides a polynucleotide encoding a protein comprising a fragment of the amino acid sequence of SEQ ID NO:76 having biological activity, the fragment preferably comprising eight (more preferably twenty, most preferably thirty) contiguous amino acids of SEQ ID NO:76, or a polynucleotide encoding a protein comprising a fragment of the amino acid sequence of SEQ ID NO:76 having biological activity, the fragment comprising the amino acid sequence from amino acid 223 to amino acid 232 of SEQ ID NO:76.

Other embodiments provide the gene corresponding to the cDNA sequence of SEQ ID NO:75.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:75, but excluding the poly(A) tail at the 3′ end of SEQ ID NO:75; and

(ab) the nucleotide sequence of the cDNA insert of clone dw665_{—}4 deposited under accession number ATCC 98818;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:75, but excluding the poly(A) tail at the 3′ end of SEQ ID NO:75; and

(bb) the nucleotide sequence of the cDNA insert of clone dw665_{—}4 deposited under accession number ATCC 98818;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:75, and extending contiguously from a nucleotide sequence corresponding to the 5′ end of SEQ ID NO:75 to a nucleotide sequence corresponding to the 3′ end of SEQ ID NO:75, but excluding the poly(A) tail at the 3′ end of SEQ ID NO:75. Also preferably the polynucleotide isolated according to the above process comprises a nucleotide sequence corresponding to the cDNA sequence of SEQ ID NO:75 from nucleotide 71 to nucleotide 1441, and extending contiguously from a nucleotide sequence corresponding to the 5′ end of said sequence of SEQ ID NO:75 from nucleotide 71 to nucleotide 1441, to a nucleotide sequence corresponding to the 3′ end of said sequence of SEQ ID NO:75 from nucleotide 71 to nucleotide 1441. Also preferably the polynucleotide isolated according to the above process comprises a nucleotide sequence corresponding to the cDNA sequence of SEQ ID NO:75 from nucleotide 152 to nucleotide 1441, and extending contiguously from a nucleotide sequence corresponding to the 5′ end of said sequence of SEQ ID NO:75 from nucleotide 152 to nucleotide 1441, to a nucleotide sequence corresponding to the 3′ end of said sequence of SEQ ID NO:75 from nucleotide 152 to nucleotide 1441.

In other embodiments, the present invention provides a composition comprising a protein, wherein said protein comprises an amino acid sequence selected from the group consisting of:

(a) the amino add sequence of SEQ ID NO:76;

(b) a fragment of the amino acid sequence of SEQ ID NO:76, the fragment comprising eight contiguous amino acids of SEQ ID NO:76; and

(c) the amino acid sequence encoded by the cDNA insert of clone dw665_{—}4 deposited under accession number ATCC 98818;

the protein being substantially free from other mammalian proteins. Preferably such protein comprises the amino add sequence of SEQ ID NO:76. In further preferred embodiments, the present invention provides a protein comprising a fragment of the amino add sequence of SEQ ID NO:76 having biological activity, the fragment preferably comprising eight (more preferably twenty, most preferably thirty) contiguous amino acids of SEQ ID NO:76, or a protein comprising a fragment of the amino acid sequence of SEQ ID NO:76 having biological activity, the fragment comprising the amino acid sequence from amino acid 223 to amino acid 232 of SEQ ID NO:76.

In one embodiment, the present invention provides a composition comprising an isolated polynucleotide selected from the group consisting of:

(a) a polynucleotide comprising the nucleotide sequence of SEQ ID NO:77;

(b) a polynucleotide comprising the nucleotide sequence of SEQ ID NO:77 from nucleotide 78 to nucleotide 1592;

(c) a polynucleotide comprising the nucleotide sequence of the full-length protein coding sequence of clone dx146_{—}12 deposited under accession number ATCC 98818;

(d) a polynucleotide encoding the full-length protein encoded by the cDNA insert of clone dx146_{—}12 deposited under accession number ATCC 98818;

(e) a polynucleotide comprising the nucleotide sequence of a mature protein coding sequence of clone dx146_{—}12 deposited under accession number ATCC 98818;

(f) a polynucleotide encoding a mature protein encoded by the cDNA insert of clone dx146_{—}12 deposited under accession number ATCC 98818;

(g) a polynucleotide encoding a protein comprising the amino acid sequence of SEQ ID NO:78;

(h) a polynucleotide encoding a protein comprising a fragment of the amino acid sequence of SEQ ID NO:78 having biological activity, the fragment comprising eight contiguous amino acids of SEQ ID NO:78;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:77.

Preferably, such polynucleotide comprises the nucleotide sequence of SEQ ID NO:77 from nucleotide 78 to nucleotide 1592; the nucleotide sequence of the full-length protein coding sequence of clone dx146_{—}12 deposited under accession number ATCC 98818; or the nucleotide sequence of a mature protein coding sequence of clone dx146_{—}12 deposited under accession number ATCC 98818. In other preferred embodiments, the polynucleotide encodes the full-length or a mature protein encoded by the cDNA insert of clone dx146_{—}12 deposited under accession number ATCC 98818. In further preferred embodiments, the present invention provides a polynucleotide encoding a protein comprising a fragment of the amino acid sequence of SEQ ID NO:78 having biological activity, the fragment preferably comprising eight (more preferably twenty, most preferably thirty) contiguous amino acids of SEQ ID NO:78, or a polynucleotide encoding a protein comprising a fragment of the amino acid sequence of SEQ ID NO:78 having biological activity, the fragment comprising the amino acid sequence from amino acid 247 to amino acid 256 of SEQ ID NO:78. Other embodiments provide the gene corresponding to the cDNA sequence of SEQ ID NO:77.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:77, but excluding the poly(A) tail at the 3′ end of SEQ ID NO:77; and

(ab) the nucleotide sequence of the cDNA insert of clone dx146_{—}12 deposited under accession number ATCC 98818;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:77, but excluding the poly(A) tail at the 3′ end of SEQ ID NO:77; and

(bb) the nucleotide sequence of the cDNA insert of clone dx146_{—}12 deposited under accession number ATCC 98818;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:77, and extending contiguously from a nucleotide sequence corresponding to the 5′ end of SEQ ID NO:77 to a nucleotide sequence corresponding to the 3′ end of SEQ ID NO:77, but excluding the poly(A) tail at the 3′ end of SEQ ID NO:77. Also preferably the polynucleotide isolated according to the above process comprises a nucleotide sequence corresponding to the cDNA sequence of SEQ ID NO:77 from nucleotide 78 to nucleotide 1592, and extending contiguously from a nucleotide sequence corresponding to the 5′ end of said sequence of SEQ ID NO:77 from nucleotide 78 to nucleotide 1592, to a nucleotide sequence corresponding to the 3′ end of said sequence of SEQ ID NO:77 from nucleotide 78 to nucleotide 1592.

In other embodiments, the present invention provides a composition comprising a protein, wherein said protein comprises an amino acid sequence selected from the group consisting of:

(a) the amino acid sequence of SEQ ID NO:78;

(b) a fragment of the amino acid sequence of SEQ ID NO:78, the fragment comprising eight contiguous amino acids of SEQ ID NO:78; and

(c) the amino acid sequence encoded by the cDNA insert of clone dx146_{—}12 deposited under accession number ATCC 98818;

the protein being substantially free from other mammalian proteins. Preferably such protein comprises the amino acid sequence of SEQ ID NO:78. In further preferred embodiments, the present invention provides a protein comprising a fragment of the amino add sequence of SEQ ID NO:78 having biological activity, the fragment preferably comprising eight (more preferably twenty, most preferably thirty) contiguous amino acids of SEQ ID NO:78, or a protein comprising a fragment of the amino acid sequence of SEQ ID NO:78 having biological activity, the fragment comprising the amino acid sequence from amino acid 247 to amino acid 256 of SEQ ID NO:78.

In one embodiment, the present invention provides a composition comprising an isolated polynucleotide selected from the group consisting of:

(a) a polynucleotide comprising the nucleotide sequence of SEQ ID NO:79;

(b) a polynucleotide comprising the nucleotide sequence of SEQ ID NO:79 from nucleotide 19 to nucleotide 948;

(c) a polynucleotide comprising the nucleotide sequence of SEQ ID NO:79 from nucleotide 337 to nucleotide 948;

(d) a polynucleotide comprising the nucleotide sequence of the full-length protein coding sequence of clone dx219_{—}13 deposited under accession number ATCC 98818;

(e) a polynucleotide encoding the full-length protein encoded by the cDNA insert of clone dx219_{—}13 deposited under accession number ATCC 98818;

(f) a polynucleotide comprising the nucleotide sequence of a mature protein coding sequence of clone dx219_{—}13 deposited under accession number ATCC 98818;

(g) a polynucleotide encoding a mature protein encoded by the cDNA insert of clone dx219_{—}13 deposited under accession number ATCC 98818;

(h) a polynucleotide encoding a protein comprising the amino acid sequence of SEQ ID NO:80;

(i) a polynucleotide encoding a protein comprising a fragment of the amino acid sequence of SEQ ID NO:80 having biological activity, the fragment comprising eight contiguous amino acids of SEQ ID NO:80;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:79.

Preferably, such polynucleotide comprises the nucleotide sequence of SEQ ID NO:79 from nucleotide 19 to nucleotide 948; the nucleotide sequence of SEQ ID NO:79 from nucleotide 337 to nucleotide 948; the nucleotide sequence of the full-length protein coding sequence of clone dx219_{—}13 deposited under accession number ATCC 98818; or the nucleotide sequence of a mature protein coding sequence of clone dx219_{—}13 deposited under accession number ATCC 98818. In other preferred embodiments, the polynucleotide encodes the full-length or a mature protein encoded by the cDNA insert of clone dx219_{—}13 deposited under accession number ATCC 98818. In further preferred embodiments, the present invention provides a polynucleotide encoding a protein comprising a fragment of the amino acid sequence of SEQ ID NO:80 having biological activity, the fragment preferably comprising eight (more preferably twenty, most preferably thirty) contiguous amino acids of SEQ ID NO:80, or a polynucleotide encoding a protein comprising a fragment of the amino acid sequence of SEQ ID NO:80 having biological activity, the fragment comprising the amino acid sequence from amino acid 150 to amino acid 159 of SEQ ID NO:80.

Other embodiments provide the gene corresponding to the cDNA sequence of SEQ ID NO:79.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:79, but excluding the poly(A) tail at the 3′ end of SEQ ID NO:79; and

(ab) the nucleotide sequence of the cDNA insert of clone dx219_{—}13 deposited under accession number ATCC 98818;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:79, but excluding the poly(A) tail at the 3′ end of SEQ ID NO:79; and

(bb) the nucleotide sequence of the cDNA insert of clone dx219_{—}13 deposited under accession number ATCC 98818;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:79, and extending contiguously from a nucleotide sequence corresponding to the 5′ end of SEQ ID NO:79 to a nucleotide sequence corresponding to the 3′ end of SEQ ID NO:79, but excluding the poly(A) tail at the 3′ end of SEQ ID NO:79. Also preferably the polynucleotide isolated according to the above process comprises a nucleotide sequence corresponding to the cDNA sequence of SEQ ID NO:79 from nucleotide 19 to nucleotide 948, and extending contiguously from a nucleotide sequence corresponding to the 5′ end of said sequence of SEQ ID NO:79 from nucleotide 19 to nucleotide 948, to a nucleotide sequence corresponding to the 3′ end of said sequence of SEQ ID NO:79 from nucleotide 19 to nucleotide 948. Also preferably the polynucleotide isolated according to the above process comprises a nucleotide sequence corresponding to the cDNA sequence of SEQ ID NO:79 from nucleotide 337 to nucleotide 948, and extending contiguously from a nucleotide sequence corresponding to the 5′ end of said sequence of SEQ ID NO:79 from nucleotide 337 to nucleotide 948, to a nucleotide sequence corresponding to the 3′ end of said sequence of SEQ ID NO:79 from nucleotide 337 to nucleotide 948.

In other embodiments, the present invention provides a composition comprising a protein, wherein said protein comprises an amino acid sequence selected from the group consisting of:

(a) the amino acid sequence of SEQ ID NO:80;

(b) a fragment of the amino acid sequence of SEQ ID NO:80, the fragment comprising eight contiguous amino acids of SEQ ID NO:80; and

(c) the amino acid sequence encoded by the cDNA insert of clone dx219_{—}13 deposited under accession number ATCC 98818;

the protein being substantially free from other mammalian proteins. Preferably such protein comprises the amino acid sequence of SEQ ID NO:80. In further preferred embodiments, the present invention provides a protein comprising a fragment of the amino acid sequence of SEQ ID NO:80 having biological activity, the fragment preferably comprising eight (more preferably twenty, most preferably thirty) contiguous amino acids of SEQ ID NO:80, or a protein comprising a fragment of the amino acid sequence of SEQ ID NO:80 having biological activity, the fragment comprising the amino acid sequence from amino acid 150 to amino acid 159 of SEQ ID NO:80.

In one embodiment, the present invention provides a composition comprising an isolated polynucleotide selected from the group consisting of:

(a) a polynucleotide comprising the nucleotide sequence of SEQ ID NO:81;

(b) a polynucleotide comprising the nucleotide sequence of SEQ ID NO:81 from nucleotide 5 to nucleotide 286;

(c) a polynucleotide comprising the nucleotide sequence of SEQ ID NO:81 from nucleotide 62 to nucleotide 286;

(d) a polynucleotide comprising the nucleotide sequence of the full-length protein coding sequence of clone fm3_{—}1 deposited under accession number ATCC 98818;

(e) a polynucleotide encoding the full-length protein encoded by the cDNA insert of clone fm3_{—}1 deposited under accession number ATCC 98818;

(f) a polynucleotide comprising the nucleotide sequence of a mature protein coding sequence of clone fm3_{—}1 deposited under accession number ATCC 98818;

(g) a polynucleotide encoding a mature protein encoded by the cDNA insert of clone fm3_{—}1 deposited under accession number ATCC 98818;

(h) a polynucleotide encoding a protein comprising the amino acid sequence of SEQ ID NO:82;

(i) a polynucleotide encoding a protein comprising a fragment of the amino acid sequence of SEQ ID NO:82 having biological activity, the fragment comprising eight contiguous amino acids of SEQ ID NO:82;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:81.

Preferably, such polynucleotide comprises the nucleotide sequence of SEQ ID NO:81 from nucleotide 5 to nucleotide 286; the nucleotide sequence of SEQ ID NO:81 from nucleotide 62 to nucleotide 286; the nucleotide sequence of the full-length protein coding sequence of clone fm3_{—}1 deposited under accession number ATCC 98818; or the nucleotide sequence of a mature protein coding sequence of clone fm3_{—}1 deposited under accession number ATCC 98818. In other preferred embodiments, the polynucleotide encodes the full-length or a mature protein encoded by the cDNA insert of clone fm3_{—}1 deposited under accession number ATCC 98818. In further preferred embodiments, the present invention provides a polynucleotide encoding a protein comprising a fragment of the amino acid sequence of SEQ ID NO:82 having biological activity, the fragment preferably comprising eight (more preferably twenty, most preferably thirty) contiguous amino acids of SEQ ID NO:82, or a polynucleotide encoding a protein comprising a fragment of the amino acid sequence of SEQ ID NO:82 having biological activity, the fragment comprising the amino acid sequence from amino acid 42 to amino acid 51 of SEQ ID NO:82.

Other embodiments provide the gene corresponding to the cDNA sequence of SEQ ID NO:81.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:81, but excluding the poly(A) tail at the 3′ end of SEQ ID NO:81; and

(ab) the nucleotide sequence of the cDNA insert of clone fm3_{—}1 deposited under accession number ATCC 98818;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide: primers that hybridize in 6×SSC at 65 degrees C. to a nucleotide sequence selected from the group consisting of:

(ba) SEQ ID NO:81, but excluding the poly(A) tail at the 3′ end of SEQ ID NO:81; and

(bb) the nucleotide sequence of the cDNA insert of clone fm3_{—}1 deposited under accession number ATCC 98818;

(ii) hybridizing said primer(s) to human genomic DNA in conditions at least as stringent as 4×SSC at 50 degrees C.;

(ii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:81, and extending contiguously from a nucleotide sequence corresponding to the 5′ end of SEQ ID NO:81 to a nucleotide sequence corresponding to the 3′ end of SEQ ID NO:81, but excluding the poly(A) tail at the 3′ end of SEQ ID NO:81. Also preferably the polynucleotide isolated according to the above process comprises a nucleotide sequence corresponding to the cDNA sequence of SEQ ID NO:81 from nucleotide 5 to nucleotide 286, and extending contiguously from a nucleotide sequence corresponding to the 5′ end of said sequence of SEQ ID NO:81 from nucleotide 5 to nucleotide 286, to a nucleotide sequence corresponding to the 3′ end of said sequence of SEQ ID NO:81 from nucleotide 5 to nucleotide 286. Also preferably the polynucleotide isolated according to the above process comprises a nucleotide sequence corresponding to the cDNA sequence of SEQ ID NO:81 from nucleotide 62 to nucleotide 286, and extending contiguously from a nucleotide sequence corresponding to the 5′ end of said sequence of SEQ ID NO:81 from nucleotide 62 to nucleotide 286, to a nucleotide sequence corresponding to the 3′ end of said sequence of SEQ ID NO:81 from nucleotide 62 to nucleotide 286.

In other embodiments, the present invention provides a composition comprising a protein, wherein said protein comprises an amino acid sequence selected from the group consisting of:

(a) the amino acid sequence of SEQ ID NO:82;

(b) a fragment of the amino acid sequence of SEQ ID NO:82, the fragment comprising eight contiguous amino acids of SEQ ID NO:82; and

(c) the amino acid sequence encoded by the cDNA insert of clone fm3_{—}1 deposited under accession number ATCC 98818;

the protein being substantially free from other mammalian proteins. Preferably such protein comprises the amino acid sequence of SEQ ID NO:82. In further preferred embodiments, the present invention provides a protein comprising a fragment of the amino acid sequence of SEQ ID NO:82 having biological activity, the fragment preferably comprising eight (more preferably twenty, most preferably thirty) contiguous amino acids of SEQ ID NO:82, or a protein comprising a fragment of the amino acid sequence of SEQ ID NO:82 having biological activity, the fragment comprising the amino acid sequence from amino acid 42 to amino acid 51 of SEQ ID NO:82.

In one embodiment, the present invention provides a composition comprising an isolated polynucleotide selected from the group consisting of:

(a) a polynucleotide comprising the nucleotide sequence of SEQ ID NO:83;

(b) a polynucleotide comprising the nucleotide sequence of SEQ ID NO:83 from nucleotide 141 to nucleotide 572;

(c) a polynucleotide comprising the nucleotide sequence of SEQ ID NO:83 from nucleotide 333 to nucleotide 572;

(d) a polynucleotide comprising the nucleotide sequence of the full-length protein coding sequence of clone h225_{—}1 deposited under accession number ATCC 98818;

(e) a polynucleotide encoding the full-length protein encoded by the cDNA insert of clone h225_{—}1 deposited under accession number ATCC 98818;

(f) a polynucleotide comprising the nucleotide sequence of a mature protein coding sequence of clone h225_{—}1 deposited under accession number ATCC 98818;

(g) a polynucleotide encoding a mature protein encoded by the cDNA insert of clone 225_{—}1 deposited under accession number ATCC 98818;

(h) a polynucleotide encoding a protein comprising the amino acid sequence of SEQ ID NO:84;

(i) a polynucleotide encoding a protein comprising a fragment of the amino acid sequence of SEQ ID NO:84 having biological activity, the fragment comprising eight contiguous amino acids of SEQ ID NO:84;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:83.

Preferably, such polynucleotide comprises the nucleotide sequence of SEQ ID NO:83 from nucleotide 141 to nucleotide 572; the nucleotide sequence of SEQ ID NO:83 from nucleotide 333 to nucleotide 572; the nucleotide sequence of the full-length protein coding sequence of clone h225_{—}1 deposited under accession number ATCC 98818; or the nucleotide sequence of a mature protein coding sequence of clone h225_{—}1 deposited under accession number ATCC 98818. In other preferred embodiments, the polynucleotide encodes the full-length or a mature protein encoded by the cDNA insert of clone h225_{—}1 deposited under accession number ATCC 98818. In further preferred embodiments, the present invention provides a polynucleotide encoding a protein comprising a fragment of the amino acid sequence of SEQ ID NO:84 having biological activity, the fragment preferably comprising eight (more preferably twenty, most preferably thirty) contiguous amino acids of SEQ ID NO:84, or a polynucleotide encoding a protein comprising a fragment of the amino acid sequence of SEQ ID NO:84 having biological activity, the fragment comprising the amino acid sequence from amino acid 67 to amino acid 76 of SEQ ID NO:84.

Other embodiments provide the gene corresponding to the cDNA sequence of SEQ ID NO:83.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:83; and

(ab) the nucleotide sequence of the cDNA insert of clone h225_{—}1 deposited under accession number ATCC 98818;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:83; and

(bb) the nucleotide sequence of the cDNA insert of clone h225_{—}1 deposited under accession number ATCC 98818;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:83, and extending contiguously from a nucleotide sequence corresponding to the 5′ end of SEQ ID NO:83 to a nucleotide sequence corresponding to the 3′ end of SEQ ID NO:83. Also preferably the polynucleotide isolated according to the above process comprises a nucleotide sequence corresponding to the cDNA sequence of SEQ ID NO:83 from nucleotide 141 to nucleotide 572, and extending contiguously from a nucleotide sequence corresponding to the 5′ end of said sequence of SEQ ID NO:83 from nucleotide 141 to nucleotide 572, to a nucleotide sequence corresponding to the 3′ end of said sequence of SEQ ID NO:83 from nucleotide 141 to nucleotide 572. Also preferably the polynucleotide isolated according to the above process comprises a nucleotide sequence corresponding to the cDNA sequence of SEQ ID NO:83 from nucleotide 333 to nucleotide 572, and extending contiguously from a nucleotide sequence corresponding to the 5′ end of said sequence of SEQ ID NO:83 from nucleotide 333 to nucleotide 572, to a nucleotide sequence corresponding to the 3′ end of said sequence of SEQ ID NO:83 from nucleotide 333 to nucleotide 572.

In other embodiments, the present invention provides a composition comprising a protein, wherein said protein comprises an amino acid sequence selected from the group consisting of:

(a) the amino acid sequence of SEQ ID NO:84;

(b) a fragment of the amino acid sequence of SEQ ID NO:84, the fragment comprising eight contiguous amino acids of SEQ ID NO:84; and

(c) the amino acid sequence encoded by the cDNA insert of clone h225_{—}1 deposited under accession number ATCC 98818;

the protein being substantially free from other mammalian proteins. Preferably such protein comprises the amino acid sequence of SEQ ID NO:84. In further preferred embodiments, the present invention provides a protein comprising a fragment of the amino acid sequence of SEQ ID NO:84 having biological activity, the fragment preferably comprising eight (more preferably twenty, most preferably thirty) contiguous amino acids of SEQ ID NO:84, or a protein comprising a fragment of the amino acid sequence of SEQ ID NO:84 having biological activity, the fragment comprising the amino add sequence from amino acid 67 to amino acid 76 of SEQ ID NO:84.

In one embodiment, the present invention provides a composition comprising an isolated polynucleotide selected from the group consisting of:

(a) a polynucleotide comprising the nucleotide sequence of SEQ ID NO:85;

(b) a polynucleotide comprising the nucleotide sequence of SEQ ID NO:85 from nucleotide 391 to nucleotide 3210;

(c) a polynucleotide comprising the nucleotide sequence of SEQ ID NO:85 from nucleotide 505 to nucleotide 3210;

(d) a polynucleotide comprising the nucleotide sequence of the full-length protein coding sequence of clone kj320_{—}1 deposited under accession number ATCC 98818;

(e) a polynucleotide encoding the full-length protein encoded by the cDNA insert of clone kj320_{—}1 deposited under accession number ATCC 98818;

(f) a polynucleotide comprising the nucleotide sequence of a mature protein coding sequence of clone kj320_{—}1 deposited under accession number ATCC 98818;

(g) a polynucleotide encoding a mature protein encoded by the cDNA insert of clone kj320_{—}1 deposited under accession number ATCC 98818;

(h) a polynucleotide encoding a protein comprising the amino acid sequence of SEQ ID NO:86;

(i) a polynucleotide encoding a protein comprising a fragment of the amino acid sequence of SEQ ID NO:86 having biological activity, the fragment comprising eight contiguous amino acids of SEQ ID NO:86;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:85.

Preferably, such polynucleotide comprises the nucleotide sequence of SEQ ID NO:85 from nucleotide 391 to nucleotide 3210; the nucleotide sequence of SEQ ID NO:85 from nucleotide 505 to nucleotide 3210; the nucleotide sequence of the full-length protein coding sequence of clone kj320_{—}1 deposited under accession number ATCC 98818; or the nucleotide sequence of a mature protein coding sequence of clone kj320_{—}1 deposited under accession number ATCC 98818. In other preferred embodiments, the polynucleotide encodes the full-length or a mature protein encoded by the cDNA insert of clone kj320_{—}1 deposited under accession number ATCC 98818. In further preferred embodiments, the present invention provides a polynucleotide encoding a protein comprising a fragment of the amino acid sequence of SEQ ID NO:86 having biological activity, the fragment preferably comprising eight (more preferably twenty, most preferably thirty) contiguous amino acids of SEQ ID NO:86, or a polynucleotide encoding a protein comprising a fragment of the amino acid sequence of SEQ ID NO:86 having biological activity, the fragment comprising the amino acid sequence from amino acid 465 to amino acid 474 of SEQ ID NO:86.

Other embodiments provide the gene corresponding to the cDNA sequence of SEQ ID NO:85.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:85, but excluding the poly(A) tail at the 3′ end of SEQ ID NO:85; and

(ab) the nucleotide sequence of the cDNA insert of clone kj320_{—}1 deposited under accession number ATCC 98818;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:85, but excluding the poly(A) tail at the 3′ end of SEQ ID NO:85; and

(bb) the nucleotide sequence of the cDNA insert of clone kj320_{—}1 deposited under accession number ATCC 98818;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:85, and extending contiguously from a nucleotide sequence corresponding to the 5′ end of SEQ ID NO:85 to a nucleotide sequence corresponding to the 3′ end of SEQ ID NO:85, but excluding the poly(A) tail at the 3′ end of SEQ ID NO:85. Also preferably the polynucleotide isolated according to the above process comprises a nucleotide sequence corresponding to the cDNA sequence of SEQ ID NO:85 from nucleotide 391 to nucleotide 3210, and extending contiguously from a nucleotide sequence corresponding to the 5′ end of said sequence of SEQ ID NO:85 from nucleotide 391 to nucleotide 3210, to a nucleotide sequence corresponding to the 3′ end of said sequence of SEQ ID NO:85 from nucleotide 391 to nucleotide 3210. Also preferably the polynucleotide isolated according to the above process comprises a nucleotide sequence corresponding to the cDNA sequence of SEQ ID NO:85 from nucleotide 505 to nucleotide 3210, and extending contiguously from a nucleotide sequence corresponding to the 5′ end of said sequence of SEQ ID NO:85 from nucleotide 505 to nucleotide 3210, to a nucleotide sequence corresponding to the 3′ end of said sequence of SEQ ID NO:85 from nucleotide 505 to nucleotide 3210.

In other embodiments, the present invention provides a composition comprising a protein, wherein said protein comprises an amino acid sequence selected from the group consisting of:

(a) the amino acid sequence of SEQ ID NO:86;

(b) a fragment of the amino acid sequence of SEQ ID NO:86, the fragment comprising eight contiguous amino acids of SEQ ID NO:86; and

(c) the amino acid sequence encoded by the cDNA insert of clone kj320_{—}1 deposited under accession number ATCC 98818;

the protein being substantially free from other mammalian proteins. Preferably such protein comprises the amino acid sequence of SEQ ID NO:86. In further preferred embodiments, the present invention provides a protein comprising a fragment of the amino acid sequence of SEQ ID NO:86 having biological activity, the fragment preferably comprising eight (more preferably twenty, most preferably thirty) contiguous amino acids of SEQ ID NO:86, or a protein comprising a fragment of the amino acid sequence of SEQ ID NO:86 having biological activity, the fragment comprising the amino acid sequence from amino acid 465 to amino add 474 of SEQ ID NO:86.

In one embodiment, the present invention provides a composition comprising an isolated polynucleotide selected from the group consisting of:

(a) a polynucleotide comprising the nucleotide sequence of SEQ ID NO:87;

(b) a polynucleotide comprising the nucleotide sequence of SEQ ID NO:87 from nucleotide 42 to nucleotide 899;

(c) a polynucleotide comprising the nucleotide sequence of SEQ ID NO:87 from nucleotide 522 to nucleotide 899;

(d) a polynucleotide comprising the nucleotide sequence of the full-length protein coding sequence of clone ml236_{—}5 deposited under accession number ATCC 98818;

(e) a polynucleotide encoding the full-length protein encoded by the cDNA insert of clone ml236_{—}5 deposited under accession number ATCC 98818;

(f) a polynucleotide comprising the nucleotide sequence of a mature protein coding sequence of clone ml236_{—}5 deposited under accession number ATCC 98818;

(g) a polynucleotide encoding a mature protein encoded by the cDNA insert of clone ml236_{—}5 deposited under accession number ATCC 98818;

(h) a polynucleotide encoding a protein comprising the amino acid sequence of SEQ ID NO:88;

(i) a polynucleotide encoding a protein comprising a fragment of the amino acid sequence of SEQ ID NO:88 having biological activity, the fragment comprising eight contiguous amino acids of SEQ ID NO:88;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:87.

Preferably, such polynucleotide comprises the nucleotide sequence of SEQ. ID NO:87 from nucleotide 42 to nucleotide 899; the nucleotide sequence of SEQ ID NO:87 from nucleotide 522 to nucleotide 899; the nucleotide sequence of the full-length protein coding sequence of clone ml236_{—}5 deposited under accession number ATCC 98818; or the nucleotide sequence of a mature protein coding sequence of clone ml236_{—}5 deposited under accession number ATCC 98818. In other preferred embodiments, the polynucleotide encodes the full-length or a mature protein encoded by the cDNA insert of clone ml236_{—}5 deposited under accession number ATCC 98818. In further preferred embodiments, the present invention provides a polynucleotide encoding a protein comprising a fragment of the amino add sequence of SEQ ID NO:88 having biological activity, the fragment preferably comprising eight (more preferably twenty, most preferably thirty) contiguous amino acids of SEQ ID NO:88, or a polynucleotide encoding a protein comprising a fragment of the amino acid sequence of SEQ ID NO:88 having biological activity, the fragment comprising the amino add sequence from amino acid 138 to amino acid 147 of SEQ ID NO:88.

Other embodiments provide the gene corresponding to the cDNA sequence of SEQ ID NO:87.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:87, but excluding the poly(A) tail at the 3′ end of SEQ ID NO:87; and

(ab) the nucleotide sequence of the cDNA insert of clone ml236_{—}5 deposited under accession number ATCC 98818;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:87, but excluding the poly(A) tail at the 3′ end of SEQ ID NO:87; and

(bb) the nucleotide sequence of the cDNA insert of clone ml236_{—}5 deposited under accession number ATCC 98818;

(ii) hybridizing said primer(s) to human genomic DNA in conditions at least as stringent as 4×SSC at 50 degrees C.;

(ii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:87, and extending contiguously from a nucleotide sequence corresponding to the 5′ end of SEQ ID NO:87 to a nucleotide sequence corresponding to the 3′ end of SEQ ID NO:87, but excluding the poly(A) tail at the 3′ end of SEQ ID NO:87. Also preferably the polynucleotide isolated according to the above process comprises a nucleotide sequence corresponding to the cDNA sequence of SEQ ID NO:87 from nucleotide 42 to nucleotide 899, and extending contiguously from a nucleotide sequence corresponding to the 5′ end of said sequence of SEQ ID NO:87 from nucleotide 42 to nucleotide 899, to a nucleotide sequence corresponding to the 3′ end of said sequence of SEQ ID NO:87 from nucleotide 42 to nucleotide 899. Also preferably the polynucleotide isolated according to the above process comprises a nucleotide sequence corresponding to the cDNA sequence of SEQ ID NO:87 from nucleotide 522 to nucleotide 899, and extending contiguously from a nucleotide sequence corresponding to the 5′ end of said sequence of SEQ ID NO:87 from nucleotide 522 to nucleotide 899, to a nucleotide sequence corresponding to the 3′ end of said sequence of SEQ ID NO:87 from nucleotide 522 to nucleotide 899.

In other embodiments, the present invention provides a composition comprising a protein, wherein said protein comprises an amino add sequence selected from the group consisting of:

(a) the amino acid sequence of SEQ ID NO:88;

(b) a fragment of the amino acid sequence of SEQ ID NO:88, the fragment comprising eight contiguous amino acids of SEQ ID NO:88; and

(c) the amino acid sequence encoded by the cDNA insert of clone ml236_{—}5 deposited under accession number ATCC 98818;

the protein being substantially free from other mammalian proteins. Preferably such protein comprises the amino acid sequence of SEQ ID NO:88. In further preferred embodiments, the present invention provides a protein comprising a fragment of the amino add sequence of SEQ ID NO:88 having biological activity, the fragment preferably comprising eight (more preferably twenty, most preferably thirty) contiguous amino acids of SEQ ID NO:88, or a protein comprising a fragment of the amino acid sequence of SEQ ID NO:88 having biological activity, the fragment comprising the amino acid sequence from amino acid 138 to amino acid 147 of SEQ ID NO:88.

In one embodiment, the present invention provides a composition comprising an isolated polynucleotide selected from the group consisting of:

(a) a polynucleotide comprising the nucleotide sequence of SEQ ID NO:89;

(b) a polynucleotide comprising the nucleotide sequence of SEQ ID NO:89 from nucleotide 6 to nucleotide 452;

(c) a polynucleotide comprising the nucleotide sequence of SEQ ID NO:89 from nucleotide 399 to nucleotide 452;

(d) a polynucleotide comprising the nucleotide sequence of the full-length protein coding sequence of clone pu282_{—}10 deposited under accession number ATCC 98818;

(e) a polynucleotide encoding the full-length protein encoded by the cDNA insert of clone pu282_{—}10 deposited under accession number ATCC 98818;

(f) a polynucleotide comprising the nucleotide sequence of a mature protein coding sequence of clone pu282_{—}10 deposited under accession number ATCC 98818;

(g) a polynucleotide encoding a mature protein encoded by the cDNA insert of clone pu282_{—}10 deposited under accession number ATCC 98818;

(h) a polynucleotide encoding a protein comprising the amino acid sequence of SEQ ID NO:90;

(i) a polynucleotide encoding a protein comprising a fragment of the amino acid sequence of SEQ ID NO:90 having biological activity, the fragment comprising eight contiguous amino acids of SEQ ID NO:90;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:89.

Preferably, such polynucleotide comprises the nucleotide sequence of SEQ ID NO:89 from nucleotide 6 to nucleotide 452; the nucleotide sequence of SEQ ID NO:89 from nucleotide 399 to nucleotide 452; the nucleotide sequence of the full-length protein coding sequence of clone pu282_{—}10 deposited under accession number ATCC 98818; or the nucleotide sequence of a mature protein coding sequence of clone pu282_{—}10 deposited under accession number ATCC 98818. In other preferred embodiments, the polynucleotide encodes the full-length or a mature protein encoded by the cDNA insert of clone pu282_{—}10 deposited under accession number ATCC 98818. In further preferred embodiments, the present invention provides a polynucleotide encoding a protein comprising a fragment of the amino acid sequence of SEQ ID NO:90 having biological activity, the fragment preferably comprising eight (more preferably twenty, most preferably thirty) contiguous amino acids of SEQ ID NO:90, or a polynucleotide encoding a protein comprising a fragment of the amino acid sequence of SEQ ID NO:90 having biological activity, the fragment comprising the amino acid sequence from amino acid 69 to amino acid 78 of SEQ ID NO:90.

Other embodiments provide the gene corresponding to the cDNA sequence of SEQ ID NO:89.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:89, but excluding the poly(A) tail at the 3′ end of SEQ ID NO:89; and

(ab) the nucleotide sequence of the cDNA insert of clone pu282_{—}10 deposited under accession number ATCC 98818;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:89, but excluding the poly(A) tail at the 3′ end of SEQ ID NO:89; and

(bb) the nucleotide sequence of the cDNA insert of clone pu282_{—}10 deposited under accession number ATCC 98818;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:89, and extending contiguously from a nucleotide sequence corresponding to the 5′ end of SEQ ID NO:89 to a nucleotide sequence corresponding to the 3′ end of SEQ ID NO:89, but excluding the poly(A) tail at the 3′ end of SEQ ID NO:89. Also preferably the polynucleotide isolated according to the above process comprises a nucleotide sequence corresponding to the cDNA sequence of SEQ ID NO:89 from nucleotide 6 to nucleotide 452, and extending contiguously from a nucleotide sequence corresponding to the 5′ end of said sequence of SEQ ID NO:89 from nucleotide 6 to nucleotide 452, to a nucleotide sequence corresponding to the 3′ end of said sequence of SEQ ID NO:89 from nucleotide 6 to nucleotide 452. Also preferably the polynucleotide isolated according to the above process comprises a nucleotide sequence corresponding to the cDNA sequence of SEQ ID NO:89 from nucleotide 399 to nucleotide 452, and extending contiguously from a nucleotide sequence corresponding to the 5′ end of said sequence of SEQ ID NO:89 from nucleotide 399 to nucleotide 452, to a nucleotide sequence corresponding to the 3′ end of said sequence of SEQ ID NO:89 from nucleotide 399 to nucleotide 452.

In other embodiments, the present invention provides a composition comprising a protein, wherein said protein comprises an amino acid sequence selected from the group consisting of:

(a) the amino acid sequence of SEQ ID NO:90;

(b) a fragment of the amino acid sequence of SEQ ID NO:90, the fragment comprising eight contiguous amino acids of SEQ ID NO:90; and

(c) the amino acid sequence encoded by the cDNA insert of clone pu282_{—}10 deposited under accession number ATCC 98818;

the protein being substantially free from other mammalian proteins. Preferably such protein comprises the amino acid sequence of SEQ ID NO:90. In further preferred embodiments, the present invention provides a protein comprising a fragment of the amino acid sequence of SEQ ID NO:90 having biological activity, the fragment preferably comprising eight (more preferably twenty, most preferably thirty) contiguous amino acids of SEQ ID NO:90, or a protein comprising a fragment of the amino acid sequence of SEQ ID NO:90 having biological activity, the fragment comprising the amino acid sequence from amino acid 69 to amino acid 78 of SEQ ID NO:90.

In one embodiment, the present invention provides a composition comprising an isolated polynucleotide selected from the group consisting of:

(a) a polynucleotide comprising the nucleotide sequence of SEQ ID NO:91;

(b) a polynucleotide comprising the nucleotide sequence of SEQ ID NO:91 from nucleotide 4 to nucleotide 1179;

(c) a polynucleotide comprising the nucleotide sequence of SEQ ID NO:91 from nucleotide 682 to nucleotide 1179;

(d) a polynucleotide comprising the nucleotide sequence of the full-length protein coding sequence of clone at94_{—}2 deposited under accession number ATCC 98822;

(e) a polynucleotide encoding the full-length protein encoded by the cDNA insert of clone at94_{—}2 deposited under accession number ATCC 98822;

(f) a polynucleotide comprising the nucleotide sequence of a mature protein coding sequence of clone at94_{—}2 deposited under accession number ATCC 98822;

(g) a polynucleotide encoding a mature protein encoded by the cDNA insert of clone at94_{—}2 deposited under accession number ATCC 98822;

(h) a polynucleotide encoding a protein comprising the amino acid sequence of SEQ ID NO:92;

(i) a polynucleotide encoding a protein comprising a fragment of the amino acid sequence of SEQ ID NO:92 having biological activity, the fragment comprising eight contiguous amino acids of SEQ ID NO:92;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:91.

Preferably, such polynucleotide comprises the nucleotide sequence of SEQ ID NO:91 from nucleotide 4 to nucleotide 1179; the nucleotide sequence of SEQ ID NO:91 from nucleotide 682 to nucleotide 1179; the nucleotide sequence of the full-length protein coding sequence of clone at94_{—}2 deposited under accession number ATCC 98822; or the nucleotide sequence of a mature protein coding sequence of clone at94_{—}2 deposited under accession number ATCC 98822. In other preferred embodiments, the polynucleotide encodes the full-length or a mature protein encoded by the cDNA insert of clone at94_{—}2 deposited under accession number ATCC 98822. In further preferred embodiments, the present invention provides a polynucleotide encoding a protein comprising a fragment of the amino acid sequence of SEQ ID NO:92 having biological activity, the fragment preferably comprising eight (more preferably twenty, most preferably thirty) contiguous amino acids of SEQ ID NO:92, or a polynucleotide encoding a protein comprising a fragment of the amino acid sequence of SEQ ID NO:92 having biological activity, the fragment comprising the amino acid sequence from amino acid 191 to amino acid 200 of SEQ ID NO:92.

Other embodiments provide the gene corresponding to the cDNA sequence of SEQ ID NO:91.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:91, but excluding the poly(A) tail at the 3′ end of SEQ ID NO:91; and

(ab) the nucleotide sequence of the cDNA insert of clone at94_{—}2 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:91, but excluding the poly(A) tail at the 3′ end of SEQ ID NO:91; and

(bb) the nucleotide sequence of the cDNA insert of clone at94_{—}2 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:91, and extending contiguously from a nucleotide sequence corresponding to the 5′ end of SEQ ID NO:91 to a nucleotide sequence corresponding to the 3′ end of SEQ ID NO:91, but excluding the poly(A) tail at the 3′ end of SEQ ID NO:91. Also preferably the polynucleotide isolated according to the above process comprises a nucleotide sequence corresponding to the cDNA sequence of SEQ ID NO:91 from nucleotide 4 to nucleotide 1179, and extending contiguously from a nucleotide sequence corresponding to the 5′ end of said sequence of SEQ ID NO:91 from nucleotide 4 to nucleotide 1179, to a nucleotide sequence corresponding to the 3′ end of said sequence of SEQ ID NO:91 from nucleotide 4 to nucleotide 1179. Also preferably the polynucleotide isolated according to the above process comprises a nucleotide sequence corresponding to the cDNA sequence of SEQ ID NO:91 from nucleotide 682 to nucleotide 1179, and extending contiguously from a nucleotide sequence corresponding to the 5′ end of said sequence of SEQ ID NO:91 from nucleotide 682 to nucleotide 1179, to a nucleotide sequence corresponding to the 3′ end of said sequence of SEQ ID NO:91 from nucleotide 682 to nucleotide 1179.

In other embodiments, the present invention provides a composition comprising a protein, wherein said protein comprises an amino add sequence selected from the group consisting of:

(a) the amino acid sequence of SEQ ID NO:92;

(b) a fragment of the amino acid sequence of SEQ ID NO:92, the fragment comprising eight contiguous amino acids of SEQ ID NO:92; and

(c) the amino acid sequence encoded by the cDNA insert of clone at94_{—}2 deposited under accession number ATCC 98822;

the protein being substantially free from other mammalian proteins. Preferably such protein comprises the amino acid sequence of SEQ ID NO:92. In further preferred embodiments, the present invention provides a protein comprising a fragment of the amino add sequence of SEQ ID NO:92 having biological activity, the fragment preferably comprising eight (more preferably twenty, most preferably thirty) contiguous amino adds of SEQ ID NO:92, or a protein comprising a fragment of the amino acid sequence of SEQ ID NO:92 having biological activity, the fragment comprising the amino add sequence from amino acid 191 to amino acid 200 of SEQ ID NO:92.

In one embodiment, the present invention provides a composition comprising an isolated polynucleotide selected from the group consisting of:

(a) a polynucleotide comprising the nucleotide sequence of SEQ ID NO:93;

(b) a polynucleotide comprising the nucleotide sequence of SEQ ID NO:93 from nucleotide 56 to nucleotide 2077;

(c) a polynucleotide comprising the nucleotide sequence of the full-length protein coding sequence of clone bf169_{—}13 deposited under accession number ATCC 98822;

(d) a polynucleotide encoding the full-length protein encoded by the cDNA insert of clone bf169_{—}13 deposited under accession number ATCC 98822;

(e) a polynucleotide comprising the nucleotide sequence of a mature protein coding sequence of clone bf169_{—}13 deposited under accession number ATCC 98822;

(f) a polynucleotide encoding a mature protein encoded by the cDNA insert of clone bf169_{—}13 deposited under accession number ATCC 98822;

(g) a polynucleotide encoding a protein comprising the amino acid sequence of SEQ ID NO:94;

(h) a polynucleotide encoding a protein comprising a fragment of the amino acid sequence of SEQ ID NO:94 having biological activity, the fragment comprising eight contiguous amino acids of SEQ ID NO:94;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:93.

Preferably, such polynucleotide comprises the nucleotide sequence of SEQ ID NO:93 from nucleotide 56 to nucleotide 2077; the nucleotide sequence of the full-length protein coding sequence of clone bf169_{—}13 deposited under accession number ATCC 98822; or the nucleotide sequence of a mature protein coding sequence of clone bf169_{—}13 deposited under accession number ATCC 98822. In other preferred embodiments, the polynucleotide encodes the full-length or a mature protein encoded by the cDNA insert of clone bf169_{—}13 deposited under accession number ATCC 98822. In further preferred embodiments, the present invention provides a polynucleotide encoding a protein comprising a fragment of the amino acid sequence of SEQ ID NO:94 having biological activity, the fragment preferably comprising eight (more preferably twenty, most preferably thirty) contiguous amino acids of SEQ ID NO:94, or a polynucleotide encoding a protein comprising a fragment of the amino acid sequence of SEQ ID NO:94 having biological activity, the fragment comprising the amino acid sequence from amino acid 332 to amino acid 341 of SEQ ID NO:94.

Other embodiments provide the gene corresponding to the cDNA sequence of SEQ ID NO:93.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:93, but excluding the poly(A) tail at the 3′ end of SEQ ID NO:93; and

(ab) the nucleotide sequence of the cDNA insert of clone bf169_{—}13 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:93, but excluding the poly(A) tail at the 3′ end of SEQ ID NO:93; and

(bb) the nucleotide sequence of the cDNA insert of clone bf169_{—}13 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:93, and extending contiguously from a nucleotide sequence corresponding to the 5′ end of SEQ ID NO:93 to a nucleotide sequence corresponding to the 3′ end of SEQ ID NO:93, but excluding the poly(A) tail at the 3′ end of SEQ ID NO:93. Also preferably the polynucleotide isolated according to the above process comprises a nucleotide sequence corresponding to the cDNA sequence of SEQ ID NO:93 from nucleotide 56 to nucleotide 2077, and extending contiguously from a nucleotide sequence corresponding to the 5′ end of said sequence of SEQ ID NO:93 from nucleotide 56 to nucleotide 2077, to a nucleotide sequence corresponding to the 3′ end of said sequence of SEQ ID NO:93 from nucleotide 56 to nucleotide 2077.

In other embodiments, the present invention provides a composition comprising a protein, wherein said protein comprises an amino acid sequence selected from the group consisting of:

(a) the amino acid sequence of SEQ ID NO:94;

(b) a fragment of the amino acid sequence of SEQ ID NO:94, the fragment comprising eight contiguous amino acids of SEQ ID NO:94; and

(c) the amino acid sequence encoded by the cDNA insert of clone bf169_{—}13 deposited under accession number ATCC 98822;

the protein being substantially free from other mammalian proteins. Preferably such protein comprises the amino acid sequence of SEQ ID NO:94. In further preferred embodiments, the present invention provides a protein comprising a fragment of the amino acid sequence of SEQ ID NO:94 having biological activity, the fragment preferably comprising eight (more preferably twenty, most preferably thirty) contiguous amino acids of SEQ ID NO:94, or a protein comprising a fragment of the amino acid sequence of SEQ ID NO:94 having biological activity, the fragment comprising the amino acid sequence from amino acid 332 to amino acid 341 of SEQ ID NO:94.

In one embodiment, the present invention provides a composition comprising an isolated polynucleotide selected from the group consisting of:

(a) a polynucleotide comprising the nucleotide sequence of SEQ ID NO:95;

(b) a polynucleotide comprising the nucleotide sequence of SEQ ID NO:95 from nucleotide 124 to nucleotide 735;

(c) a polynucleotide comprising the nucleotide sequence of the full-length protein coding sequence of clone bl152_{—}12 deposited under accession number ATCC 98822;

(d) a polynucleotide encoding the full-length protein encoded by the cDNA insert of clone bl152_{—}12 deposited under accession number ATCC 98822;

(e) a polynucleotide comprising the nucleotide sequence of a mature protein coding sequence of clone bl152_{—}12 deposited under accession number ATCC 98822;

(f) a polynucleotide encoding a mature protein encoded by the cDNA insert of clone bl152_{—}12 deposited under accession number ATCC 98822;

(g) a polynucleotide encoding a protein comprising the amino acid sequence of SEQ ID NO:96;

(h) a polynucleotide encoding a protein comprising a fragment of the amino acid sequence of SEQ ID NO:96 having biological activity, the fragment comprising eight contiguous amino acids of SEQ ID NO:96;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-h) and that has a length that is at least 25% of the length of SEQ ID NO:95.

Preferably, such polynucleotide comprises the nucleotide sequence of SEQ ID NO:95 from nucleotide 124 to nucleotide 735; the nucleotide sequence of the full-length protein coding sequence of clone bl152_{—}12 deposited under accession number ATCC 98822; or the nucleotide sequence of a mature protein coding sequence of clone bl152_{—}12 deposited under accession number ATCC 98822. In other preferred embodiments, the polynucleotide encodes the full-length or a mature protein encoded by the cDNA insert of clone bl152_{—}12 deposited under accession number ATCC 98822. In further preferred embodiments, the present invention provides a polynucleotide encoding a protein comprising a fragment of the amino acid sequence of SEQ ID NO:96 having biological activity, the fragment preferably comprising eight (more preferably twenty, most preferably thirty) contiguous amino adds of SEQ ID NO:96, or a polynucleotide encoding a protein comprising a fragment of the amino acid sequence of SEQ ID NO:96 having biological activity, the fragment comprising the amino acid sequence from amino acid 97 to amino acid 106 of SEQ ID NO:96.

Other embodiments provide the gene corresponding to the cDNA sequence of SEQ ID NO:95.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:95, but excluding the poly(A) tail at the 3′ end of SEQ ID NO:95; and

(ab) the nucleotide sequence of the cDNA insert of clone bl152_{—}12 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:95, but excluding the poly(A) tail at the 3′ end of SEQ ID NO:95; and

(bb) the nucleotide sequence of the cDNA in set of clone bl152_{—}12 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:95, and extending contiguously from a nucleotide sequence corresponding to the 5′ end of SEQ ID NO:95 to a nucleotide sequence corresponding to the 3′ end of SEQ ID NO:95, but excluding the poly(A) tail at the 3′ end of SEQ ID NO:95. Also preferably the polynucleotide isolated according to the above process comprises a nucleotide sequence corresponding to the cDNA sequence of SEQ ID NO:95 from nucleotide 124 to nucleotide 735, and extending contiguously from a nucleotide sequence corresponding to the 5′ end of said sequence of SEQ ID NO:95 from nucleotide 124 to nucleotide 735, to a nucleotide sequence corresponding to the 3′ end of said sequence of SEQ ID NO:95 from nucleotide 124 to nucleotide 735.

In other embodiments, the present invention provides a composition comprising a protein, wherein said protein comprises an amino acid sequence selected from the group consisting of:

(a) the amino acid sequence of SEQ ID NO:96;

(b) a fragment of the amino acid sequence of SEQ ID NO:96, the fragment comprising eight contiguous amino acids of SEQ ID NO:96; and

(c) the amino acid sequence encoded by the cDNA insert of clone bl152_{—}12 deposited under accession number ATCC 98822;

the protein being substantially free from other mammalian proteins. Preferably such protein comprises the amino acid sequence of SEQ ID NO:96. In further preferred embodiments, the present invention provides a protein comprising a fragment of the amino acid sequence of SEQ ID NO:96 having biological activity, the fragment preferably comprising eight (more preferably twenty, most preferably thirty) contiguous amino acids of SEQ ID NO:96, or a protein comprising a fragment of the amino acid sequence of SEQ ID NO:96 having biological activity, the fragment comprising the amino acid sequence from amino acid 97 to amino acid 106 of SEQ ID NO:96.

In one embodiment, the present invention provides a composition comprising an isolated polynucleotide selected from the group consisting of:

(a) a polynucleotide comprising the nucleotide sequence of SEQ ID NO:97;

(b) a polynucleotide comprising the nucleotide sequence of SEQ ID NO:97 from nucleotide 526 to nucleotide 816;

(c) a polynucleotide comprising the nucleotide sequence of the full-length protein coding sequence of clone bz578_{—}1 deposited under accession number ATCC 98822;

(d) a polynucleotide encoding the full-length protein encoded by the cDNA insert of clone bz578_{—}1 deposited under accession number ATCC 98822;

(e) a polynucleotide comprising the nucleotide sequence of a mature protein coding sequence of clone bz578_{—}1 deposited under accession number ATCC 98822;

(f) a polynucleotide encoding a mature protein encoded by the cDNA insert of clone bz578_{—}1 deposited under accession number ATCC 98822;

(g) a polynucleotide encoding a protein comprising the amino acid sequence of SEQ ID NO:98;

(h) a polynucleotide encoding a protein comprising a fragment of the amino acid sequence of SEQ ID NO:98 having biological activity, the fragment comprising eight contiguous amino acids of SEQ ID NO:98;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:97.

Preferably, such polynucleotide comprises the nucleotide sequence of SEQ ID NO:97 from nucleotide 526 to nucleotide 816; the nucleotide sequence of the full-length protein coding sequence of clone bz578_{—}1 deposited under accession number ATCC 98822; or the nucleotide sequence of a mature protein coding sequence of clone bz578_{—}1 deposited under accession number ATCC 98822. In other preferred embodiments, the polynucleotide encodes the full-length or a mature protein encoded by the cDNA insert of clone bz578_{—}1 deposited under accession number ATCC 98822. In further preferred embodiments, the present invention provides a polynucleotide encoding a protein comprising a fragment of the amino acid sequence of SEQ ID NO:98 having biological activity, the fragment preferably comprising eight (more preferably twenty, most preferably thirty) contiguous amino acids of SEQ ID NO:98, or a polynucleotide encoding a protein comprising a fragment of the amino acid sequence of SEQ ID NO:98 having biological activity, the fragment comprising the amino acid sequence from amino acid 43 to amino acid 52 of SEQ ID NO:98.

Other embodiments provide the gene corresponding to the cDNA sequence of SEQ ID NO:97.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:97, but excluding the poly(A) tail at the 3′ end of SEQ ID NO:97; and

(ab) the nucleotide sequence of the cDNA insert of clone bz578_{—}1 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:97, but excluding the poly(A) tail at the 3′ end of SEQ ID NO:97; and

(bb) the nucleotide sequence of the cDNA insert of clone bz578_{—}1 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:97, and extending contiguously from a nucleotide sequence corresponding to the 5′ end of SEQ ID NO:97 to a nucleotide sequence corresponding to the 3′ end of SEQ ID NO:97, but excluding the poly(A) tail at the 3′ end of SEQ ID NO:97. Also preferably the polynucleotide isolated according to the above process comprises a nucleotide sequence corresponding to the cDNA sequence of SEQ ID NO:97 from nucleotide 526 to nucleotide 816, and extending contiguously from a nucleotide sequence corresponding to the 5′ end of said sequence of SEQ ID NO:97 from nucleotide 526 to nucleotide 816, to a nucleotide sequence corresponding to the 3′ end of said sequence of SEQ ID NO:97 from nucleotide 526 to nucleotide 816.

In other embodiments, the present invention provides a composition comprising a protein, wherein said protein comprises an amino add sequence selected from the group consisting of:

(a) the amino acid sequence of SEQ ID NO:98;

(b) a fragment of the amino acid sequence of SEQ ID NO:98, the fragment comprising eight contiguous amino acids of SEQ ID NO:98; and

(c) the amino acid sequence encoded by the cDNA insert of clone bz578_{—}1 deposited under accession number ATCC 98822;

the protein being substantially free from other mammalian proteins. Preferably such protein comprises the amino acid sequence of SEQ ID NO:98. In further preferred embodiments, the present invention provides a protein comprising a fragment of the amino acid sequence of SEQ ID NO:98 having biological activity, the fragment preferably comprising eight (more preferably twenty, most preferably thirty) contiguous amino acids of SEQ ID NO:98, or a protein comprising a fragment of the amino acid sequence of SEQ ID NO:98 having biological activity, the fragment comprising the amino add sequence from amino acid 43 to amino acid 52 of SEQ ID NO:98.

In one embodiment, the present invention provides a composition comprising an isolated polynucleotide selected from the group consisting of:

(a) a polynucleotide comprising the nucleotide sequence of SEQ ID NO:99;

(b) a polynucleotide comprising the nucleotide sequence of SEQ ID NO:99 from nucleotide 597 to nucleotide 992;

(c) a polynucleotide comprising the nucleotide sequence of SEQ ID NO:99 from nucleotide 765 to nucleotide 992;

(d) a polynucleotide comprising the nucleotide sequence of the full-length protein coding sequence of clone cb123_{—}1 deposited under accession number ATCC 98822;

(e) a polynucleotide encoding the full-length protein encoded by the cDNA insert of clone cb123_{—}1 deposited under accession number ATCC 98822;

(f) a polynucleotide comprising the nucleotide sequence of a mature protein coding sequence of clone cb123_{—}1 deposited under accession number ATCC 98822;

(g) a polynucleotide encoding a mature protein encoded by the cDNA insert of clone cb123_{—}1 deposited under accession number ATCC 98822;

(h) a polynucleotide encoding a protein comprising the amino acid sequence of SEQ ID NO:100;

(i) a polynucleotide encoding a protein comprising a fragment of the amino acid sequence of SEQ ID NO:100 having biological activity, the fragment comprising eight contiguous amino acids of SEQ ID NO:100;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:99.

Preferably, such polynucleotide comprises the nucleotide sequence of SEQ ID NO:99 from nucleotide 597 to nucleotide 992; the nucleotide sequence of SEQ ID NO:99 from nucleotide 765 to nucleotide 992; the nucleotide sequence of the full-length protein coding sequence of clone cb123_{—}1 deposited under accession number ATCC 98822; or the nucleotide sequence of a mature protein coding sequence of clone cb123_{—}1 deposited under accession number ATCC 98822. In other preferred embodiments, the polynucleotide encodes the full-length or a mature protein encoded by the cDNA insert of clone cb123_{—}1 deposited under accession number ATCC 98822. In further preferred embodiments, the present invention provides a polynucleotide encoding a protein comprising a fragment of the amino acid sequence of SEQ ID NO:100 having biological activity, the fragment preferably comprising eight (more preferably twenty, most preferably thirty) contiguous amino acids of SEQ ID NO:100, or a polynucleotide encoding a protein comprising a fragment of the amino acid sequence of SEQ ID NO:100 having biological activity, the fragment comprising the amino add sequence from amino acid 61 to amino acid 70 of SEQ ID NO:100.

Other embodiments provide the gene corresponding to the cDNA sequence of SEQ ID NO:99.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:99, but excluding the poly(A) tail at the 3′ end of SEQ ID NO:99; and

(ab) the nucleotide sequence of the cDNA insert of clone cb123_{—}11 deposited under accession number ATCC. 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:99, but excluding the poly(A) tail at the 3′ end of SEQ ID NO:99; and

(bb) the nucleotide sequence of the cDNA insert of clone cb123_{—}1 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:99, and extending contiguously from a nucleotide sequence corresponding to the 5′ end of SEQ ID NO:99 to a nucleotide sequence corresponding to the 3′ end of SEQ ID NO:99, but excluding the poly(A) tail at the 3′ end of SEQ ID NO:99. Also preferably the polynucleotide isolated according to the above process comprises a nucleotide sequence corresponding to the cDNA sequence of SEQ ID NO:99 from nucleotide 597 to nucleotide 992, and extending contiguously from a nucleotide sequence corresponding to the 5′ end of said sequence of SEQ ID NO:99 from nucleotide 597 to nucleotide 992, to a nucleotide sequence corresponding to the 3′ end of said sequence of SEQ ID NO:99 from nucleotide 597 to nucleotide 992. Also preferably the polynucleotide isolated according to the above process comprises a nucleotide sequence corresponding to the cDNA sequence of SEQ ID NO:99 from nucleotide 765 to nucleotide 992, and extending contiguously from a nucleotide sequence corresponding to the 5′ end of said sequence of SEQ ID NO:99 from nucleotide 765 to nucleotide 992, to a nucleotide sequence corresponding to the 3′ end of said sequence of SEQ ID NO:99 from nucleotide 765 to nucleotide 992.

In other embodiments, the present invention provides a composition comprising a protein, wherein said protein comprises an amino acid sequence selected from the group consisting of:

(a) the amino acid sequence of SEQ ID NO:100;

(b) a fragment of the amino acid sequence of SEQ ID NO:100, the fragment comprising eight contiguous amino adds of SEQ ID NO:100; and

(c) the amino acid sequence encoded by the cDNA insert of clone cb123_{—}1 deposited under accession number ATCC 98822;

the protein being substantially free from other mammalian proteins. Preferably such protein comprises the amino acid sequence of SEQ ID NO:100. In further preferred embodiments, the present invention provides a protein comprising a fragment of the amino acid sequence of SEQ ID NO:100 having biological activity, the fragment preferably comprising eight (more preferably twenty, most preferably thirty) contiguous amino acids of SEQ ID NO:100, or a protein comprising a fragment of the amino acid sequence of SEQ ID NO:100 having biological activity, the fragment comprising the amino acid sequence from amino acid 61 to amino acid 70 of SEQ ID NO:100.

In one embodiment, the present invention provides a composition comprising an isolated polynucleotide selected from the group consisting of:

(a) a polynucleotide comprising the nucleotide sequence of SEQ ID NO:101;

(b) a polynucleotide comprising the nucleotide sequence of SEQ ID NO: 101 from nucleotide 181 to nucleotide 480;

(c) a polynucleotide comprising the nucleotide sequence of the full-length protein coding sequence of clone ch245_{—}1 deposited under accession number ATCC 98822;

(d) a polynucleotide encoding the full-length protein encoded by the cDNA insert of clone ch245_{—}1 deposited under accession number ATCC 98822;

(e) a polynucleotide comprising the nucleotide sequence of a mature protein coding sequence of clone ch245_{—}1 deposited under accession number ATCC 98822;

(f) a polynucleotide encoding a mature protein encoded by the cDNA insert of clone ch245_{—}1 deposited under accession number ATCC 98822;

(g) a polynucleotide encoding a protein comprising the amino acid sequence of SEQ ID NO:102;

(h) a polynucleotide encoding a protein comprising a fragment of the amino acid sequence of SEQ ID NO:102 having biological activity, the fragment comprising eight contiguous amino acids of SEQ ID NO:102;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:101.

Preferably, such polynucleotide comprises the nucleotide sequence of SEQ ID NO:101 from nucleotide 181 to nucleotide 480; the nucleotide sequence of the full-length protein coding sequence of clone ch245_{—}1 deposited under accession number ATCC 98822; or the nucleotide sequence of a mature protein coding sequence of clone ch245_{—}1 deposited under accession number ATCC 98822. In other preferred embodiments, the polynucleotide encodes the full-length or a mature protein encoded by the cDNA insert of clone ch245_{—}1 deposited under accession number ATCC 98822. In further preferred embodiments, the present invention provides a polynucleotide encoding a protein comprising a fragment of the amino acid sequence of SEQ ID NO:102 having biological activity, the fragment preferably comprising eight (more preferably twenty, most preferably thirty) contiguous amino acids of SEQ ID NO:102, or a polynucleotide encoding a protein comprising a fragment of the amino acid sequence of SEQ ID NO:102 having biological activity, the fragment comprising the amino acid sequence from amino acid 45 to amino acid 54 of SEQ ID NO:102.

Other embodiments provide the gene corresponding to the cDNA sequence of SEQ ID NO:101.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:101, but excluding the poly(A) tail at the 3′ end of SEQ ID NO:101; and

(ab) the nucleotide sequence of the cDNA insert of clone ch245_{—}1 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:101, but excluding the poly(A) tail at the 3′ end of SEQ ID NO:101; and

(bb) the nucleotide sequence of the cDNA insert of clone ch2451 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:101, and extending contiguously from a nucleotide sequence corresponding to the 5′ end of SEQ ID NO:101 to a nucleotide sequence corresponding to the 3′ end of SEQ ID NO: 101, but excluding the poly(A) tail at the 3′ end of SEQ ID NO:101. Also preferably the polynucleotide isolated according to the above process comprises a nucleotide sequence corresponding to the cDNA sequence of SEQ ID NO:101 from nucleotide 181 to nucleotide 480, and extending contiguously from a nucleotide sequence corresponding to the 5′ end of said sequence of SEQ ID NO:101 from nucleotide 181 to nucleotide 480, to a nucleotide sequence corresponding to the 3′ end of said sequence of SEQ ID NO:101 from nucleotide 181 to nucleotide 480.

In other embodiments, the present invention provides a composition comprising a protein, wherein said protein comprises an amino acid sequence selected from the group consisting of:

(a) the amino acid sequence of SEQ ID NO:102;

(b) a fragment of the amino acid sequence of SEQ ID NO:102, the fragment comprising eight contiguous amino acids of SEQ ID NO:102; and

(c) the amino acid sequence encoded by the cDNA insert of clone ch245_{—}1 deposited under accession number ATCC 98822;

the protein being substantially free from other mammalian proteins. Preferably such protein comprises the amino acid sequence of SEQ ID NO:102. In further preferred embodiments, the present invention provides a protein comprising a fragment of the amino acid sequence of SEQ ID NO:102 having biological activity, the fragment preferably comprising eight (more preferably twenty, most preferably thirty) contiguous amino acids of SEQ ID NO:102, or a protein comprising a fragment of the amino acid sequence of SEQ ID NO:102 having biological activity, the fragment comprising the amino acid sequence from amino acid 45 to amino acid 54 of SEQ ID NO:102.

In one embodiment, the present invention provides a composition comprising an isolated polynucleotide selected from the group consisting of:

(a) a polynucleotide comprising the nucleotide sequence of SEQ ID NO:103;

(b) a polynucleotide comprising the nucleotide sequence of SEQ ID NO:103 from nucleotide 281 to nucleotide 541;

(c) a polynucleotide comprising the nucleotide sequence of the full-length protein coding sequence of clone cj378_{—}3 deposited under accession number ATCC 98822;

(d) a polynucleotide encoding the full-length protein encoded by the cDNA insert of clone cj378_{—}3 deposited under accession number ATCC 98822;

(e) a polynucleotide comprising the nucleotide sequence of a mature protein coding sequence of clone cj378_{—}3 deposited under accession number ATCC 98822;

(f) a polynucleotide encoding a mature protein encoded by the cDNA insert of clone cj378_{—}3 deposited under accession number ATCC 98822;

(g) a polynucleotide encoding a protein comprising the amino acid sequence of SEQ ID NO:104;

(h) a polynucleotide encoding a protein comprising a fragment of the amino acid sequence of SEQ ID NO:104 having biological activity, the fragment comprising eight contiguous amino acids of SEQ ID NO:104;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO: 103.

Preferably, such polynucleotide comprises the nucleotide sequence of SEQ ID NO:103 from nucleotide 281 to nucleotide 541; the nucleotide sequence of the full-length protein coding sequence of clone cj378_{—}3 deposited under accession number ATCC 98822; or the nucleotide sequence of a mature protein coding sequence of clone cj378_{—}3 deposited under accession number ATCC 98822. In other preferred embodiments, the polynucleotide encodes the full-length or a mature protein encoded by the cDNA insert of clone cj378_{—}3 deposited under accession number ATCC 98822. In further preferred embodiments, the present invention provides a polynucleotide encoding a protein comprising a fragment of the amino acid sequence of SEQ ID NO:104 having biological activity, the fragment preferably comprising eight (more preferably twenty, most preferably thirty) contiguous amino acids of SEQ ID NO:104, or a polynucleotide encoding a protein comprising a fragment of the amino acid sequence of SEQ ID NO:104 having biological activity, the fragment comprising the amino acid sequence from amino acid 38 to amino acid 47 of SEQ ID NO:104.

Other embodiments provide the gene corresponding to the cDNA sequence of SEQ ID NO:103.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:103, but excluding the poly(A) tail at the 3′ end of SEQ ID NO:103; and

(ab) the nucleotide sequence of the cDNA insert of clone cj378_{—}3 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:103, but excluding the poly(A) tail at the 3′ end of SEQ ID NO:103; and

(bb) the nucleotide sequence of the cDNA insert of clone cj378_{—}3 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:103, and extending contiguously from a nucleotide sequence corresponding to the 5′ end of SEQ ID NO:103 to a nucleotide sequence corresponding to the 3′ end of SEQ ID NO: 103, but excluding the poly(A) tail at the 3′ end of SEQ ID NO:103. Also preferably the polynucleotide isolated according to the above process comprises a nucleotide sequence corresponding to the cDNA sequence of SEQ ID NO: 103 from nucleotide 281 to nucleotide 541, and extending contiguously from a nucleotide sequence corresponding to the 5′ end of said sequence of SEQ ID NO:103 from nucleotide 281 to nucleotide 541, to a nucleotide sequence corresponding to the 3′ end of said sequence of SEQ ID NO:103 from nucleotide 281 to nucleotide 541.

In other embodiments, the present invention provides a composition comprising a protein, wherein said protein comprises an amino acid sequence selected from the group consisting of:

(a) the amino acid sequence of SEQ ID NO:104;

(b) a fragment of the amino acid sequence of SEQ ID NO:104, the fragment comprising eight contiguous amino acids of SEQ ID NO:104; and

(c) the amino acid sequence encoded by the cDNA insert of clone cj378_{—}3 deposited under accession number ATCC 98822;

the protein being substantially free from other mammalian proteins. Preferably such protein comprises the amino acid sequence of SEQ ID NO:104. In further preferred embodiments, the present invention provides a protein comprising a fragment of the amino acid sequence of SEQ ID NO:104 having biological activity, the fragment preferably comprising eight (more preferably twenty, most preferably thirty) contiguous amino acids of SEQ ID NO:104, or a protein comprising a fragment of the amino acid sequence of SEQ ID NO:104 having biological activity, the fragment comprising the amino acid sequence from amino acid 38 to amino acid 47 of SEQ ID NO:104.

In one embodiment, the present invention provides a composition comprising an isolated polynucleotide selected from the group consisting of:

(a) a polynucleotide comprising the nucleotide sequence of SEQ ID NO: 105;

(b) a polynucleotide comprising the nucleotide sequence of SEQ ID NO:105 from nucleotide 586 to nucleotide 2202;

(c) a polynucleotide comprising the nucleotide sequence of SEQ ID NO:105 from nucleotide 401 to nucleotide 2349;

(d) a polynucleotide comprising the nucleotide sequence of the full-length protein coding sequence of clone cw1481_{—}1 deposited under accession number ATCC 98822;

(e) a polynucleotide encoding the full-length protein encoded by the cDNA insert of clone cw1481_{—}1 deposited under accession number ATCC 98822;

(f) a polynucleotide comprising the nucleotide sequence of a mature protein coding sequence of clone cw1481_{—}1 deposited under accession number ATCC 98822;

(g) a polynucleotide encoding a mature protein encoded by the cDNA insert of clone cw1481_{—}1 deposited under accession number ATCC 98822;

(h) a polynucleotide encoding a protein comprising the amino acid sequence of SEQ ID NO:106;

(i) a polynucleotide encoding a protein comprising a fragment of the amino acid sequence of SEQ ID NO:106 having biological activity, the fragment comprising eight contiguous amino acids of SEQ ID NO:106;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:105.

Preferably, such polynucleotide comprises the nucleotide sequence of SEQ ID NO:105 from nucleotide 586 to nucleotide 2202; the nucleotide sequence of SEQ ID NO:105 from nucleotide 401 to nucleotide 2349; the nucleotide sequence of the full-length protein coding sequence of clone cw1481_{—}1 deposited under accession number ATCC 98822; or the nucleotide sequence of a mature protein coding sequence of clone cw1481_{—}1 deposited under accession number ATCC 98822. In other preferred embodiments, the polynucleotide encodes the full-length or a mature protein encoded by the cDNA insert of clone cw1481_{—}1 deposited under accession number ATCC 98822. In further preferred embodiments, the present invention provides a polynucleotide encoding a protein comprising a fragment of the amino acid sequence of SEQ ID NO:106 having biological activity, the fragment preferably comprising eight (more preferably twenty, most preferably thirty) contiguous amino acids of SEQ ID NO:106, or a polynucleotide encoding a protein comprising a fragment of the amino acid sequence of SEQ ID NO:106 having biological activity, the fragment comprising the amino acid sequence from amino acid 264 to amino acid 273 of SEQ ID NO:106.

Other embodiments provide the gene corresponding to the cDNA sequence of SEQ ID NO:105.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:105, but excluding the poly(A) tail at the 3′ end of SEQ ID NO:105; and

(ab) the nucleotide sequence of the cDNA insert of clone cw1481_{—}1 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of

(ba) SEQ ID NO:105, but excluding the poly(A) tail at the 3′ end of SEQ ID NO:105; and

(bb) the nucleotide sequence of the cDNA insert of clone cw1481 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:105, and extending contiguously from a nucleotide sequence corresponding to the 5′ end of SEQ ID NO 105 to a nucleotide sequence corresponding to the 3′ end of SEQ ID NO:105, but excluding the poly(A) tail at the 3′ end of SEQ ID NO:105. Also preferably the polynucleotide isolated according to the above process comprises a nucleotide sequence corresponding to the cDNA sequence of SEQ ID NO:105 from nucleotide 586 to nucleotide 2202, and extending contiguously from a nucleotide sequence corresponding to the 5′ end of said sequence of SEQ ID NO:105 from nucleotide 586 to nucleotide 2202, to a nucleotide sequence corresponding to the 3′ end of said sequence of SEQ ID NO:105 from nucleotide 586 to nucleotide 2202. Also preferably the polynucleotide isolated according to the above process comprises a nucleotide sequence corresponding to the cDNA sequence of SEQ ID NO:105 from nucleotide 401 to nucleotide 2349, and extending contiguously from a nucleotide sequence corresponding to the 5′ end of said sequence of SEQ ID NO:105 from nucleotide 401 to nucleotide 2349, to a nucleotide sequence corresponding to the 3′ end of said sequence of SEQ ID NO:105 from nucleotide 401 to nucleotide 2349.

In other embodiments, the present invention provides a composition comprising a protein, wherein said protein comprises an amino add sequence selected from the group consisting of:

(a) the amino acid sequence of SEQ ID NO:106;

(b) a fragment of the amino add sequence of SEQ ID NO:106, the fragment comprising eight contiguous amino adds of SEQ ID NO:106; and

(c) the amino acid sequence encoded by the cDNA insert of clone cw1481_{—}1 deposited under accession number ATCC 98822;

the protein being substantially free from other mammalian proteins. Preferably such protein comprises the amino acid sequence of SEQ ID NO:106. In further preferred embodiments, the present invention provides a protein comprising a fragment of the amino acid sequence of SEQ ID NO:106 having biological activity, the fragment preferably comprising eight (more preferably twenty, most preferably thirty) contiguous amino acids of SEQ ID NO:106, or a protein comprising a fragment of the amino acid sequence of SEQ ID NO:106 having biological activity, the fragment comprising the amino acid sequence from amino acid 264 to amino acid 273 of SEQ ID NO:106.

In one embodiment, the present invention provides a composition comprising an isolated polynucleotide selected from the group consisting of:

(a) a polynucleotide comprising the nucleotide sequence of SEQ ID NO:107;

(b) a polynucleotide comprising the nucleotide sequence of SEQ ID NO:107 from nucleotide 29 to nucleotide 2905;

(c) a polynucleotide comprising the nucleotide sequence of SEQ ID NO:107 from nucleotide 146 to nucleotide 2905;

(d) a polynucleotide comprising the nucleotide sequence of the full-length protein coding sequence of clone dd119_{—}4 deposited under accession number ATCC 98822;

(e) a polynucleotide encoding the full-length protein encoded by the cDNA insert of clone dd119_{—}4 deposited under accession number ATCC 98822;

(f) a polynucleotide comprising the nucleotide sequence of a mature protein coding sequence of clone dd119_{—}4 deposited under accession number ATCC 98822;

(g) a polynucleotide encoding a mature protein encoded by the cDNA insert of clone dd119_{—}4 deposited under accession number ATCC 98822;

(h) a polynucleotide encoding a protein comprising the amino add sequence of SEQ ID NO:108;

(i) a polynucleotide encoding a protein comprising a fragment of the amino acid sequence of SEQ ID NO:108 having biological activity, the fragment comprising eight contiguous amino adds of SEQ ID NO:108;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:107.

Preferably, such polynucleotide comprises the nucleotide sequence of SEQ ID NO:107 from nucleotide 29 to nucleotide 2905; the nucleotide sequence of SEQ ID NO:107 from nucleotide 146 to nucleotide 2905; the nucleotide sequence of the full-length protein coding sequence of clone dd119_{—}4 deposited under accession number ATCC 98822; or the nucleotide sequence of a mature protein coding sequence of clone dd119_{—}4 deposited under accession number ATCC 98822. In other preferred embodiments, the polynucleotide encodes the full-length or a mature protein encoded by the cDNA insert of clone dd119_{—}4 deposited under accession number ATCC 98822. In further preferred embodiments, the present invention provides a polynucleotide encoding a protein comprising a fragment of the amino acid sequence of SEQ ID NO:108 having biological activity, the fragment preferably comprising eight (more preferably twenty, most preferably thirty) contiguous amino acids of SEQ ID NO:108, or a polynucleotide encoding a protein comprising a fragment of the amino acid sequence of SEQ ID NO:108 having biological activity, the fragment comprising the amino acid sequence from amino acid 474 to amino acid 483 of SEQ ID NO:108.

Other embodiments provide the gene corresponding to the cDNA sequence of SEQ ID NO:107.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:107, but excluding the poly(A) tail at the 3′ end of SEQ ID NO:107; and

(ab) the nucleotide sequence of the cDNA insert of clone dd1194 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:107, but excluding the poly(A) tail at the 3′ end of SEQ ID NO:107; and

(bb) the nucleotide sequence of the cDNA insert of clone dd119_{—}4 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:107, and extending contiguously from a nucleotide sequence corresponding to the 5′ end of SEQ ID NO:107 to a nucleotide sequence corresponding to the 3′ end of SEQ ID NO:0.107, but excluding the poly(A) tail at the 3′ end of SEQ ID NO:107. Also preferably the polynucleotide isolated according to the above process comprises a nucleotide sequence corresponding to the cDNA sequence of SEQ ID NO:107 from nucleotide 29 to nucleotide 2905, and extending contiguously from a nucleotide sequence corresponding to the 5′ end of said sequence of SEQ ID NO:107 from nucleotide 29 to nucleotide 2905, to a nucleotide sequence corresponding to the 3′ end of said sequence of SEQ ID NO:107 from nucleotide 29 to nucleotide 2905. Also preferably the polynucleotide isolated according to the above process comprises a nucleotide sequence corresponding to the cDNA sequence of SEQ ID NO:107 from nucleotide 146 to nucleotide 2905, and extending contiguously from a nucleotide sequence corresponding to the 5′ end of said sequence of SEQ ID NO:107 from nucleotide 146 to nucleotide 2905, to a nucleotide sequence corresponding to the 3′ end of said sequence of SEQ ID NO:107 from nucleotide 146 to nucleotide 2905.

In other embodiments, the present invention provides a composition comprising a protein, wherein said protein comprises an amino acid sequence selected from the group consisting of:

(a) the amino acid sequence of SEQ ID NO:108;

(b) a fragment of the amino acid sequence of SEQ ID NO:108, the fragment comprising eight contiguous amino adds of SEQ ID NO:108; and

(c) the amino acid sequence encoded by the cDNA insert of clone dd119_{—}4 deposited under accession number ATCC 98822;

the protein being substantially free from other mammalian proteins. Preferably such protein comprises the amino acid sequence of SEQ ID NO:108. In further preferred embodiments, the present invention provides a protein comprising a fragment of the amino acid sequence of SEQ ID NO:108 having biological activity, the fragment preferably comprising eight (more preferably twenty, most preferably thirty) contiguous amino acids of SEQ ID NO:108, or a protein comprising a fragment of the amino acid sequence of SEQ ID NO:108 having biological activity, the fragment comprising the amino acid sequence from amino acid 474 to amino acid 483 of SEQ ID NO 108.

In one embodiment, the present invention provides a composition comprising an isolated polynucleotide selected from the group consisting of:

(a) a polynucleotide comprising the nucleotide sequence of SEQ ID NO:109;

(b) a polynucleotide comprising the nucleotide sequence of SEQ ID NO:109 from nucleotide 16 to nucleotide 369;

(c) a polynucleotide comprising the nucleotide sequence of SEQ ID NO:109 from nucleotide 103 to nucleotide 369;

(d) a polynucleotide comprising the nucleotide sequence of the full-length protein coding sequence of clone df202_{—}3 deposited under accession number ATCC 98822;

(e) a polynucleotide encoding the full-length protein encoded by the cDNA insert of clone df202_{—}3 deposited under accession number ATCC 98822;

(f) a polynucleotide comprising the nucleotide sequence of a mature protein coding sequence of clone df202_{—}3 deposited under accession number ATCC 98822;

(g) a polynucleotide encoding a mature protein encoded by the cDNA insert of clone df202_{—}3 deposited under accession number ATCC 98822;

(h) a polynucleotide encoding a protein comprising the amino acid sequence of SEQ ID NO:110;

(i) a polynucleotide encoding a protein comprising a fragment of the amino acid sequence of SEQ ID NO:110 having biological activity, the fragment comprising eight contiguous amino acids of SEQ ID NO:110;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:109.

Preferably, such polynucleotide comprises the nucleotide sequence of SEQ ID NO:109 from nucleotide 16 to nucleotide 369; the nucleotide sequence of SEQ ID NO:109 from nucleotide 103 to nucleotide 369; the nucleotide sequence of the full-length protein coding sequence of clone df202_{—}3 deposited under accession number ATCC 98822; or the nucleotide sequence of a mature protein coding sequence of clone df202_{—}3 deposited under accession number ATCC 98822. In other preferred embodiments, the polynucleotide encodes the full-length or a mature protein encoded by the cDNA insert of clone df202_{—}3 deposited under accession number ATCC 98822. In further preferred embodiments, the present invention provides a polynucleotide encoding a protein comprising a fragment of the amino acid sequence of SEQ ID NO:110 having biological activity, the fragment preferably comprising eight (more preferably twenty, most preferably thirty) contiguous amino acids of SEQ ID NO:110, or a polynucleotide encoding a protein comprising a fragment of the amino acid sequence of SEQ ID NO:110 having biological activity, the fragment comprising the amino acid sequence from amino acid 54 to amino acid 63 of SEQ ID NO:110.

Other embodiments provide the gene corresponding to the cDNA sequence of SEQ ID NO:109.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:109, but excluding the poly(A) tail at the 3′ end of SEQ ID NO:109; and

(ab) the nucleotide sequence of the cDNA insert of clone df202_{—}3 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:109, but excluding the poly(A) tail at the 3′ end of SEQ ID NO:109; and

(bb) the nucleotide sequence of the cDNA insert of clone df202_{—}3 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:109, and extending contiguously from a nucleotide sequence corresponding to the 5′ end of. SEQ ID NO:109 to a nucleotide sequence corresponding to the 3′ end of SEQ ID NO:109, but excluding the poly(A) tail at the 3′ end of SEQ ID NO:109. Also preferably the polynucleotide isolated according to the above process comprises a nucleotide sequence corresponding to the cDNA sequence of SEQ ID NO:109 from nucleotide 16 to nucleotide 369, and extending contiguously from a nucleotide sequence corresponding to the 5′ end of said sequence of SEQ ID NO:109 from nucleotide 16 to nucleotide 369, to a nucleotide sequence corresponding to the 3′ end of said sequence of SEQ ID NO:109 from nucleotide 16 to nucleotide 369. Also preferably the polynucleotide isolated according to the above process comprises a nucleotide sequence corresponding to the cDNA sequence of SEQ ID NO:109 from nucleotide 103 to nucleotide 369, and extending contiguously from a nucleotide sequence corresponding to the 5′ end of said sequence of SEQ ID NO:109 from nucleotide 103 to nucleotide 369, to a nucleotide sequence corresponding to the 3′ end of said sequence of SEQ ID NO:109 from nucleotide 103 to nucleotide 369.

In other embodiments, the present invention provides a composition comprising a protein, wherein said protein comprises an amino acid sequence selected from the group consisting of:

(a) the amino acid sequence of SEQ ID NO:110;

(b) a fragment of the amino acid sequence of SEQ ID NO:110, the fragment comprising eight contiguous amino acids of SEQ ID NO:110; and

(c) the amino acid sequence encoded by the cDNA insert of clone df202_{—}3 deposited under accession number ATCC 98822;

the protein being substantially free from other mammalian proteins. Preferably such protein comprises the amino acid sequence of SEQ ID NO:110. In further preferred embodiments, the present invention provides a protein comprising a fragment of the amino acid sequence of SEQ ID NO:110 having biological activity, the fragment preferably comprising eight (more preferably twenty, most preferably thirty) contiguous amino acids of SEQ ID NO:110, or a protein comprising a fragment of the amino acid sequence of SEQ ID NO:110 having biological activity, the fragment comprising the amino acid sequence from amino acid 54 to amino acid 63 of SEQ ID NO:110.

In one embodiment, the present invention provides a composition comprising an isolated polynucleotide selected from the group consisting of:

(a) a polynucleotide comprising the nucleotide sequence of SEQ ID NO:111;

(b) a polynucleotide comprising the nucleotide sequence of SEQ ID NO:111 from nucleotide 2192 to nucleotide 2539;

(c) a polynucleotide comprising the nucleotide sequence of SEQ ID NO 111 from nucleotide 2255 to nucleotide 2539;

(d) a polynucleotide comprising the nucleotide sequence of the full-length protein coding sequence of clone km225_{—}1 deposited under accession number ATCC 98822;

(e) a polynucleotide encoding the full-length protein encoded by the cDNA insert of clone km225_{—}1 deposited under accession number ATCC 98822;

(f) a polynucleotide comprising the nucleotide sequence of a mature protein coding sequence of clone km225_{—}1 deposited under accession number ATCC 98822;

(g) a polynucleotide encoding a mature protein encoded by the cDNA insert of clone km2251 deposited under accession number ATCC 98822;

(h) a polynucleotide encoding a protein comprising the amino acid sequence of SEQ ID NO:112;

(i) a polynucleotide encoding a protein comprising a fragment of the amino acid sequence of SEQ ID NO:112 having biological activity, the fragment comprising eight contiguous amino acids of SEQ ID NO:112;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:111.

Preferably, such polynucleotide comprises the nucleotide sequence of SEQ ID NO:111 from nucleotide 2192 to nucleotide 2539; the nucleotide sequence of SEQ ID NO:111 from nucleotide 2255 to nucleotide 2539; the nucleotide sequence of the full-length protein coding sequence of clone kn225_{—}1 deposited under accession number ATCC 98822; or the nucleotide sequence of a mature protein coding sequence of clone km225_{—}1 deposited under accession number ATCC 98822. In other preferred embodiments, the polynucleotide encodes the full-length or a mature protein encoded by the cDNA insert of clone km225_{—}1 deposited under accession number ATCC 98822. In further preferred embodiments, the present invention provides a polynucleotide encoding a protein comprising a fragment of the amino acid sequence of SEQ ID NO:112 having biological activity, the fragment preferably comprising eight (more preferably twenty, most preferably thirty) contiguous amino acids of SEQ ID NO:112, or a polynucleotide encoding a protein comprising a fragment of the amino acid sequence of SEQ ID NO:112 having biological activity, the fragment comprising the amino add sequence from amino acid 53 to amino acid 62 of SEQ ID NO:112.

Other embodiments provide the gene corresponding to the cDNA sequence of SEQ ID NO:111.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:111, but excluding the poly(A) tail at the 3′ end of SEQ ID NO:111; and

(ab) the nucleotide sequence of the cDNA insert of clone km225_{—}1 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:111, but excluding the poly(A) tail at the 3′ end of SEQ ID NO:111; and

(bb) the nucleotide sequence of the cDNA insert of clone km225_{—}1 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:111, and extending contiguously from a nucleotide sequence corresponding to the 5′ end of SEQ ID NO:111 to a nucleotide sequence corresponding to the 3′ end of SEQ ID NO:111, but excluding the poly(A) tail at the 3′ end of SEQ ID NO:111. Also preferably the polynucleotide isolated according to the above process comprises a nucleotide sequence corresponding to the cDNA sequence of SEQ ID NO:111 from nucleotide 2192 to nucleotide 2539, and extending contiguously from a nucleotide sequence corresponding to the 5′ end of said sequence of SEQ ID NO:111 from nucleotide 2192 to nucleotide 2539, to a nucleotide sequence corresponding to the 3′ end of said sequence of SEQ ID NO:111 from nucleotide 2192 to nucleotide 2539. Also preferably the polynucleotide isolated according to the above process comprises a nucleotide sequence corresponding to the cDNA sequence of SEQ ID NO:111 from nucleotide 2255 to nucleotide 2539, and extending contiguously from a nucleotide sequence corresponding to the 5′ end of said sequence of SEQ ID NO:111 from nucleotide 2255 to nucleotide 2539, to a nucleotide sequence corresponding to the 3′ end of said sequence of SEQ ID NO:111 from nucleotide 2255 to nucleotide 2539.

In other embodiments, the present invention provides a composition comprising a protein, wherein said protein comprises an amino acid sequence selected from the group consisting of:

(a) the amino acid sequence of SEQ ID NO:112;

(b) a fragment of the amino acid sequence of SEQ ID NO:112, the fragment comprising eight contiguous amino acids of SEQ ID NO:112; and

(c) the amino add sequence encoded by the cDNA insert of clone km225_{—}1 deposited under accession number ATCC 98822;

the protein being substantially free from other mammalian proteins. Preferably such protein comprises the amino acid sequence of SEQ ID NO:112. In further preferred embodiments, the present invention provides a protein comprising a fragment of the amino add sequence of SEQ ID NO:112 having biological activity, the fragment preferably comprising eight (more preferably twenty, most preferably thirty) contiguous amino acids of SEQ ID NO:112, or a protein comprising a fragment of the amino acid sequence of SEQ ID NO:112 having biological activity, the fragment comprising the amino acid sequence from amino acid 53 to amino acid 62 of SEQ ID NO:112.

In one embodiment, the present invention provides a composition comprising an isolated polynucleotide selected from the group consisting of:

(a) a polynucleotide comprising the nucleotide sequence of SEQ ID NO:113;

(b) a polynucleotide comprising the nucleotide sequence of SEQ ID NO:113 from nucleotide 1734 to nucleotide 2030;

(c) a polynucleotide comprising the nucleotide sequence of SEQ ID NO:113 from nucleotide 1965 to nucleotide 2030;

(d) a polynucleotide comprising the nucleotide sequence of the full-length protein coding sequence of clone mj301_{—}1 deposited under accession number ATCC 98822;

(e) a polynucleotide encoding the full-length protein encoded by the cDNA insert of clone mj301_{—}1 deposited under accession number ATCC 98822;

(f) a polynucleotide comprising the nucleotide sequence of a mature protein coding sequence of clone mj301_{—}1 deposited under accession number ATCC 98822;

(g) a polynucleotide encoding a mature protein encoded by the cDNA insert of clone mj301_{—}1 deposited under accession number ATCC 98822,

(h) a polynucleotide encoding a protein comprising the amino acid sequence of SEQ ID NO:114;

(i) a polynucleotide encoding a protein comprising a fragment of the amino acid sequence of SEQ ID NO:114 having biological activity, the fragment comprising eight contiguous amino acids of SEQ ID NO:114;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:113.

Preferably, such polynucleotide comprises the nucleotide sequence of SEQ ID NO:113 from nucleotide 1734 to nucleotide 2030; the nucleotide sequence of SEQ ID NO:113 from nucleotide 1965 to nucleotide 2030; the nucleotide sequence of the full-length protein coding sequence of clone mj301_{—}1 deposited under accession number ATCC 98822; or the nucleotide sequence of a mature protein coding sequence of clone mj301_{—}1 deposited under accession number ATCC 98822. In other preferred embodiments, the polynucleotide encodes the full-length or a mature protein encoded by the cDNA insert of clone mj301_{—}1 deposited under accession number ATCC 98822. In further preferred embodiments, the present invention provides a polynucleotide encoding a protein comprising a fragment of the amino acid sequence of SEQ ID NO:114 having biological activity, the fragment preferably comprising eight (more preferably twenty, most preferably thirty) contiguous amino acids of SEQ ID NO:114, or a polynucleotide encoding a protein comprising a fragment of the amino acid sequence of SEQ ID NO:114 having biological activity, the fragment comprising the amino acid sequence from amino acid 44 to amino acid 53 of SEQ ID NO:114.

Other embodiments provide the gene corresponding to the cDNA sequence of SEQ ID NO:113.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:113, but excluding the poly(A) tail at the 3′ end of SEQ ID NO:113; and

(ab) the nucleotide sequence of the cDNA insert of clone mj301_{—}1 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:113, but excluding the poly(A) tail at the 3′ end of SEQ ID NO:113; and

(bb) the nucleotide sequence of the cDNA insert of clone mj301_{—}1 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:113, and extending contiguously from a nucleotide sequence corresponding to the 5′ end of SEQ ID NO:113 to a nucleotide sequence corresponding to the 3′ end of SEQ ID NO:113, but excluding the poly(A) tail at the 3′ end of SEQ ID NO:113. Also preferably the polynucleotide isolated according to the above process comprises a nucleotide sequence corresponding to the cDNA sequence of SEQ ID NO:113 from nucleotide 1734 to nucleotide 2030, and extending contiguously from a nucleotide sequence corresponding to the 5′ end of said sequence of SEQ ID NO:113 from nucleotide 1734 to nucleotide 2030, to a nucleotide sequence corresponding to the 3′ end of said sequence of SEQ ID NO:113 from nucleotide 1734 to nucleotide 2030. Also preferably the polynucleotide isolated according to the above process comprises a nucleotide sequence corresponding to the cDNA sequence of SEQ ID NO:113 from nucleotide 1965 to nucleotide 2030, and extending contiguously from a nucleotide sequence corresponding to the 5′ end of said sequence of SEQ ID NO:113 from nucleotide 1965 to nucleotide 2030, to a nucleotide sequence corresponding to the 3′ end of said sequence of SEQ ID NO:113 from nucleotide 1965 to nucleotide 2030.

In other embodiments, the present invention provides a composition comprising a protein, wherein said protein comprises an amino acid sequence selected from the group consisting of:

(a) the amino acid sequence of SEQ ID NO:114;

(b) a fragment of the amino acid sequence of SEQ ID NO:114, the fragment comprising eight contiguous amino acids of SEQ ID NO:114; and

(c) the amino acid sequence encoded by the cDNA insert of clone mj301_{—}1 deposited under accession number ATCC 98822;

the protein being substantially free from other mammalian proteins. Preferably such protein comprises the amino acid sequence of SEQ ID NO:114. In further preferred embodiments, the present invention provides a protein comprising a fragment of the amino acid sequence of SEQ ID NO:114 having biological activity, the fragment preferably comprising eight (more preferably twenty, most preferably thirty) contiguous amino acids of SEQ ID NO:114, or a protein comprising a fragment of the amino acid sequence of SEQ ID NO:114 having biological activity, the fragment comprising the amino acid sequence from amino acid 44 to amino acid 53 of SEQ ID NO:114.

In one embodiment, the present invention provides a composition comprising an isolated polynucleotide selected from the group consisting of:

(a) a polynucleotide comprising the nucleotide sequence of SEQ ID NO:115;

(b) a polynucleotide comprising the nucleotide sequence of SEQ ID NO:115 from nucleotide 799 to nucleotide 1350;

(c) a polynucleotide comprising the nucleotide sequence of SEQ ID NO:115 from nucleotide 925 to nucleotide 1350;

(d) a polynucleotide comprising the nucleotide sequence of the full-length protein coding sequence of clone ml10_{—}7 deposited under accession number ATCC 98822;

(e) a polynucleotide encoding the full-length protein encoded by the cDNA insert of clone ml10_{—}7 deposited under accession number ATCC 98822;

(f) a polynucleotide comprising the nucleotide sequence of a mature protein coding sequence of clone ml10_{—}7 deposited under accession number ATCC 98822;

(g) a polynucleotide encoding a mature protein encoded by the cDNA insert of clone ml10_{—}7 deposited under accession number ATCC 98822;

(h) a polynucleotide encoding a protein comprising the amino acid sequence of SEQ ID NO:116;

(i) a polynucleotide encoding a protein comprising a fragment of the amino acid sequence of SEQ ID NO:116 having biological activity, the fragment comprising eight contiguous amino acids of SEQ ID NO:116;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:115.

Preferably, such polynucleotide comprises the nucleotide sequence of SEQ ID NO:115 from nucleotide 799 to nucleotide 1350; the nucleotide sequence of SEQ ID NO:115 from nucleotide 925 to nucleotide 1350; the nucleotide sequence of the full-length protein coding sequence of clone ml10_{—}7 deposited under accession number ATCC 98822; or the nucleotide sequence of a mature protein coding sequence of clone ml10_{—}7 deposited under accession number ATCC 98822. In other preferred embodiments, the polynucleotide encodes the full-length or a mature protein encoded by the cDNA insert of clone ml10_{—}7 deposited under accession number ATCC 98822. In further preferred embodiments, the present invention provides a polynucleotide encoding a protein comprising a fragment of the amino acid sequence of SEQ ID NO:116 having biological activity, the fragment preferably comprising eight (more preferably twenty, most preferably thirty) contiguous amino acids of SEQ ID NO:116, or a polynucleotide encoding a protein comprising a fragment of the amino acid sequence of SEQ ID NO:116 having biological activity, the fragment comprising the amino acid sequence from amino acid 87 to amino acid 96 of SEQ ID NO:116.

Other embodiments provide the gene corresponding to the cDNA sequence of SEQ ID NO:115.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:115, but excluding the poly(A) tail at the 3′ end of SEQ ID NO:115; and

(ab) the nucleotide sequence of the cDNA insert of clone ml107_{—}7 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of

(ba) SEQ ID NO:115, but excluding the poly(A) tail at the 3′ end of SEQ ID NO:115; and

(bb) the nucleotide sequence of the cDNA insert of clone ml10_{—}7 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:115, and extending contiguously from a nucleotide sequence corresponding to the 5′ end of SEQ ID NO:115 to a nucleotide sequence corresponding to the 3′ end of SEQ ID NO:115, but excluding the poly(A) tail at the 3′ end of SEQ ID NO:115. Also preferably the polynucleotide isolated according to the above process comprises a nucleotide sequence corresponding to the cDNA sequence of SEQ ID NO:115 from nucleotide 799 to nucleotide 1350, and extending contiguously from a nucleotide sequence corresponding to the 5′ end of said sequence of SEQ ID NO:115 from nucleotide 799 to nucleotide 1350, to a nucleotide sequence corresponding to the 3′ end of said sequence of SEQ ID NO:115 from nucleotide 799 to nucleotide 1350. Also preferably the polynucleotide isolated according to the above process comprises a nucleotide sequence corresponding to the cDNA sequence of SEQ ID NO:115 from nucleotide 925 to nucleotide 1350, and extending contiguously from a nucleotide sequence corresponding to the 5′ end of said sequence of SEQ ID NO:115 from nucleotide 925 to nucleotide 1350, to a nucleotide sequence corresponding to the 3′ end of said sequence of SEQ ID NO:115 from nucleotide 925 to nucleotide 1350.

In other embodiments, the present invention provides a composition comprising a protein, wherein said protein comprises an amino acid sequence selected from the group consisting of:

(a) the amino add sequence of SEQ ID NO:116;

(b) a fragment of the amino acid sequence of SEQ ID NO:116, the fragment comprising eight contiguous amino adds of SEQ ID NO:116; and

(c) the amino acid sequence encoded by the cDNA insert of clone ml10_{—}7 deposited under accession number ATCC 98822;

the protein being substantially free from other mammalian proteins. Preferably such protein comprises the amino add sequence of SEQ ID NO:116. In further preferred embodiments, the present invention provides a protein comprising a fragment of the amino acid sequence of SEQ ID NO:116 having biological activity, the fragment preferably comprising eight (more preferably twenty, most preferably thirty) contiguous amino adds of SEQ ID NO:116, or a protein comprising a fragment of the amino acid sequence of SEQ ID NO:116 having biological activity, the fragment comprising the amino acid sequence from amino acid 87 to amino acid 96 of SEQ ID NO:116.

In one embodiment, the present invention provides a composition comprising an isolated polynucleotide selected from the group consisting of:

(a) a polynucleotide comprising the nucleotide sequence of SEQ ID NO:117;

(b) a polynucleotide comprising the nucleotide sequence of SEQ ID NO:117 from nucleotide 837 to nucleotide 1094;

(c) a polynucleotide comprising the nucleotide sequence of the full-length protein coding sequence of clone my340_{—}1 deposited under accession number ATCC 98822;

(d) a polynucleotide encoding the full-length protein encoded by the cDNA insert of clone my340_{—}1 deposited under accession number ATCC 98822;

(e) a polynucleotide comprising the nucleotide sequence of a mature protein coding sequence of clone my340_{—}1 deposited under accession number ATCC 98822;

(f) a polynucleotide encoding a mature protein encoded by the cDNA insert of clone my340_{—}1 deposited under accession number ATCC 98822;

(g) a polynucleotide encoding a protein comprising the amino acid sequence of SEQ ID NO:118;

(h) a polynucleotide encoding a protein comprising a fragment of the amino acid sequence of SEQ ID NO:118 having biological activity, the fragment comprising eight contiguous amino acids of SEQ ID NO:118;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:117.

Preferably, such polynucleotide comprises the nucleotide sequence of SEQ ID NO:117 from nucleotide 837 to nucleotide 1094; the nucleotide sequence of the full-length protein coding sequence of clone my340_{—}1 deposited under accession number ATCC 98822; or the nucleotide sequence of a mature protein coding sequence of clone my340_{—}1 deposited under accession number ATCC 98822. In other preferred embodiments, the polynucleotide encodes the full-length or a mature protein encoded by the cDNA insert of clone my340_{—}1 deposited under accession number ATCC 98822. In further preferred embodiments, the present invention provides a polynucleotide encoding a protein comprising a fragment of the amino acid sequence of SEQ ID NO:118 having biological activity, the fragment preferably comprising eight (more preferably twenty, most preferably thirty) contiguous amino acids of SEQ ID NO:118, or a polynucleotide encoding a protein comprising a fragment of the amino acid sequence of SEQ ID NO:118 having biological activity, the fragment comprising the amino acid sequence from amino acid 38 to amino acid 47 of SEQ ID NO:118.

Other embodiments provide the gene corresponding to the cDNA sequence of SEQ ID NO:117. Further embodiments of the invention provide isolated polynucleotides produced according to a process selected from the group consisting of:

(a) a process comprising the steps of:

(i) preparing one or more polynucleotide probes that hybridize in 6×SSC at 65 degrees C. to a nucleotide sequence selected from the group consisting of:

(aa) SEQ ID NO:117, but excluding the poly(A) tail at the 3′ end of SEQ ID NO:117; and

(ab) the nucleotide sequence of the cDNA insert of clone my340_{—}1 deposited under accession number ATCC 98822;

(ii) hybridizing said probe(s) to human genomic DNA in conditions at least as stringent as 4×SSC at 50 degrees C.; and

(iii) isolating the DNA polynucleotides detected with the probe(s); and

(b) a process comprising the steps of:

(i) preparing one or more polynucleotide primers that hybridize in 6×SSC at 65 degrees C. to a nucleotide sequence selected from the group consisting of:

(ba) SEQ ID NO:117, but excluding the poly(A) tail at the 3′ end of SEQ ID NO:117; and

(bb) the nucleotide sequence of the cDNA insert of clone my340_{—}1 deposited under accession number ATCC 98822;

(ii) hybridizing said primer(s) to human genomic DNA in conditions at least as stringent as 4×SSC at 50 degrees C.;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:117, and extending contiguously from a nucleotide sequence corresponding to the 5′ end of SEQ ID NO:117 to a nucleotide sequence corresponding to the 3′ end of SEQ ID NO:117, but excluding the poly(A) tail at the 3′ end of SEQ ID NO:117. Also preferably the polynucleotide isolated according to the above process comprises a nucleotide sequence corresponding to the cDNA sequence of SEQ ID NO:117 from nucleotide 837 to nucleotide 1094, and extending contiguously from a nucleotide sequence corresponding to the 5′ end of said sequence of SEQ ID NO:117 from nucleotide 837 to nucleotide 1094, to a nucleotide sequence corresponding to the 3′ end of said sequence of SEQ ID NO:117 from nucleotide 837 to nucleotide 1094.

In other embodiments, the present invention provides a composition comprising a protein, wherein said protein comprises an amino acid sequence selected from the group consisting of:

(a) the amino acid sequence of SEQ ID NO:118;

(b) a fragment of the amino acid sequence of SEQ ID NO:118, the fragment comprising eight contiguous amino acids of SEQ ID NO:118; and

(c) the amino acid sequence encoded by the cDNA insert of clone my340_{—}1 deposited under accession number ATCC 98822;

the protein being substantially free from other mammalian proteins. Preferably such protein comprises the amino acid sequence of SEQ ID NO:118. In further preferred embodiments, the present invention provides a protein comprising a fragment of the amino acid sequence of SEQ ID NO:118 having biological activity, the fragment preferably comprising eight (more preferably twenty, most preferably thirty) contiguous amino adds of SEQ ID NO:118, or a protein comprising a fragment of the amino add sequence of SEQ ID NO:118 having biological activity, the fragment comprising the amino acid sequence from amino acid 38 to amino acid 47 of SEQ ID NO:118.

In certain preferred embodiments, the polynucleotide is operably linked to an expression control sequence. The invention also provides a host cell, including bacterial, yeast, insect and mammalian cells, transformed with such polynucleotide compositions. Also provided by the present invention are organisms that have enhanced, reduced, or modified expression of the gene(s) corresponding to the polynucleotide sequences disclosed herein.

Processes are also provided for producing a protein, which comprise:

(a) growing a culture of the host cell transformed with such polynucleotide compositions in a suitable culture medium; and

(b) purifying the protein from the culture.

The protein produced according to such methods is also provided by the present invention.

Protein compositions of the present invention may further comprise a pharmaceutically acceptable carrier. Compositions comprising an antibody which specifically reacts with such protein are also provided by the present invention.

Methods are also provided for preventing, treating or ameliorating a medical condition which comprises administering to a mammalian subject a therapeutically effective amount of a composition comprising a protein of the present invention and a pharmaceutically acceptable carrier.

### BRIEF DESCRIPTION OF THE DRAWINGS

FIGS. 1A and 1B are schematic representations of the pED6 and pNOTs vectors, respectively, used for deposit of clones disclosed herein.

### DETAILED DESCRIPTION

Isolated Proteins and Polynucleotides

Nucleotide and amino acid sequences, as presently determined, are reported below for each done and protein disclosed in the present application. The nucleotide sequence of each done can readily be determined by sequencing of the deposited done in accordance with known methods. The predicted amino acid sequence (both full-length and mature forms) can then be determined from such nucleotide sequence. The amino acid sequence of the protein encoded by a particular clone can also be determined by expression of the done in a suitable host cell, collecting the protein and determining its sequence. For each disclosed protein applicants have identified what they have determined to be the reading frame best identifiable with sequence information available at the time of filing.

As used herein a “secreted” protein is one which, when expressed in a suitable host cell, is transported across or through a membrane, including transport as a result of signal sequences in its amino acid sequence. “Secreted” proteins include without limitation proteins secreted wholly (e.g., soluble proteins) or partially (e.g., receptors) from the cell in which they are expressed. “Secreted” proteins also include without limitation proteins which are transported across the membrane of the endoplasmic reticulum.

Clone bn365_{—}53”

A polynucleotide of the present invention has been identified as done “bn365_{—}53”. bn365_{—}53 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bn365_{—}53 is a full-length clone, including the entire coding sequence of a secreted protein (also referred to herein as “bn36553 protein”).

The nucleotide sequence of bn365_{—}53 as presently determined is reported in SEQ ID NO:1, and includes a poly(A) tail. What applicants presently believe to be the proper reading frame and the predicted amino acid sequence of the bn365_{—}53 protein corresponding to the foregoing nucleotide sequence is reported in SEQ ID NO:2.

The EcoRI/NotI restriction fragment obtainable from the deposit containing done bn365_{—}53 should be approximately 650 bp.

The nucleotide sequence disclosed herein for bn365_{—}53 was searched against the GenBank and GeneSeq nucleotide sequence databases using BLASTN/BLASTX and FASTA search protocols. bn365_{—}53 demonstrated at least some similarity with sequences identified as AA242967 (zr65g11.r1 Soares NhHu S1 Homo sapiens cDNA done 668324 5′) and N40141 (yw73c12.r1 Homo sapiens cDNA done 257878 5′). The predicted amino acid sequence disclosed herein for bn365_{—}53 was searched against the GenPept and GeneSeq amino acid sequence databases using the BLASTX search protocol. The predicted bn365_{—}53 protein demonstrated at least some similarity to sequences identified as D63484 (KIAA0150 protein [Homo sapiens]) and to the GAGE-1 to GAGE-6 family of human proteins expressed in tumors (GenBank Accession Numbers U19142-U19147). The amino acid sequence of SEQ ID NO:2 contains two RGD (Arg-Gly-Asp) motifs (around residues 12 and 75): the sequence Arg-Gly-Asp, found in fibronectin, is crucial for its interaction with its cell surface receptor, an integrin. What has been called the ‘RGD’ tripeptide is also found in the sequences of a number of other proteins, where it has been shown to play a role in cell adhesion. These proteins are: some forms of collagens, fibrinogen, vitronectin, von Willebrand factor (VWF), snake disintegrins, and slime mold discoidins. Based upon sequence similarity, bn365_{—}53 proteins and each similar protein or peptide may share at least some activity. The nucleotide sequence of bn365_{—}53 indicates that it may contain one or more repetitive elements.

Clone “bo342_{—}2”

A polynucleotide of the present invention has been identified as clone “bo342_{—}2”. bo342_{—}2 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bo342_{—}2 is a full-length done, including the entire coding sequence of a secreted protein (also referred to herein as “bo342_{—}2 protein”).

The nucleotide sequence of bo342_{—}2 as presently determined is reported in SEQ ID NO:3, and includes a poly(A) tail. What applicants presently believe to be the proper reading frame and the predicted amino acid sequence of the bo342_{—}2 protein corresponding to the foregoing nucleotide sequence is reported in SEQ ID NO:4. Amino acids 372 to 384 of SEQ ID NO:4 are a predicted leader/signal sequence, with the predicted mature amino acid sequence beginning at amino acid 385. Amino acids 1 to 13 are also a possible leader/signal sequence, with the predicted mature amino acid sequence beginning in that case at amino acid 14. Due to the hydrophobic nature of these predicted leader/signal sequences, each is likely to act as a transmembrane domain should it not be separated from the remainder of the bo342_{—}2 protein.

The EcoRI/NotI restriction fragment obtainable from the deposit containing clone bo342_{—}2 should be approximately 2600 bp.

The nucleotide sequence disclosed herein for bo342_{—}2 was searched against the GenBank and GeneSeq nucleotide sequence databases using BLASTN/BLASTX and FASTA search protocols. bo342_{—}2 demonstrated at least some similarity with sequences identified as AA306000 (EST177027 Jurkat T-cells VI Homo sapiens cDNA 5′ end) and W94256 (ze12b02.s1 Soares fetal heart NbHH19W Homo sapiens cDNA clone 358731 3′ similar to contains Alu repetitive element). Based upon sequence similarity, bo342_{—}2 proteins and each similar protein or peptide may share at least some activity. The TopPredII computer program predicts six potential transmembrane domains within the bo342_{—}2 protein sequence, centered around amino acids 300, 320, 380, 410, 430, and 490 of SEQ ID NO:4, respectively. The nucleotide sequence of bo342_{—}2 indicates that it may contain Alu or other repetitive elements.

Clone “dn721_{—}8”

A polynucleotide of the present invention has been identified as done “dn721_{—}8”. dn721_{—}8 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino add sequence of the encoded protein. dn721_{—}8 is a full-length clone, including the entire coding sequence of a secreted protein (also referred to herein as “dn721_{—}8 protein”).

The nucleotide sequence of dn721_{—}8 as presently determined is reported in SEQ ID NO:5, and includes a poly(A) tail. What applicants presently believe to be the proper reading frame and the predicted amino acid sequence of the dn721_{—}8 protein corresponding to the foregoing nucleotide sequence is reported in SEQ ID NO:6.

The EcoRI/NotI restriction fragment obtainable from the deposit containing done dn721_{—}8 should be approximately 2900 bp.

The nucleotide sequence disclosed herein for dn721_{—}8 was searched against the GenBank and GeneSeq nucleotide sequence databases using BLASTN/BLASTX and FASTA search protocols. dn721_{—}8 demonstrated at least some similarity with sequences identified as H63637 (yr34b12.r1 Homo sapiens cDNA done 207167 5′), N31598 (yy20b12.s1 Homo sapiens cDNA done 271775 3′), and R61419 (yh15e05x1 Homo sapiens cDNA done 37671 5′). Based upon sequence similarity, dn721_{—}8 proteins and each similar protein or peptide may share at least some activity. The TopPredII computer program predicts two possible transmembrane domains within the dn7218 protein sequence, one centered around amino acid 269 and another around amino acid 457 of SEQ ID NO:6.

Clone “dn834_{—}1”

A polynucleotide of the present invention has been identified as done “dn834_{—}1”. dn834_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dn834_{—}1 is a full-length done, including the entire coding sequence of a secreted protein (also referred to herein as “dn834_{—}1 protein”).

The nucleotide sequence of dn834_{—}1 as presently determined is reported in SEQ ID NO:7, and includes a poly(A) tail. What applicants presently believe to be the proper reading frame and the predicted amino acid sequence of the dn834_{—}1 protein corresponding to the foregoing nucleotide sequence is reported in SEQ ID NO:8.

The EcoRI/NotI restriction fragment obtainable from the deposit containing done dn834_{—}1 should be approximately 900 bp.

The nucleotide sequence disclosed herein for dn834_{—}1 was searched against the GenBank and GeneSeq nucleotide sequence databases using BLASTN/BLASTX and FASTA search protocols. dn834_{—}1 demonstrated at least some similarity with sequences identified as AA544005 (vj83h07.r1 Soares mouse mammary gland NbMMG Mus musculus cDNA done 935677 5′), AL022163 (Human DNA sequence *** SEQUENCING IN PROGRESS *** from clone 551E13; HTGS phase 1), L44560 (Homo sapiens thymus mRNA (randomly primed, normalized), single-pass sequence), and T72271 (Human B cell surface antigen cDNA). The predicted amino acid sequence disclosed herein for dn834_{—}1 was searched against the GenPept and GeneSeq amino acid sequence databases using the BLASTX search protocol. The predicted dn834_{—}1 protein demonstrated at least some similarity to sequences identified as R47496 (Translated sequence of domains I and II of celD cDNA in done pCNP4). Based upon sequence similarity, dn834_{—}1 proteins and each similar protein or peptide may share at least some activity. The TopPredII computer program predicts three potential transmembrane domains within the dn834_{—}1 protein sequence, centered around amino acids 59, 84, and 145 of SEQ ID NO:8, respectively.

dn834_{—}1 protein was expressed in a COS cell expression system, and an expressed protein band of approximately 18 kDa was detected in membrane fractions using SDS polyacrylamide gel electrophoresis.

Clone “pd278_{—}5”

A polynucleotide of the present invention has been identified as clone “pd278_{—}5”. A cDNA done was first isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. This cDNA clone was then used to isolate pd278_{—}5 from a human adult kidney cDNA library. pd278_{—}5 is a full-length done, including the entire coding sequence of a secreted protein (also referred to herein as “pd278_{—}5 protein”).

The nucleotide sequence of pd278_{—}5 as presently determined is reported in SEQ ID NO:9, and includes a poly(A) tail. What applicants presently believe to be the proper reading frame and the predicted amino acid sequence of the pd278_{—}5 protein corresponding to the foregoing nucleotide sequence is reported in SEQ ID NO:10. Amino acids 61 to 73 of SEQ ID NO:10 are a predicted leader/signal sequence, with the predicted mature amino acid sequence beginning at amino acid 74. Due to the hydrophobic nature of the predicted leader/signal sequence, it is likely to act as a transmembrane domain should the predicted leader/signal sequence not be separated from the remainder of the pd278_{—}5 protein.

There are two additional and mutually overlapping possible open reading frames dose to the 5′ end of SEQ ID NO:9 (bases 82-420 and bases 119-414). The translated open reading frame of bases 119-414 has a predicted leader/signal sequence from amino acid 49 to amino acid 61, with the predicted mature amino acid sequence beginning at amino acid 62. Each of the additional possible open reading frames has a predicted transmembrane domain.

The EcoRI/NotI restriction fragment obtainable from the deposit containing done pd278_{—}5 should be approximately 2000 bp.

The nucleotide sequence disclosed herein for pd278_{—}5 was searched against the GenBank and GeneSeq nucleotide sequence databases using BLASTN/BLASTX and FASTA search protocols. pd278_{—}5 demonstrated at least some similarity with sequences identified as AA292241 (zt50d11.r1 Soares ovary tumor NbHOT Homo sapiens cDNA clone 725781 5′), AA428245 zw51d10.s1 Soares total fetus Nb2HF8 9w Homo sapiens cDNA done 773587 3′), AA599487 (ag23f05.s1 Jia bone marrow stroma Homo sapiens cDNA done 1071201 3′), AA827135 (ob53b03.s1 NCI_CGAP_GCB1 Homo sapiens cDNA clone IMAGE 1335053 3′), H54322 yq90d03.s1 Homo sapiens cDNA done 203045 3′), and T22170 (Human gene signature HUMGS03741). The predicted amino acid sequence disclosed herein for pd278_{—}5 was searched against the GenPept and GeneSeq amino acid sequence databases using the BLASTX search protocol. The predicted pd278_{—}5 protein demonstrated at least some similarity to sequences identified as R13144 (Deleted in Colorectal Carcinomas) and X13885 (extensin (AA 1-620) [Nicotiana tabacum]). Based upon sequence similarity, pd278_{—}5 proteins and each similar protein or peptide may share at least some activity.

Clone “pe80_{—}1”

A polynucleotide of the present invention has been identified as clone “pe80_{—}1”. pe80_{—}1 was isolated from a human adult blood (chronic myelogenous leukemia K562) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pe80_{—}1 is a full-length clone, including the entire coding sequence of a secreted protein (also referred to herein as “pe80_{—}1 protein”).

The nucleotide sequence of pe80_{—}1 as presently determined is reported in SEQ ID NO:11, and includes a poly(A) tail. What applicants presently believe to be the proper reading frame and the predicted amino acid sequence of the pe80_{—}1 protein corresponding to the foregoing nucleotide sequence is reported in SEQ ID NO:12.

The EcoRI/NotI restriction fragment obtainable from the deposit containing done pe80_{—}1 should be approximately 2300 bp.

The nucleotide sequence disclosed herein for pe80_{—}1 was searched against the GenBank and GeneSeq nucleotide sequence databases using BLASTN/BLASTX and FASTA search protocols. pe80_{—}1 demonstrated at least some similarity with sequences identified as AA291078 (zs47b04.r1 NCI_CGAP_GCB1 Homo sapiens cDNA clone IMAGE:700591 5′), AA429912 (zw66e06.s1 Soares testis NHT Homo sapiens cDNA done 781186 3′), H82367 (yv79d06.r1 Homo sapiens cDNA done 248939 5′ similar to contains Alu repetitive element contains OFR repetitive element), Q60627 (Human brain Expressed Sequence Tag EST02640), and R20261 (yg20a02.r1 Homo sapiens cDNA clone 32587 5′). Based upon sequence similarity, pe80_{—}1 proteins and each similar protein or peptide may share at least some activity. The TopPredII computer program predicts two possible transmembrane domains within the pe80_{—}1 protein sequence, one centered around amino acid 58 and another around amino acid 109 of SEQ ID NO 12. The nucleotide sequence of pe80_{—}1 indicates that it may contain an Alu repetitive element.

Clone “pm113_{—}1”

A polynucleotide of the present invention has been identified as clone “pm113_{—}1”. pm113_{—}1 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pm113_{—}1 is a full-length done, including the entire coding sequence of a secreted protein (also referred to herein as “pm113_{—}1 protein”).

The nucleotide sequence of pm113_{—}1 as presently determined is reported in SEQ ID NO: 13, and includes a poly(A) tail. What applicants presently believe to be the proper reading frame and the predicted amino acid sequence of the pm113_{—}1 protein corresponding to the foregoing nucleotide sequence is reported in SEQ ID NO:14. Amino acids 41 to 53 of SEQ ID NO:14 are a predicted leader/signal sequence, with the predicted mature amino acid sequence beginning at amino acid 54. Due to the hydrophobic nature of the predicted leader/signal sequence, it is likely to act as a transmembrane domain should the predicted leader/signal sequence not be separated from the remainder of the pm113_{—}1 protein.

The EcoRI/NotI restriction fragment obtainable from the deposit containing clone pm113_{—}1 should be approximately 1700 bp.

The nucleotide sequence disclosed herein for pm113_{—}1 was searched against the GenBank and GeneSeq nucleotide sequence databases using BLASTN/BLASTX and FASTA search protocols. pm113_{—}1 demonstrated at least some similarity with sequences identified as AA009482 (zi04c03.r1 Soares fetal liver spleen 1NFLS S1 Homo sapiens cDNA clone 429796 5′), AA350890 (EST58401 Infant brain Homo sapiens cDNA 3′ end), AC003030 (Human DNA from chromosome 19-ecific cosmid R29828, genomic sequence, complete sequence), H98961 (yx11b02.s1 Homo sapiens cDNA clone 261387 3′), R07796 (yf15e05.r1 Homo sapiens cDNA done), T22151 (Human gene signature HUMGS03721), and W68491 (zd34h02.r1 Soares fetal heart NbHH19W Homo sapiens cDNA clone 342579 5′). Based upon sequence similarity, pm113_{—}1 proteins and each similar protein or peptide may share at least some activity.

Clone “pm749_{—}8”

A polynucleotide of the present invention has been identified as clone “pm749_{—}8”. pm749_{—}8 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pm749_{—}8 is a full-length clone, including the entire coding sequence of a secreted protein (also referred to herein as “pm749_{—}8 protein”).

The nucleotide sequence of pm749_{—}8 as presently determined is reported in SEQ ID NO:15, and includes a poly(A) tail. What applicants presently believe to be the proper reading frame and the predicted amino acid sequence of the pm749_{—}8 protein corresponding to the foregoing nucleotide sequence is reported in SEQ ID NO:16.

The EcoRI/NotI restriction fragment obtainable from the deposit containing done pm749_{—}8 should be approximately 2300 bp.

The nucleotide sequence disclosed herein for pm749_{—}8 was searched against the GenBank and GeneSeq nucleotide sequence databases using BLASTN/BLASTX and FASTA search protocols. pm749_{—}8 demonstrated at least some similarity with sequences identified as AA314025 (EST185879 Colon carcinoma (HCC) cell line II Homo sapiens cDNA 5′ end) and AA374458 (EST86612 HSC172 cells I Homo sapiens cDNA 5′ end). The predicted amino acid sequence disclosed herein for pm749_{—}8 was searched against the GenPept and GeneSeq amino acid sequence databases using the BLASTX search protocol. The predicted pm749_{—}8 protein demonstrated at least some similarity to sequences identified as D89169 (similar to Saccharomyces cerevisiae SCD6 protein, SWISS PROT Accession Number P45978 [Schizosaccharomyces pombe]) and U30384 (Scd6p [Saccharomyces cerevisiae]). Based upon sequence similarity, pm749_{—}8 proteins and each similar protein or peptide may share at least some activity. The TopPredII computer program predicts a potential transmembrane domain within the pm749_{—}8 protein sequence centered around amino acid 138 of SEQ ID NO:16.

Clone “pt31_{—}4”

A polynucleotide of the present invention has been identified as clone “pt31_{—}4”. pt31_{—}4 was isolated from a human adult blood Oymphoblastic leukemia MOLT-4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pt31_{—}4 is a full-length clone, including the entire coding sequence of a secreted protein (also referred to herein as “pt31_{—}4 protein”).

The nucleotide sequence of pt31_{—}4 as presently determined is reported in SEQ ID NO:17, and includes a poly(A) tail. What applicants presently believe to be the proper reading frame and the predicted amino acid sequence of the pt31_{—}4 protein corresponding to the foregoing nucleotide sequence is reported in SEQ ID NO:18. Amino acids 19 to 31 of SEQ ID NO:18 are a predicted leader/signal sequence, with the predicted mature amino acid sequence beginning at amino acid 32. Due to the hydrophobic nature of the predicted leader/signal sequence, it is likely to act as a transmembrane domain should the predicted leader/signal sequence not be separated from the remainder of the pt31_{—}4 protein.

The EcoRI/NotI restriction fragment obtainable from the deposit containing done pt31_{—}4 should be approximately 3200 bp.

The nucleotide sequence disclosed herein for pt31_{—}4 was searched against the GenBank and GeneSeq nucleotide sequence databases using BLASTN/BLASTX and FASTA search protocols. pt31_{—}4 demonstrated at least some similarity with sequences identified as AA348130 (EST54532 Fetal heart II Homo sapiens cDNA 5′ end), AA350691 (EST58082 Infant brain Homo sapiens cDNA 5′ end), AC001226 (Genomic sequence from Human 13, complete sequence), H22773 (ym54c06.r1 Homo sapiens cDNA done 52351 5′), and R21869 (yh22b10.s1 Homo sapiens cDNA done 130459 3′). The predicted amino acid sequence disclosed herein for pt31_{—}4 was searched against the GenPept and GeneSeq amino add sequence databases using the BLASTX search protocol. The predicted pt31_{—}4 protein demonstrated at least some similarity to sequences identified as U53147 (C01B7.6 [Caenorhabditis elegans]). Based upon sequence similarity, pt31_{—}4 proteins and each similar protein or peptide may share at least some activity. The TopPredII computer program predicts five potential transmembrane domains within the pt31_{—}4 protein sequence, centered around amino adds 90, 110, 210, 410, and 590 of SEQ ID NO:18, respectively.

Clone “pv296_{—}5”

A polynucleotide of the present invention has been identified as clone “pv296_{—}5”. pv296_{—}5 was isolated from a human adult brain (cerebellum) cDNA library using methods which are selective for cDNAs encoding secreted proteins (see U.S. Pat. No. 5,536,637), or was identified as encoding a secreted or transmembrane protein on the basis of computer analysis of the amino add sequence of the encoded protein. pv296_{—}5 is a full-length done, including the entire coding sequence of a secreted protein (also referred to herein as “pv296_{—}5 protein”).

The nucleotide sequence of pv296_{—}5 as presently determined is reported in SEQ ID NO:19, and includes a poly(A) tail. What applicants presently believe to be the proper reading frame and the predicted amino acid sequence of the pv296_{—}5 protein corresponding to the foregoing nucleotide sequence is reported in SEQ ID NO:20.

The EcoRI/NotI restriction fragment obtainable from the deposit containing done pv296_{—}5 should be approximately 1800 bp.

The nucleotide sequence disclosed herein for pv296_{—}5 was searched against the GenBank and GeneSeq nucleotide sequence databases using BLASTN/BLASTX and FASTA search protocols. pv296_{—}5 demonstrated at least some similarity with sequences identified as AA022471 (ze70c01.s1 Soares fetal heart NbHH19W Homo sapiens cDNA clone 364320 3′), AA335246 (EST39647 Epididymus Homo sapiens cDNA 5′ end), and AA481308 (zv06a05.r1 Soares NhHMPu S1 Homo sapiens cDNA done 752816 5′). Based upon sequence similarity, pv296_{—}5 proteins and each similar protein or peptide may share at least some activity. The TopPredII computer program predicts a potential transmembrane domain within the pv296_{—}5 protein sequence centered around amino acid 32 of SEQ ID NO:20.

Clone “er311_{—}20”

A polynucleotide of the present invention has been identified as clone “er311_{—}20”. er311_{—}20 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. er311_{—}20 is a full-length done, including the entire coding sequence of a secreted protein (also referred to herein as “er311_{—}20 protein”).

The nucleotide sequence of er311_{—}20 as presently determined is reported in SEQ ID NO:21, and includes a poly(A) tail. What applicants presently believe to be the proper reading frame and the predicted amino acid sequence of the er311_{—}20 protein corresponding to the foregoing nucleotide sequence is reported in SEQ ID NO 22. Amino acids 654 to 666 of SEQ ID NO:22 are a possible leader/signal sequence; with the predicted mature amino acid sequence beginning at amino acid 667. Due to the hydrophobic nature of the predicted leader/signal sequence, it is likely to act as a transmembrane domain should the predicted leader/signal sequence not be separated from the remainder of the er31120 protein.

The EcoRI/NotI restriction fragment obtainable from the deposit containing done er311_{—}20 should be approximately 2800 bp.

The nucleotide sequence disclosed herein for er311_{—}20 was searched against the GenBank and GeneSeq nucleotide sequence databases using BLASTN/BLASTX and FASTA search protocols. er311_{—}20 demonstrated at least some similarity with sequences identified as AF035526 (Mus musculus kanadaptin mRNA, complete cds), R18277 (yg01c06.r1 Homo sapiens cDNA clone 31018 5′ similar to SP:ZK632.2 CE00419 COILED COIL PROTEIN), R47371 (Hf060-r Homo sapiens cDNA clone f060-r), and Z40133 (H. sapiens partial cDNA sequence; clone c-1sh08). The predicted amino acid sequence disclosed herein for er311_{—}20 was searched against the GenPept and GeneSeq amino acid sequence databases using the BLASTX search protocol. The predicted er311_{—}20 protein demonstrated at least some similarity to sequences identified as AF035526 (kanadaptin [Mus musculus]) and Z22181 (ZK632.2 [Caenorhabditis elegans]). The mouse kanadaptin protein and the predicted er311120 protein both contain poly-glutamic acid stretches within their C-terminal portions. Based upon sequence similarity, er311_{—}20 proteins and each similar protein or peptide may share at least some activity. The TopPredII computer program predicts two potential transmembrane domains within the er311_{—}20 protein sequence, one centered around amino acid 667 and another at the extreme C-terminus of SEQ ID NO:22.

er311_{—}20 protein was expressed in a COS cell expression system, and an expressed protein band of approximately 91 kDa was detected in conditioned medium and membrane fractions using SDS polyacrylamide gel electrophoresis.

Clone “fh149_{—}12”

A polynucleotide of the present invention has been identified as done “fh149_{—}12”. fh149_{—}12 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fh149_{—}12 is a full-length clone, including the entire coding sequence of a secreted protein (also referred to herein as “fh149_{—}12 protein”).

The nucleotide sequence of fh149_{—}12 as presently determined is reported in SEQ ID NO:23, and includes a poly(A) tail. What applicants presently believe to be the proper reading frame and the predicted amino acid sequence of the fh149_{—}12 protein corresponding to the foregoing nucleotide sequence is reported in SEQ ID NO:24. Amino acids 133 to 145 of SEQ ID NO:24 are a predicted leader/signal sequence, with the predicted mature amino acid sequence beginning at amino acid 146. Due to the hydrophobic nature of the predicted leader/signal sequence, it is likely to act as a transmembrane domain should the predicted leader/signal sequence not be separated from the remainder of the fh149_{—}12 protein.

The EcoRI/NotI restriction fragment obtainable from the deposit containing done fh149_{—}12 should be approximately 2500 bp.

The nucleotide sequence disclosed herein for fh149_{—}12 was searched against the GenBank and GeneSeq nucleotide sequence databases using BLASTN/BLASTX and FASTA search protocols. fh149_{—}12 demonstrated at least some similarity with sequences identified as AA653557 (ag67b07.s1 Gessler Wilms tumor Homo sapiens cDNA clone 1127989 3′), AA191185 (zq45b09.r1 Stratagene hNT neuron (#937233) Homo sapiens cDNA clone 632633 5′), H20588 (yn63d06.r1 Homo sapiens cDNA clone 173099 5′), R16294 (yf93b09.r1 Homo sapiens cDNA clone 30087 5′), T08702 (Rat OCT-1 gene), T25120 (Human gene signature HUMGS07278), U38652 (Mus musculus transmembrane transporter (Lx1) mRNA, complete cds), U77086 (Human organic cation transporter 1 (hOCT1) mRNA, complete cds), and Z66539 (H. sapiens creatine transporter gene). The predicted amino acid sequence disclosed herein for fh149_{—}12 was searched against the GenPept and GeneSeq amino acid sequence databases using the BLASTX search protocol. The predicted fh149_{—}12 protein demonstrated at least some similarity to sequences identified as D17546 (Collagen [Mus musculus]), R77676 (Rat OCT-1 protein), and U77086 (organic cation transporter 1 [Homo sapiens]). The fh149_{—}12 protein also shows some homology to organic cation transporters from rat (GenBank L27651) and pig (GenBank Y09400) cells. Based upon sequence similarity, fh149_{—}12 proteins and each similar protein or peptide may share at least some activity. The TopPredII computer program predicts eleven potential transmembrane domains within the fh149_{—}12 protein sequence, centered around amino acids 40, 112, 139, 162, 200, 229, 349, 376, 405, 436, and 467 of SEQ ID NO:24, respectively.

Clone “p201_{—}6”

A polynucleotide of the present invention has been identified as done “pc2016”. pc201_{—}6 was isolated from a human adult retina (retinoblasoma WER1-Rb1) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pc201_{—}6 is a full-length done, including the entire coding sequence of a secreted protein (also referred to herein as “pc2016 protein”).

The nucleotide sequence of pc201_{—}6 as presently determined is reported in SEQ ID NO:25, and includes a poly(A) tail. What applicants presently believe to be the proper reading frame and the predicted amino acid sequence of the pc201_{—}6 protein corresponding to the foregoing nucleotide sequence is reported in SEQ ID NO:26. Amino acids 20 to 32 of SEQ ID NO:26 are a predicted leader/signal sequence, with the predicted mature amino acid sequence beginning at amino acid 33. Due to the hydrophobic nature of the predicted leader/signal sequence, it is likely to act as a transmembrane domain should the predicted leader/signal sequence not be separated from the remainder of the pc201_{—}6 protein.

A partial cDNA done related to pc201_{—}6, pc201_SP, was also isolated from a human adult retina (retinoblasoma WER1-Rb1) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. The pc201_SP done appears to encode a splice variant of the pc201_{—}6 protein. The amino acid sequence of the predicted pc201_SP splice variant protein comprises the amino acid sequence reported in SEQ ID NO:177.

The EcoRI/NotI restriction fragment obtainable from the deposit containing done pc201_{—}6 should be approximately 2500 bp.

The nucleotide sequence disclosed herein for pc201_{—}6 was searched against the GenBank and GeneSeq nucleotide sequence databases using BLASTN/BLASTX and FASTA search protocols. pc21_{—}6 demonstrated at least some similarity with sequences identified as AA256414 (zr80d11.r1 Soares NhHMPu S1 Homo sapiens cDNA clone 682005 5′ similar to WP FFFD8.9 CE01893), AA342139 (EST47690 Fetal spleen Homo sapiens cDNA 3′ end), AC004085 (Homo sapiens; HTGS phase 1, 72 unordered pieces), AF035950 (Homo sapiens putative DDB p127-associated protein mRNA, partial cds), and H10436 (ym08d09.s1 Homo sapiens cDNA clone 47394 3′). The predicted amino acid sequence disclosed herein for pc201_{—}6 was searched against the GenPept and GeneSeq amino acid sequence databases using the BLASTX search protocol. The predicted pc201_{—}6 protein demonstrated at least some similarity to sequences identified as AF035950 (putative DDB p127-associated protein [Homo sapiens]) and U23484 (EEED8.5 [Caenorhabditis elegans]). Based upon sequence similarity, pc2016 proteins and each similar protein or peptide may share at least some activity.

Clone “p187_{—}1”

A polynucleotide of the present invention has been identified as clone “pl87_{—}1”. pl87_{—}1 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pl87_{—}1 is a full-length clone, including the entire coding sequence of a secreted protein (also referred to herein as “pl87_{—}1 protein”).

The nucleotide sequence of pl87_{—}1 as presently determined is reported in SEQ ID NO:27, and includes a poly(A) tail. What applicants presently believe to be the proper reading frame and the predicted amino acid sequence of the pl87_{—}1 protein corresponding to the foregoing nucleotide sequence is reported in SEQ ID NO:28.

The EcoRI/NotI restriction fragment obtainable from the deposit containing done pl87_{—}1 should be approximately 700 bp.

The nucleotide sequence disclosed herein for pl87_{—}1 was searched against the

GenBank and GeneSeq nucleotide sequence databases using BLASTN/BLASTX and FASTA search protocols. pl87_{—}1 demonstrated at least some similarity with sequences identified as AA371861 (ESI83927 Parathyroid gland tumor I Homo sapiens cDNA 5′ end) and AA861863 (ak39e11.s1 Soares testis NHT Homo sapiens cDNA done IMAGE:1408364 3′). Based upon sequence similarity, p187_{—}1 proteins and each similar protein or peptide may share at least some activity. The TopPredII computer program predicts a potential transmembrane domains within the pl87_{—}1 protein sequence centered around amino acid 50 of SEQ ID NO:28.

pl87_{—}1 protein was expressed in a COS cell expression system, and an expressed protein band of approximately 22 kDa was detected in conditioned medium and membrane fractions using SDS polyacrylamide gel electrophoresis.

Clone “pm514_{—}4”

A polynucleotide of the present invention has been identified as done “pm514_{—}4”. pm514_{—}4 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pm514_{—}4 is a full-length done, including the entire coding sequence of a secreted protein (also referred to herein as “pm514_{—}4 protein”).

The nucleotide sequence of pm514_{—}4 as presently determined is reported in SEQ ID NO:29, and includes a poly(A) tail. What applicants presently believe to be the proper reading frame and the predicted amino acid sequence of the pm514_{—}4 protein corresponding to the foregoing nucleotide sequence is reported in SEQ ID NO:30.

The EcoRI/NotI restriction fragment obtainable from the deposit containing done pm514_{—}4 should be approximately 3000 bp.

The nucleotide sequence disclosed herein for pm514_{—}4 was searched against the GenBank and GeneSeq nucleotide sequence databases using BLASTN/BLASTX and FASTA search protocols. pm514_{—}4 demonstrated at least some similarity with sequences identified as AA393855 (zv64g11.r1 Soares total fetus Nb2HF8 9w Homo sapiens cDNA clone 758468 5′ similar to WP ZK1248.14 CE02898), AA427943 (zw53d10.s1 Soares total fetus Nb2HF8 9w Homo sapiens cDNA clone 773779 3′), AA434561 (zwS3d10.r1 Soares total fetus Nb2HF8 9w Homo sapiens cDNA clone 773779 5′), W49736 (zc41a03.r1 Soares senescent fibroblasts NbHSF Homo sapiens cDNA clone 324844 5′), and U95822 (Human putative transmembrane GTPase mRNA, partial cds). The predicted amino acid sequence disclosed herein for pm514_{—}4 was searched against the GenPept and GeneSeq amino add sequence databases using the BLASTX search protocol. The predicted pm514_{—}4 protein demonstrated at least some similarity to sequences identified as U95822 (putative transmembrane GTPase [Homo sapiens]). Based upon sequence similarity, pm514_{—}4 proteins and each similar protein or peptide may share at least some activity. The TopPredII computer program predicts a potential transmembrane domain within the pm514_{—}4 protein sequence, centered around amino acid 600 of SEQ ID NO:30.

Clone “co155_{—}12”

A polynucleotide of the present invention has been identified as clone “co155_{—}12”. co155_{—}12 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. co155_{—}12 is a full-length done, including the entire coding sequence of a secreted protein (also referred to herein as “co155_{—}12 protein”).

The nucleotide sequence of co155_{—}12 as presently determined is reported in SEQ ID NO:31, and includes a poly(A) tail. What applicants presently believe to be the proper reading frame and the predicted amino acid sequence of the co155_{—}12 protein corresponding to the foregoing nucleotide sequence is reported in SEQ ID NO:32. Amino acids 21 to 33 of SEQ ID NO:32 are a predicted leader/signal sequence, with the predicted mature amino acid sequence beginning at amino acid 34. Due to the hydrophobic nature of the predicted leader/signal sequence, it is likely to act as a transmembrane domain should the predicted leader/signal sequence not be separated from the remainder of the co155_{—}2 protein.

The EcoRI/NotI restriction fragment obtainable from the deposit containing done co155_{—}12 should be approximately 2700 bp.

The nucleotide sequence disclosed herein for co155_{—}12 was searched against the GenBank and GeneSeq nucleotide sequence databases using BLASTN/BLASTX and FASTA search protocols. co155_{—}12 demonstrated at least some similarity with sequences identified as AA578373 (nl23d11.s1 NCI_CGAP_HSC1 Homo sapiens cDNA clone IMAGE: 1041525, mRNA sequence), N43800 (yy42h09.r1 Homo sapiens cDNA clone 273953 5′), and W40418 (zc82c10.r1 Pancreatic Islet Homo sapiens cDNA clone 328818 5′, mRNA sequence). The predicted amino acid sequence disclosed herein for co155_{—}12 was searched against the GenPept and GeneSeq amino acid sequence databases using the BLASTX search protocol. The predicted co155_{—}12 protein demonstrated at least some similarity to the sequences identified as L12721 (transmembrane domain encoded by 1099-1167) and AF004849 (human serine/threonin protein kinase). Based upon sequence similarity, co155_{—}12 proteins and each similar protein or peptide may share at least some activity. The TopPredII computer program predicts five additional potential trans-membrane domains within the col155_{—}12 protein sequence, centered around amino acids 90, 180, 470, 580, and 610 of SEQ ID NO:32, respectively.

Clone “fn189_{—}13”

A polynucleotide of the present invention has been identified as done “fn189_{—}13”. fn189_{—}13 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fn189_{—}13 is a full-length done, including the entire coding sequence of a secreted protein (also referred to herein as “fn189_{—}13 protein”).

The nucleotide sequence of fn189_{—}13 as presently determined is reported in SEQ ID NO:33, and includes a poly(A) tail. What applicants presently believe to be the proper reading frame and the predicted amino acid sequence of the fn189_{—}13 protein corresponding to the foregoing nucleotide sequence is reported in SEQ ID NO:34. Amino acids 9 to 21 of SEQ ID NO:34 are a predicted leader/signal sequence, with the predicted mature amino acid sequence beginning at amino acid 22. Due to the hydrophobic nature of the predicted leader/signal sequence, it is likely to act as a transmembrane domain should the predicted leader/signal sequence not be separated from the remainder of the fn189_{—}13 protein.

The EcoRI/NotI restriction fragment obtainable from the deposit containing done fn189_{—}13 should be approximately 3800 bp.

The nucleotide sequence disclosed herein for fn189_{—}13 was searched against the GenBank and GeneSeq nucleotide sequence databases using BLASTN/BLASTX and FASTA search protocols. fn189_{—}13 demonstrated at least some similarity with sequences identified as AA144270 (mr14d12.r1 Soares mouse 3NbMS Mus musculus cDNA clone 597431 5′) and N27605 (yx44e10.r1 Homo sapiens cDNA clone 264618 5′). The predicted amino acid sequence disclosed herein for fn189_{—}13 was searched against the GenPept, GeneSeq, and SWISS_PROT amino acid sequence databases using the BLASTX search protocol. The predicted fn189_{—}13 protein demonstrated at least some similarity to sequences identified as P32857 (PROTEIN PM1 PRECURSOR [Saccharomyces cerevisiae]) and U64598 (weakly similar to S. cervisiae PTM1 precursor (SP:P32857) [Caenorhabditis elegans]). Based upon sequence similarity, fn189_{—}13 proteins and each similar protein or peptide may share at least some activity. The TopPredII computer program predicts five additional potential transmembrane domains within the fn189_{—}13 protein sequence, centered around amino acids 225, 260, 340, 360, and 420 of SEQ ID NO:34, respectively.

Clone “lv2_{—}47”

A polynucleotide of the present invention has been identified as done “lv2_{—}47”. lv2_{—}47 was isolated from a human adult thyroid cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. lv2_{—}47 is a full-length clone, including the entire coding sequence of a secreted protein (also referred to herein as “lv2_{—}47 protein”).

The nucleotide sequence of lv2_{—}47 as presently determined is reported in SEQ ID Nb:35, and includes a poly(A) tail. What applicants presently believe to be the proper reading frame and the predicted amino acid sequence of the lv2_{—}47 protein corresponding to the foregoing nucleotide sequence is reported in SEQ ID NO:36. The TopPredII computer program predicts a potential transmembrane domain within the lv2_{—}47 protein sequence of SEQ ID NO:36, centered around amino acid 60.

Another potential lv2_{—}47 reading frame and predicted amino acid sequence is encoded by basepairs 365 to 880 of SEQ ID NO:35 and is reported in SEQ ID NO:178. Amino acids 49 to 61 of SEQ ID NO:178 are a predicted leader/signal sequence, with the predicted mature amino acid sequence beginning at amino acid 62. Due to the hydrophobic nature of this predicted leader/signal sequence, it is likely to act as a transmembrane domain should it not be separated from the remainder of the protein of SEQ ID NO:178. The TopPredII computer program predicts two additional potential transmembrane domains within the SEQ ID NO:178 amino acid sequence.

The EcoRI/NotI restriction fragment obtainable from the deposit containing done lv2_{—}47 should be approximately 1950 bp.

The nucleotide sequence disclosed herein for lv2_{—}47 was searched against the GenBank and GeneSeq nucleotide sequence databases using BLASTN/BLASTX and FASTA search protocols. lv2_{—}47 demonstrated at least some similarity with sequences identified as AA007293 (zh97f07.r1 Soares fetal liver spleen 1NFLS S 1 Homo sapiens cDNA clone 429253 5′), AA447347 (zw93g06.r1 Soares total fetus Nb2HF8 9w Homo sapiens cDNA clone 784570 5′ similar to WP:F43E2.7 CE07243), AA522451 (ng30h09.s1 NCI_CGAP_Co3 Homo sapiens cDNA clone IMAGE:936353), AA526614 (ni52g12.s1 NCI_CGAP_Ov2 Homo sapiens cDNA clone 980518), F18178 (H. sapiens EST sequence (002-T4-28) from skeletal muscle, mRNA sequence), H46569 (yo20f10.s1 Homo sapiens cDNA clone 178507 3′), and T22574 (Human gene signature HUMGS04190). Based upon sequence similarity, lv2_{—}47 proteins and each similar protein or peptide may share at least some activity.

Clone “ml243_{—}1”

A polynucleotide of the present invention has been identified as clone “ml243_{—}1”. ml243_{—}1 was isolated from a human adult brain (caudate nucleus) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ml243_{—}1 is a full-length done, including the entire coding sequence of a secreted protein (also referred to herein as “ml243_{—}1 protein”).

The nucleotide sequence of ml243_{—}1 as presently determined is reported in SEQ ID NO:37, and includes a poly(A) tail What applicants presently believe to be the proper reading frame and the predicted amino acid sequence of the ml243_{—}1 protein corresponding to the foregoing nucleotide sequence is reported in SEQ ID NO:38. Amino acids 25 to 37 of SEQ ID NO:38 are a predicted leader/signal sequence, with the predicted mature amino acid sequence beginning at amino acid 38. Due to the hydrophobic nature of the predicted leader/signal sequence, it is likely to act as a transmembrane domain should the predicted leader/signal sequence not be separated from the remainder of the ml243_{—}1 protein.

The EcoRI/NotI restriction fragment obtainable from the deposit containing done ml243_{—}1 should be approximately 1600 bp.

The nucleotide sequence disclosed herein for ml243_{—}1 was searched against the GenBank and GeneSeq nucleotide sequence databases using BLASTN/BLASTX and FASTA search protocols. ml243_{—}1 demonstrated at least some similarity with sequences identified as N66656 (yy71a06.sl Homo sapiens cDNA clone 278962 3′), R17513 (yg02g12.r1 Homo sapiens cDNA clone 31064 5), Z83837 (Human DNA sequence from Fosmid 113D11 on chromosome 22q11.2-qter contains ESTs, CpG island), and Z84468 (Human DNA sequence from clone 299D3; HTGS phase 1). Based upon sequence similarity, ml1243_{—}1 proteins and each similar protein or peptide may share at least some activity.

ml243_{—}1 protein was expressed in a COS cell expression system, and an expressed protein band of approximately 16 kDa was detected in conditioned medium using SDS polyacrylamide gel electrophoresis.

Clone “pm96_{—}9”

A polynucleotide of the present invention has been identified as clone “pm96_{—}9”. pm96_{—}9 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pm96_{—}9 is a full-length done, including the entire coding sequence of a secreted protein (also referred to herein as “pm96_{—}9 protein”).

The nucleotide sequence of pm96_{—}9 as presently determined is reported in SEQ ID NO:39, and includes a poly(A) tail. What applicants presently believe to be the proper reading frame and the predicted amino acid sequence of the pm96_{—}9 protein corresponding to the foregoing nucleotide sequence is reported in SEQ ID NO:40.

The EcoRI/NotI restriction fragment obtainable from the deposit containing done pm96_{—}9 should be approximately 3600 bp.

The nucleotide sequence disclosed herein for pm96_{—}9 was searched against the GenBank and GeneSeq nucleotide sequence databases using BLASTN/BLASTX and FASTA search protocols. pm96_{—}9 demonstrated at least some similarity with sequences identified as AA444024 (zv44d12.r1 Soares ovary tumor NbHOT Homo sapiens cDNA clone 756503 5′), AA488901 (aa55h09.s1 NCI_CGAP_GCB1 Homo sapiens cDNA clone IMAGE:824897 3′), R16408 (yf40b02.r1 Homo sapiens cDNA clone 129291 5′), T19732 (Human gene signature HUMGS00806), U52112 (Homo sapiens Xq28 genomic DNA in the region of the L1CAM locus containing the genes for neural cell adhesion molecule L1 (L1CAM), arginine-vasopressin receptor (AVPR2), C1 p115 (C1), ARD1 N-acetyltransferase related protein (TE2), renin-binding protein (RbP), host cell factor 1 (HCF1), and interleukin-1 receptor-associated kinase (IRAK) genes, complete cds, and Xq28lu2 gene), and Z82250 (Human DNA sequence from cosmid N86D4 on chromosome 22q12-qter contains STS). Based upon sequence similarity, pm96_{—}9 proteins and each similar protein or peptide may share at least some activity. The TopPredII computer program predicts a potential transmembrane domain at the extreme C-terminus of the pm96_{—}9 protein sequence (SEQ ID NO:40).

Clone “pu261_{—}1”

A polynucleotide of the present invention has been identified as clone “pu261_{—}1”. pu261_{—}1 was isolated from a human adult blood (promyelocytic leukemia HL-60) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pu261_{—}1 is a full-length done, including the entire coding sequence of a secreted protein (also referred to herein as “pu261_{—}1 protein”).

The nucleotide sequence of pu261_{—}1 as presently determined is reported in SEQ ID NO:41, and includes a poly(A) tail. What applicants presently believe to be the proper reading frame and the predicted amino acid sequence of the pu261_{—}1 protein corresponding to the foregoing nucleotide sequence is reported in SEQ ID NO:42. Amino acids 116 to 128 of SEQ ID NO:42 are a predicted leader/signal sequence, with the predicted mature amino acid sequence beginning at amino acid 129. Due to the hydrophobic nature of the predicted leader/signal sequence, it is likely to act as a transmembrane domain should the predicted leader/signal sequence not be separated from the remainder of the pu261_{—}1 protein.

The EcoRI/NotI restriction fragment obtainable from the deposit containing done. pu261_{—}1 should be approximately 1800 bp.

The nucleotide sequence disclosed herein for pu261_{—}1 was searched against the GenBank and GeneSeq nucleotide sequence databases using BLASTN/BLASTX and FASTA search protocols. pu261_{—}1 demonstrated at least some similarity with sequences identified as H16093 (ym20g10.r1 Homo sapiens cDNA clone 48582 5′). Based upon sequence similarity, pu261_{—}1 proteins and each similar protein or peptide may share at least some activity. The TopPredII computer program predicts an additional potential transmembrane domain within the pu261_{—}1 protein sequence centered around amino add 70 of SEQ ID NO:42.

Clone ““pw214_{—}15”

A polynucleotide of the present invention has been identified as done “pw214_{—}15”. pw214_{—}15 was isolated from a human adult brain (cerebellum) cDNA library using methods which are selective for cDNAs encoding secreted proteins (see U.S. Pat. No. 5,536,637), or was identified as encoding a secreted or transmembrane protein on the basis of computer analysis of the amino add sequence of the encoded protein. pw214_{—}15 is a full-length. done, including the entire coding sequence of a secreted protein (also referred to herein as “pw214_{—}15 protein”).

The nucleotide sequence of pw214_{—}15 as presently determined is reported in SEQ ID NO:43, and includes a poly(A) tail. What applicants presently believe to be the proper reading frame and the predicted amino acid sequence of the pw214_{—}15 protein corresponding to the foregoing nucleotide sequence is reported in SEQ ID NO:44.

The EcoRI/NotI restriction fragment obtainable from the deposit containing done pw214_{—}15 should be approximately 1800 bp.

The nucleotide sequence disclosed herein for pw214_{—}15 was searched against the GenBank and GeneSeq nucleotide sequence databases using BLASTN/BLASTX and FASTA search protocols. pw214_{—}15 demonstrated at least some similarity with sequences identified as AA173391 (zp03a07.r1 Stratagene ovarian cancer (#937219) Homo sapiens cDNA clone 595284 5′), AA253067 (zrS2a10.r1 Soares NhHMP S1 Homo sapiens cDNA clone 667002 5′), AA523652 ni64d09.s1 NCI_CGAP_Pr12 Homo sapiens cDNA clone 981617), and H41832 (yo07b08.r1 Homo sapiens cDNA clone 177207 5′). Based upon sequence similarity, pw214_{—}15 proteins and each similar protein or peptide may share at least some activity. The TopPredII computer program predicts a potential transmembrane domain within the pw214_{—}15 protein sequence centered around amino acid 15 of SEQ ID NO:44.

Clone “qb56_{—}19”

A polynucleotide of the present invention has been identified as clone “qb56_{—}19”. qb56_{—}19 was isolated from a human adult bladder (carcinoma 5637) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. qb56_{—}19 is a full-length clone, including the entire coding sequence of a secreted protein (also referred to herein as “qb56_{—}19 protein”).

The nucleotide sequence of qb56_{—}19 as presently determined is reported in SEQ ID NO:45, and includes a poly(A) tail. What applicants presently believe to be the proper reading frame and the predicted amino acid sequence of the qb56_{—}19 protein corresponding to the foregoing nucleotide sequence is reported in SEQ ID NO-46. Amino acids 18 to 40 of SEQ ID NO:46 are a possible leader/signal sequence, with the predicted mature amino acid sequence beginning at amino acid 41. Due to the hydrophobic nature of the predicted leader/signal sequence, it is likely to act as a transmembrane domain should the predicted leader/signal sequence not be separated from the remainder of the qb56_{—}19 protein.

The EcoRI/NotI restriction fragment obtainable from the deposit containing done qb56_{—}19 should be approximately 1200 bp.

The nucleotide sequence disclosed herein for qb56_{—}19 was searched against the GenBank and GeneSeq nucleotide sequence databases using BLASTN/BLASTX and FASTA search protocols. qb56_{—}19 demonstrated at least some similarity with sequences identified as AA632658 (np87c12.s1 NCI_CGAP_Thy1 Homo sapiens cDNA clone IMAGE:1133302), N56430 (JJ8973F Homo sapiens cDNA clone JJ8973 5′), and WO5470 (za87f11.r1 Soares fetal lung NbHL19W Homo sapiens cDNA clone 299565 5′). Based upon sequence similarity, qb56_{—}19 proteins and each similar protein or peptide may share at least some activity.

qb56_{—}19 protein was expressed in a COS cell expression system, and an expressed protein band of approximately 14 kDa was detected in conditioned medium and membrane fractions using SDS polyacrylamide gel electrophoresis.

Clone “qc646_{—}1

A polynucleotide of the present invention has been identified as clone “qc646_{—}1”. qc646_{—}1 was isolated from a human adult neural tissue (neuroepithelioma HTB-10 line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. qc646_{—}1 is a full-length done, including the entire coding sequence of a secreted protein (also referred to herein as “qc646_{—}1 protein”).

The nucleotide sequence of qc646_{—}1 as presently determined is reported in SEQ ID NO:47, and includes a poly(A) tail. What applicants presently believe to be the proper reading frame and the predicted amino acid sequence of the qc646_{—}1 protein corresponding to the foregoing nucleotide sequence is reported in SEQ ID NO:48. Amino adds 12 to 24 of SEQ ID NO:48 are a predicted leader/signal sequence, with the predicted mature amino acid sequence beginning at amino acid 25. Amino adds 32 to 44 are also a predicted leader/signal sequence, with the predicted mature amino acid sequence beginning at amino acid 45, or are a transmembrane domain. Due to the hydrophobic nature of these predicted leader/signal sequences, each is likely to act as a transmembrane domain should it not be separated from the remainder of the qc64_{—}1 protein.

The EcoRI/NotI restriction fragment obtainable from the deposit containing clone qc646_{—}1 should be approximately 1800 bp.

The nucleotide sequence disclosed herein for qc646_{—}1 was searched against the GenBank and GeneSeq nucleotide sequence databases using BLASTN/BLASTX and FASTA search protocols. qc646_{—}1 demonstrated at least some similarity with sequences identified as AA470035 (zt94a07.r1 Soares testis NHT Homo sapiens cDNA clone 729972 5), and AA483957 (ne76e11.s1 NCI_CGAP_Ew1 Homo sapiens cDNA clone IMAGE:910220). The predicted amino acid sequence disclosed herein for qc646_{—}1 was searched against the GenPept and GeneSeq amino acid sequence databases using the BLASTX search protocol. The predicted qc64_{—}1 protein demonstrated at least some similarity to sequences identified as D88666 (PS-PLA1 (serine phospholipid-specific phospholipase A) [Rattus norvegicus]), M93284 (lipase related protein 2 [Homo sapiens]), and R30739 (C-terminally truncated GPL(1-319)), as well as lipases from various other species. Rat PS-PLA1, serine phospholipid-specific phospholipase A, is a member of the lipase family and is secreted from activated platelets. Based upon sequence similarity, qc646_{—}1 proteins and each similar protein or peptide may share at least some activity. The TopPredIII computer program predicts two additional potential transmembrane domains within the qc646_{—}1 protein sequence, one centered around amino acid 190 and another around amino acid 325 of SEQ ID NO:48. The nucleotide sequence of qc646_{—}1 indicates that it may contain Alu repetitive elements.

Clone “qf116_{—}2”

A polynucleotide of the present invention has been identified as clone “qf116_{—}2”. qf116_{—}2 was isolated from a human adult bladder (carcinoma 5637) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. qf116_{—}2 is a full-length done, including the entire coding sequence of a secreted protein (also referred to herein as “qf116_{—}2 protein”).

The nucleotide sequence of qf116_{—}2 as presently determined is reported in SEQ ID NO:49, and includes a poly(A) tail. What applicants presently believe to be the proper reading frame and the predicted amino acid sequence of the qf116_{—}2 protein corresponding to the foregoing nucleotide sequence is reported in SEQ ID NO:50.

The EcoRI/NotI restriction fragment obtainable from the deposit containing done qf116_{—}2 should be approximately 1200 bp.

The nucleotide sequence disclosed herein for qf116_{—}2 was searched against the GenBank and GeneSeq nucleotide sequence databases using BLASTN/BLASTX and FASTA search protocols. qf116_{—}2 demonstrated at least some similarity with sequences identified as D50810 (placental leucine aminopeptidase [Homo sapiens]), R94512 (GTVap (short version), insulin-cleaving aminopeptidase from GLUT-4 vesicles), and U32990 (vp165 [Rattus norvegicus]). Human placental leucine aminopeptidase/oxytocinase (P-LAP), a member of the type II membrane-spanning zinc metallopeptidase family, degrades several peptide hormones such as oxytocin and vasopresin, suggesting a role in maintaining homeostasis during pregnancy. The predicted P-LAP amino acid sequence contains the HEXXH consensus sequence of zinc metallopeptidases, indicating that the enzyme belongs to this family, which includes aminopeptidase N and aminopeptidase A. The deduced P-LAP amino add sequence also contains a hydrophobic region near the N-terminus, suggesting that the enzyme is a type II integral membrane protein. Results suggest that the enzyme is synthesized as an integral membrane protein and is released into blood under some physiological conditions. (See Rogi et al., 1996, J. Biol. Chem. 271(1): 56-61, which is incorporated by reference herein.) Based upon sequence similarity, qf116_{—}2 proteins and each similar protein or peptide may share at least some activity. The TopPredIII computer program predicts two potential transmembrane domains within the qf 16_{—}2 protein sequence, one centered around amino acid 25 and another around amino acid 290 of SEQ ID NO:50.

Clone “qf662_{—}3”

A polynucleotide of the present invention has been identified as done “qf662_{—}3”. qf662_{—}3 was isolated from a human adult bladder (carcinoma 5637) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. qf662_{—}3 is a full-length done, including the entire coding sequence of a secreted protein (also referred to herein as “qf662_{—}3 protein”).

The nucleotide sequence of qf662_{—}3 as presently determined is reported in SEQ ID NO:51, and includes a poly(A) tail. What applicants presently believe to be the proper reading frame and the predicted amino acid sequence of the qf662_{—}3 protein corresponding to the foregoing nucleotide sequence is reported in SEQ ID NO:52. Amino acids 133 to 145 of SEQ ID NO:52 are a predicted leader/signal sequence, with the predicted mature amino acid sequence beginning at amino acid 146. Due to the hydrophobic nature of the predicted leader/signal sequence, it is likely to act as a transmembrane domain should the predicted leader/signal sequence not be separated from the remainder of the qf662_{—}3 protein.

The EcoRI/NotI restriction fragment obtainable from the deposit containing done qf662_{—}3 should be approximately 1000 bp.

The nucleotide sequence disclosed herein for qf662_{—}3 was searched against the GenBank and GeneSeq nucleotide sequence databases using BLASTN/BLASTX and FASTA search protocols. qf662_{—}3 demonstrated no significant similarity with sequences in these databases. The nucleotide sequence of qf662_{—}3 indicates that it may contain repetitive elements.

Clone “am748_{—}5”

A polynucleotide of the present invention has been identified as done “am748_{—}5”. am748_{—}5 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. am748_{—}5 is a full-length clone, including the entire coding sequence of a secreted protein (also referred to herein as “am748_{—}5 protein”).

The nucleotide sequence of am748_{—}5 as presently determined is reported in SEQ ID NO:53, and includes a poly(A) tail. What applicants presently believe to be the proper reading frame and the predicted amino acid sequence of the am748_{—}5 protein corresponding to the foregoing nucleotide sequence is reported in SEQ ID NO:54. Amino acids 14 to 26 of SEQ ID NO:54 are a predicted leader/signal sequence, with the predicted mature amino acid sequence beginning at amino acid 27. Due to the hydrophobic nature of the predicted leader/signal sequence, it is likely to act as a transmembrane domain should the predicted leader/signal sequence not be separated from the remainder of the am748_{—}5 protein.

The EcoRI/NotI restriction fragment obtainable from the deposit containing done am748_{—}5 should be approximately 1550 bp.

The nucleotide sequence disclosed herein for am748_{—}5 was searched against the GenBank and GeneSeq nucleotide sequence databases using BLASTN/BLASTX and FASTA search protocols. am748_{—}5 demonstrated at least some similarity with sequences identified as AA418860 (zv98g04.r1 Soares NhHMPu S1 Homo sapiens cDNA clone 767862 5′ similar to gb:X14008_rna1 LYSOZYME C PRECURSOR (HUMAN); contains Alu repetitive element; contains element PTR5 repetitive element), AC003007 (Human Chromosome 16 BAC clone CIT987SK-A-61E3, complete sequence), H73304 (yu27c10.r1 Homo sapiens cDNA clone 235026 5′ similar to contains Alu repetitive element), N35175 (yx83d10.r1 Homo sapiens cDNA clone 268339 5′ similar to gb X14008_rnal LYSOZYME C PRECURSOR (HUMAN); contains Alu repetitive element), N41479 (yy05a11.r1 Homo sapiens cDNA clone 270332 5′ similar to gb:X14008_mal LYSOZYME C PRECURSOR (HUMAN)), Q81139 (HPLA2-8 gene), T04964 (EST02852 Homo sapiens cDNA clone HFBCI77), and U18391 (Human Alu sequence clone A8). The predicted amino acid sequence disclosed herein for am748_{—}5 was searched against the GenPept and GeneSeq amino acid sequence databases using the BLASD (search protocol. The predicted am748_{—}5 protein demonstrated at least some similarity to sequences identified as X55777 (put. ORF [Homo sapiens]) and R13556 (Protein encoded downstream of hhc_M oncoprotein). Based upon sequence similarity, am748_{—}5 proteins and each similar protein or peptide may share at least some activity. The nucleotide sequence of am748_{—}5 indicates that it may contain one or more of the following repetitive elements: Alu, L1.

Clone “gj507_{—}1”

A polynucleotide of the present invention has been identified as clone “cj507_{—}1”. cj507_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cj507_{—}1 is a full-length done, including the entire coding sequence of a secreted protein (also referred to herein as “cj507_{—}1 protein”).

The nucleotide sequence of c507_{—}1 as presently determined is reported in SEQ ID NO:55, and includes a poly(A) tail. What applicants presently believe to be the proper reading frame and the predicted amino acid sequence of the cj507_{—}1 protein corresponding to the foregoing nucleotide sequence is reported in SEQ ID NO:56.

The EcoRI/NotI restriction fragment obtainable from the deposit containing clone cj507_{—}1 should be approximately 2100 bp.

The nucleotide sequence disclosed herein for cj507_{—}1 was searched against the GenBank and GeneSeq nucleotide sequence databases using BLASTN/BLASTX and FASTA search protocols. cj507_{—}1 demonstrated at least some similarity with sequences identified as AA100356 (zn46a02.r1 Stratagene HeLa cell s3 937216 Homo sapiens cDNA clone 550442 5′ similar to contains element PTR5 repetitive element), AA228100 (zr56g04.s1 Soares NhHMPu S1 Homo sapiens cDNA clone 667446 3′), AA479997 (zy18b07.r1 Soares NhHMPu S1 Homo sapiens cDNA clone 753973 5′ similar to contains element PTR5 repetitive element, mRNA sequence), and X85324 (H. sapiens mRNA for non polymorphic CAG repeat (CAG12)). Based upon sequence similarity, cj507_{—}1 proteins and each similar protein or peptide may share at least some activity. The TopPredIII computer program predicts a potential transmembrane domain within the cj507_{—}1 protein sequence centered around amino acid 265 of SEQ ID NO:56. The nucleotide sequence of cj507_{—}1 indicates that it may contain a GCA simple repeat region.

cj507_{—}1 protein was expressed in a COS cell expression system, and an expressed protein band of approximately 47 kDa was detected in membrane fractions using SDS polyacrylamide gel electrophoresis.

Clone “cn922_{—}5”

A polynucleotide of the present invention has been identified as done “cn922_{—}5”. cn922_{—}5 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cn922_{—}5 is a full-length done, including the entire coding sequence of a secreted protein (also referred to herein as “cn922_{—}5 protein”).

The nucleotide sequence of cn922_{—}5 as presently determined is reported in SEQ ID NO:57, and includes a poly(A) tail. What applicants presently believe to be the proper reading frame and the predicted amino acid sequence of the cn922_{—}5 protein corresponding to the foregoing nucleotide sequence is reported in SEQ ID NO:58.

The EcoRI/NotI restriction fragment obtainable from the deposit containing done cn922_{—}5 should be approximately 2200 bp.

The nucleotide sequence disclosed herein for cn922_{—}5 was searched against the GenBank and GeneSeq nucleotide sequence databases using BLASTN/BLASTX and FASTA search protocols. cn922_{—}5 demonstrated at least some similarity with sequences identified as H34191 (EST1 10864 Rattus sp. cDNA 5′ end), R18707 (yf98f02.r1 Homo sapiens cDNA clone 30546 5′), T26556 (Human gene signature HUMGS08801), and Z83230 (Caenorhabditis elegans cosmid F56A8). The predicted amino acid sequence disclosed herein for cn922_{—}5 was searched against the GenPept and GeneSeq amino acid sequence databases using the BLASTX search protocol. The predicted cn9225 protein demonstrated at least some similarity to sequences identified as AB004535 (HYPOTHETICAL 105.9 KD PROTEIN IN AAC3-RFC5 INTERGENIC REGION [Schizosaccharomyces pombe]) and Z83230 (F56A8.a and F56A8.1 [Caenorhabditis elegans]). Based upon sequence similarity, cn922_{—}5 proteins and each similar protein or peptide may share at least some activity. The TopPredII computer program predicts six potential transmembrane domains within the cn922_{—}5 protein sequence, centered around amino acids 25, 100, 135, 190, 290, and 370 of SEQ ID NO:58, respectively. The nucleotide sequence of cn922_{—}5 indicates that it may contain one or more of the following repetitive elements: MER, L1.

Clone “cw691_{—}11”

A polynucleotide of the present invention has been identified as done “cw691_{—}11”. cw691_{—}11 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cw691_{—}11 is a full-length done, including the entire coding sequence of a secreted protein (also referred to herein as “cw691_{—}11 protein”).

The nucleotide sequence of cw691_{—}11 as presently determined is reported in SEQ ID NO:59, and includes a poly(A) tail. What applicants presently believe to be the proper reading frame and the predicted amino add sequence of the cw691_{—}11 protein corresponding to the foregoing nucleotide sequence is reported in SEQ ID NO:60.

Another potential cw691_{—}11 reading frame and predicted amino acid sequence is encoded by basepairs 542 to 970 of SEQ ID NO:59 and is reported in SEQ ID NO:179. Amino adds 34 to 46 of SEQ ID NO:179 are a predicted leader/signal sequence, with the predicted mature amino acid sequence beginning at amino acid 47. Due to the hydrophobic nature of the predicted leader/signal sequence, it is likely to act as a transmembrane domain should the predicted leader/signal sequence not be separated from the remainder of the protein of SEQ ID NO:179.

The EcoRI/NotI restriction fragment obtainable from the deposit containing done cw691_{—}11 should be approximately 1600 bp.

The nucleotide sequence disclosed herein for cw691_{—}11 was searched against the GenBank and GeneSeq nucleotide sequence databases using BLASTN/BLASTX and FASTA search protocols. cw691_{—}11 demonstrated at least some similarity with sequences identified as AA363712 (EST74158 Pancreas I Homo sapiens cDNA 5′ end similar to similar to C. elegans hypothetical protein R10E12.1), AA521201 (aa74c10.s1 NCI_CGAP_GCB1 Homo sapiens cDNA clone 826674 3′), AA527142 (ni07a10.s1 NCI_CGAP_Br2 Homo sapiens cDNA clone IMAGE 967290, mRNA sequence), AA745501 (ny64d03.s 1 NCI_CGAP_GCB1 Homo sapiens cDNA clone IMAGE: 1283045, “mRNA sequence), N73108 (yv69a09.r1 Homo sapiens cDNA clone 247960 5′), T19938 (Human gene signature HUMGS01070), and W77963 (zd70d09.r1 Soares fetal heart NbHH19W Homo sapiens cDNA clone 346001 5′ similar to WP:R10E12.1 CE00310). The predicted amino acid sequence disclosed herein for cw691_{—}11 was searched against the GenPept and GeneSeq amino acid sequence databases using the BLASTX search protocol. The predicted cw691_{—}11 protein demonstrated at least some similarity to sequences identified as P82971 (Bioadhesive precursor protein from cDNA 52), U73679 (YNK1-a [Caenorhabditis elegans]), and Z29561 (R10E12.1 [Caenorhabditis elegans]). Based upon sequence similarity, cw691_{—}111 proteins and each similar protein or peptide may share at least some activity.

Clone “cw1000_{—}2”

A polynucleotide of the present invention has been identified as done “cw1000_{—}2”. cw1000_{—}2 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cw1000_{—}2 is a full-length done, including the entire coding sequence of a secreted protein (also referred to herein as “cw100012 protein”).

The nucleotide sequence of cw1000_{—}2 as presently determined is reported in SEQ ID NO:61, and includes a poly(A) tail. What applicants presently believe to be the proper reading frame and the predicted amino acid sequence of the cw1000_{—}2 protein corresponding to the foregoing nucleotide sequence is reported in SEQ ID NO:62. Amino acids 24 to 36 of SEQ ID NO:62 are a predicted leader/signal sequence, with the predicted mature amino acid sequence beginning at amino acid 37. Due to the hydrophobic nature of the predicted leader/signal sequence, it is likely to act as a transmembrane domain should the predicted leader/signal sequence not be separated from the remainder of the cw1000_{—}2 protein.

The EcoRI/NotI restriction fragment obtainable from the deposit containing clone cw1000_{—}2 should be approximately 1500 bp.

The nucleotide sequence disclosed herein for cw1000_{—}2 was searched against the GenBank and GeneSeq nucleotide sequence databases using BLASTN/BLASTX and FASTA search protocols. cw1000_{—}2 demonstrated at least some similarity with sequences identified as AA446779 (zw89d11.r1 Soares total fetus Nb2HF8 9w Homo sapiens cDNA clone 784149 5′, mRNA sequence), AA493561 (nh04f07.s1 NCI_CGAP_Thy1 Homo sapiens cDNA clone 943333 similar to WP:F15G9.4 CE01552 IG SUPERFAMILY REPEATS; contains element MSR1 repetitive element), H35690 (EST111696 Rattus sp. cDNA similar to Opioid binding protein/cell adhesion-like molecule), R18502 (yf96a05.r1 Homo sapiens cDNA clone 30376 5′), T21582 (Human gene signature HUMGS02965), T39504 (ya06g11.r1 Homo sapiens cDNA clone 60740 5′), T46848 (yb94b01.r1 Homo sapiens cDNA clone 78793 5′), T51129 (yb94b01.s1 Homo sapiens cDNA clone 78793 3), and W67535 (zd40g11.s1 Soares fetal heart NbHH19W Homo sapiens cDNA clone 343172 3′ similar to PIR SO₅₅₃₉ SO₅₅₃₉ glycophorin C—human; contains element MSR1 repetitive element). The predicted amino acid sequence disclosed herein for cw1000_{—}2 was searched against the GenPept and GeneSeq amino acid sequence databases using the BLASTX search protocol. The predicted cw1000_{—}2 protein demonstrated at least some similarity to sequences identified as M24406 (poliovirus receptor [Homo sapiens]), R07130 (H₂OB receptor), WO4404 (Human CRTAM; Cytotoxic or Regulatory T-cel associated Mol.; CRTAM), X13890 (glycophorin C [Homo sapiens]), and X90569 (elastic titin [Homo sapiens]). Based upon sequence similarity, cw1000_{—}2 proteins and each similar protein or peptide may share at least some activity. The TopPredII computer program predicts an additional potential transmembrane domain within the cw1000_{—}2 protein sequence centered around amino acid 358 of SEQ ID NO:62. The nucleotide sequence of cw1000_{—}2 indicates that it may contain a GCC1 repeat element.

cw100_{—}2 protein was expressed in a COS cell expression system, and an expressed protein band of approximately 57 kDa was detected in membrane fractions using SDS polyacrylamide gel electrophoresis.

Clone “cw1640_{—}1”

A polynucleotide of the present invention has been identified as done “cw1640_{—}1”. cw1640_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cw1640_{—}1 is a full-length done, including the entire coding sequence of a secreted protein (also referred to herein as “cw1640_{—}1 protein”).

The nucleotide sequence of cw1640_{—}1 as presently determined is reported in SEQ ID NO:63, and includes a poly(A) tail. What applicants presently believe to be the proper reading frame and the predicted amino acid sequence of the cw1640_{—}1 protein corresponding to the foregoing nucleotide sequence is reported in SEQ ID NO:64. Amino acids 123 to 135 of SEQ ID NO:64 are a predicted leader/signal sequence, with the predicted mature amino acid sequence beginning at amino acid 136. Due to the hydrophobic nature of the predicted leader/signal sequence, it is likely to act as a transmembrane domain should the predicted leader/signal sequence not be separated from the remainder of the cw1640_{—}1 protein.

The EcoRI/NotI restriction fragment obtainable from the deposit containing done cw1640_{—}1 should be approximately 1400 bp.

The nucleotide sequence disclosed herein for cw1640_{—}1 was searched against the GenBank and GeneSeq nucleotide sequence databases using BLASTN/BLASTX and FASTA search protocols. cw1640_{—}1 demonstrated at least some similarity with sequences identified as AA075643 (zm88a12.r1 Stratagene ovarian cancer (#937219) Homo sapiens cDNA clone 544990 5′ similar to SW:ACT_EUPCR P20360 ACTIN), AA411334 (zv29e11.r1 Soares ovary tumor NbHOT Homo sapiens cDNA clone 755084 5′ similar to WP:C₄₉H_{3.8} CE04234 ACTIN-=KE PROTEIN), AA913364 (ol37b07.s1 Soares NFL_T_GBC_S1 Homo sapiens cDNA clone IMAGE:1525621 3′ similar to WP:C₄₉H_{3.8} CE04234 ACTIN-LIKE PROTEIN, mRNA sequence), N25416 (yx40g10.r1 Homo sapiens cDNA clone 264258 5′ similar to SP ACT2_PLAFA P14883 ACTIN), R96887 (yq61g10.r1 Homo sapiens cDNA clone 200322 5′), W37097 (zb98h03.r1 Soares parathyroid tumor NbHPA Homo sapiens cDNA clone 320885 5′), W44778 (zb98h03.s1 Soares parathyroid tumor NbHPA Homo sapiens cDNA clone 320885 3′), W61038 (zc54g09.r1 Soares senescent fibroblasts NbHSF Homo), W76570 (zd66f12.r1 Soares fetal heart NbHH19W Homo sapiens cDNA clone 345647 5′ similar to SW:ACT_PROCL P45521 ACTIN), and W82519 (mf05b01.r1 Soares mouse p3NMF19.5 Mus musculus cDNA clone). The predicted amino acid sequence disclosed herein for cw1640_{—}1 was searched against the GenPept and GeneSeq amino acid sequence databases using the BLASTX search protocol. The predicted cw1640_{—}1 protein demonstrated at least some”, similarity to sequences identified as J00068 (alpha-actin [Homo sapiens]), J01163 (actin [Oxytricha fallax]), R22026 (A. chrysogenum actin), R50328 (Drug resistant structural protein), U42436 (Similar to actin-like protein [Caenorhabditis elegans]), and U90439 (actin isolog [Arabidopsis thaliana]). Based upon sequence similarity, cw1640_{—}1 proteins and each similar protein or peptide may share at least some activity.

Clone “d24_{—}1”

A polynucleotide of the present invention has been identified as done “d24_{—}1”. A cDNA done was first isolated from a human adult blood (peripheral blood mononuclear cells treated with concanavalin A and phorbol myristate acetate) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. This cDNA done was then used to isolate d24_{—}1 from a human adult blood (peripheral blood mononuclear cells treated with phytohemagglutinin, phorbol myristate acetate, and mixed lymphocyte reaction) cDNA library. d24_{—}1 is a full-length done, including the entire coding sequence of a secreted protein (also referred to herein as “d24_{—}1 protein”).

The nucleotide sequence of d24_{—}1 as presently determined is reported in SEQ ID NO:65, and includes a poly(A) tail. What applicants presently believe to be the proper reading frame and the predicted amino acid sequence of the d24_{—}1 protein corresponding to the foregoing nucleotide sequence is reported in SEQ ID NO:66. Amino acids 124 to 136 of SEQ ID NO:66 are a predicted leader/signal sequence, with the predicted mature amino acid sequence beginning at amino acid 137. Due to the hydrophobic nature of the predicted leader/signal sequence, it is likely to act as a transmembrane domain should the predicted leader/signal sequence not be separated from the remainder of the d24_{—}1 protein. The mRNA sequence encoding amino acids 172 to 175 of SEQ ID NO:66 may not be present in alternatively-spliced forms of d24_{—}1 mRNA molecules.

The EcoRI/NotI restriction fragment obtainable from the deposit containing done d24_{—}1 should be approximately 2000 bp.

The nucleotide sequence disclosed herein for d24_{—}1 was searched against the GenBank and GeneSeq nucleotide sequence databases using BLASTN/BLASTX and FASTA search protocols. d24_{—}1 demonstrated at least some similarity with sequences identified as AA478740 (zy14g12.s1 Soares NhHMPu S1 Homo sapiens cDNA clone 753670 3′), AA479444 (zy14g12.r1 Soares NhHMPu S1 Homo sapiens cDNA clone 753670 5′, mRNA sequence), AA278581 (zs76f09.r1 Soares NbHTGBC Homo sapiens cDNA clone 703433 5′ similar to WP T04A8.12 CE01067 YEAST 107.9 KD PGK1-MAK32 INTERGENIC HYPOTHETICAL PROTEIN), H05202 (yl85h02.r1 Homo sapiens cDNA clone 45213 5′ similar to SP T04A8.12m CE01067 YEAST 107.9 KD PGK1-MAK32 INTERGENIC HYPOTHETICAL PROTEIN), R74287 (yl57e07.r1 Homo sapiens cDNA clone 143364 5), U57715 (Rattus norvegicus FGF receptor activating protein FRAG1 (FRAG1) mRNA, complete CDs), and Z35663 (C. elegans protein of unknown function). The predicted amino acid sequence disclosed herein for d24_{—}1 was searched against the GenPept and GeneSeq amino acid sequence databases using the BLASTX search protocol. The predicted d24_{—}1 protein demonstrated at least some similarity to the sequence identified as U57715 (FGF receptor activating protein FRAG1 [Rattus norvegicus]). Lorenzi et al. (1996, Proc. Natl. Acad. Sci. USA 93:8956, incorporated by reference herein) studied the FRAG1 gene in rat osteosarcoma cells. They concluded that the FRAG1 gene product gets fused to FGF receptor 2 (FGFR2). This fusion “drastically stimulates the transforming activity and autophosphorylation of the receptor” and causes oncogenicity. Based upon sequence similarity, d24_{—}1 proteins and each similar protein or peptide may share at least some activity. The TopPredII computer program predicts three additional potential transmembrane domains within the d24_{—}1 protein sequence, centered around amino acids 34, 154, and 194 of SEQ ID NO:66, respectively.

d24_{—}1 protein was expressed in a COS cell expression system, and an expressed protein band of approximately 24 kDa was detected in membrane fractions using SDS polyacrylamide gel electrophoresis.

Clone “dd426_{—}1”

A polynucleotide of the present invention has been identified as clone “dd426_{—}1”. A cDNA clone was first isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. This cDNA done was then used to isolate dd426_{—}1 from a human adult testes (teratocarcinoma NCCIT) cDNA library. dd426_{—}1 is a full-length done, including the entire coding sequence of a secreted protein (also referred to herein as “dd426_{—}1 protein”).

The nucleotide sequence of dd426_{—}1 as presently determined is reported in SEQ ID NO:67, and includes a poly(A) tail. What applicants presently believe to be the proper reading frame and the predicted amino acid sequence of the dd426_{—}1 protein corresponding to the foregoing nucleotide sequence is reported in SEQ ID NO:68. Amino acids 76 to 88 of SEQ ID NO:68 are a predicted leader/signal sequence, with the predicted mature amino acid sequence beginning at amino acid 89. Due to the hydrophobic nature of the predicted leader/signal sequence, it is likely to act as a transmembrane domain should the predicted leader/signal sequence not be separated from the remainder of the dd426_{—}1 protein.

The EcoRI/NotI restriction fragment obtainable from the deposit containing done dd426_{—}1 should be approximately 800 bp.

The nucleotide sequence disclosed herein for dd426_{—}1 was searched against the GenBank and GeneSeq nucleotide sequence databases using BLASTN/BLASTX and FASTA search protocols. dd426_{—}1 demonstrated at least some similarity with sequences identified as AA760716 (nz13d06.s1 NCI_CGAP_GCB1 Homo sapiens cDNA clone IMAGE:1287659 similar to WP:F13H10.3 CE05624 YEAST YEH4 LIKE PROTEIN; mRNA sequence), H11919 (ym10e10.r1 Homo sapiens cDNA clone 47462 5′), and Z68748 (Caenorhabditis elegans cosmid F13H10). The predicted amino acid sequence disclosed herein for dd426_{—}1 was searched against the GenPept and GeneSeq amino acid sequence databases using the BLASTX search protocol. The predicted dd426_{—}1 protein demonstrated at least some similarity to sequences identified as U39782 (lysine and histidine specific transporter [Arabidopsis thaliana]) and Z68748 (F13H10.3 [Caeno-rhabditis elegans]). Based upon sequence similarity, dd426_{—}1 proteins and each similar protein or peptide may share at least some activity. The TopPredII computer program predicts an additional potential transmembrane domain within the dd426_{—}1 protein sequence centered around amino acid 30 of SEQ ID NO:68, which may also function as a leader/signal sequence.

dd426_{—}1 protein was expressed in a COS cell expression system, and an expressed protein band of approximately 12 kDa was detected in membrane fractions using SDS polyacrylamide gel electrophoresis.

Clone “di393_{—}2”

A polynucleotide of the present invention has been identified as clone “di393_{—}2”. di393_{—}2 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. di393_{—}2 is a full-length done, including the entire coding sequence of a secreted protein (also referred to herein as “di393_{—}2 protein”).

The nucleotide sequence of di393_{—}2 as presently determined is reported in SEQ ID NO:69, and includes a poly(A) tail. What applicants presently believe to be the proper reading frame and the predicted amino acid sequence of the di393_{—}2 protein corresponding to the foregoing nucleotide sequence is reported in SEQ ID NO:70. Amino acids 7 to 19 of SEQ ID NO:70 are a predicted leader/signal sequence, with the predicted mature amino acid sequence beginning at amino acid 20. Due to the hydrophobic nature of the predicted leader/signal sequence, it is likely to act as a transmembrane domain should the predicted leader/signal sequence not be separated from the remainder of the di393_{—}2 protein.

The EcoRI/NotI restriction fragment obtainable from the deposit containing clone di393_{—}2 should be approximately 600 bp.

The nucleotide sequence disclosed herein for di393_{—}2 was searched against the GenBank and GeneSeq nucleotide sequence databases using BLASTN/BLASTX and FASTA search protocols. di393_{—}2 demonstrated at least some similarity with sequences identified as AA669506 (zu85g08.s1 Soares testis NHT Homo sapiens cDNA clone 744830 3′, mRNA sequence). Based upon sequence similarity, di393_{—}2 proteins and each similar protein or peptide may share at least some activity. The TopPredII computer program predicts an additional potential transmembrane domain within the di393_{—}2 protein sequence centered around amino acid 66 of SEQ ID NO:70.

di393_{—}2 protein was expressed in a COS cell expression system, and an expressed protein band of approximately 20 kDa was detected in membrane fractions using SDS polyacrylamide gel electrophoresis.

Clone “dj167_{—}2”

A polynucleotide of the present invention has been identified as done “dj167_{—}2”. dj167_{—}2 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dj167_{—}2 is a full-length done, including the entire coding sequence of a secreted protein (also referred to herein as “dj167_{—}2 protein”).

The nucleotide sequence of dj167_{—}2 as presently determined is reported in SEQ ID NO:71, and includes a poly(A) tail. What applicants presently believe to be the proper reading frame and the predicted amino acid sequence of the dj167_{—}2 protein corresponding to the foregoing nucleotide sequence is reported in SEQ ID NO:72.

The EcoRI/NotI restriction fragment obtainable from the deposit containing done dj167_{—}2 should be approximately 1550 bp.

The nucleotide sequence disclosed herein for dj167_{—}2 was searched against the GenBank and GeneSeq nucleotide sequence databases using BLASTN/BLASTX and FASTA search protocols. dj167_{—}2 demonstrated at least some similarity with sequences identified as H49161 (yq18d05.r1 Soares fetal liver spleen 1NFLS Homo sapiens cDNA clone 274208 5′), L12350 (Human thrombospondin 2 (THBS2) mRNA, complete cds), T98917 (ye66b03.s1 Homo sapiens cDNA clone 122669 3′ similar to SP:TSP1_CHICK P35440 THROMBOSPONDIN 1), and X87620 (B. taurus mRNA for complete thrombospondin). The predicted amino acid sequence disclosed herein for dj167_{—}2 was searched against the GenPept and GeneSeq amino acid sequence databases using the BLASTX search protocol. The predicted dj167_{—}2 protein demonstrated at least some similarity to sequences identified as L12350 (thrombospondin 2 [Homo sapiens]), M60853 (thrombospondin [Gallus gallus]), R40823 (Human thrombospondin 1), U48245 (protein kinase C-binding protein Nel [Rattus norvegicus]), X87620 (thrombospondin [Bos taurus]), and Z71178 (B0024.14 [Caenorhabditis elegans]). Based upon sequence similarity, dj167_{—}2 proteins and each similar protein or peptide may share at least some activity. The TopPredII computer program predicts three potential transmembrane domains within the dj167_{—}2 protein sequence, centered around amino acids 140, 215, and 315 of SEQ ID NO:72, respectively.

Clone “dj167_{—}19”

A polynucleotide of the present invention has been identified as done “dj167_{—}19”. dj167_{—}19 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dj167_{—}19 is a full-length done, including the entire coding sequence of a secreted protein (also referred to herein as “dj167_{—}19 protein”).

The nucleotide sequence of dj167_{—}19 as presently determined is reported in SEQ ID NO:73, and includes a poly(A) tail. What applicants presently believe to be the proper reading frame and the predicted amino acid sequence of the dj167_{—}19 protein corresponding to the foregoing nucleotide sequence is reported in SEQ ID NO:74. Amino acids 22 to 34 of SEQ ID NO:74 are a predicted leader/signal sequence, with the predicted mature amino acid sequence beginning at amino acid 35. Due to the hydrophobic nature of the predicted leader/signal sequence, it is likely to act as a transmembrane domain should the predicted leader/signal sequence not be separated from the remainder of the dj167_{—}19 protein. The dj167_{—}19 clone is related to that of dj167_{—}2, and extends further 5′. The dj167_{—}19 done appears to contain coding sequences for chorionic somato-mammotropin in the opposite orientation at its 5′ end between Sfi restriction sites (at nucleotides 16 and 839 of SEQ ID NO:73). The dj167_{—}2 and dj167_{—}19 clones may represent alternatively spliced messenger RNA molecules encoding two different forms of a secreted protein.

The EcoRI/NotI restriction fragment obtainable from the deposit containing done dj167_{—}19 should be approximately 4500 bp.

Analysis of the dj167_{—}19 amino acid sequence (SEQ ID NO:74) reveals the following domains: IGFBP cysteine-rich domain at amino acids 60-75; VWF-B cysteine-rich domains at amino adds 174-210, 212-247, 255-291, and 293-328; Chordin cysteine-rich domains at amino acids 336-390, 403456, 608-662, 679-734, 753-808, and 819-873; Antistatin (protease inhibitor) cysteine-rich domains at amino acids 469-498, 505-532, 539-564, and 567-592; RGD cell attachment sequence at amino acids 314-316, and Asn glycosylation sites at amino acids 71, 113, 330, 474, and 746. The cysteine-rich domains listed above are similar to domains found in the C domain of Von Willebrand Factor (VWF), and in procollagen and thrombospondin. In addition, the amino acid sequence of SEQ ID NO:74 from amino acid 938 to amino acid 960 appears to be a transmembrane domain.

The dj167_{—}19 transcript is expressed in several cell types, including kidney, pancreas, spleen, and ovary, and is most abundantly expressed in placental tissue.

Clone “dw665_{—}4”

A polynucleotide of the present invention has been identified as clone “dw665_{—}4”. dw665_{—}4 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dw665_{—}4 is a full-length done, including the entire coding sequence of a secreted protein (also referred to herein as “dw665_{—}4 protein”).

The nucleotide sequence of dw665_{—}4 as presently determined is reported in SEQ ID NO:75, and includes a poly(A) tail. What applicants presently believe to be the proper reading frame and the predicted amino acid sequence of the dw665_{—}4 protein corresponding to the foregoing nucleotide sequence is reported in SEQ ID NO:76. Amino acids 15 to 27 of SEQ ID NO:76 are a predicted leader/signal sequence, with the predicted mature amino acid sequence beginning at amino add 28. Amino acids 16 to 28 of SEQ ID NO:76 are also a predicted leader/signal sequence, with the predicted mature amino acid sequence in that case beginning at amino acid 29. Due to the hydrophobic nature of these predicted leader/signal sequences, each is likely to act as a transmembrane domain should it not be separated from the remainder of the dw665_{—}4 protein.

The EcoRI/NotI restriction fragment obtainable from the deposit containing done dw665_{—}4 should be approximately 3750 bp.

The nucleotide sequence disclosed herein for dw665_{—}4 was searched against the GenBank and GeneSeq nucleotide sequence databases using BLASTN/BLASTX and FASTA search protocols. dw665_{—}4 demonstrated at least some similarity with sequences identified as AA029053 (zk09f06.s1 Soares pregnant uterus NbHPU Homo sapiens cDNA clone 470051 3′), H77289 (EST27o17 WATM1 Homo sapiens cDNA clone 27o17, mRNA sequence), and T21722 (Human gene signature HUMGS03170). The predicted amino add sequence disclosed herein for dw665_{—}4 was searched against the GenPept and GeneSeq amino acid sequence databases using the BLASTX search protocol. The predicted dw665_{—}4 protein demonstrated at least some similarity to sequences identified as 135764 (chordin [Xenopus laevis]) and W31559 (Xenopus frog protein “chordin”). Analysis of motifs within the predicted dw665_{—}4 protein revealed the presence of Chordin cysteine-rich domains at amino acids 37-99, 115-178, and 260-322 of SEQ ID NO:76; an “RGD” cell-attachment sequence (at amino acids 179-181 of SEQ ID NO:76), which in some proteins has been shown to play a role in cell adhesion; and Asp glycosylation sites at amino acids 118 and 291. Based upon sequence similarity, dw665_{—}4 proteins and each similar protein or peptide may share at least some activity. The nucleotide sequence of dw665_{—}4 indicates that it may contain an AC repetitive element.

dw665_{—}4 transcripts are expressed in many tissues including kidney, adrenal gland, and prostate tissues, and are most abundantly expressed in pancreas; however, little or no dw665_{—}4 transcript expression is observed in liver or peripheral blood cells. dw665_{—}4 protein was expressed in a COS cell expression system, and an expressed protein band of approximately 75 kDa was detected in conditioned medium using SDS polyacrylamide gel electrophoresis; two additional bands at approximately 26 and 30 kDa were also observed. BIACORE binding experiments indicate that dw665_{—}4 protein has a Chordin-like protein-binding profile, and binds to BMP-2, BMP4, BMP-7, BMP-12, and GDF-5.

Clone “dx146_{—}12”

A polynucleotide of the present invention has been identified as done “dx146_{—}12”. dx146_{—}12 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dx146_{—}12 is a full-length done, including the entire coding sequence of a secreted protein (also referred to herein as “dx146_{—}12 protein”).

The nucleotide sequence of dx146_{—}12 as presently determined is reported in SEQ ID NO:77, and includes a poly(A) tail. What applicants presently believe to be the proper reading frame and the predicted amino acid sequence of the dx146_{—}12 protein corresponding to the foregoing nucleotide sequence is reported in SEQ ID NO:78.

The EcoRI/NotI restriction fragment obtainable from the deposit containing done dx146_{—}12 should be approximately 2250 bp.

The nucleotide sequence disclosed herein for dx146_{—}12 was searched against the GenBank and GeneSeq nucleotide sequence databases using BLASTN/BLASTX and FASTA search protocols. dx146_{—}12 demonstrated at least some similarity with sequences identified as AA090429 (y0527.seq.F Fetal heart, Lambda ZAP Express Homo sapiens cDNA 5′), AA232068 (zr24a01.r1 Stratagene NT2 neuronal precursor 937230 Homo sapiens cDNA clone 664296 5′), AA886679 (oj47h07.s1 NCI_CGAP_Kid3 Homo sapiens cDNA clone IMAGE:1501501 3′ similar to WP:F16A11.2 CE09424 METHANO-COCCUS HYPOTHETICAL PROTEIN 0682 LIKE; mRNA sequence), R61436 (yh15g06.r1 Homo sapiens cDNA clone 37884 5′), and Z81505 (Caenorhabditis elegans cosmid F16A11, complete sequence). The predicted amino acid sequence disclosed herein for dx146_{—}12 was searched against the GenPept and GeneSeq amino acid sequence databases using the BLASTX search protocol. The predicted dx146_{—}12 protein demonstrated at least some similarity to sequences identified as U67515 (hypothetical protein (SP P46850) [Methanococcus jannaschii]) and Z81505 (F16A11.2 [Caenorhabditis elegans]). Based upon sequence similarity, dx146_{—}12 proteins and each similar protein or peptide may share at least some activity. The TopPredII computer program predicts a potential transmembrane domain within the dx146_{—}12 protein sequence centered around amino acid 405 of SEQ ID NO:78.

dx146_{—}12 protein was expressed in a COS cell expression system, and an expressed protein band of approximately 50 kDa was detected in conditioned medium and membrane fractions using SDS polyacrylamide gel electrophoresis.

Clone “dx219_{—}13”

A polynucleotide of the present invention has been identified as clone “dx219_{—}13”. dx219_{—}13 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dx219_{—}13 is a full-length done, including the entire coding sequence of a secreted protein (also referred to herein as “dx219_{—}13 protein”).

The nucleotide sequence of dx219_{—}13 as presently determined is reported in SEQ ID NO:79, and includes a poly(A) tail. What applicants presently believe to be the proper reading frame and the predicted amino acid sequence of the dx219_{—}13 protein corresponding to the foregoing nucleotide sequence is reported in SEQ ID NO:80. Amino acids 94 to 106 of SEQ ID NO:80 are a predicted leader/signal sequence, with the predicted mature amino acid sequence beginning at amino acid 107. Due to the hydrophobic nature of the predicted leader/signal sequence, it is likely to act as a transmembrane domain should the predicted leader/signal sequence not be separated from the remainder of the dx219_{—}13 protein.

The EcoRI/NotI restriction fragment obtainable from the deposit containing done dx219_{—}13 should be approximately 1200 bp.

The nucleotide sequence disclosed herein for dx219_{—}13 was searched against the GenBank and GeneSeq nucleotide sequence databases using BLASTN/BLASTX and FASIA search protocols. dx219_{—}13 demonstrated at least some similarity with sequences identified as AA429731 (zw66g05.s1 Soares testis NHT Homo sapiens cDNA clone 781208 3′), AA446067 (zw66e06.r1 Soares testis NHT Homo sapiens cDNA clone 781186 5′, mRNA sequence), T23212 (standard; cDNA to mRNA; 161 BP, Human gene signature HUMGS05005), W29299 (mb99f03.r1 Soares mouse p3NMF19.5 Mus musculus cDNA clone 337565 5′), W87852 (zh68b05.r1 Soares fetal liver spleen 1NFLS S1 Homo sapiens cDNA clone 417201 5′), and Y13897 (Homo sapiens partial mRNA for hypothetical protein). Based upon sequence similarity, dx219_{—}13 proteins and each similar protein or peptide may share at least some activity. The TopPredII computer program predicts two additional potential transmembrane domains within the dx219_{—}13 protein sequence, one centered around amino acid 160 and another around amino acid 275 of SEQ ID NO:80.

dx219_{—}13 protein was expressed in a COS cell expression system, and an expressed protein band of approximately 37 kDa was detected in membrane fractions using SDS polyacrylamide gel electrophoresis.

Clone “fm3_{—}1”

A polynucleotide of the present invention has been identified as done “fm3_{—}1”. fm3_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fm3_{—}1 is a full-length done, including the entire coding sequence of a secreted protein (also referred to herein as “fn3_{—}1 protein”).

The nucleotide sequence of fm3_{—}1 as presently determined is reported in SEQ ID NO:81, and includes a poly(A) tail. What applicants presently believe to be the proper reading frame and the predicted amino add sequence of the fm3_{—}1 protein corresponding to the foregoing nucleotide sequence is reported in SEQ ID NO:82. Amino acids 7 to 19 of SEQ ID NO:82 are a predicted leader/signal sequence, with the predicted mature amino acid sequence beginning at amino acid 20. Due to the hydrophobic nature of the predicted leader/signal sequence, it is likely to act as a transmembrane domain should the predicted leader/signal sequence not be separated from the remainder of the fm3_{—}1 protein.

The EcoRI/NotI restriction fragment obtainable from the deposit containing done fm3_{—}1 should be approximately 600 bp.

The nucleotide sequence disclosed herein for fm3_{—}1 was searched against the GenBank and GeneSeq nucleotide sequence databases using BLASTN/BLASTX and FASTA search protocols. fm3_{—}1 demonstrated at least some similarity with sequences identified as T15669 (IB1718 Infant brain, Bento Soares Homo sapiens cDNA 3′ end). Based upon sequence similarity, fm3_{—}1 proteins and each similar protein or peptide may share at least some activity. The TopPredII computer program predicts an additional potential transmembrane domains within the fm3_{—}1 protein sequence centered around amino acid 85 of SEQ ID NO:82.

fm3_{—}1 protein was expressed in a COS cell expression system, and an expressed protein band of approximately 9 kDa was detected in membrane fractions using SDS polyacrylamide gel electrophoresis.

Clone “h225_{—}1”

A polynucleotide of the present invention has been identified as clone “h225_{—}1”. h225_{—}1 was isolated from a human adult blood (peripheral blood mononuclear cells treated with phytohemagglutinin and phorbol myristate acetate and mixed lymphocyte reaction) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. h225_{—}1 is a full-length done, including the entire coding sequence of a secreted protein (also referred to herein as “h225_{—}1 protein”).

The nucleotide sequence of h225_{—}1 as presently determined is reported in SEQ ID NO:83. What applicants presently believe to be the proper reading frame and the predicted amino acid sequence of the h225_{—}1 protein corresponding to the foregoing nucleotide sequence is reported in SEQ ID NO:84. Amino acids 52 to 64 of SEQ ID NO:84 are a predicted leader/signal sequence, with the predicted mature amino acid sequence beginning at amino acid 65. Due to the hydrophobic nature of the predicted leader/signal sequence, it is likely to act as a transmembrane domain should the predicted leader/signal sequence not be separated from the remainder of the h225_{—}1 protein.

The EcoRI/NotI restriction fragment obtainable from the deposit containing done h225_{—}1 should be approximately 832 bp.

The nucleotide sequence disclosed herein for h225_{—}1 was searched against the GenBank and GeneSeq nucleotide sequence databases using BLASTN/BLASTX and FASTA search protocols. h225_{—}1 demonstrated at least some similarity with sequences identified as AA604374 (no87e01.s1 NCI_CGAP_AA1 Homo sapiens cDNA clone IMAGE:1113816 similar to WP:ZK757.1 CE00467; mRNA sequence), H18393 (yn49a12.r1 Homo sapiens cDNA clone 171742 5′ similar to SP:ZK757.1 CE00467), and R23642 (yh35e03.r1 Homo sapiens cDNA clone 131740 5′). The predicted amino acid sequence disclosed herein for h225_{—}1 was searched against the GenPept and GeneSeq amino acid sequence databases using the BLASTX search protocol. The predicted h225_{—}1 protein demonstrated at least some similarity to sequences identified as AL022600 (hypothetical protein [Schizosaccharomyces pombe]) and Z48758 (SC9727_{—}21 unknown [Saccharomyces cerevisiae]). Based upon sequence similarity, h225_{—}1 proteins and each similar protein or peptide may share at least some activity.

Clone kj320_{—}1”

A polynucleotide of the present invention has been identified as done “kj320_{—}1”. kj320_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. kj320_{—}1 is a full-length done, including the entire coding sequence of a secreted protein (also referred to herein as kj320_{—}1 protein”).

The nucleotide sequence of kj320_{—}1 as presently determined is reported in SEQ ID NO:85, and includes a poly(A) tail. What applicants presently believe to be the proper reading frame and the predicted amino acid sequence of the kj320_{—}1 protein corresponding to the foregoing nucleotide sequence is reported in SEQ ID NO:86. Amino acids 26 to 38 of SEQ ID NO:86 are a predicted leader/signal sequence, with the predicted mature amino acid sequence beginning at amino acid 39. Due to the hydrophobic nature of the predicted leader/signal sequence, it is likely to act as a transmembrane domain should the predicted leader/signal sequence not be separated from the remainder of the kj320_{—}1 protein.

The EcoRI/NotI restriction fragment obtainable from the deposit containing done kj320_{—}1 should be approximately 4900 bp.

The nucleotide sequence disclosed herein for kj320_{—}1 was searched against the GenBank and GeneSeq nucleotide sequence databases using BLASTN/BLASTX and FASTA search protocols. kj320_{—}1 demonstrated at least some similarity with sequences identified as A45343 (Sequence 13 from Patent WO9517522), AA284111 (zc36f08.T7 Soares senescent fibroblasts NbHSF Homo sapiens cDNA clone 324423 3′ similar to WP ZK688.8 CE00544 UDP-GALNAC; mRNA sequence), AA375707 (EST88026 HSC172 cells II Homo sapiens cDNA 5′ end), AA534406 (nf76b08.s1 NCI_CGAP_Co3 Homo sapiens cDNA clone IMAGE 925815), D39885 (Rice cDNA, partial sequence (S1531_{—1}A)), G10010 (human STS CHLC.GCT16E06.P18287 clone GCT16E06), Q75104 (Cattle GalNAc-transferase), Q95187 (Simple tandem repeat (SfR) corresponding to wg1d10), and U35890 (Rattus norvegicus polypeptide GalNAc transferase Ti mRNA, complete cds). The predicted amino acid sequence disclosed herein for kj320_{—}1 was searched against the GenPept and GeneSeq amino acid sequence databases using the BLASTX search protocol. The predicted kj320_{—}1 protein demonstrated at least some similarity to sequences identified as R66397 (Cattle GalNAc-transferase), U41514 (UDP-GalNAc polypeptide N-acetylgalactosaminyltransferase [Homo sapiens]), and X85018 (UDP-GalNAc polypeptide N-acetylgalactosaminyl transferase [Homo sapiens]). Analysis of motifs within kj320_{—}1 reveals the presence of the alpha-2-macroglobulin family thiolester region signature. The proteinase-binding alpha-macroglobulins (A2M) are large glycoproteins found in the plasma of vertebrates, in the hemolymph of some invertebrates, and in reptilian and avian egg white. They inhibit all four classes of proteinases by trapping a proteinase with a peptide stretch containing the specific cleavage site (the ‘bait’ region) which upon proteinase binding induces a conformational change in the protein, trapping the proteinase. Upon cleavage of the ‘bait’ region, a covalent bond (a thiol-ester bond between the side chains of a cysteine and a glutamine) is formed between the A2M and the proteinase. Based upon sequence similarity, kj320_{—}1 proteins and each similar protein or peptide may share at least some activity. The nucleotide sequence of kj320_{—}1 indicates that it may contain one or more repetitive elements.

kj320_{—}1 protein was expressed in a COS cell expression system, and an expressed protein band of approximately 136 kDa was detected in conditioned medium using SDS polyacrylamide gel electrophoresis.

Clone “ml236_{—}5”

A polynucleotide of the present invention has been identified as done “ml236_{—}5”. ml236_{—}5 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ml236_{—}5 is a full-length done, including the entire coding sequence of a secreted protein (also referred to herein as “ml365 protein”).

The nucleotide sequence of ml236_{—}5 as presently determined is reported in SEQ ID NO:87, and includes a poly(A) tail. What applicants presently believe to be the proper reading frame and the predicted amino acid sequence of the ml236_{—}5 protein corresponding to the foregoing nucleotide sequence is reported in SEQ ID NO:88. Amino adds 148 to 160 of SEQ ID NO:88 are a predicted leader/signal sequence, with the predicted mature amino acid sequence beginning at amino acid 161. Due to the hydrophobic nature of the predicted leader/signal sequence, it is likely to act as a transmembrane domain should the predicted leader/signal sequence not be separated from the remainder of the ml236_{—}5 protein.

The EcoRI/NotI restriction fragment obtainable from the deposit containing done ml236_{—}5 should be approximately 1300 bp.

The nucleotide sequence disclosed herein for ml236_{—}5 was searched against the GenBank and GeneSeq nucleotide sequence databases using BLASTN/BLASTX and FASTA search protocols. ml236_{—}5 demonstrated at least some similarity with sequences identified as AA137204 (z123h11.s1 Soares pregnant uterus NbHPU Homo sapiens cDNA clone 502821 3), AA307966 (EST17887 Aorta endothelial cells, TNF alpha-treated Homo sapiens cDNA 5′ end, mRNA sequence), AA434504 (zw31c03.r1 Soares ovary tumor NbHOT Homo sapiens cDNA clone 770884 5′ similar to WP C45G9.7 CE01858), AA525971 (ni93g09.s1 NCI_CGAP_Pr21 Homo sapiens cDNA clone 984448), AA526490 (ni96c11.s1 NCI_CGAP_Pr21 Homo sapiens cDNA clone MAGE 984692, mRNA sequence), AF028823 (Homo sapiens Tax interaction protein 1 mRNA, partial cds), U90913 (Human clone 23665 mRNA sequence), and W73114 (zd55c12.r1 Soares fetal heart NbHH19W Homo sapiens cDNA clone 344566 5′). The predicted amino acid sequence disclosed herein for ml236_{—}5 was searched against the GenPept and GeneSeq amino acid sequence databases using the BLASTX search protocol. The predicted ml236_{—}5 protein demonstrated at least some similarity to sequences identified as AF028823 (Tax interaction protein 1 [Homo sapiens]) and U21323 (similar to tight junction protein (Z0-1) (SP Z01_HUMAN, Q07157) [Caenorhabditis elegans]). Based upon sequence similarity, ml236_{—}5 proteins and each similar protein or peptide may share at least some activity.

ml236_{—}5 protein was expressed in a COS cell expression system, and an expressed protein band of approximately 14 kDa was detected in conditioned medium and membrane fractions using SDS polyacrylamide gel electrophoresis.

Clone “pu282_{—}10”

A polynucleotide of the present invention has been identified as clone “pu282_{—}10”. pu282_{—}10 was isolated from a human adult blood (promyelocytic leukemia HL-60) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pu282_{—}10 is a full-length done, including the entire coding sequence of a secreted protein (also referred to herein as “pu82_{—}10 protein”).

The nucleotide sequence of pu282_{—}10 as presently determined is reported in SEQ ID NO:89, and includes a poly(A) tail. What applicants presently believe to be the proper reading frame and the predicted amino acid sequence of the pu282_{—}10 protein corresponding to the foregoing nucleotide sequence is reported in SEQ ID NO:90. Amino acids 119 to 131 of SEQ ID NO:90 are a predicted leader/signal sequence, with the predicted mature amino add sequence beginning at amino acid 132. Due to the hydrophobic nature of the predicted leader/signal sequence, it is likely to act as a transmembrane domain should the predicted leader/signal sequence not be separated from the remainder of the pu282_{—}10 protein.

The EcoRI/NotI restriction fragment obtainable from the deposit containing done pu282_{—}10 should be approximately 1050 bp.

The nucleotide sequence disclosed herein for pu282_{—}10 was searched against the GenBank and GeneSeq nucleotide sequence databases using BLASTN/BLASTX and FASTA search protocols. pu82_{—}10 demonstrated at least some similarity with sequences identified as AA311503 (EST182442 Jurkat T-cells VI Homo sapiens cDNA 5′ end), AA336709 (EST41341 Endometrial tumor Homo sapiens cDNA 5′ end), AA336890 (EST41572 Endometrial tumor), AA385588 (EST99290 Thyroid Homo sapiens cDNA 5′ end), AA526889 (ni09e05.s1 NCI_CGAP_Br2 Homo sapiens cDNA clone IMAGE:967520), AC003058 (Arabidopsis thaliana “unknown” protein), and T19726 (Human gene signature HUMGS00800). Based upon sequence similarity, pu282_{—}10 proteins and each similar protein or peptide may share at least some activity. The TopPredII computer program predicts two additional potential transmembrane domains within the pu282_{—}10 protein sequence, one centered around amino acid 39 and another around amino add 95 of SEQ ID NO:90.

pu282_{—}10 protein was expressed in a COS cell expression system, and an expressed protein band of approximately 16 kDa was detected in conditioned medium and membrane fractions using SDS polyacrylamide gel electrophoresis.

Clone “at94_{—}2”

A polynucleotide of the present invention has been identified as done “at94_{—}2”. at94_{—}2 was isolated from a human adult blood (lymphocytes and dendritic cells treated with mixed lymphocyte reaction) cDNA library using methods which are selective for cDNAs encoding secreted proteins (see U.S. Pat. No. 5,536,637), or was identified as encoding a secreted or transmembrane protein on the basis of computer analysis of the amino add sequence of the encoded protein. at94_{—}2 is a full-length done, including the entire coding sequence of a secreted protein (also referred to herein as “at94_{—}2 protein”).

The nucleotide sequence of at942 as presently determined is reported in SEQ ID NO:91, and includes a poly(A) tail. What applicants presently believe to be the proper reading frame and the predicted amino acid sequence of the at94_{—}2 protein corresponding to the foregoing nucleotide sequence is reported in SEQ ID NO:92. Amino acids 214 to 226 of SEQ ID NO:92 are a predicted leader/signal sequence, with the predicted mature amino acid sequence beginning at amino add 227. Due to the hydrophobic nature of the predicted leader/signal sequence, it is likely to act as a transmembrane domain should the predicted leader/signal sequence not be separated from the remainder of the at94_{—}2 protein.

The EcoRI/NotI restriction fragment obtainable from the deposit containing done at94_{—}2 should be approximately 4300 bp.

The nucleotide sequence disclosed herein for at94_{—}2 was searched against the GenBank and GeneSeq nucleotide sequence databases using BLASTN/BLASTX and FASTA search protocols. at94_{—}2 demonstrated at least some similarity with sequences identified as N24317 (yx23d12.r1 Homo sapiens cDNA clone 262583 5′), T30988 (EST25695 Homo sapiens cDNA 5′ end similar to None), and U37026 (Rattus norvegicusbrain sodium channel beta 2 subunit (SCNB2) mRNA, complete cds). The predicted amino acid sequence disclosed herein for a94_{—}2 was searched against the GenPept and GeneSeq amino acid sequence databases using the BLASTX search protocol. The predicted at94_{—}2 protein demonstrated at least some similarity to the sequence identified as Z49912 (T24F1.2 [Caenorhabditis elegans]). Based upon sequence similarity, at94_{—}2 proteins and each similar protein or peptide may share at least some activity. The TopPredII computer program predicts four additional potential transmembrane domains within the at94_{—}2 protein sequence, centered around amino acids 23, 306, 332, and 364 of SEQ ID NO:92, respectively.

Clone “bf169_{—}13”

A polynucleotide of the present invention has been identified as done “bf169_{—}13”. bf169_{—}13 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bf169_{—}13 is a full-length clone, including the entire coding sequence of a secreted protein (also referred to herein as “bf169_{—}13 protein”).

The nucleotide sequence of bf169_{—}13 as presently determined is reported in SEQ ID NO:93, and includes a poly(A) tail. What applicants presently believe to be the proper reading frame and the predicted amino acid sequence of the bf169_{—}13 protein corresponding to the foregoing nucleotide sequence is reported in SEQ ID NO:94. Amino acids 342 to 354 are a possible leader/signal sequence, with the predicted mature amino acid sequence beginning at amino acid 355. Due to the hydrophobic nature of this possible leader/signal sequence, it is likely to act as a transmembrane domain should it not be separated from the remainder of the bf169_{—}13 protein.

The EcoRI/NotI restriction fragment obtainable from the deposit containing done bf169_{—}13 should be approximately 3000 bp.

The nucleotide sequence disclosed herein for bf169_{—}13 was searched against the GenBank and GeneSeq nucleotide sequence databases using BLASTN/BLASTX and FASTA search protocols. bf169_{—}13 demonstrated at least some similarity with sequences identified as AA227952 (zr56b06.s1 Soares NhHMPu S1 Homo sapiens cDNA clone 667379 3′), AA453914 (zx32e11.r1 Soares total fetus Nb2HF8 9w Homo sapiens cDNA clone 788204 5′ similar to contains element TAR1 repetitive element; mRNA sequence), H46157 (yo13f11.r1 Homo sapiens cDNA clone 177837 5′), H18792 (yn52e02.r1 Homo sapiens cDNA clone 172058 5′), and N24601 (yx72e01.s1 Homo sapiens cDNA clone 267288 3′). The predicted amino acid sequence disclosed herein for bf169_{—}13 was searched against the GenPept and GeneSeq amino acid sequence databases using the BLASTX search protocol. The predicted bf169_{—}13 protein demonstrated at least some similarity to sequences identified as L41834 (plant nuclear protein [Ensis minor]) and Z75539 (F28C1.1 [Caenorhabditis elegans]). Analysis of motifs in the predicted bf169_{—}13 protein revealed a “mitochondrial energy transfer proteins” signature at amino acid 574 of SEQ ID NO:94. Based upon sequence similarity, bf169_{—}13 proteins and each similar protein or peptide may share at least some activity. The nucleotide sequence of bf169_{—}13 indicates that it may contain one or more GCCCCA, GCCC, GGA and/or GC repeat sequences.

bf169_{—}13 protein was expressed in a COS cell expression system, and an expressed protein band of approximately 109 kDa was detected in membrane fractions using SDS polyacrylamide gel electrophoresis.

Clone “bl152_{—}12”

A polynucleotide of the present invention has been identified as done “bl152_{—}12”. bl152_{—}12 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino add sequence of the encoded protein. bl152_{—}12 is a full-length clone, including the entire coding sequence of a secreted protein (also referred to herein as “bl152_{—}12 protein”).

The nucleotide sequence of bl152_{—}12 as presently determined is reported in SEQ ID NO:95, and includes a poly(A) tail. What applicants presently believe to be the proper reading frame and the predicted amino add sequence of the bl152_{—}12 protein corresponding to the foregoing nucleotide sequence is reported in SEQ ID NO:96.

The EcoRI/NotI restriction fragment obtainable from the deposit containing clone bl152_{—}12 should be approximately 1100 bp.

The nucleotide sequence disclosed herein for bl152_{—}12 was searched against the GenBank and GeneSeq nucleotide sequence databases using BLASTN/BLASTX and FASTA search protocols. bl152_{—}12 demonstrated at least some similarity with sequences identified as AA280876 (zs97d04.s1 NCI_CGAP_GCB1 Soares NbHTGBC Homo sapiens cDNA clone 711559 3′ similar to contains element MER22 repetitive element), AA280956 (zs97d04.r1 NCI_CGAP_GCB1 Soares NbHTGBC Homo sapiens cDNA clone 711559 5′), R21512 (yh19b03.s1 Homo sapiens cDNA clone 130157 3′), R67018 (yl26e05.s1 Homo sapiens cDNA clone 140384 3′ similar to contains MER22 repetitive element), R71877 (yj87d11.s1 Homo sapiens cDNA clone 155733 3′ similar to contains MER22 repetitive element), T22941 (Human gene signature HUMGS04666), W46539 (zc30g03.s1 Soares senescent fibroblasts NbHSF Homo sapiens cDNA clone 323860 3′, mRNA sequence), and W70065 (zd49c04.s1 Soares fetal heart NbHH19W Homo sapiens cDNA clone). The predicted amino acid sequence disclosed herein for bl152_{—}12 was searched against the GenPept and GeneSeq amino add sequence databases using the BLASTX search protocol. The predicted bl152_{—}12 protein demonstrated at least some similarity to the sequence identified as Z82256 (B0513.2 [Caenorhabditis elegans]). Based upon sequence similarity, bl152_{—}12 proteins and each similar protein or peptide may share at least some activity. The nucleotide sequence of bl152_{—}12 indicates that it may contain one or more GCC repeat sequences.

bl152_{—}12 protein was expressed in a COS cell expression system, and an expressed protein band of approximately 25 kDa was detected in conditioned medium using SDS polyacrylamide gel electrophoresis.

Clone “bz578_{—}1”

A polynucleotide of the present invention has been identified as done “bz578_{—}1”. bz578_{—}1 was isolated from a human fetal kidney cDNA library using methods and was identified as encoding a novel protein on the basis of computer analysis of the amino acid sequence of the encoded protein. bz578_{—}1 is a full-length done, including the entire coding sequence of a novel protein (also referred to herein as “bz578_{—}1 protein”).

The nucleotide sequence of bz578_{—}1 as presently determined is reported in SEQ ID NO:97, and includes a poly(A) tail. What applicants presently believe to be the proper reading frame and the predicted amino acid sequence of the bz578_{—}1 protein corresponding to the foregoing nucleotide sequence is reported in SEQ ID NO:98.

The EcoRI/NotI restriction fragment obtainable from the deposit containing done bz578_{—}1 should be approximately 1000 bp.

The nucleotide sequence disclosed herein for bz578_{—}1 was searched against the GenBank and GeneSeq nucleotide sequence databases using BLASTN/BLASTX and FASTA search protocols. bz578_{—}1 demonstrated at least some similarity with sequences identified as T47038 (yb12e08.r1 Homo sapiens cDNA clone 70982 5′ contains L1 repetitive element) and Z82975 (Human DNA sequence from PAC 36J3, between markers DXS 1192 and DXS 102 on chromosome X). The predicted amino acid sequence disclosed herein for bz578_{—}1 was searched against the GenPept and GeneSeq amino acid sequence databases using the BLASTX search protocol. The predicted bz578_{—}1 protein demonstrated at least some similarity to sequences identified as AF051782 (diaphanous 1 [Homo sapiens]), U96963 (diaphanous 1 [mouse]), and U93572 (putative p150 [Homo sapiens]). Based upon sequence similarity, bz578_{—}1 proteins and each similar protein or peptide may share at least some activity. The nucleotide sequence of bz578_{—}1 indicates that it may contain one or more L1 repeat sequences.

Clone “cb123_{—}1”

A polynucleotide of the present invention has been identified as clone “cb123_{—}1”. cb123_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cb123_{—}1 is a full-length clone, including the entire coding sequence of a secreted protein (also referred to herein as “cb123_{—}1 protein”).

The nucleotide sequence of cb123_{—}1 as presently determined is reported in SEQ ID NO:99, and includes a poly(A) tail. What applicants presently believe to be the proper reading frame and the predicted amino acid sequence of the cb123_{—}1 protein corresponding to the foregoing nucleotide sequence is reported in SEQ ID NO:100. Amino acids 44 to 56 of SEQ ID NO:100 are a predicted leader/signal sequence, with the predicted mature amino acid sequence beginning at amino acid 57. Due to the hydrophobic nature of the predicted leader/signal sequence, it is likely to act as a transmembrane domain should the predicted leader/signal sequence not be separated from the remainder of the cb123_{—}1 protein.

The EcoRI/NotI restriction fragment obtainable from the deposit containing done cb123_{—}1 should be approximately 1500 bp.

The nucleotide sequence disclosed herein for cb123_{—}1 was searched against the GenBank and GeneSeq nucleotide sequence databases using BLASTN/BLASTX and FASTA search protocols. cb123_{—}1 demonstrated at least some similarity with sequences identified as AA309020 (EST179803 Colon carcinoma (Caco-2) cell line I Homo sapiens cDNA 5′ end, mRNA sequence), R89617 (ym98b08.s1 Homo sapiens cDNA clone 166935 3′), T16814 (NB 1893 Normalized infant brain, Bento Soares Homo sapiens cDNA 3′ end similar to EST02882H. sapiens cDNA clone HFBCL71), T24092 (Human gene signature HUMGS06080), and T55187 (yb43e06.s1 Homo sapiens cDNA clone 73954 3′). The predicted amino acid sequence disclosed herein for cb123_{—}1 was searched against the GenPept and GeneSeq amino acid sequence databases using the BLASTX search protocol. The predicted cb123_{—}1 protein demonstrated at least some similarity to the sequence identified as U33331 (orf UL133 [Human cytomegalovirus]). Based upon sequence similarity, cb123_{—}1 proteins and each similar protein or peptide may share at least some activity. The TopPredII computer program predicts two additional potential transmembrane domains within the cb123_{—}1 protein sequence, one centered around amino acid 15 and another around amino acid 80 of SEQ ID NO: 100.

Clone “ch245_{—}1”

A polynucleotide of the present invention has been identified as clone “ch245_{—}1”. ch245_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ch245_{—}1 is a full-length done, including the entire coding sequence of a secreted protein (also referred to herein as “ch245_{—}1 protein”).

The nucleotide sequence of ch245_{—}1 as presently determined is reported in SEQ ID NO:101, and includes a poly(A) tail. What applicants presently believe to be the proper reading frame and the predicted amino acid sequence of the ch245_{—}1 protein corresponding to the foregoing nucleotide sequence is reported in SEQ ID NO:102. The TopPredII computer program predicts a potential transmembrane domain within the ch245_{—}1 protein sequence centered around amino acid 87 of SEQ ID NO:102.

Another potential ch245_{—}1 reading frame and predicted amino acid sequence is encoded by basepairs 533 to 778 of SEQ ID NO:101 and is reported in SEQ ID NO:180. The TopPredII computer program predicts a potential transmembrane domain within the SEQ ID NO:180 amino acid sequence centered around amino acid 34 of SEQ ID NO:180.

The EcoRI/NotI restriction fragment obtainable from the deposit containing done ch245_{—}1 should be approximately 1350 bp.

The nucleotide sequence disclosed herein for ch245_{—}1 was searched against the GenBank and GeneSeq nucleotide sequence databases using BLASTN/BLASTX and FASTA search protocols. ch245_{—}1 demonstrated at least some similarity with sequences identified as AA402307 (zu48f03.r1 Soares ovary tumor NbHOT Homo sapiens cDNA clone 741245 5′, mRNA sequence), H19032 (ym44e04.r1 Homo sapiens cDNA clone 50921 5′), H19323 (ym44e04.s1 Homo sapiens cDNA clone 50921 3′), and N36070 (yy02g11.r1 Homo sapiens cDNA clone 270116 5′). The predicted amino acid sequence disclosed herein for ch245_{—}1 was searched against the GenPept and GeneSeq amino acid sequence databases using the BLASTX search protocol. The predicted ch245_{—}1 protein demonstrated at least some similarity to the sequence identified as M58597 (ELAM-1 ligand fucosyltransferase [Homo sapiens]) and U36763 (fatty acid synthase [Mycobacterium bovis]). Based upon sequence similarity, ch245_{—}1 proteins and each similar protein or peptide may share at least some activity.

Clone “gj378_{—}3”

A polynucleotide of the present invention has been identified as clone “cj378_{—}3”. cj378_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cj378_{—}3 is a full-length done, including the entire coding sequence of a secreted protein (also referred to herein as “cj378_{—}3 protein”).

The nucleotide sequence of cj378_{—}3 as presently determined is reported in SEQ ID NO:103, and includes a poly(A) tail. What applicants presently believe to be the proper reading frame and the predicted amino add sequence of the cj378_{—}3 protein corresponding to the foregoing nucleotide sequence is reported in SEQ ID NO:104.

The EcoRI/NotI restriction fragment obtainable from the deposit containing done cj378_{—}3 should be approximately 1400 bp.

The nucleotide sequence disclosed herein for cj378_{—}3 was searched against the GenBank and GeneSeq nucleotide sequence databases using BLASTN/BLASTX and FASTA search protocols. cj378_{—}3 demonstrated at least some similarity with sequences identified as D60138 (Human fetal brain cDNA 5′-end GEN-088A04, mRNA sequence), H19318 (ym44d06.s1 Homo sapiens cDNA clone 51231 3′), H41859 (yo07g06.r1 Homo sapiens cDNA clone 177274 5′), T25386 (Human gene signature HUMGS07551), and T75383 (yc89g05.r1 Homo sapiens cDNA clone 23351 5′). Based upon sequence similarity, cj378_{—}3 proteins and each similar protein or peptide may share at least some activity. The TopPredII computer program predicts a potential transmembrane domain at the N-terminus of the the cj378_{—}3 protein sequence (SEQ ID NO:104).

Clone “cw1481_{—}1”

A polynucleotide of the present invention has been identified as clone “cw1481_{—}1”. cw1481_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cw1481_{—}1 is a full-length done, including the entire coding sequence of a secreted protein (also referred to herein as “cw1481_{—}1 protein”).

The nucleotide sequence of cw1481_{—}1 as presently determined is reported in SEQ ID NO:105, and includes a poly(A) tail. What applicants presently believe to be the proper reading frame and the predicted amino acid sequence of the cw1481_{—}1 protein corresponding to the foregoing nucleotide sequence is reported in SEQ ID NO:106.

The EcoRI/NotI restriction fragment obtainable from the deposit containing done cw1481_{—}1 should be approximately 2380 bp.

The nucleotide sequence disclosed herein for cw1481_{—}1 was searched against the GenBank and GeneSeq nucleotide sequence databases using BLASTN/BLASTX and FASTA search protocols. cw1481_{—}1 demonstrated at least some similarity with sequences identified as AA027927 (zk05a10.r1 Soares pregnant uterus NbHPU Homo sapiens cDNA clone 469626 5′), AA027928 (zk05a10.s1 Soares pregnant uterus NbHPU Homo sapiens cDNA clone 469626 3′ similar to contains MER28.b2 MER28 repetitive element), AA113357 (zn69g06.s1 Stratagene HeLa cell s3 937216 Homo sapiens cDNA clone 563482 3′), AA252304 (zs12b08.s1 Soares NbHTGBC Homo sapiens cDNA clone 684951 3′ similar to contains element MER22 repetitive element), AA976744 (oq09a09.s1 NCI_CGAP_GC4 Homo sapiens cDNA clone IMAGE 1585816 3′ similar to TR O15025 O15025 KIAA0308; contains element MER22 repetitive element; mRNA sequence), R55084 (yg87a06.r1 Homo sapiens cDNA clone 40244 5′), U00930 (Human clone C4E 1.63 (CAC)n/(GTG)ₙ repeat-containing mRNA), U00955 (Human clone CE29 8.1 (CAC)n/(GTG)ₙ repeat-containing mRNA), and W16808 (zb93a09.s1 Soares parathyroid tumor NbHPA Homo sapiens cDNA clone 320344 3′). The predicted amino acid sequence disclosed herein for cw1481_{—}1 was searched against the GenPept and GeneSeq amino acid sequence databases using the BLASTX search protocol. The predicted cw1481_{—}1 protein demonstrated at least some similarity to sequences identified as AB002306 (KIAA0308 [Homo sapiens]), X15906 (precursor polypeptide), and Z68751 (F01G4.1 [Caenorhabditis elegans]). Based upon sequence similarity, cw1481_{—}1 proteins and each similar protein or peptide may share at least some activity. The TopPredII computer program predicts a potential transmembrane domain within the cw1481_{—}1 protein sequence centered around amino acid 431 of SEQ ID NO:106, and a putative transmembrane domain within the cw1481_{—}1 protein sequence centered around amino acid 395 of SEQ ID NO:106. The amino acid sequence of cw1481_{—}1 indicates that it has a histidine-rich region and a serine-rich region, and it is strongly internally repeated.

Clone “dd119_{—}4”

A polynucleotide of the present invention has been identified as clone “dd119_{—}4”. dd119_{—}4 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dd119_{—}4 is a full-length done, including the entire coding sequence of a secreted protein (also referred to herein as “dd119_{—}4 protein”).

The nucleotide sequence of dd119_{—}4 as presently determined is reported in SEQ ID NO:107, and includes a poly(A) tail. What applicants presently believe to be the proper reading frame and the predicted amino acid sequence of the dd119_{—}4 protein corresponding to the foregoing nucleotide sequence is reported in SEQ ID NO:108. Amino acids 27 to 39 of SEQ ID NO:108 are a predicted leader/signal sequence, with the predicted mature amino acid sequence beginning at amino acid 40. Due to the hydrophobic nature of the predicted leader/signal sequence, it is likely to act as a transmembrane domain should the predicted leader/signal sequence not be separated from the remainder of the dd119_{—}4 protein.

The EcoRI/NotI restriction fragment obtainable from the deposit containing clone dd119_{—}4 should be approximately 3350 bp.

The nucleotide sequence disclosed herein for dd119_{—}4 was searched against the GenBank and GeneSeq nucleotide sequence databases using BLASTN/BLASTX and FASTA search protocols. dd119_{—}4 demonstrated at least some similarity with sequences identified as AA151924 (zo30e05.r1 Stratagene colon (#937204) Homo sapiens cDNA clone 588416 5′ similar to SW SLIT_DROME P24014 SLIT PROTEIN PRECURSOR; mRNA sequence), AA193464 (zr41c06.s1 Soares NHMPu S1 Homo sapiens cDNA clone 665962 3′), AB011135 (Homo sapiens mRNA for KIAA0563 protein, complete cds), G23888 (human STS WI-12393), H04996 (yl74c12.s1 Homo sapiens cDNA clone 43851 3′), M86526 (Rat proline-rich protein (PRP) gene, 5′ end, and containing several Alu-like repetitive elements), M86514 (Rat proline-rich protein mRNA, 3′ end), W68823 (zd37f04.r1 Soares fetal heart NbHH19W Homo sapiens cDNA clone 342847 5′), and Z54386 (H. sapiens CpG island DNA genomic Mse1 fragment, clone 10g3, forward read cpg10g3.ft1a). The predicted amino acid sequence disclosed herein for dd119_{—}4 was searched against the GenPept and GeneSeq amino acid sequence databases using the BLASTX search protocol. The predicted dd119_{—}4 protein demonstrated at least some similarity to sequences identified as AB011135 (KIAA0563 protein ([Homo sapiens]) and M86526 (proline-rich protein [Rattus norvegicus]). The rat proline-rich protein (PRP) is encoded by a single-copy gene and is expressed in the ventral prostate of the rat, with the precursor protein product being cleaved into multiple proline-rich polypeptides. Based upon sequence similarity, dd119_{—}4 proteins and each similar protein or peptide may share at least some activity. The TopPredII computer program predicts an additional potential transmembrane domain within the dd119_{—}4 protein sequence centered around amino add 928 of SEQ ID NO:108.

dd119_{—}4 protein was expressed in a COS cell expression system, and an expressed protein band of approximately 166 kDa was detected in conditioned medium and membrane fractions using SDS polyacrylamide gel electrophoresis.

Clone “df202_{—}3”

A polynucleotide of the present invention has been identified as done “df202_{—}3”. dfM02_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino add sequence of the encoded protein. df202_{—}3 is a full-length done, including the entire coding sequence of a secreted protein (also referred to herein as “df202_{—}3 protein”).

The nucleotide sequence of df202_{—}3 as presently determined is reported in SEQ ID NO:109, and includes a poly(A) tail. What applicants presently believe to be the proper reading frame and the predicted amino acid sequence of the df02_{—}3 protein corresponding to the foregoing nucleotide sequence is reported in SEQ ID NO:110. Amino acids 17 to 29 of SEQ ID NO:110 are a predicted leader/signal sequence, with the predicted mature amino acid sequence beginning at amino add 30. Due to the hydrophobic nature of the predicted leader/signal sequence, it is likely to act as a transmembrane domain should the predicted leader/signal sequence not be separated from the remainder of the df202_{—}3 protein.

The EcoRI/NotI restriction fragment obtainable from the deposit containing done df02_{—}3 should be approximately 1600 bp.

The nucleotide sequence disclosed herein for df202_{—}3 was searched against the GenBank and GeneSeq nucleotide sequence databases using BLASTN/BLASTX and FASTA search protocols. df202_{—}3 demonstrated at least some similarity with sequences identified as AA138679 (mq76g03.r1 Stratagene mouse melanoma (#937312) Mus musculus cDNA clone 584692 5′), AA283121 (zt17b05.s1 Soares ovary tumor NbHOT Homo sapiens cDNA clone 713361 3′), AA286996 (zs58c10.r1 NCI_CGAP_GCB1 Soares NbHTGBC Homo sapiens cDNA clone IMAGE 701682 5′), N54968 (yv38g01.s1 Homo sapiens cDNA clone 245040 3′), T20071 (Human gene signature HUMGS01213), and W28275 (44g12 Human retina cDNA randomly primed sublibrary Homo sapiens cDNA). The predicted amino acid sequence disclosed herein for df02_{—}3 was searched against the GenPept and GeneSeq amino acid sequence databases using the BLASTX search protocol. The predicted df202_{—}3 protein demonstrated at least some similarity to the sequence identified as Z81137 (WO2D9.h [Caenorhabditis elegans]). Based upon sequence similarity, df02_{—}3 proteins and each similar protein or peptide may share at least some activity. The TopPredII computer program predicts three additional potential transmembrane domains within the df202_{—}3 protein sequence, centered around amino acids 55, 80, and 108 of SEQ ID NO:110, respectively.

Clone “km225_{—}1”

A polynucleotide of the present invention has been identified as done “km225_{—}1”. km225_{—}1 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. km225_{—}1 is a full-length done, including the entire coding sequence of a secreted protein (also referred to herein as “km225_{—}1 protein”).

The nucleotide sequence of km225_{—}1 as presently determined is reported in SEQ ID NO:111, and includes a poly(A) tail. What applicants presently believe to be the proper reading frame and the predicted amino acid sequence of the km225_{—}1 protein corresponding to the foregoing nucleotide sequence is reported in SEQ ID NO:112. Amino acids 9 to 21 of SEQ ID NO:112 are a predicted leader/signal sequence, with the predicted mature amino acid sequence beginning at amino acid 22. Due to the hydrophobic nature of the predicted leader/signal sequence, it is likely to act as a transmembrane domain should the predicted leader/signal sequence not be separated from the remainder of the km225_{—}1 protein.

The EcoRI/NotI restriction fragment obtainable from the deposit containing done km225_{—}1 should be approximately 2300 bp.

The nucleotide sequence disclosed herein for km225_{—}1 was searched against the GenBank and GeneSeq nucleotide sequence databases using BLASTN/BLASTX and FASTA search protocols. k225_{—}1 demonstrated at least some similarity with sequences identified as AA101603 (zk94h09.s1 Soares pregnant uterus NbHPU Homo sapiens cDNA clone 490529 3′ similar to contains Alu repetitive element; mRNA sequence). Based upon sequence similarity, km225_{—}1 proteins and each similar protein or peptide may share at least some activity.

Clone “mj301_{—}1”

A polynucleotide of the present invention has been identified as done “mj301_{—}1”. mj301_{—}1 was isolated from a human adult lymph node cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. mj3011 is a full-length done, including the entire coding sequence of a secreted protein (also referred to herein as “mj301_{—}1 protein”).

The nucleotide sequence of mj301_{—}1 as presently determined is reported in SEQ ID NO:113, and includes a poly(A) tail. What applicants presently believe to be the proper reading frame and the predicted amino acid sequence of the mj301_{—}1 protein corresponding to the foregoing nucleotide sequence is reported in SEQ ID NO:114. Amino adds 65 to 77 of SEQ ID NO:114 are a predicted leader/signal sequence, with the predicted mature amino acid sequence beginning at amino acid 78. Due to the hydrophobic nature of the predicted leader/signal sequence, it is likely to act as a transmembrane domain should the predicted leader/signal sequence not be separated from the remainder of the mj301_{—}1 protein.

The EcoRI/NotI restriction fragment obtainable from the deposit containing done mj301_should be approximately 2760 bp; however, a band of 550 bp has been detected in restriction digests, possibly due to an internal EcoRI or NotI restriction site in the done.

The nucleotide sequence disclosed herein for mj301_{—}1 was searched against the GenBank and GeneSeq nucleotide sequence databases using BLASTN/BLASTX and FASTA search protocols. mj301_{—}1 demonstrated at least some similarity with sequences identified as AA053085 (z173d01.s1 Stratagene colon (#937204) Homo sapiens cDNA clone 510241 3′), AA347293 (EST53566 Fetal heart II[Homo sapiens cDNA 5′ end), AA813287 (ai76a07.s1 Soares testis NHT Homo sapiens cDNA clone 1376724 3′, mRNA sequence), R45713 (Ha117-f Homo sapiens cDNA clone a117-f), T20114 (Human gene signature HUMGS01258), U46278 (Human clone xs252 mRNA sequence), Z36823 (H. sapiens (xs170) mRNA), and Z36832 (H. sapiens (xs170) mRNA). The human xs170 sequence is differentially expressed in pancreatic cancer cells. The predicted amino acid sequence disclosed herein for mj301_{—}1 was searched against the GenPept and GeneSeq amino acid sequence databases using the BLASTX search protocol. The predicted mj301_{—}1 protein demonstrated at least some similarity to the sequence identified as U07818 (putative phospho-beta-glucosidase [Bacillus stearothermophilus]). Based upon sequence similarity, mj301_{—}1 proteins and each similar protein or peptide may share at least some activity. The TopPredII computer program predicts an additional potential trans-membrane domain within the mj301_{—}1 protein sequence centered around amino acid 60 of SEQ ID NO:114.

Clone “ml10_{—}7”

A polynucleotide of the present invention has been identified as done “ml10_{—}7”. ml10_{—}7 was isolated from a human adult brain (caudate nucleus) cDNA library using methods which are selective for cDNAs encoding secreted proteins (see U.S. Pat. No. 5,536,637), or was identified as encoding a secreted or transmembrane protein on the basis of computer analysis of the amino add sequence of the encoded protein. ml10_{—}7 is a full-length done, including the entire coding sequence of a secreted protein (also referred to herein as “ml10_{—}7 protein”).

The nucleotide sequence of ml10_{—}7 as presently determined is reported in SEQ ID NO:115, and includes a poly(A) tail. What applicants presently believe to be the proper reading frame and the predicted amino acid sequence of the ml10_{—}7 protein corresponding to the foregoing nucleotide sequence is reported in SEQ ID NO:116. Amino acids 30 to 42 of SEQ ID NO:116 are a predicted leader/signal sequence, with the predicted mature amino add sequence beginning at amino acid 43. Due to the hydrophobic nature of the predicted leader/signal sequence, it is likely to act as a transmembrane domain should the predicted leader/signal sequence not be separated from the remainder of the ml10_{—}7 protein.

The EcoRI/NotI restriction fragment obtainable from the deposit containing done ml10_{—}7 should be approximately 1600 bp.

The nucleotide sequence disclosed herein for ml10_{—}7 was searched against the GenBank and GeneSeq nucleotide sequence databases using BLASTN/BLASTX and FASTA search protocols. ml10_{—}7 demonstrated at least some similarity with sequences identified as AA411457 (zv30f06.s1 Soares ovary tumor NbHOT Homo sapiens cDNA clone 755171 3′), AA41 1585 (zv30f06.r1 Soares ovary tumor NbHOT Homo sapiens cDNA clone 755171 5′, mRNA sequence), AA485512 (zx90b02.r1 Soares ovary tumor NbHOT Homo sapiens cDNA clone 810987 5′), R97588 (yq59b05.r1 Homo sapiens cDNA clone 200049 5′ similar to contains MSR1 repetitive element), and T23020 (Human gene signature HUMGS04748). The predicted amino acid sequence disclosed herein for ml10_{—}7 was searched against the GenPept and GeneSeq amino add sequence databases using the BLASTX search protocol. The predicted ml10_{—}7 protein demonstrated at least some similarity to the sequence identified as R56978 (Human myotonic dystrophy gene protein). Based upon sequence similarity, ml10_{—}7 proteins and each similar protein or peptide may share at least some activity. The TopPredII computer program predicts four additional potential transmembrane domains within the ml10_{—}7 protein sequence, centered approximately around amino acids 20, 55 (between residues 50 and 60), 85 (between residues 80 and 89), and 175 (between residues 169 and 180) of SEQ ID NO: 116, respectively. ml10_{—}7 appears to represent one member of a group of multiple alternatively spliced transcripts.

Clone “my340_{—}1”

A polynucleotide of the present invention has been identified as done “my340_{—}1”. my340_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. my340_{—}1 is a full-length clone, including the entire coding sequence of a secreted protein (also referred to herein as “my340_{—}1 protein”).

The nucleotide sequence of my340_{—}1 as presently determined is reported in SEQ ID NO:117, and includes a poly(A) tail. What applicants presently believe to be the proper reading frame and the predicted amino acid sequence of the my340_{—}1 protein corresponding to the foregoing nucleotide sequence is reported in SEQ ID NO:118.

The EcoRI/NotI restriction fragment obtainable from the deposit containing done my340_{—}1 should be approximately 1800 bp.

The nucleotide sequence disclosed herein for my340_{—}1 was searched against the GenBank and GeneSeq nucleotide sequence databases using BLASTN/BLASTX and FASTA search protocols. my340_{—}1 demonstrated at least some similarity with sequences identified as AA469015 (nc79g10.r1 NCI_CGAP_Pr2 Homo sapiens cDNA clone IMAGE:783618), H85290 (yv86f01.r1 Homo sapiens cDNA clone 249625 5′), L29074 (Homo sapiens fragile X mental retardation protein (FMR-1) gene (6 alternative splices), complete cds), M86699 (Human kinase (TTK) mRNA, complete cds), W19755 (zb38f08.r1 Soares parathyroid tumor NbHPA Homo sapiens cDNA clone 305895 5′), W63667 (zc57h10.r1 Soares parathyroid tumor NbHPA Homo sapiens cDNA clone 326467 5′, mRNA sequence), and Z84478 (Human DNA sequence). The predicted amino acid sequence disclosed herein for my340_{—}1 was searched against the GenPept and GeneSeq amino acid sequence databases using the BLASTX search protocol. The predicted my340_{—}1 protein demonstrated at least some similarity to the sequence identified as M86699 (kinase [Homo sapiens]). The human TTK kinase can phosphorylate serine, threonine, and tyrosine hydroxyamino acids. Based upon sequence similarity, my340_]proteins and each similar protein or peptide may share at least some activity. The TopPredII computer program predicts a potential transmembrane domain within the my340_{—}1 protein sequence centered around amino acid 50 of SEQ ID NO:28.

Deposit of Clones

Clones bn365_{—}53, bo342_{—}2, dn721_{—}8, dn834_{—}1, pd278_{—}5, pe80_{—}1, pm113_{—}1, pm749_{—}8, pt31_{—}4, and pv296_{—}5 were deposited on May 7, 1998 with the American Type Culture Collection (10801 University Boulevard, Manassas, Va. 20110-2209 U.S.A.) as an original deposit under the Budapest Treaty and were given the accession number ATCC 98752, from which each done comprising a particular polynucleotide is obtainable.

Clones er311_{—}20, fh149_{—}12, pc201_{—}6, p187_{—}1, and pm514_{—}4 were deposited on Jun. 2, 1998 with the American Type Culture Collection (10801 University Boulevard, Manassas, Va. 20110-2209 U.S.A) as an original deposit under the Budapest Treaty and were given the accession number ATCC 98781, from which each clone comprising a particular polynucleotide is obtainable.

Clones co155_{—}12, fn189_{—}13, lv2_{—}47, m1243_{—}1, pm96_{—}9, pu261_{—}1, pw214_{—}15, qb56_{—}19, qc646_{—}1, qf116_{—}2, and qf662_{—}3 were deposited on Jul. 2, 1998 with the American Type Culture Collection (10801 University Boulevard, Manassas, Va. 20110-2209 U.S.A.) as an original deposit under the Budapest Treaty and were given the accession number ATCC 98808, from which each done comprising a particular polynucleotide is obtainable.

Clones am748_{—}5, cj507_{—}1, cn922_{—}5, cw691_{—}11, cw1000_{—}2, cw1640_{—}1, d24_{—}1, dd426_{—}1, and di393_{—}2 were deposited on Jul. 16, 1998 with the American Type Culture Collection (10801 University Boulevard, Manassas, Va. 20110-2209 U.S.A.) as an original deposit under the Budapest Treaty and were given the accession number ATCC 98817, from which each done comprising a particular polynucleotide is obtainable.

Clones dj167_{—}2, dw665_{—}4, dx146_{—}12, dx219_{—}13, fm3 1, h225 1, kj320_{—}1, ml236_{—}5, and pu282_{—}10, were deposited on Jul. 16, 1998 with the American Type Culture Collection (10801 University Boulevard, Manassas, Va. 20110-2209 U.S.A.) as an original deposit under the Budapest Treaty and were given the accession number ATCC 98818, from which each done comprising a particular polynucleotide is obtainable.

Clones at94_{—}2, bf169_{—}13, bl152_{—}12, bz578_{—}1, cb123_{—}1, ch245_{—}1, cj378_{—}3, cw148_{—}1, dd119_{—}4, df202_{—}3, km225_{—}1, mj301_{—}1, ml10_{—}7, and my340_{—}1 were deposited on Jul. 22, 1998 with the American Type Culture Collection (10801 University Boulevard, Manassas, Va. 20110-2209 U.S.A.) as an original deposit under the Budapest Treaty and were given the accession number ATCC 98822, from which each clone comprising a particular polynucleotide is obtainable.

Clone dj167_{—}19 was deposited on Feb. 5, 1999 with the American Type Culture Collection (10801 University Boulevard, Manassas, Va. 20110-2209 U.S.A.) as an original deposit under the Budapest Treaty and was given the accession number ATCC 207090, from which the dj167_{—}19 done comprising a particular polynucleotide is obtainable.

All restrictions on the availability to the public of the deposited material will be irrevocably removed upon the granting of the patent, except for the requirements specified in 37 C.F.R. § 1.808(b), and the term of the deposit will comply with 37 C.F.R. § 1.806.

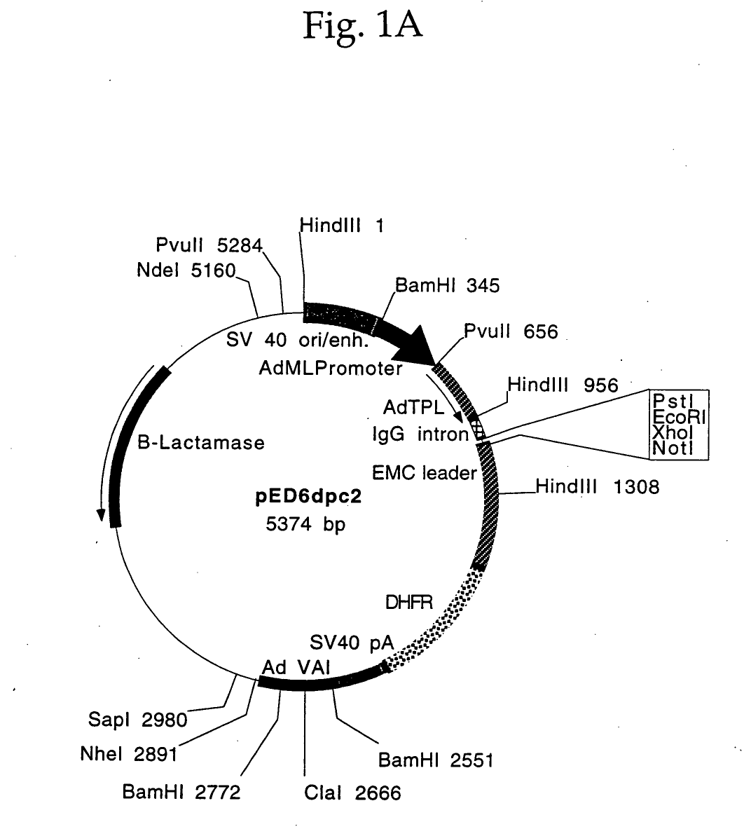

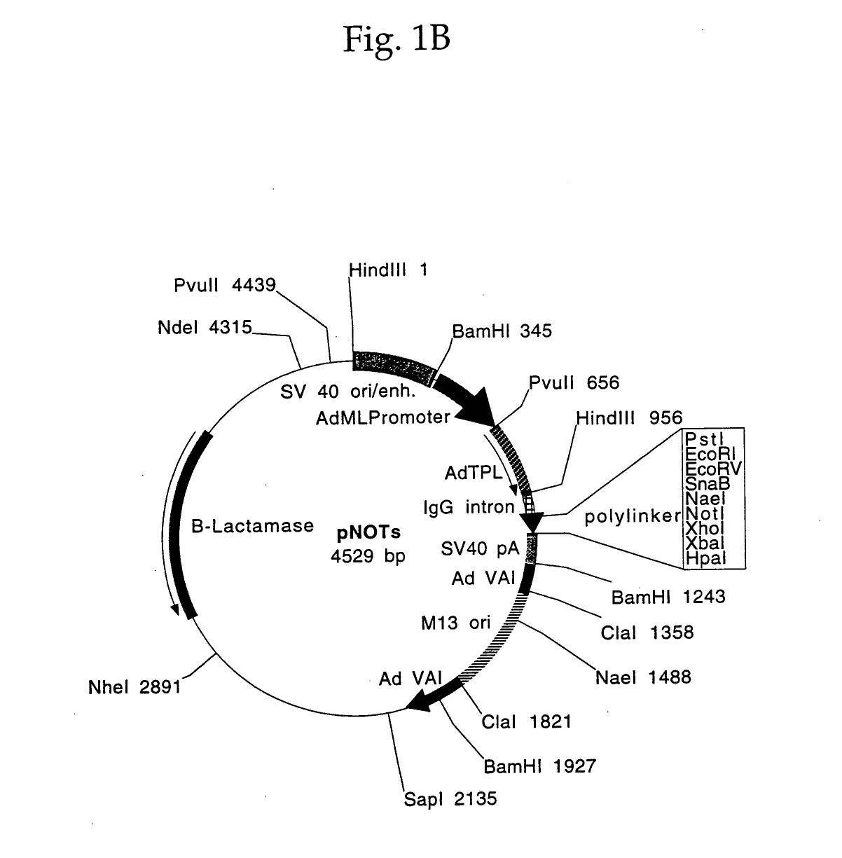

Each done has been transfected into separate bacterial cells (E. coli) in the composite deposits above. Each done can be removed from the vector in which it was deposited by performing an EcoRI/NotI digestion (5′ site, EcORI; 3′ site, NotI) to produce the appropriate fragment for such done. Each done was deposited in either the pED6 or pNOTs vector depicted in FIGS. 1A and 1B, respectively. The pED6dp¢ vector (“pED6”) was derived from pED6dpc1 by insertion of a new polylinker to facilitate cDNA cloning (Kaufman et al., 1991, Nucleic Acids Res. 19:4485_{—}4490); the pNOTs vector was derived from pMT2 (Kaufman et al., 1989, Mol. Cell. Biol. 9: 946-958) by deletion of the DHFR sequences, insertion of a new polylinker, and insertion of the M13 origin of replication in the ClaI site. In some instances, the deposited done can become “flipped” (i.e., in the reverse orientation) in the deposited isolate. In such instances, the cDNA insert can still be isolated by digestion with EcoRI and NotI. However, NotI will then produce the 5′ site and EcoRI will produce the 3′ site for placement of the cDNA in proper orientation for expression in a suitable vector. The cDNA may also be expressed from the vectors in which they were deposited.

Bacterial cells containing a particular done can be obtained from the composite deposit as follows:

An oligonucleotide probe or probes should be designed to the sequence that is known for that particular done. This sequence can be derived from the sequences provided herein, or from a combination of those sequences. The sequence of an oligonucleotide probe that was used to isolate or to sequence each full-length clone is identified below, and should be most reliable in isolating the clone of interest.

| Clone | Probe Sequence |
|---|---|
| bn365_53 | SEQ ID NO: 119 |
| bo342_2 | SEQ ID NO: 120 |
| dn721_8 | SEQ ID NO: 121 |
| dn834_1 | SEQ ID NO: 122 |
| pd278_5 | SEQ ID NO: 123 |
| pe80_1 | SEQ ID NO: 124 |
| pm113_1 | SEQ ID NO: 125 |
| pm749_8 | SEQ ID NO: 126 |
| pt31_4 | SEQ ID NO: 127 |
| pv296_5 | SEQ ID NO: 128 |
| er311_20 | SEQ ID NO: 129 |
| fh149_12 | SEQ ID NO: 130 |
| pc201_6 | SEQ ID NO: 131 |
| pl87_1 | SEQ ID NO: 132 |
| pm514_4 | SEQ ID NO: 133 |
| co155_12 | SEQ ID NO: 134 |
| fn189_13 | SEQ ID NO: 135 |
| lv2_47 | SEQ ID NO: 136 |
| ml243_1 | SEQ ID NO: 137 |
| pm96_9 | SEQ ID NO: 138 |
| pu261_1 | SEQ ID NO: 139 |
| pw214_15 | SEQ ID NO: 140 |
| qb56_19 | SEQ ID NO: 141 |
| qc646_1 | SEQ ID NO: 142 |
| qf116_2 | SEQ ID NO: 143 |
| qf662_3 | SEQ ID NO: 144 |
| am748_5 | SEQ ID NO: 145 |
| cj507_1 | SEQ ID NO: 146 |
| cn922_5 | SEQ ID NO: 147 |
| cw691_11 | SEQ ID NO: 148 |
| cw1000_2 | SEQ ID NO: 149 |
| cw1640_1 | SEQ ID NO: 150 |
| d24_1 | SEQ ID NO: 151 |
| dd426_1 | SEQ ID NO: 152 |
| di393_2 | SEQ ID NO: 153 |
| dj167_2 | SEQ ID NO: 154 |
| dw665_4 | SEQ ID NO: 155 |
| dx146_12 | SEQ ID NO: 156 |
| dx219_13 | SEQ ID NO: 157 |
| fm3_1 | SEQ ID NO: 158 |
| h225_1 | SEQ ID NO: 159 |
| kj320_1 | SEQ ID NO: 160 |
| ml236_5 | SEQ ID NO: 161 |
| pu282_10 | SEQ ID NO: 162 |
| at94_2 | SEQ ID NO: 163 |
| bf169_13 | SEQ ID NO: 164 |
| bl152_12 | SEQ ID NO: 165 |
| bz578_1 | SEQ ID NO: 166 |
| cb123_1 | SEQ ID NO: 167 |
| ch245_1 | SEQ ID NO: 168 |
| cj378_3 | SEQ ID NO: 169 |
| cw1481_1 | SEQ ID NO: 170 |
| dd119_4 | SEQ ID NO: 171 |
| df202_3 | SEQ ID NO: 172 |
| km225_1 | SEQ ID NO: 173 |
| mj301_1 | SEQ ID NO: 174 |
| ml10_7 | SEQ ID NO: 175 |
| my340_1 | SEQ ID NO: 176 |

In the sequences listed above which include an N at position 2, that position is occupied in preferred probes/primers by a biotinylated phosphoaramidite residue rather than a nucleotide (such as, for example, that produced by use of biotin phosphoramidite (1-dimethoxytrityloxy-2-(N-biotinyl-4-aminobutyl)-propyl-3-O-(2-cyanoethyl)-(N,N-diisopropyl)-phosphoramadite) (Glen Research, cat. no. 10-1953)).

The design of the oligonucleotide probe should preferably follow these parameters:

(a) It should be designed to an area of the sequence which has the fewest ambiguous bases (“N's”), if any;

(b) It should be designed to have a Tₘ of approx. 80° C. (assuming 2° for each A or T and 4 degrees for each G or C).

The oligonucleotide should preferably be labeled with γ-³²P ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4e+6 dpm/pmole.

The bacterial culture containing the pool of full-length clones should preferably be thawed and 100 μl of the stock used to inoculate a sterile culture flask containing 25 ml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters and lyse, denature and bake them.

The filter is then preferably incubated at 65° C. for 1 hour with gentle agitation in 6×SSC (20× stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately 10 mL per 150 mm filter). Preferably, the probe is then added to the hybridization mix at a concentration greater than or equal to 1e+6 dpm/mL. The filter is then preferably incubated at 65° C. with gentle agitation overnight. The filter is then preferably washed in 500 mL of 2×SSC/0.5% SDS at room temperature without agitation, preferably followed by 500 mL of 2×SSC/0.1% SDS at room temperature with gentle shaking for 15 minutes. A third wash with 0.1×SSC/0.5% SDS at 65° C. for 30 minutes to 1 hour is optional. The filter is then preferably dried and subjected to autoradiography for sufficient time to visualize the positives on the X-ray film. Other known hybridization methods can also be employed.

The positive colonies are picked, grown in culture, and plasmid DNA isolated using standard procedures. The clones can then be verified by restriction analysis, hybridization analysis, or DNA sequencing.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10 773-778 (1992) and in R. S. McDowell, et al., J. Amer. Chem. Soc. 114, 9245-9253 (1992), both of which are incorporated herein by reference. Such fragments may be fused to carrier molecules such as immunoglobulins for many purposes, including increasing the valency of protein binding sites. For example, fragments of the protein may be fused through “linker” sequences to the Fc portion of an immunoglobulin. For a bivalent form of the protein, such a fusion could be to the Fc portion of an IgG molecule. Other immunoglobulin isotypes may also be used to generate such fusions. For example, a protein-IgM fusion would generate a decavalent form of the protein of the invention.

The present invention also provides both full-length and mature forms of the disclosed proteins. The full-length form of the such proteins is identified in the sequence listing by translation of the nucleotide sequence of each disclosed clone. The mature form(s) of such protein may be obtained by expression of the disclosed full-length polynucleotide (preferably those deposited with ATCC) in a suitable mammalian cell or other host cell. The sequence(s) of the mature form(s) of the protein may also be determinable from the amino acid sequence of the full-length form.

The present invention also provides genes corresponding to the polynucleotide sequences disclosed herein. “Corresponding genes” are the regions of the genome that are transcribed to produce the mRNAs from which cDNA polynucleotide sequences are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated.

The chromosomal location corresponding to the polynucleotide sequences disclosed herein may also be determined, for example by hybridizing appropriately labeled polynucleotides of the present invention to chromosomes in situ. It may also be possible to determine the corresponding chromosomal location for a disclosed polynucleotide by identifying significantly similar nucleotide sequences in public databases, such as expressed sequence tags (ESTs), that have already been mapped to particular chromosomal locations. For at least some of the polynucleotide sequences disclosed herein, public database sequences having at least some similarity to the polynucleotide of the present invention have been listed by database accession number. Searches using the GenBank accession numbers of these public database sequences can then be performed at an Internet site provided by the National Center for Biotechnology Information having the address http://www ncbi.nlm.nih.gov/UniGene/, in order to identify “UniGene clusters” of overlapping sequences. Many of the “UniGene dusters” so identified will already have been mapped to particular chromosomal sites.

Organisms that have enhanced, reduced, or modified expression of the gene(s) corresponding to the polynucleotide sequences disclosed herein are provided. The desired change in gene expression can be achieved through the use of antisense polynucleotides or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39; all of which are incorporated by reference herein). Transgenic animals that have multiple copies of the gene(s) corresponding to the polynucleotide sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1, incorporated by reference herein). In addition, organisms are provided in which the gene(s) corresponding to the polynucleotide sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629_{—}3; Zwaal et al., 1993, Proc. Natl. Acad. Sci. USA 90(16): 7431-7435; Clark et al., 1994, Proc. Natl. Acad. Sci. USA 91(2): 719-722; all of which are incorporated by reference herein), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523; all of which are incorporated by reference herein). These organisms with altered gene expression are preferably eukaryotes and more preferably are mammals. Such organisms are useful for the development of non-human models for the study of disorders involving the corresponding gene(s), and for the development of assay systems for the identification of molecules that interact with the protein product(s) of the corresponding gene(s).

Where the protein of the present invention is membrane-bound (e.g., is a receptor), the present invention also provides for soluble forms of such protein. In such forms, part or all of the intracellular and transmembrane domains of the protein are deleted such that the protein is fully secreted from the cell in which it is expressed. The intracellular and transmembrane domains of proteins of the invention can be identified in accordance with known techniques for determination of such domains from sequence information. For example, the TopPredII computer program can be used to predict the location of transmembrane domains in an amino acid sequence, domains which are described by the location of the center of the transmembrane domain, with at least ten transmembrane amino acids on each side of the reported central residue(s).

Proteins and protein fragments of the present invention include proteins with amino acid sequence lengths that are at least 25%(more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino add sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous amino acids that shares at least 75% sequence identity (more preferably, at least 85% identity; most preferably at least 95% identity) with any such segment of any of the disclosed proteins.

In particular, sequence identity may be determined using WU-BLAST (Washington University BLAST) version 2.0 software, which builds upon WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873-5877; all of which are incorporated by reference herein). WU-BLAST version 2.0 executable programs for several UNIX platforms can be downloaded from ftp://blastwustl.edu/blast/executables. The complete suite of search programs (BLASTP, BLASTN, BLASTX, TBLASTN, and TBLASTX) is provided at that site, in addition to several support programs. WU-BLAST 2.0 is copyrighted and may not be sold or redistributed in any form or manner without the express written consent of the author; but the posted executables may otherwise be freely used for commercial, nonprofit, or academic purposes. In all search programs in the suite—BLASTP, BLASTN, BLASTX, TBLASTN and TBLASTX—the gapped alignment routines are integral to the database search itself, and thus yield much better sensitivity and selectivity while producing the more easily interpreted output. Gapping can optionally be turned off in all of these programs, if desired. The default penalty (Q) for a gap of length one is Q=9 for proteins and BLASTP, and Q=10 for BLASTN, but may be changed to any integer value including zero, one through eight, nine, ten, eleven, twelve through twenty, twenty-one through fifty, fifty-one through one hundred, etc. The default per-residue penalty for extending a gap (R) is R=2 for proteins and BLASTP, and R=10 for BLASTN, but may be changed to any integer value including zero, one, two, three, four, five, six, seven, eight, nine, ten, eleven, twelve through twenty, twenty-one through fifty, fifty-one through one hundred, etc. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Species homologues of the disclosed polynucleotides and proteins are also provided by the present invention. As used herein, a “species homologue” is a protein or polynucleotide with a different species of origin from that of a given protein or polynucleotide, but with significant sequence similarity to the given protein or polynucleotide. Preferably, polynucleotide species homologues have at least 60% sequence identity (more preferably, at least 75% identity; most preferably at least 90% identity) with the given polynucleotide, and protein species homologues have at least 30% sequence identity (more preferably, at least 45% identity; most preferably at least 60% identity) with the given protein, where sequence identity is determined by comparing the nucleotide sequences of the polynucleotides or the amino acid sequences of the proteins when aligned so as to maximize overlap and identity while minimizing sequence gaps. Species homologues may be isolated and identified by making suitable probes or primers from the sequences provided herein and screening a suitable nucleic acid source from the desired species. Preferably, species homologues are those isolated from mammalian species. Most preferably, species homologues are those isolated from certain mammalian species such as, for example, Pan troglodytes, Gorilla gorilla, Pongo pygmaeus, Hylobates concolor, Macaca mulatta, Papio papio, Papio hamadryas, Cercopithecus aethiops, Cebus capucinus, Aotus trivirgatus, Sanguinus oedipus, Microcebus murinus, Mus musculus, Rattus norvegicus, Cricetulus grieus, Felis catus, Mustela vison, Canis familiaris, Oryctolagus cuniculus, Bos taurus, Ovis aries, Sus scrofa, and Equus caballus, for which genetic maps have been created allowing the identification of syntenic relationships between the genomic organization of genes in one species and the genomic organization of the related genes in another species (O'Brien and Seuanez, 1988, Ann. Rev. Genet. 22: 323-351; O'Brien et al., 1993, Nature Genetics 3:103-112; Johansson et al., 1995, Genomics 25: 682-690; Lyons et al., 1997, Nature Genetics 15: 47-56; O'Brien et al., 1997, Trends in Genetics 13(10): 393-399; Carver and Stubbs, 1997, Genome Research 7:1123-1137; all of which are incorporated by reference herein).

The invention also encompasses allelic variants of the disclosed polynucleotides or proteins; that is, naturally-occurring alternative forms of the isolated polynucleotides which also encode proteins which are identical or have significantly similar sequences to those encoded by the disclosed polynucleotides. Preferably, allelic variants have at least 60% sequence identity (more preferably, at least 75% identity; most preferably at least 90% identity) with the given polynucleotide, where sequence identity is determined by comparing the nucleotide sequences of the polynucleotides when aligned so as to maximize overlap and identity while minimizing sequence gaps. Allelic variants may be isolated and identified by making suitable probes or primers from the sequences provided herein and screening a suitable nucleic acid source from individuals of the appropriate species.

The invention also includes polynucleotides with sequences complementary to those of the polynucleotides disclosed herein.

The present invention also includes polynucleotides that hybridize under reduced stringency conditions, more preferably stringent conditions, and most preferably highly stringent conditions, to polynucleotides described here in Examples of stringency conditions are shown in the table below: highly stringent conditions are those that are at least as stringent as, for example, conditions A-F; stringent conditions are at least as stringent as, for example, conditions GL; and reduced stringency conditions are at least as stringent as, for example, conditions M-R.

| | | Hybrid | | Wash |
|---|---|---|---|---|
| Stringency | Polynucleotide | Length | Hybridization Temperature and | Temperature |
| Condition | Hybrid | (bp)^{‡} | Buffer^{†} | and Buffer^{†} |
| A | DNA:DNA | ≧50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | ≧50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | ≧50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | ≧50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | ≧50 | 67° C.; 4xSSC -or- | 67° C; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | ≧50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | ≧50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | ≧50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | ≧50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ^{‡}The hybrid length is that anticipated for the hybridized region(s) of the hybridizing polynucleotides. When hybridizing a polynudeotide to a target polynucleotide of unknown sequence, the hybrid length is assumed to be that of the hybridizing polynucleotide. When polynucleotides of known sequence are hybridized, the | | | | |
| #hybrid length can be determined by aligning the sequences of the polynucleotides and identifying the region or regions of optimal sequence complementarity. | | | | |
| ^{†}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| *T_{B} − T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C | | | | |
| #bases). For hybrids between 18 and 49 base pairs in length, Tₘ(° C.) = 81.5 + 16.6(log₁₀[Na⁺] + 0.41(% G + C) − (600/N), where N is the number of bases in the hybrid, and [Na⁺] is the concentration of sodium ions in the hybridization buffer ([Na⁺] for 1xSSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25% (more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

The isolated polynucleotide of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisae, Schizosaccaromyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLabs (Beverly, Mass.), Pharmacia (Piscataway, N.J.) and Invitrogen Corporation (Carlsbad, Calif.), respectively. The protein can also be tagged with an epitope and subsequently purified by using a specific antibody directed to such epitope. One such epitope (“Flag”) is commercially available from the Eastman Kodak Company (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The protein of the invention may also be expressed as a product of transgenic animals, e.g., as a component of the milk of transgenic cows, goats, pigs, or sheep which are characterized by somatic or germ cells containing a nucleotide sequence encoding the protein.

The protein may also be produced by known conventional chemical synthesis. Methods for constructing the proteins of the present invention by synthetic means are known to those skilled in the art. The synthetically-constructed protein sequences, by virtue of sharing primary, secondary or tertiary structural and/or conformational characteristics with proteins may possess biological properties in common therewith, including protein activity. Thus, they may be employed as biologically active or immunological substitutes for natural, purified proteins in screening of therapeutic compounds and in immunological processes for the development of antibodies.

The proteins provided herein also include proteins characterized by amino acid sequences similar to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are believed to be encompassed by the present invention.

Uses and Biological Activity

The polynucleotides and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, those described in Gyuris et al., 1993, Cell 75: 791-803 and in Rossi et al., 1997, Proc. Natl. Acad. Sci. USA 94: 8405-8410, all of which are incorporated by reference herein) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor-dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK.

The activity of a protein of the invention may, among other means, be measured by the following methods:

Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnofli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J.E.e.a. Coligan eds. Vol 1 pp. 3.12.1-3.12.14, John Wiley and Sons, Toronto. 1994; and Measurement of mouse and human Interferon γ, Schreiber, R. D. In Current Protocols in Immunology. J.E.e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K, Davis, L. S. and Lipsky, P. E. In Current Protocols in Immunology. J.E.e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleulin 6—Nordan, R. In Current Protocols in Immunology. J.E.e.a. Coligan eds. Vol 1 pp. 6.6.1-6.65, John Wiley and Sons, Toronto. 1991; Smith et al., Proc. Natl. Acad. Sci. U.S.A. 83:1857-1861, 1986; Measurement of human Interleukin 11—Bennett, F., Giannotti, J., Clark, S. C. and Turner, K. J. In Current Protocols in Immunology. J. E.e.a. Coligan eds. Vol 1 pp. 6.15.1 John Wiley and Sons, Toronto. 1991; Measurement of mouse and human Interleukin 9—Ciarletta, A., Giannotti, J., Clark, S. C. and Turner, K. J. In Current Protocols in Immunology. J. E. e.a. Coligan eds. Vol 1 pp. 6.13.1, John Wiley and Sons, Toronto. 1991.

Assays for T-cell done responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Immune Stimulating or Suppressing Activity

A protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HUV, hepatitis viruses, herpesvirues, mycobacteria, Leishmania spp., malaria spp. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to regulate immune responses in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), eg., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II α chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et. al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J.E.e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kuisbeek, D. H. Margulies, E. M. Shevadh, W Strober, Pub. Greene Publishing Associates and Wiley-Interscience (Chapter 3, In Vitro assays for Mouse Lymphocyte Function 3.1-3.19; Chapter 7, Immunologic studies in Hurans); Takai et al., J. Immunol. 137:3494-3500, 1986; Takai et al., J. Immunol. 140:508-512, 1988; Bertagnori et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others; proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:40374045, 1990; Zamai et al., Cytometry 14:891-897, 1993; Gorczyca et al., International Journal of Oncology 1:639-648,1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. USA 88:7548-7551, 1991.

Hematopoiesis Regulating Activity

A protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopotetic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissue's, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing pps. 71-112 (Maibach, H I and Rovee, D T, eds.), Year Book Medical Publishers, Inc., Chicago, as modified by Eaglstein and Mertz, J. Invest. Dermatol 71:382-84 (1978).

Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin α family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-β group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 1744-1748; Gruber et al. J. of Immunol. 152:5860-5867, 1994; Johnston et al. J. of Immunol. 153: 1762-1768, 1994.

Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26:131-140, 1986; Burdick et al., Thrombosis Res. 45:413419, 1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selectins, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Cadherin/Tumor Invasion Suppressor Activity

Cadherins are calcium-dependent adhesion molecules that appear to play major roles during development, particularly in defining specific cell types. Loss or alteration of normal cadherin expression can lead to changes in cell adhesion properties linked to tumor growth and metastasis. Cadherin malfunction is also implicated in other human diseases, such as pemphigus vulgaris and pemphigus foliaceus (auto-immune blistering skin diseases), Crohn's disease, and some developmental abnormalities.

The cadherin superfamily includes well over forty members, each with a distinct pattern of expression. All members of the superfamily have in common conserved extracellular repeats (cadherin domains), but structural differences are found in other parts of the molecule. The cadherin domains bind calcium to form their tertiary structure and thus calcium is required to mediate their adhesion. Only a few amino acids in the first cadherin domain provide the basis for homophilic adhesion; modification of this recognition site can change the specificity of a cadherin so that instead of recognizing only itself, the mutant molecule can now also bind to a different cadherin. In addition, some cadherins engage in heterophilic adhesion with other cadherins.

E-cadherin, one member of the cadherin superfamily, is expressed in epithelial cell types. Pathologically, if E-cadherin expression is lost in a tumor, the malignant cells become invasive and the cancer metastasizes. Transfection of cancer cell lines with polynucleotides expressing E-cadherin has reversed cancer-associated changes by returning altered cell shapes to normal, restoring cells' adhesiveness to each other and to their substrate, decreasing the cell growth rate, and drastically reducing anchorage-independent cell growth. Thus, reintroducing E-cadherin expression reverts carcinomas to a less advanced stage. It is likely that other cadherins have the same invasion suppressor role in carcinomas derived from other tissue types. Therefore, proteins of the present invention with cadherin activity, and polynucleotides of the present invention encoding such proteins, can be used to treat cancer. Introducing such proteins or polynucleotides into cancer cells can reduce or eliminate the cancerous changes observed in these cells by providing normal cadherin expression.

Cancer cells have also been shown to express cadherins of a different tissue type than their origin, thus allowing these cells to invade and metastasize in a different tissue in the body. Proteins of the present invention with cadherin activity, and polynucleotides of the present invention encoding such proteins, can be substituted in these cells for the inappropriately expressed cadherins, restoring normal cell adhesive properties and reducing or eliminating the tendency of the cells to metastasize.

Additionally, proteins of the present invention with cadherin activity, and polynucleotides of the present invention encoding such proteins, can used to generate antibodies recognizing and binding to cadherins. Such antibodies can be used to block the adhesion of inappropriately expressed tumor-cell cadherins, preventing the cells from forming a tumor elsewhere. Such an anti-cadherin antibody can also be used as a marker for the grade, pathological type, and prognosis of a cancer, i.e. the more progressed the cancer, the less cadherin expression there will be, and this decrease in cadherin expression can be detected by the use of a cadherin-binding antibody.

Fragments of proteins of the present invention with cadherin activity, preferably a polypeptide comprising a decapeptide of the cadherin recognition site, and poly-nucleotides of the present invention encoding such protein fragments, can also be used to block cadherin function by binding to cadherins and preventing them from binding in ways that produce undesirable effects. Additionally, fragments of proteins of the present invention with cadherin activity, preferably truncated soluble cadherin fragments which have been found to be stable in the circulation of cancer patients, and polynucleotides encoding such protein fragments, can be used to disturb proper cell-cell adhesion.

Assays for cadherin adhesive and invasive suppressor activity include, without limitation, those described in: Hortsch et al. J Biol Chem 270 (32): 18809-18817, 1995; Miyali et al. Oncogene 11: 2547-2552, 1995; Ozawa et al. Cell 63: 1033-1038, 1990.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via antibody-dependent cell-mediated cytotoxicity (ADCC)). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhyms or caricadic cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Administration and Dosing

A protein of the present invention (from whatever source derived, including without limitation from recombinant and non-recombinant sources) may be used in a pharmaceutical composition when combined with a pharmaceutically acceptable carrier. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL1, IL-2, IL-3, IL-4, IL-5, IL-6 IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, and erythropoietin. The pharmaceutical composition may further contain other agents which either enhance the activity of the protein or compliment its activity or use in treatment. Such additional factors and/or agents may be included in the pharmaceutical composition to produce a synergistic effect with protein of the invention, or to minimize side effects. Conversely, protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent.

A protein of the present invention may be active in multimers (e.g., heterodimers or homodimers) or complexes with itself or other proteins. As a result, pharmaceutical compositions of the invention may comprise a protein of the invention in such multimeric or complexed form.

The pharmaceutical composition of the invention may be in the form of a complex of the protein(s) of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunolgobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skillful in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether adminstered in combination, serially or simultaneously.

In practicing the method of treatment or use of the present invention, a therapeutically effective amount of protein of the present invention is administered to a mammal having a condition to be treated. Protein of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein of the present invention in combination with cytoidne(s), lymphokine(s), other hematopoietic factor(s), thrombolytic or anti-thrombotic factors.

Administration of protein of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 05 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of protein of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 ng to about 10 mg, more preferably about 0.1 μg to about 1 mg) of protein of the present invention per kg body weight.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Protein of the invention may also be used to immunize animals to obtain polyclonal and monoclonal antibodies which specifically react with the protein. As used herein, the term “antibody” includes without limitation a polyclonal antibody, a monoclonal antibody, a chimeric antibody, a single-chain antibody, a CDR-grafted antibody, a humanized antibody, or fragments thereof which bind to the indicated protein. Such term also includes any other species derived from an antibody or antibody sequence which is capable of binding the indicated protein.

Antibodies to a particular protein can be produced by methods well known to those skilled in the art. For example, monoclonal antibodies can be produced by generation of antibody-producing hybridomas in accordance with known methods (see for example, Goding, 1983, Monoclonal antibodies: principles and practice, Academic Press Inc., New York; and Yokoyama, 1992, “Production of Monoclonal Antibodies” in Current Protocols in Immunology, Unit 2.5, Greene Publishing Assoc. and John Wiley & Sons). Polyclonal sera and antibodies can be produced by inoculation of a mammalian subject with the relevant protein or fragments thereof in accordance with known methods. Fragments of antibodies, receptors, or other reactive peptides can be produced from the corresponding antibodies by cleavage of and collection of the desired fragments in accordance with known methods (see for example, Goding, supra; and Andrew et al., 1992, “Fragmentation of Immunoglobulins” in Current Protocols in Immunology, Unit 2.8, Greene Publishing Assoc. and John Wiley & Sons). Chimeric antibodies and single chain antibodies can also be produced in accordance with known recombinant methods (see for example, 5,169,939, 5,194,594, and 5,576,184). Humanized antibodies can also be made from corresponding murine antibodies in accordance with well known methods (see for example, U.S. Pat. Nos. 5,530,101, 5,585,089, and 5,693,762). Additionally, human antibodies may be produced in non-human animals such as mice that have been genetically altered to express human antibody molecules (see for example Fishwild et al., 1996, Nature Biotechnology 14: 845-851; Mendez et al., 1997, Nature Genetics 15: 146-156 (erratum Nature Genetics 16: 410); and U.S. Pat. Nos. 5,877,397 and 5,625,126). Such antibodies may be obtained using either the entire protein or fragments thereof as an immunogen. The peptide immunogens additionally may contain a cysteine residue at the carboxyl terminus, and, are conjugated to a hapten such as keyhole limpet hemocyanin (KLH). Methods for synthesizing such peptides are known in the art, for example, as in R. P. Merrifield, J. Amer. Chem. Soc. 85 2149-2154 (1963); J. L. Krstenansky, et al., FEBS Lett. 211, 10 (1987).

Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing composition to the site of bone and/or cartilage damage, providing a structure for the developing bone and cartilage and optimally capable of being resorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methykellulose, ethylcellulose, hydroxyethylcellulose, hydroxypropylcellulose, hydroxypropyl-methylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the protein from the polymer matrix and to provide appropriate handling of the composition, yet not so much that the progenitor cells are prevented from infiltrating the matrix, thereby providing the protein the opportunity to assist the osteogenic activity of the progenitor cells.

In further compositions, proteins of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications. Particularly domestic animals and thoroughbred horses, in addition to humans, are desired patients for such treatment with proteins of the present invention.

The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject Polynucleotides of the invention may also be administered by other known methods for introduction of nucleic acid into a cell or organism (including, without limitation, in the form of viral vectors or naked DNA).

Cells may also be cultured ex vivo in the presence of proteins of the present invention in order to proliferate or to produce a desired effect on or activity in such cells. Treated cells can then be introduced in vivo for therapeutic purposes.

Patent and literature references cited herein are incorporated by reference as if fully set forth.

1.-38. (Cancelled).

39. An isolated polynucleotide comprising a coding sequence consisting of the nucleotide sequence of SEQ ID NO: 75.

40. The polynucleotide of claim 39, wherein said polynucleotide is operably linked to at least one expression control sequence.

41. A host cell transformed with the polynucleotide of claim 40.

42. The host cell of claim 41, wherein said cell is a mammalian cell.

43. An isolated polynucleotide comprising a coding sequence consisting of the cDNA insert of clone dw665_{—}4 deposited under accession number ATCC 98818.

44. An isolated polynucleotide comprising a coding sequence consisting of the nucleotide sequence of SEQ ID NO:75 from nucleotide 71 to nucleotide 1441.

45. An isolated polynucleotide coding sequence that encodes a protein consisting of the amino acid sequence of SEQ ID NO:76.

46. An isolated polynucleotide that hybridizes under conditions at least as stringent as 1×SSC at 65 degrees C., or 1×SSC at 42 degrees C. with 50% formamide, followed by washing in 0.3×SSC at 65 degrees C., to a complement of the polynucleotide of claim 39.

47. An isolated polynucleotide that hybridizes under conditions at least as stringent as 1×SSC at 67 degrees C., or 1×SSC at 45 degrees C. with 50% formamide, followed by washing in 0.3×SSC at 67 degrees C., to a complement of the polynucleotide of claim 39.

48. An isolated polynucleotide having at least 90% sequence identity to the polynucleotide of claim 39.

49. An isolated polynucleotide having at least 95% sequence identity to the polynucleotide of claim 39.

50. A process for producing a protein encoded by the polynucleotide of any one of claims 39 and 43 to 49, which process comprises:
(a) growing a culture of a host cell transformed with said polynucleotide in a suitable culture medium; and
(b) purifying said protein from the culture.

51. An isolated polynucleotide comprising a coding sequence consisting of the nucleotide sequence of SEQ ID NO:85.

52. The polynucleotide of claim 51, wherein said polynucleotide is operably linked to at least one expression control sequence.

53. A host cell transformed with the polynucleotide of claim 52.

54. The host cell of claim 53, wherein said cell is a mammalian cell.

55. An isolated polynucleotide comprising a coding sequence consisting of the cDNA insert of clone kj320_{—}1 deposited under accession number ATCC 98818.

56. An isolated polynucleotide comprising a coding sequence consisting of the nucleotide sequence of SEQ ID NO:85 from nucleotide 391 to nucleotide 3210.

57. An isolated polynucleotide coding sequence that encodes a protein consisting of the amino acid sequence of SEQ ID NO:86.

58. An isolated polynucleotide that hybridizes under conditions at least as stringent as 1×SSC at 65 degrees C., or 1×SSC at 42 degrees C. with 50% formamide, followed by washing in 0.3×SSC at 65 degrees C., to a complement of the polynucleotide of claim 51.

59. An isolated polynucleotide that hybridizes under conditions at least as stringent as 1×SSC at 67 degrees C., or 1×SSC at 45 degrees C. with 50% formamide, followed by washing in 0.3×SSC at 67 degrees C., to a complement of the polynucleotide of claim 51.

60. An isolated polynucleotide having at least 90% sequence identity to the polynucleotide of claim 51.

61. An isolated polynucleotide having at least 95% sequence identity to the polynucleotide of claim 51.

62. A process for producing a protein encoded by the polynucleotide of any one of claims 51 and 55 to 61, which process comprises:
(a) growing a culture of a host cell transformed with said polynucleotide in a suitable culture medium; and
(b) purifying said protein from the culture.


SEQUENCE LISTING (180 sequences)

SEQ ID NO: 1 - DNA, 571 bp, Homo sapiens
ttcttcgccaggctctctgctgactcaagttcttcagttcacgatcttctagttgcagcgatgagtgcacgagtgagatcaagatccagaggaagaggagatggtcaggaggctcccgatgtggttgcattcgtggctcccggtgaatctcagcaagaggaaccaccaactgacaatcaggatattgaacctggacaagagagagaaggaacacctccgatcgaagaacgtaaagtagaaggtgattgccaggaaatggatctggaaaagactcggagtgagcgtggagatggctctgatgtaaaagagaagactccacctaatcctaagcatgctaagactaaagaagcaggagatgggcagccataagttaaaaagaagacaagctgaagctacacacatggctgatgtcacattgaaaatgtgactgaaaatttgaaaattctctcaataaagtttgagttttctctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 102 aa, Homo sapiens
MSARVRSRSRGRGDGQEAPDVVAFVAPGESQQEEPPTDNQDIEPGQEREGTPPIEERKVEGDCQEMDLEKTRSERGDGSDVKEKTPPNPKHAKTKEAGDGQP

SEQ ID NO: 3 - DNA, 2709 bp, Homo sapiens
gaggaaacctctcgctggggctaggagttcggcggggcgcgcgccggcggctgcggagctggcaggtgcgaagcgtctgcacctggcgggcgatggcgcccgatgcgggcgccccgggatagcgtgggcgaggctgcggggccccggcgcgcacgcccgcacctctccccagccctggcgtgggcccagcccggcccaggcagcaatggggttcctgcagctgctggtcgtagcggtgctggcatccgaacaccgggtggctggtgcagccgaggtcttcgggaattccagcgagggtcttattgaattttctgtggggaaatttagatacttcgagctcaataggccctttccagaggaagctattttgcatgatatttcaagcaatgtgacttttcttattttccaaatacactcacagtatcagaatacaactgtttccttttctccgactctcctttccaattcctcggaaacaggcactgccagtggactggttttcatccttagaccagagcagagtacatgcacttggtacttggggacttcaggcatacagcctgtccagaatatggctatcctactctcctactcagaaagagatcctgtccctggaggctgtaatttggagttcgatttagatattgatcccaacatttacttggagtataatttctttgaaacgactatcaagtttgccccagcaaacctaggctatgcgagaggcgtagatcccccaccatgtgacgctgggacagaccaggactccaggtggaggttgcagtatgatgtctatcagtattttctgcctgagaatgacctcactgaggagatgttgctgaagcatctgcagaggatggtcagtgtgccccaggtgaaggccagtgctctcaaggtggttaccctaacagctaatgataagacaagtgtttccttctcctccctcccgggacaaggtgtcatatacaatgtcattgtttgggacccgtttctaaatacatctgctgcctacattcctgctcacacatacgcttgcagctttgaggcaggagagggtagttgtgcttccctaggaagagtgtcttccaaagtgttcttcactctttttgccctgcttggtttcttcatttgtttctttggacacagattctggaaaacagaattattcttcataggctttatcatcatgggattcttcttttatatactgattacaagactgacacctatcaagtatgatgtgaatctgattctgacagctgtcactggaagcgtcggtggaatgttcttggtagctgtgtggtggcgatttggaatcctctcgatctgcatgctctgtgttggactagtgctggggttcctcatctcgtcagtgactttctttactccactgggaaacctaaagatttttcatgatgatggtgtattctgggtcactttctcttgcatagctatcctcattccagtagttttcatgggctgcctaagaatactgaacatactgacttgtggagtcattggctcctattcggtggttttagccattgacagttactggtccacaagcctttcctacatcactttgaacgtactcaagagagcgctcaacaaggatttccacagagctttcacaaatgtgccttttcaaactaatgacttcattatcctggcagtatggggcatgctggctgtaagtggaattacgttacagattcgaagagagagaggacgaccgttcttccctccccacccatacaagttatggaagcaagagagagagcgccgagtgacaaacattctggaccctagctaccacattcctccattgagagagaggctctatggccgattaacccagattaaagggctcttccagaaggagcagccagctggagagagaacgcctttgcttctgtagatgcccaggggcttggtcagtgtgcctcagctttggagttcatgcctggagtggttcaacagtctctggtgcaagtctaataagagatcaggcatatatatctgttctttgcataatattatggtgcccttattgatatatggtaagggtgtactaggggattaggatgattgtaagagaatgagaaagatgaccaaaaggttggtggtagggaggctttttcttatttccaaatacttgagaaattaccttttggtttacaaatctatgatcaacttattccattaaatagatacattaaaaaaattaaaaactgattcttctgcagagcactggtgtttctttttataaccccttgaaacaagtctctcacstgagcctgtctaaactttcggagggagtttattattgagtctttatctgtgacagtatttggagatttagggatttgatacttaggcctttgaattttagaatacaaaaagagaagcaagccagacatggtggctcacacctgtaatcccaatactgggaggccaaggtgggagtatcgcttgagcccaggagtttgagaccgacatgggcaacatgacaagaccccatctctgcaaaaagattaaaaagttggccaggcatggtggcacatgcctgctcccagctcccggggagactgagatggggggatcccctggagccctgaagattgaggctgcagtgagccttgattgtgtcactgcactccagcttgggtgacagagaccctgtctcgagaaattaaaaaaaaaaaaaaa

SEQ ID NO: 4 - protein, 570 aa, Homo sapiens
MGFLQLLVVAVLASEHRVAGAAEVFGNSSEGLIEFSVGKFRYFELNRPFPEEAILHDISSNVTFLIFQIHSQYQNTTVSFSPTLLSNSSETGTASGLVFILRPEQSTCTWYLGTSGIQPVQNMAILLSYSERDPVPGGCNLEFDLDIDPNIYLEYNFFETTIKFAPANLGYARGVDPPPCDAGTDQDSRWRLQYDVYQYFLPENDLTEEMLLKHLQRMVSVPQVKASALKVVTLTANDKTSVSFSSLPGQGVIYNVIVWDPFLNTSAAYIPAHTYACSFEAGEGSCASLGRVSSKVFFTLFALLGFFICFFGHRFWKTELFFIGFIIMGFFFYILITRLTPIKYDVNLILTAVTGSVGGMFLVAVWWRFGILSICMLCVGLVLGFLISSVTFFTPLGNLKIFHDDGVFWVTFSCIAILIPVVFMGCLRILNILTCGVIGSYSVVLAIDSYWSTSLSYITLNVLKRALNKDFHRAFTNVPFQTNDFIILAVWGMLAVSGITLQIRRERGRPFFPPHPYKLWKQERERRVTNILDPSYHIPPLRERLYGRLTQIKGLFQKEQPAGERTPLLL

SEQ ID NO: 5 - DNA, 3063 bp, Homo sapiens
cgaggcgcgggtggtgccggtggcggcggcgggggagcgcgggacaggaggcttcggggaagatggacccggcgccctcgctgggctgcagcctcaaggatgtgaagtggagctcggtggccgtgccgctcgacctcctggtcagcacttaccggctgccccagatcgcgcgcctggacaacggagagtgcgtagaagggctgcgggaaaatgactatctgctgattcattcctgccgccagtggaccaccatcactgcccacagcttggaggagggtcactatgtcattgggccaaagatagagattccggtacattatgcagggcaattcaagctgctggaacaagaccgagatataaaggagccagtgcaatatttcaacagtgtggaggaggtggctaaggcatttcctgaacgcgtgtacgtcatggaggatatcacattcaacgtgaaggttgcttcaggtgaatgcaatgaagacactgaagtttacaacatcaccctgtgtactggggatgaactcactctaatgggggcaggcagaaatccttcatgcaaagacattcaaggaaaagtcacgactcaacacaatcttcaaaaagattgggaagctcaattccatcagcaagctgggaaaaggcaaaatgccgtgcctcatttgtatgaatcaccggaccaacgaaagcattagccttccattccagtgcaagggcagatttagcaccccgaagtcccctggaacttcagatgcaaaagggcgaacacaccatccgccacattgtggagaaaaccaggcttcctgtgaatgtgactgtgccaagccctccaccgagaaacccatacgacctccacttcatccgtgaggggcaccgctataagtttgtgaacatccagaccaagacggtggtggtttgctgtgtgctgcgggacaacaagatcctccccatgcactttcctttgcacttgactgtccccaagttcagcctcccagaacacctggtgaagggagagagctggcccgaaaccctggtccatcactggctaggtatctgccaagaacagttcgacatcgatgagtattcacgggctgtccgtgatgtgaaaaccgactggaatgaagaatgcaagagccccaagaagggtcggtgctctggccacaaccacgtgcccaattcgctcagctacgcccgcgatgagctcacccagtccttccaccgactctcggtctgtgtgtatggcaacaatctccatggcaacagtgaggtgaaccttcatggttgcagggacctggggggagattgggctccctttcctcatgacatcctgccctatcaggactctggagatagtgggagcgactaccttttcccagaagctagtgaagaatcagcaggcatcccgggaaagtcagaacttccctacgaagagctgtggctggaggaaggcaagcccagccatcagcctctcactcgctctctgagcgagaagaacagatgtgatcagtttagaggttctgtccgatccaaatgtgcgacttctcctcttcccatccctgggactctgggagcagcagtgaagtcttcagatactgccctacctccacctccagtgcctcccaaatctgaagccgtcagagaagaatgccggctcctgaacgccccacctgttccaccccgaagcgcaaagcctttgtccaccagtccctccatccctcctcgcacagtcaagccagcgcggcaacagactcgctctcccagccccaccttgtcctactattcttcagggctacacaacatcgtcactaaaactgacacaaatccttctgaaagcactcctgtttcctgctatccatgtaaccgagtgaaaactgattctgtggacctgaaatccccgtttggaagtccttctgctgaagctgtgtcctctcggctctcatggcctaaccattattcaggagcatcagaaagccagaccaggagtgacttcctgctggatccaagcaggagttatagttaccctagacaaaagacgccaggcacaccaaagagaaactgcccagcaccttttgattttgatggctgtgagctcctggccagccccactagcccagtcactgcagaattcagtagcagcgtctctggttgtcccaagtcagccagctactctctggagagcacagatgtgaaatctcttgcagctggtgtgacaaagcagagtacgtcatgccctgccttaccccccagggctccaaaactagtggaagagaaggtcgcctccgaaacatctcctttgcctctgaaaattgatggtgctgaggaagaccccaagtctgggtcaccagatctctcggaggaccagtattttgttaaaaagggcatgcaggacatcttctctgcctcctaccctttctcatctccgctccatctccagctggcccccagatcctgtggcgacggttccccatggcagccacctgctgacctatcaggactctctatagaggaagtgtccaagtcactacggttcattggtttgtccgaagatgtcatatcattctttgttactgaaaagattgatgggaacctgcttgttcagctaacggaagaaatcctctcagaggatttcaaattgagcaaattgcaggtgaagaagataatgcaattcattaatggctggaggcccaaaatatagccaaataacccccggccagcatggaacaaaactgatcaatgcgtgtgctagaaggggtgggctgggacacaatttcatgtttttgcactaaaaaccttctctgtaaatagggataagagaaactcttactatgcagattacgtttttgaatggtgaacaggctattttgtacatcaataaaaatgctgtacagaacacttggaggtgtgccttgtacgtcactcaacaaacactcagcagctgctaaaagaaaaaaaggcatgtgcagagaaatcattcttacccaagtaggtttatgtgagaaggtatgatatttattacaaaatagccaaagctgaaagacataaaaatctttaaaaaaaaaaaaaaaa

SEQ ID NO: 6 - protein, 647 aa, Homo sapiens
MQKGEHTIRHIVEKTRLPVNVTVPSPPPRNPYDLHFIREGHRYKFVNIQTKTVVVCCVLRDNKILPMHFPLHLTVPKFSLPEHLVKGESWPETLVHHWLGICQEQFDIDEYSRAVRDVKTDWNEECKSPKKGRCSGHNHVPNSLSYARDELTQSFHRLSVCVYGNNLHGNSEVNLHGCRDLGGDWAPFPHDILPYQDSGDSGSDYLFPEASEESAGIPGKSELPYEELWLEEGKPSHQPLTRSLSEKNRCDQFRGSVRSKCATSPLPIPGTLGAAVKSSDTALPPPPVPPKSEAVREECRLLNAPPVPPRSAKPLSTSPSIPPRTVKPARQQTRSPSPTLSYYSSGLHNIVTKTDTNPSESTPVSCYPCNRVKTDSVDLKSPFGSPSAEAVSSRLSWPNHYSGASESQTRSDFLLDPSRSYSYPRQKTPGTPKRNCPAPFDFDGCELLASPTSPVTAEFSSSVSGCPKSASYSLESTDVKSLAAGVTKQSTSCPALPPRAPKLVEEKVASETSPLPLKIDGAEEDPKSGSPDLSEDQYFVKKGMQDIFSASYPFSSPLHLQLAPRSCGDGSPWQPPADLSGLSIEEVSKSLRFIGLSEDVISFFVTEKIDGNLLVQLTEEILSEDFKLSKLQVKKIMQFINGWRPKI

SEQ ID NO: 7 - DNA, 892 bp, Homo sapiens
ggcacgagctcgtgcactcatggcgacccggaacccccctccccaagactatgaaagtgatgacgactcttatgaagtgttggatttaactgagtatgccagaagacaccagtggtggaatcgagtgtttggccacagttcgggacctatggtagaaaaatactcagtagctacccagattgtaatgggtggcgttactggctggtgtgcaggatttctgttccagaaagttggaaaacttgcagcaactgcagtaggtggtggctttcttcttcttcagattgctagtcatagtggctatgtgcagattgactggaagagagttgaaaaagatgtaaataaagcaaaaagacagattaagaaacgagcgaacaaagcagcacctgaaatcaacaatttaattgaagaagcaacagaatttatcaagcagaacattgtgatatccagtggatttgtgggaggctttttgctcggacttgcatcttaaggacatgaatattctcccataacggattcaactatgagaagagaagtggcagcaataaggcagtctctcaaaagtcatactgccagagtctctagggcaaggagaaacaactagctggacaatactcaattcacaacttagcattttgccatctgaagcttggcaaactagtatctgctgtaaaacaacctatatggtatgtgaaccgtagtattcctgagcaaaacgtggctttcatcgctttgtaaaaatttgcatctgtttagaaactagcctataaaatatcaccattggatgtagatatggagagaaaagaaatatgttgggtttattgcttagcgaaatattctctttttatttaaataaaatgttcttcattgtgttttaaaaaaaaaaaaaaaaaaa

SEQ ID NO: 8 - protein, 155 aa, Homo sapiens
MATRNPPPQDYESDDDSYEVLDLTEYARRHQWWNRVFGHSSGPMVEKYSVATQIVMGGVTGWCAGFLFQKVGKLAATAVGGGFLLLQIASHSGYVQIDWKRVEKDVNKAKRQIKKRANKAAPEINNLIEEATEFIKQNIVISSGFVGGFLLGLAS

SEQ ID NO: 9 - DNA, 1850 bp, Homo sapiens
cactcctactgcggctgctatgaagcttactggttgtgatgtgttataatttagtctgtttttttgattgaatgcagtttaatgtttccagaaagccaaagtaattttcttttcagatatgcaaggctttggtgggtccaaaaaatgtctatcacaagccattttttccttttcctctctcgaaaagttaaaatatctatgtgttattcccaaaccctcttacctatgtatctgcctgtctgtccatcatcttccttcctccctatctctgtgtatctggatggcagccgctgcccargggagtggctgtggggagggcaggtactgtctttgcctgtgggtccagctgagccatccctgctgggtgatgctgggcaagacccttggcccgtctgggccttggcttcctcacttgtgaaatgagcgggaagatgactctcagttccttccacctcttagacatggtgaggtaacagacatcaaaagcttttctgaaatcttcagaagaaatagttccattacagaaaactcttcaaaataaatagtagtgaaaacttttaaaaactctcattggagtaagtcttttcaagatgatcctccacaatggaggcagcgttcctacttgtcatcacacagctgaagacattgtttcttaggtgtgaaatcggggacaaaggacaaacagagacacagggcattgttcatgggaggcatcgtcaccctcctgggtgttctgtgggaatttcctgtgtgaggaaaacgtggccacagggttgtgctgtacccacccttccccggcgagatggccctcggcctgtgccgctgcttccaccctcgccactccatggcagcttttggtctgtttccggctctgccctctgccctgaactctcatccggcttgtacctgcctgctggacccctccacctggaggccagcccatgtctcaggcccagccctagcctcttctcctcaaattctaagtgttttctctttaggtttccctggctttgtgaatggatcatgtgtctctaggtataaacctgacatcatctctccacccggcttacctccaccagatctccccagttctgtctccatcttctacctgcagctgctctgttctcatggtcactgctgcatcactgagtctggacccttgttatcattttcaaactggcctccttccctcgttccccacttcttaaagtcacctgtccattgccaccagattaagctttctccagccagatcacctctctctgagaaacctccattgacatggaaacaccattgtctggcacacatactcacatactcaccttcccgtcttgatccccacacatctttccagcctcccctcccactccactccctgctccctcctccacctccccatcctcttgtctcccctcccctctgaatccagcccagcggggcttctcctgcctccatcacatcacagaagtacctcctgcttctggttttaattagagccttccccgattacattttcctctgaattttttcctatctacatttgatctgtcatgtttaaaccccctacttctaagggaacttctctaatctcttatcctcatccccaaatagtgttttcttcctctgggttcttataatgttggtatcaatctcacagcatttagtgcttcctgcctggtgtgacagttacctgtgtgcatgtgcaatttctaatttcccacgctagactgtgagcttcctaaggcaagaatcatgccttgttggtttctgtattcctcatggtgccaaacacagtgccttctacattgcaggcgctgaataaacatttttaaagcaaaaaaaaaaaaaaa

SEQ ID NO: 10 - protein, 206 aa, Homo sapiens
MALGLCRCFHPRHSMAAFGLFPALPSALNSHPACTCLLDPSTWRPAHVSGPALASSPQILSVFSLGFPGFVNGSCVSRYKPDIISPPGLPPPDLPSSVSIFYLQLLCSHGHCCITESGPLLSFSNWPPSLVPHFLKSPVHCHQIKLSPARSPLSEKPPLTWKHHCLAHILTYSPSRLDPHTSFQPPLPLHSLLPPPPPHPLVSPPL

SEQ ID NO: 11 - DNA, 2216 bp, Homo sapiens
cttgtaagttactgttagtgaattgttttttacgtttcatttaataattgctgctaaaggtgatgtttactgataaatcattttaaaatttttttgttttgaaaagtaaatttatcccccatgatgttagatacatttaaattattaagtcttttcagagatgagatggggacaggaagttattttgagccttacaatattatttagcccaataaaagatgcattgaagctcttatatattatgagtttgaaaaattttgaaggtagcatattgaagtgatctataaatatcttcagtcctctctgaagtgtgggtatttcttctatctaaaaaatacatacagtgactgtcttcaaatctacttggttcttgaccaaatargagctaatgggtaatgaatacctttttgtttgtttgtttgtttgtttgttttttgtttttttttttaagggtctcactcttttgcccaggctggagtgcagtggcacaatcacggctcccaggctaatgtttttatttttaatttgtaattttttttttattttttttgttgagatggagttgctccatgttgcacaggctgttctcaaactcctaagctcaagccatctgcctgcsttggcctcccaaagtgstgggattgtagacataagccacctcacccagcctatgaatatctttctaacatkgtaagaatgaggtaatgtttccatcagtctaatacagatatatttcttccctccaaaacagtttattttgattgtttattttattttgattgtaactccgtcataactygacatggaaaatgctatatactatgaaaacttagctgaaagggaagaattgttttagaaagacaatatttaaaacaccgcactgccaatatattgatcctttatagttatttcctaaaatgctgttttcgaaacattcctttttcaccctgttgtgtggcttagacccatctcgtaatctgttaattggaaagaggctacagacaccagcagtgtgcgttctgcaggtacacgctgccaaagtaattcctgctcatccatgccctgtctctgtctcttttagagtcataccttatttgagtataggtggcttaattttgctagacttcctgaaaacactaaggtggagtatcagaagtgattttagtcacagttctgcgggagagcttagaataacatcctcctttgggaggtggtcttgggtgcgtggatcttggtatacagtctttattgtaagtctgatacaaaatgctaataaatttaatgtttttcttccttaatttattggcatagttcttcaggtagcacctcatttttattaatgatattgggattaactatgaacaagctatatgtagacatttgcatttaaggacattgcagtgtttcaaagatcccatcattgcagcttgtatcctttagatccaatcggaaacttctggagtcttacattaatgctcatttgagctaattagtaatctgtttaaacagatttggcaatactttaaagatactgtagactatttatgtatagatagatcatattacccattaaaagtctgggggaaaaaattttttaattttactcttcttatgtactgaaaactttttttaaaaaaggtgatgatgaagttcattctgtagcagcagcgcagctatgctttaaaccacacaaaaggctgtgtccaggtgcagcctccttcacccttcstgcccacggtgaggattgaataaccaggacttggggatatkgtttgttgtcagggttattctgtgtggtaaggaatatttgtttcacatttatacattttctttttccactcacgtaagtttctatcttgagagcatagtccaaagtgcaaaacttggtgtttacaaggaaaattgtcttccagaactccactgtcatcactttcaccaaagtggaagtttgcatgaatatgctcagaatctaatattcaatgttctgttacattgtaagtgaagtccagctacaaaatagatttaatatattgaatttatttgtacatatgcagagtacggtatttctgtatggaatctgctttattcctatttttcccaactctgatgagtagaatattaaatgtgttgttatggaaatacagattattgcttctataggaagataattatgaaaataaaacctgaaactatataaatataaaaaaaaaaaaaaa

SEQ ID NO: 12 - protein, 126 aa, Homo sapiens
MLFSKHSFFTLLCGLDPSRNLLIGKRLQTPAVCVLQVHAAKVIPAHPCPVSVSFRVIPYLSIGGLILLDFLKTLRWSIRSDFSHSSAGELRITSSFGRWSWVRGSWYTVFIVSLIQNANKFNVFLP

SEQ ID NO: 13 - DNA, 1426 bp, Homo sapiens
ctgggtctccagggggagagcctggccctgtcctttgctacccagggctgcccccaggcccatgaagccaataggagagcgtgtggcactggcccacaaactgtccctgtcctgtcttcctcccgagccatggcctctgctagctccaccttgaaggagccccccacatcctcccctacatcccagagatgccaccacttgtgtctccacaatgtgctcctgcccacccgggttccgcactgtccgacccctgcacaccactcatgtcaccacggcgtgcatcatgttcatccccatctatttatttaagcctttctttgcttgtagggcattttgtatgtagagcagttgaaaacagaacctcagaacttaacatctgtcctgatgttaaagtgcttttcatgaccaccctgttatctatgtatatgtaaagttaaggatgagatcttaagtttacaattaaaaactcagtactcaatatttaatattctactcgagctttatggaagccaaatcatgcatgtgtgtgtgtgcgtgtgtgcaagctttgaacctccttccacagccgcatcttctcatgacacaaagcttttgataagtactttcctgtgggtcgctcagggcctcatagcatctcattcaattacaagaatagaggccagacacggtggcgcatgcctgtagtcccagctaactgggaggctgaggcaggaggatcacttgagcccaggagattgaggctgcagtgagcatgatcgcgacactgcactccagcctgggtgacggtgagactttgtctcaaaaaaaaaaaaaaaaacaatggaaggcagacagcaagtccctgaggacacatcacacagtgtcctgtagctaagtgtctaggaaaaaacaaaaactccaaacccttcagtggatgaggacaaggtcgcagaaaggcattctgttgacagatgaacagccgaaagctggccagaccctcctgtatgcctctgcccttgtcctgtgggttgagggggtctgaccaggaggccacctacagcaggaagtgaggctgccatgtttccttgagacacagctgcctctccccagctctgtccctgtagtcacctgccggtgggcgaggatcctctccctgggataagcactcccagccccgtttatcagaaacacaggcaaggaaattggaactgccacccagcccagcatggtggctcaattggttggttgcgttgtcagttgtctcttcgttttgttaaggtttttaataagtacgtttggcataatgtcttttaatgggtttgtaatatttgtaacggttttagcagcctataacttttcagctggtgcttttacttagggaaaaaaacaatttgtaaatacagaacattgtttaaaagacataaccatagaaaaaaaaaaaaaaaaa

SEQ ID NO: 14 - protein, 80 aa, Homo sapiens
MPPLVSPQCAPAHPGSALSDPCTPLMSPRRASCSSPSIYLSLSLLVGHFVCRAVENRTSELNICPDVKVLFMTTLLSMYM

SEQ ID NO: 15 - DNA, 2364 bp, Homo sapiens
gaagcggctgctgtaggcgccgacggagcgagcgggcgtgcggagcgggcgacagtggcgtgggatctgcctctctgcgagcagctgggagcggcggcggcggcgccatgagcgggggcaccccttacatcggcagcaagatcagcctcatctccaaggcggagatccgctacgagggcatcctctacaccatcgacaccgaaaactccaccgtagcccttgccaaagttcgatcctttggtacagaagacagaccgacagatcgtccaataccacctcgagatgaagtctttgaatacattatattccgtgggagtgacattaaagaccttactgtttgtgagccaccaaaaccacagtgttctttgcctcaagacccagctattgttcagtcctcactaggctcatcgacttcttcattccagtccatgggttcttatggacctttcggcaggatgcccacatacagtcagttcagtccgagttccttagttgggcagcagtttggtgctgttggtgttgctggaagctctttgacatcctttggaacagaaacatcaaacagtggtaccttaccccaaagtagtgcggttggttctgcctttacacaggatacaagatctctaaaaacacagttatctcaaggtcgctcaagccctcagttagaccctttgagaaaaagcccaaccatggaacaagcagtgcagaccgcctcagcccacttacctgctccagcagctgttgggagaaggagtcctgtatcaaccaggcctttgccatctgccagccaaaaggcaggagagaatcaggagcacaggcaagctgaagtacacaaagtttcaaggccagaaaatgagcaactcagaaatgataacaagagacaagtagctccaggtgctccttcagctccaaggagagggcgtgggggtcatcggggtggcaggggaagatttggtattcggcgagatgggccaatgaaatttgagaaagactttgactttgaaagtgcaaatgcacaattcaacaaggaagagattgacagagagtttcataataaacttaaattaaaagaagataaacttgagaaacaggagaagcctgtaaatggtgaagataaaggagactcaggagttgatacccaaaacagtgaaggaaatgccgatgaagaagatccacttggacctaattgctattatgacaaaactaaatccttctttgataatatttcttgtgatgacaatagagaacggagaccaacctgggctgaagaaagaagattaaatgctgaaacatttggaatcccacttcgtccaaaccgtggccgtgggggatacagaggcagaggaggtcttggtttccgtggtggcagagggcgtggtggtggcagaggtggtaccttcactgcccctcgaggatttcgcggtggattcagaggaggtcgtgggggccgggagtttgcggattttgaatataggaaaaccacagcttttggaccctaaaaggtctggattgatcgtactgctttctgaaagaaagacaacaaagttgctgcatagtctacaaacaagtctctgaaaataggtgaatttctagctcttcatggtcctgaacattgatttcagtctttgcaaagaatgaagaagtgaattcgctgtacatttgtcaccagcactgggtttttgttttttgtttgtttttccgcttaatttcaaagataaaatgcagttacttttgggggtggaaggctcatcttaaaacatgagcattaaatatatttggaatagcagaaggttaagtaatttcttatgtatagttaaactaaagcagtacttcagtgggacttaacaagtattttttcatcactgaaaggtttttttttttttatcactaaattgtatttggcaattgcaagttgcctgcagatagggccgtgatactgtgttttgagccacagaaggttgtgtgtgtgtgtgtgtgtgtgtgtgtgtgtgtgtgtgtgtgtatgtgtgtgtctttttcctcctttcttttggggaatcctgtaatatgaggtagcttatttcgtcaattaattagggtgctggatggtagagaattttgtcagtcaactatgtacacacagtaaatactgtttcttaggcaaaggtaacttttttatatagttgtaaaattccattatattccattgccaaagaaacattaagacctttgtatagctgtataaaaagcaactaattttttaaagaaataaacattttaaagtccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - protein, 463 aa, Homo sapiens [sequence omitted]

SEQ ID NO: 17 - DNA, 2760 bp, Homo sapiens
tgaagatgcctcctctgatgcctactgctttgagctgctctctatggttttagcactgagtggctctaacgttggccggcaatatctggctcaacagctaaccctgcttcaggatctcttctcgctgcttcacacagcctctcctagagtccagagacaggtaacctctttactaagaagagttttgcctgaagtaacccctagtcgtctggccagcatcataggagtgaaatccctccccccagcagatatcagtgatatcattcactcaacagagaaaggagactggaataagctgggtatcttggacatgtttctaggatgcattgccaaagcactcactgtacagctaaaagccaaaggaaccaccatcactggaacagctggtaccactgtgggcaaaggagttacaacagttactcttccgatgattttcaattccagttatctccgacgaggtgaaagtcattggtggatgaagggctcaacccctacccagatctcagagatcatcattaaacttatcaaggatatggcagcaggtcatctgtcagaagcttggtcccgagtgacaaaaaatgctattgcagaaaccatcattgccttgaccaagatggaagaagaatttaggtctccagtgagatgtattgcaacaactagactctggcttgctctcgcatccctatgtgttcttgatcaggaccacgtagatcgtctctcctcggggagatggatgggaaaggatggacaacaaaaacaaatgcctatgtgtgataaccatgatgatggtgaaactgcagcaatcattttatgcaatgtctgtggaaatttatgtacagactgtgacagattccttcaccttcatcgaagaaccaaaactcatcaaagacaggtcttcaaagaagaagaagaagctataaaggttgaccttcatgaaggttgtggtagaaccaaattgttctggttgatggcactggcagattctaaaacaatgaaggcaatggtggaattccgagaacacacaggcaaacccaccacgagtagctcagaagcatgtcgcttctgtggttccaggagtggaacagagttatctgctgttggcagtgtttgttctgatgcagattgccaggaatacgctaagatagcctgtagtaagacgcatccttgtggccatccatgcgggggtgttaaaaacgaagagcactgtctgccctgtctacacggctgtgacaaaagtgccacaagcctgaagcaagacgccgatgacatgtgcatgatatgtttcaccgaagcgctctcggcagcaccagccattcagctggattgtagtcacatattccacttacagtgctgtcggcgagtattagaaaatcgatggcttggcccaaggataacatttggatttatatcttgtcccatttgcaagaacaaaattaatcacatagtactaaaagacctacttgatccaataaaagaactctatgaggatgtcagaagaaaagccttaatgagattggaatatgaaggtctgcataagagtgaagctatcacaactcctggtgtgaggttttataatgacccagctggctatgcaatgaatagatatgcatattatgtgtgctacaaatgcagaaaggcatattttggtggtgaagctcgctgcgatgctgaggctggacggggagatgattatgatcccagagagctcatttgtggtgcctgttctgatgtttccagggctcagatgtgtcccaaacatggcacagactttttggaatataaatgtcgctactgctgttcagtggctgtttttttctgttttggaacaacacatttttgtaatgcttgtcatgatgattttcaaagaatgactagcattcctaaggaagaactaccacactgtcctgcaggtcccaaaggcaagcagttagaaggaactgaatgtccactccatgttgttcatccacccactggggaagagtttgctctgggatgtggagtgtgcagaaatgcccacactttttagaacacgcagatcctttgtctacagagagaaaaattgccttcatcccccaagaggatgcggtgaagtttaaactctgctcaggataaggacgggaccatttttacatccatgaaaatgaaccattcacagtgcaagaaggataccaaataccatgtacataattcttgctatgaaaagtttccccattattttggtttatcttcttttgaacaaatgacatcaaacttgtgaggtgtttgcatgtggccattaccgtcattggcctgtgaagcattggacatttatagataattgatataaaagaatcgccatgcccatggactaagaacgatgctggctttcaagcaaaaaagaaaaataatcattgtttattgtatactgcctttttgtaatcctgtacaattgcatcacgggtggggataaaaagaggaatattctggtttatttcctagactgttatttaaaaaaaaaaaaacattgtgttaggacagcatataaatgtaataagtatcacactgtatataaacatatcaatgtttgtcctgtataagaattactaaattacaaatgcaatttcatttaaacttctaggttaagtttgagcctgaaattttaatgaagtgcaatactgagtgtgcctcattatcttgcagctgtaaacatattggaatgtacatgtcaataaaaccactgtacatttttatacagtgataaagtctaaaaaaaaaaaaaaa

SEQ ID NO: 18 - protein, 660 aa, Homo sapiens [sequence omitted]

SEQ ID NO: 19 - DNA, 1649 bp, Homo sapiens
gattgtacatagtcttgtggggcatgggggagccggctggaggtgagaaccctcccctctccccccaccccccggggagagcaaatgtaaaactactaatttttgtgctttatatattctatataaatatatctattttctttttacaaaaccagtttataaatggtaggggggtgtggggcggacacatggagctccccttgtgggggggccccctccattacccgacctaccgcccttttcctcaccccccaccccactccccaccccctggctgtgactgctgtaagatgggggtatagaggctgggcaattcccaccccctgttgtatagttggactatgttataacgcacaaaagagagctgaccccagggggagccagagggtgatgggttccttgcctccctttccttcccctttttgcccaagcttgtgctgcagttgaacctcttcctgggggtgggagtaggtaaggggtgggtgaggccccaaacccctctctggtagggaaccgtggggatgaagatgaagcttatatgcagttctcttctaggggctgtgggcaaagggcattttgtaattaatattttcaagaatcagatgtctggagtgtaggggtgggcttggtggtggtggacgggcgggcctgctggagggggagcttggtcgctgttgtgattttaggtttgtttttgttttgttttgaatttggggggttgtggattgttgggggtagggagatttttttttttttaaagctgcttcctcaactgtttcaagctgcaaatgtttaagagaataacagcccccactcccacaggaaccgctgtaattaaatcagacagtaggaagactgggctgctgccctcaaagccacagcccttggatgttccttttccgagagcagaaggtctaggctacagggagggggagattggctcccgtgagtcaggctgtgtttggggcttgggccctgggattgggaaaaggggatggggcagactttgtaagcatatgctaggtatccgatagtcctgtagaatttagtgaagaaaccttatacagtttttaatttttatataaactataactcagacccaagctacaaggttggaattttggttggttttttttttaagtaccctgcctgtataattgcatcagaatcccccaccccacccccsgccccsgtgtttgtattttgggttggtttacactcgcacatactcagttttcagttttcccctttacagtcttctcccctcacctccaggaccctccccctttttaaaaaataaatcgctgacaagtgtgaatcccgtgaagactttattttgtgttgtgtgtatcctgtacagcaaggttggtccttcgtaacaacggatgaaatggttcccttttttaaagcgccctctctccctccaccctcagcgcccctgtccttggcatgttttgtatcagcgatcattctgaactgtacatatttatgttgcgagaggcaaagggcaagttttggattttgcttcttccaagtttgtttttaaacgacaaataaaaaaagaacattttaaataaaaaaaaaaaaaaaaaaaaaaaaaaaaaaaaaaaaaaaaaaaaaaaaaaaaaaaaaaaaaaaaaaaaaaaaaa

SEQ ID NO: 20 - protein, 92 aa, Homo sapiens
MGEPAGGENPPLSPHPPGRANVKLLIFVLYIFYINISIFFLQNQFINGRGVWGGHMELPLWGGPLHYPTYRPFPHPPPHSPPPGCDCCKMGV

SEQ ID NO: 21 - DNA, 2644 bp, Homo sapiens
gttgaggatggctgacattctctctcagtcagagaccctggcgtcgcaagacctcagtggggacttcaagaagccagctctgccggtgtccccagcggcgcggagtaaggccccggccagcagttcttcaaaccctgaggaggtacagaaggaagggcccactgcgttgcaggactccaattctggggagcccgacatccctcctcctcagccggactgcggtgattttaggagtctacaggaggagcagtcgcgccccacgacagcggtttcttcccctggcggtccagcccgggctcccccctaccaagagcctccatggggtggccctgccacagccccctacagcttagagaccctgaagggcggcactatccttggcacccgtagcttgaaagggacgagttactgccttttcgggaggctgtctggctgcgacgtgtgcctggagcacccttcggtgtctcggtaccacgcagtgctgcagcacagggcgtccggccctgacggagaatgcgacagcaacgggccgggcttctacctctacgatctgggaagcacccatggcacttttctcaacaaaactcgcatcccacctcgcacctactgtcgagtccacgttgggcatgttgttcgctttggaggcagcacccggctctttatcctgcagggaccagaggaagaccgagaggcagaatccgagttaacagtaacacagttgaaggaattgcgcaagcagcagcaaatattgttggrgaagaagatgctaggagaagactcagatgaagaagaggaaatggatacctctgaaaggaagataaatgctggtagccaagatgatgagatgggttgcacctggggaatgggagaagatgcagtagaggatgatgctgaagagaaccctattgtcttagagtttcagcaggaaagggaggccttttatataaaggatcccaaaaaggctctccaaggcttttttgaccgagaaggagaagaattagaatatgaatttgatgaacagggacatagcacttggctctgcagggtgagattacctgtggacgattcaactggaaaacaactggtggctgaggccattcactcaggaaagaaaaaagaagcaatgatccagtgctcattggaagcttgtcggattcttgacactttgggattgcttcggcaggaagcagtatctcggaaaaggaaagccaagaactgggaagatgaagacttttatgatagtgatgatgacacatttcttgataggactggcctgattgagaagaagcgtctgaacagaatgaagaaggctggcaagattgatgagaagccagagacctttgaatcattggttgcaaaattaaatgatgctgaaagggaactttctgaaatttctgagagattgaaagcctcaagccaagttctatcagagtctccatctcaggattctttagatgcgttcatgtcagaaatgaaatcaggcagtacattagatggtgtgtcccggaagaaacttcacctgagaacttttgaactgaggaaagaacaacagagacttaaagggttaataaaaattgtaaagccagcagagattccagaactaaaaaagactgaaactcagactacaggtgcagaaaacaaagctaaaaagcttacattgcctctatttggtgccatgaaaggaggaagcaaattcaaattaaaaactggaacagtagggaagttaccccccaagcgtccagaactccctccaactctaatgagaatgaaagatgagcctgaagtagaagaggaggaggaagaggaagaggaagaagagaaagaaaaggaggagcatgaaaagaaaaaactggaggatggaagcctcagtaggccacagccagagatagagccagaagcagcagtgcaggaaatgaggcctcccacagatctcacacattttaaagaaacccaaacccatggtaatatctttcttctccttcctgtgttgttcagtgggcagttacattgattgtggataggttttaaaaagcaaggccagttcttgtctgtgcatttgactttgtatgtgatatactgactctgtagcaaggaaacatactttcttggtcttcttcctttgaccgccagtcattatttgtcttcattgcaaattaagggcagttatttccaatccattccagaattacagaaaattgaagggctatggaatctgaaaccatagctgctgtggaataatcctgagctgctgccactgtgtgagttggagggcagtggaacagggtacatgatggggcctgatcaggtggtctccggataagtcaacccctattcattttttcctccatccctaaaacagaggccaaaccataattgtactcattggactaaagttctcaagaaggatcttgcttcattcatttttgtgtgtttggaacctagcacaaaacctgacacatatccacccgcctcagcaaatatttgatgaaaaatgttgaaagacggaatagattgatattcatatagatatatgcatcaattaattctgtattttctatatatatattctaattacaaagggttatatgttcattttagaaactatagatcatacataaaagtccaaaggaaaaaaaaaaaaaaa

SEQ ID NO: 22 - protein, 667 aa, Homo sapiens
  UNSURE (250)
MADILSQSETLASQDLSGDFKKPALPVSPAARSKAPASSSSNPEEVQKEGPTALQDSNSGEPDIPPPQPDCGDFRSLQEEQSRPTTAVSSPGGPARAPPYQEPPWGGPATAPYSLETLKGGTILGTRSLKGTSYCLFGRLSGCDVCLEHPSVSRYHAVLQHRASGPDGECDSNGPGFYLYDLGSTHGTFLNKTRIPPRTYCRVHVGHVVRFGGSTRLFILQGPEEDREAESELTVTQLKELRKQQQILLXKKMLGEDSDEEEEMDTSERKINAGSQDDEMGCTWGMGEDAVEDDAEENPIVLEFQQEREAFYIKDPKKALQGFFDREGEELEYEFDEQGHSTWLCRVRLPVDDSTGKQLVAEAIHSGKKKEAMIQCSLEACRILDTLGLLRQEAVSRKRKAKNWEDEDFYDSDDDTFLDRTGLIEKKRLNRMKKAGKIDEKPETFESLVAKLNDAERELSEISERLKASSQVLSESPSQDSLDAFMSEMKSGSTLDGVSRKKLHLRTFELRKEQQRLKGLIKIVKPAEIPELKKTETQTTGAENKAKKLTLPLFGAMKGGSKFKLKTGTVGKLPPKRPELPPTLMRMKDEPEVEEEEEEEEEEEKEKEEHEKKKLEDGSLSRPQPEIEPEAAVQEMRPPTDLTHFKETQTHGNIFLLLPVLFSGQLH

SEQ ID NO: 23 - DNA, 2402 bp, Homo sapiens
gatcgcagagaccaaggaggcgcccgcggctgcagagctgcagagcgggatctcttcgagctgtctgtgtccgggcagccggcgcgcaactgagccagaggacagcgcatcctttcggcgcgggccggcagggcccctgcggtcggcaagctggctccccgggtggccaccgggacccccgagcccaatggcgggggcggcggcaaaatccacaacactgtagagatcacccccacctccaacggacaggtcgggaccctcggagatgcggtgcccacggagcagctgcagggtgagcgggagcgcgagcgggagggggagggagacgcgggcggcgacggactgggcagcagcctgtcgctggccgtgcccccaggccccctcagctttgaggcgctgctcgcccaggtgggggcgctgggcggcggccagcagctgcagctcggcctctgctgcctgccggtgctcttcgtggctctgggcatggcctcggaccccatcttcacgctggcgcccccgctgcattgccactacggggccttcccccctaatgcctctggctgggagcagcctcccaatgccagcggcgtcagcgtcgccagcgctgccctagcagccagcgccgccagccgtgtcgccaccagtaccgacccctcgtgcagcggcttcgccccgccggacttcaaccattgcctcaaggattgggactataatggccttcctgtgctcaccaccaacgccatcggccagtgggatctggtgtgtgacctgggctggcaggtgatcctggagcagatcctcttcatcttgggctttgcctccggctacctgttcctgggttaccccgcagacagatttggccgtcgcgggattgtgctgctgaccttggggctggtgggcccctgtggagtaggaggggctgctgcaggctcctccacaggcgtcatggccctccgattcctcttgggctttctgcttgccggtgttgacctgggtgtctacctgatgcgcctggagctgtgcgacccaacccagaggcttcgggtggccctggcaggggagttggtgggggtgggagggcacttcctgttcctgggcctggcccttgtctctaaggattggcgattcctacagcgaatgatcaccgctccctgcatcctcttcctgttttatggctggcctggtttgttcctggagtccgcacggtggctgatagtgaagcggcagattgaggaggctcagtctgtgctgaggatcctggctgagcgaaaccggccccatgggcagatgctgggggaggaggcccaggaggccctgcaggacctggagaatacctgccctctccctgcaacatcctccttttcctttgcttccctcctcaactaccgcaacatctggaaaaatctgcttatcctgggcttcaccaacttcattgcccatgccattcgccactgctaccagcctgtgggaggaggagggagcccatcggacttctacctgtgctctctgctggccagcggcaccgcagccctggcctgtgtcttcctgggggtcaccgtggaccgatttggccgccggggcatccttcttctctccatgacccttaccggcattgcttccctggtcctgctgggcctgtgggattatctgaacgaggctgccatcaccactttctctgtccttgggctcttctcctcccaagctgccgccatcctcagcaccctccttgctgctgaggtcatccccaccactgtccggggccgtggcctgggcctgatcatggctctaggggcgcttggaggactgagcggcccggcccagcgcctccacatgggccatggagccttcctgcagcacgtggtgctggcggcctgcgccctcctctgcattctcagcattatgctgctgccggagaccaagcgcaagctcctgcccgaggtgctccgggacggggagctgtgtcgccggccttccctgctgcggcagccaccccctacccgctgtgaccacgtcccgctgcttgccacccccaaccctgccctctgagcggcctctgagtaccctggcgggaggctggcccacacagaaaggtggcaagaagatcgggaagactgagtagggaaggcagggctgcccagaagtctcagaggcacctcacgccagccatcgcggagagctcagagggccgtccccaccctgcctcctccctgctgctttgcattcacttccttggccagagtcaggggacagggagagagctccacactgtaaccactgggtctgggctccatcctgcgcccaaagacatccacccagacctcattatttcttgctctatcattctgtttcaataaagacatttggaataaacgaaaaaaaaaaaaaaaaaaaaaaaaaaaaaa

SEQ ID NO: 24 - protein, 520 aa, Homo sapiens
MASDPIFTLAPPLHCHYGAFPPNASGWEQPPNASGVSVASAALAASAASRVATSTDPSCSGFAPPDFNHCLKDWDYNGLPVLTTNAIGQWDLVCDLGWQVILEQILFILGFASGYLFLGYPADRFGRRGIVLLTLGLVGPCGVGGAAAGSSTGVMALRFLLGFLLAGVDLGVYLMRLELCDPTQRLRVALAGELVGVGGHFLFLGLALVSKDWRFLQRMITAPCILFLFYGWPGLFLESARWLIVKRQIEEAQSVLRILAERNRPHGQMLGEEAQEALQDLENTCPLPATSSFSFASLLNYRNIWKNLLILGFTNFIAHAIRHCYQPVGGGGSPSDFYLCSLLASGTAALACVFLGVTVDRFGRRGILLLSMTLTGIASLVLLGLWDYLNEAAITTFSVLGLFSSQAAAILSTLLAAEVIPTTVRGRGLGLIMALGALGGLSGPAQRLHMGHGAFLQHVVLAACALLCILSIMLLPETKRKLLPEVLRDGELCRRPSLLRQPPPTRCDHVPLLATPNPAL

SEQ ID NO: 25 - DNA, 2377 bp, Homo sapiens
ttcattcttcagtggaaatccatcagttgaaatagttcatggtattatgcacctatataagacaaataagatgacctccttaaaagaagatgtgcggcgcagtgccatgctgtgtattctcacagtccctgctgcaatgaccagtcatgaccttatgaagtttgttgccccatttaacgaagtaattgaacaaatgaaaattatcagagactctactcccaaccaatatatggtgctgataaagtttcgtgcacaggctgatgcggatagtttttatatgacatgcaatggccgccagttcaactcaatagaagatgacgtttgccagctagtgtatgtggaaagagctgaagtgctcaaatctgaagatggcgccagcctcccagtgatggacctgactgaactccccaagtgcacggtgtgtctggagcgcatggacgagtctgtgaatggcatcctcacaacgttatgtaaccacagcttccacagccagtgtctacagcgctgggacgataccacgtgtcctgtttgccggtactgtcaaacgcccgagccagtagaagaaaataagtgttttgagtgtggtgttcaggaaaatctttggatttgtttaatatgcggccacataggatgtggacggtatgtcagtcgacatgcttataagcactttgaggaaacgcagcacacgtatgccatgcagcttaccaaccatcgagtctgggactatgctggagataactatgttcatcgactggttgcaagtaaaacagatggaaaaatagtacagtatgaatgtgagggggatacttgccaggaagagaaaatagatgccttacagttagagtattcatatttactaacaagccagctggaatctcagcgaatctactgggaaaacaagatagttcggatagagaaggacacagcagaggaaattaacaacatgaagaccaagtttaaagaaacaattgagaagtgtgataatctagagcacaaactaaatgatctcctaaaagaaaagcagtctgtggaaagaaagtgcactcagctaaacacaaaagtggccaaactcaaatctcagagtgggtatcctagcatctagcaagactgagtggggagatttctcatccgtgtgaaaatgtagagtgaggcctctgactagctaattgtgtattttgttgggtttagtattttctaaatgtttacaaaatattgggctgcatgttcaggttgcagctagagggagcttgggcagattttcaattacgctttcaagatataaccaaaagctgtttctaaatcctaaaattagaatttcaacagagccccctttagaacagtcatataacgcttgtgtgggccaacagaggggctgtgtactctctctggaaccataaatgtcaaataatttataacctgcagtaattgagcaaacttaaaataagacctgtgttggaatttagtttcttgaagaggtagagggataggttagtaagatgtattgttaaacaacaggttttagtttttgctttataattagccacaggttttcaaatgatcacatttcagaataggtttttagcctgtaattaggcctcatcccctttgacctaaatgtctgacatgttacttgttagcacatcaactgtatcactaatcaccatctgtttttgtgggatgtgctgcagcatttcccaaaaaactttacgtgtaatgttgcaaaatgaatgtactcagacattcttaatttttacttagggcagaccaactctttgagtctctcttggacttatatatacagatatcttaagagtgggaatgtaaagcataacctaattctctttcctatagagattctattttatttaaaatctatttttacactagttagaatcctgctgttttggccaagtacttgtcttgcatgtctgaccttgcagaagctggggtggatcatagcatactaatgaagagaattagaagtagtttacaaagctcgctcactcctcatttctctgtgatcccttctatccagtggccccaccaccacctgggaaaacagatttttcagtacaggtgggataaatgctttgaaaggctgtgcccagagcaatgagcaaataggcaagtgtttccaaactamttggaggtttacaaaaaatatgtcccagaaaaaaaaaaaatcttaccaagatacgtaaagaaaaaaaaatttttttttaaacagtcaaagagtcatgtttgaatttcacaaaatcacatcagacagaagttgttttcttcaggagggaaatgaaccacttaatatacccatactaccttgaacaatgaaattgaattaaaatagccaaactttgaaattaaaaaaaaaaaaaaa

SEQ ID NO: 26 - protein, 351 aa, Homo sapiens
MHLYKTNKMTSLKEDVRRSAMLCILTVPAAMTSHDLMKFVAPFNEVIEQMKIIRDSTPNQYMVLIKFRAQADADSFYMTCNGRQFNSIEDDVCQLVYVERAEVLKSEDGASLPVMDLTELPKCTVCLERMDESVNGILTTLCNHSFHSQCLQRWDDTTCPVCRYCQTPEPVEENKCFECGVQENLWICLICGHIGCGRYVSRHAYKHFEETQHTYAMQLTNHRVWDYAGDNYVHRLVASKTDGKIVQYECEGDTCQEEKIDALQLEYSYLLTSQLESQRIYWENKIVRIEKDTAEEINNMKTKFKETIEKCDNLEHKLNDLLKEKQSVERKCTQLNTKVAKLKSQSGYPSI

SEQ ID NO: 27 - DNA, 460 bp, Homo sapiens
cgagatgaagccggcggtggacgagatgttccccgagggcgccgggccctacgtggacctggacgaggcgggaggcagcaccgggctcttgatggacttggcagccaatgaaaagccgttcatgcagacttttttaacgattttgaagatctttttgatgatgatgacatccagtgagatgccctctggctgcaggcggggccaagcccttggtacagagccgcagtgtgagcctgcgcaggacagtttcaggtggttttaaagaacacgtggaaatcccttgaatttaggacctggttaaccagaaagataagactgttcttaacgacctagatgattctgttcatctctgaacgggatcaggttttgtcctcactccaattaaaagaaagcaatgtcacatgaaaaaaaaaaaaaaaaaaaaaaaaaaaaaaaaaaaaaaaaaaaaaaaaaaaaaaaa

SEQ ID NO: 28 - protein, 85 aa, Homo sapiens
MKPAVDEMFPEGAGPYVDLDEAGGSTGLLMDLAANEKPFMQTFLTILKIFLMMMTSSEMPSGCRRGQALGTEPQCEPAQDSFRWF

SEQ ID NO: 29 - DNA, 3204 bp, Homo sapiens
gtttggcatctgtggccgagttgctgttgccgggtgatagttggagcggagacttagcacaatggcagaacctgtttctccactgaagcactttgtgctggctaagaaggcgattactgcaatctttgaccagttactggagtttgttactgaaggatcacattttgttgaagcaacatataagaatccggaacttgatcgaatagccactgaagatgatctggtagaaatgcaaggatataaagacaagctttccatcattggtgaggtgctatctcggagacacatgaaggtggcattttttggcaggacaagcagtgggaagagctctgttatcaatgcaatgttgtgggataaagttctccctagtgggattggccatataaccaattgcttcctaagtgttgaaggaactgatggagataaagcctatcttatgacagaaggatcagatgaaaaaaagagtgtgaagacagttaatcaactggcccatgcccttcacatggacaaagatttgaaagctggctgtcttgtacgtgtgttttggccaaaagcaaaatgtgccctcttgagagatgacctggtgttagtagacagtccaggcacagatgtcactacagagctggatagctggattgataagttttgcctagatgctgatgtctttgttttggtcgcaaactctgaatcaacactaatgaatacggaaaaacacttttttcacaaggtgaatgagcggctttccaagcctaatattttcattctcaataatcgttgggatgcctctgcatcagagccagaatatatggaagacgtacgcagacagcacatggaaagatgcctgcatttcttggtggaggagctcaaagttgtaaatgctttagaagcacagaatcgtatcttctttgtttcagcaaaggaagttcttagtgctagaaagcaaaaagcacaggggatgccagaaagtggtgtggcacttgctgaaggatttcatgcaagattacaggaatttcagaattttgaacaaatctttgaggagtgtatctcgcagtcagcagtgaaaacaaagttcgaacagcacactatcagagctaaacagatactagctactgtgaaaaacataatggattcagtaaacctggcagctgaagataaaaggcattattcagtggaagagagggaagaccaaattgatagactggactttattcgaaaccagatgaaccttttaacactggatgttaagaaaaaaatcaaggaggttaccgaggaggtggcaaacaaagtttcatgtgcaatgacagatgaaatttgtcgactgtctgttttggttgatgaattttgttcagagtttcatcctaatccagatgtattaaaaatatataaaagtgaattaaataagcacatagaggatggtatgggaagaaatttggctgatcgatgcaccgatgaagtaaacgccttagtgcctcagacccagcaagaaattattgaaaatttgaagccattacttccagctggtatacaggataaactacatacactgatcccttgcaagaaatttgatctcagttataatctaaattaccacaagttatgttcagattttcaagaggatattgtatttcgtttttccctgggctggtcttcccttgtacatcgatttttgggccctagaaatgctcaaagggtgctcctaggattatcagagcctatctttcagctccctagatctttagcttctactcccactgctcctaccactccagcaacgccagataatgcatcacaggaagaactcatgattacattagtaacaggattggcgtccgttacatctagaacttctatgggcatcattattgttggaggagtgatttggaaaactataggctggaaactcctatctgtttcattaactatgtatggagctttgtatctttatgaaagactgagctggaccacccatgccaaggagcgagcctttaaacagcagtttgtaaactatgcaactgaaaaactgaggatgattgttagctccacgagtgcaaactgcagtcaccaagtaaaacaacaaatagctaccacttttgctcgcctgtgccaacaagttgatattactcaaaaacagctggaagaagaaattgctagattacccaaagaaatagatcagttggagaaaatacaaaacaattcaaagctcttaagaaataaagctgttcaacttgaaaatgagctggagaattttactaagcagtttctaccttcaagcaatgaagactcctaacaatagagattgctttggtgaccatgataggaggaaacgaaacttgtaagattggaacagttgttatttttatgaaattactttaaatatgaattgtactaactgtacctaaatagcaaagccctgtgtagattctggtaatgatctgtctcagggtatgtgtatttttgaagagtgttatgtccttagttttaattttgagtaaagaaaaggctaaatcatgaattagttacaagcaacagtaccaacttatgtgacccctgaggggtggggctgtgagctcttaatttgtttttgattctgaaaaactctgcttcctggcatccaggagttagagattgagcctttcatcttctttctcaaaactagtttttgatgctttctttcatgggaatagtcacttttttatttagtaaatcgcattgctggaaccaccaaggagtgtggaatgtccttgagtgtattatttatgcaagtcacagtcacgttgccatcatggcagctatgtgaaacactaataaatgtgtttttactttttattcccgttaaaactgatgtaaaacaggataaaggcttgttatagtcacttataagtatctgggtctaagtaatttccttagatgtttctaaagaaacattttcagctttgctcccattatgattccaataaggaacgctttcctagtgcaattttaggagtaaagtttgaagagataaaaatagccaaagataggagacgtctgaattttgaatgataaacagtgatgttttaaaaaagctgttgttcttcaggaggcatttgcctaggatattgctggattataccccattggaggcttttaattttatttgtatgaattttccaggatttcattaaaaattattattgtattttttaccttaaaaaaaaaaaaaaa

SEQ ID NO: 30 - protein, 741 aa, Homo sapiens
MAEPVSPLKHFVLAKKAITAIFDQLLEFVTEGSHFVEATYKNPELDRIATEDDLVEMQGYKDKLSIIGEVLSRRHMKVAFFGRTSSGKSSVINAMLWDKVLPSGIGHITNCFLSVEGTDGDKAYLMTEGSDEKKSVKTVNQLAHALHMDKDLKAGCLVRVFWPKAKCALLRDDLVLVDSPGTDVTTELDSWIDKFCLDADVFVLVANSESTLMNTEKHFFHKVNERLSKPNIFILNNRWDASASEPEYMEDVRRQHMERCLHFLVEELKVVNALEAQNRIFFVSAKEVLSARKQKAQGMPESGVALAEGFHARLQEFQNFEQIFEECISQSAVKTKFEQHTIRAKQILATVKNIMDSVNLAAEDKRHYSVEEREDQIDRLDFIRNQMNLLTLDVKKKIKEVTEEVANKVSCAMTDEICRLSVLVDEFCSEFHPNPDVLKIYKSELNKHIEDGMGRNLADRCTDEVNALVPQTQQEIIENLKPLLPAGIQDKLHTLIPCKKFDLSYNLNYHKLCSDFQEDIVFRFSLGWSSLVHRFLGPRNAQRVLLGLSEPIFQLPRSLASTPTAPTTPATPDNASQEELMITLVTGLASVTSRTSMGIIIVGGVIWKTIGWKLLSVSLTMYGALYLYERLSWTTHAKERAFKQQFVNYATEKLRMIVSSTSANCSHQVKQQIATTFARLCQQVDITQKQLEEEIARLPKEIDQLEKIQNNSKLLRNKAVQLENELENFTKQFLPSSNEDS

SEQ ID NO: 31 - DNA, 2483 bp, Homo sapiens
cacatgttgccccaaatacaagcacaaatctaaccatgagcttcagcaatcagctcaatacagtgcacaatcaggccagtgttctagcttccagttctactgcagcagctgctactctttctctggctaattcagatgtctcactactaaactaccagtcagctttgtacccatcatctgctgcaccagttcctggagttgcccagcagggtgtttccttgcagcctggaaccacccagatttgcactcagacagatccattccaacagacatttatagtatgtccacctgcgtttcaaactggactacaagcaacaacaaagcattctggattccctgtgaggatggataatgctgtaccgattgtaccccaggcaccagctgctcagcccactacagattcagtcaggagttctcacgcagacttgcagggaaaaaatatccagacattcttgagaaatggtcttctgaggaagctgtacaccactaatggtagcaactctccaccctcaagtagccacatcacaccgcagtatgcggtgccctttactctgagctgcgcagccggccggccggcgctggttgaacagactgccgctgtactgcaggcgtggcctggagggactcagcaaattctcctgccttcaacttggcaacagttgcctggggtagctctacacaactctgtccagcccacagcaatgattccagaggccatggggagtggacagcagctagctgactggaggaatgcccactctcatggcaaccagtacagcactatcatgcagcagccatccttgctgactaaccatgtgacattggccactgctcagcctctgaatgttggtgttgcccatgttgtcagacaacaacaatccagttccctcccttcgaagaagaataagcagtcagctccagtctcttccaagtcctctctagatgttctgccttcccaagtctattctctggttgggagcagtcccctccgcaccacatcttcttataattccttggtccctgtccaagatcagcatcagcccatcatcattccagatactcccagccctcctgtgagtgtcatcactatccgaagtgacactgatgaggaagaggacaacaaatacaagcccagtagctctggactgaagccaaggtctaatgtcatcagttatgtcactgtcaatgattctccagactctgactcttctttgagcagcccttattccactgataccctgagtgctctccgaggcaatagtggatccgttttggaggggcctggcagagttgtggcagatggcactggcacccgcactatcattgtgcctccactgaaaactcagcttggtgactgcactgtagcaacccaggcctcaggtctcctgagcaataagactaagccagtcgcttcagtgagtgggcagtcatctggatgctgtatcacccccacagggtatcgagctcaacgcggggggaccagtgcagcacaaccactcaatcttagccagaaccagcagtcatcggcggctccaacctcacaggagagaagcagcaacccagccccccgcaggcagcaggcgtttgtggcccctctctcccaagccccctacaccttccagcatggcagcccgctacactcgacagggcacccacaccttgccccggcccctgctcacctgccaagccaggctcatctgtatacgtatgctgccccgacttctgctgctgcactgggctcaaccagctccattgctcatcttttctccccacagggttcctcaaggcatgctgcagcctataccactcaccctagcactttggtgcaccaggtccctgtcagtgttgggcccagcctcctcacttctgccagcgtggcccctgctcagtaccaacaccagtttgccacccaatcctacattgggtcttcccgaggctcaacaatttacactggatacccgctgagtcctaccaagatcagccagtattcctacttatagttggtgagcatgagggaggaggaatcatggctaccttctcctggccctgcgttcttaatattgggctatggagagatcctcctttaccctcttgaaatttcttagccagcaacttgttctgcaggggcccactgaagcagaaggtttttctctgggggaacctgtctcagtgttgactgcattgttgtagtcttcccaaagtttgccctatttttaaattcattatttttgtgacagtaattttggtacttggaagagttcagatgcccatcttctgcagttaccaaggaagagagattgttctgaagttaccctctgaaaaatattttgtctctctgacttgatttctataaatgcttttaaaaacaagtgaagcccctctttatttcattttgtgttattgtgattgctggtcaggaaaaatgctgatagaaggagttgaaatctgatgacaaaaaaaaaaaaaaaaaaaaaaaaaaaaaa

SEQ ID NO: 32 - protein, 654 aa, Homo sapiens
MSFSNQLNTVHNQASVLASSSTAAAATLSLANSDVSLLNYQSALYPSSAAPVPGVAQQGVSLQPGTTQICTQTDPFQQTFIVCPPAFQTGLQATTKHSGFPVRMDNAVPIVPQAPAAQPTTDSVRSSHADLQGKNIQTFLRNGLLRKLYTTNGSNSPPSSSHITPQYAVPFTLSCAAGRPALVEQTAAVLQAWPGGTQQILLPSTWQQLPGVALHNSVQPTAMIPEAMGSGQQLADWRNAHSHGNQYSTIMQQPSLLTNHVTLATAQPLNVGVAHVVRQQQSSSLPSKKNKQSAPVSSKSSLDVLPSQVYSLVGSSPLRTTSSYNSLVPVQDQHQPIIIPDTPSPPVSVITIRSDTDEEEDNKYKPSSSGLKPRSNVISYVTVNDSPDSDSSLSSPYSTDTLSALRGNSGSVLEGPGRVVADGTGTRTIIVPPLKTQLGDCTVATQASGLLSNKTKPVASVSGQSSGCCITPTGYRAQRGGTSAAQPLNLSQNQQSSAAPTSQERSSNPAPRRQQAFVAPLSQAPYTFQHGSPLHSTGHPHLAPAPAHLPSQAHLYTYAAPTSAAALGSTSSIAHLFSPQGSSRHAAAYTTHPSTLVHQVPVSVGPSLLTSASVAPAQYQHQFATQSYIGSSRGSTIYTGYPLSPTKISQYSYL

SEQ ID NO: 33 - DNA, 2731 bp, Homo sapiens
  unsure (2173)
  unsure (2700)
ggcactccacggctgtgaagatggcggcggctgcgtggcttcaggtgttgcctgtcattcttctgcttctgggagctcacccgtcaccactgtcgtttttcagtgcgggaccggcaaccgtagctgctgccgaccggtccaaatggcacattccgataccgtcggggaaaaattattttagttttggaaagatcctcttcagaaataccactatcttcctgaagtttgatggagaaccttgtgacctgtctttgaatataacctggtatctgaaaagcgctgattgttacaatgaaatctataacttcaaggcagaagaagtagagttgtatttggaaaaacttaaggaaaaaagaggcttgtctgggaaatatcaaacatcatcaaaattgttccagaactgcagtgaactctttaaaacacagaccttttctggagattttatgcatcgactgcctcttttaggagaaaaacaggaggctaaggagaatggaacaaaccttacctttattggagacaaaaccgcaatgcatgaaccattgcaaacttggcaagatgcaccatacatttttattgtacatattggcatttcatcctcaaaggaatcatcaaaagaaaattcactgagtaatctttttaccatgactgttgaagtgaagggtccctatgaatacctcacacttgaagactatcccttgatgatttttttcatggtgatgtgtattgtatatgtcctgtttggtgttctgtggctggcatggtctgcctgctactggagagatctcctgagaattcagttttggattggtgctgtcatcttcctgggaatgcttgagaaagctgtcttctatgcggaatttcagaatatccgacacaaaggagaatctgtccagggtgctttgatccttgcagagctgctttcagcagtgaaacgctcactggctcgaaccctggtcatcatagtcagtctgggatatggcatcgtcaagccacgccttggagtcactcttcataaggttgtagtagcaggagccctctatcttttgttctctggcatggaaggggtcctcagagttactggggcccagactgatcttgcttccttggcctttatccccttggctttcctagacactgccttgtgctggtggatatttattagcctgactcaaacaatgaagctattaaaacttcggaggaacattgtaaaactctctttgtatcggcatttcaccaacacgcttattttggcagtggcagcatccattgtgtttatcatctggacaaccatgaagttcagaatagtgacatgtcagtcggtgagttataagcacatttatgaataatgtactgtcttataaacaactgatggtgttgatgacagtggtaaggttcttctaagttatataccttataaaaaattagagctaggtctctactctgagggttgtgatacttccctcctcctaagtattctgtactatcatggtgcttggtatagtacttttttgtttgttttttctgactgtattctcccagttttgggagagaattttgtaagttataactacagtgtgctataacccagtcttattttaactaaaaatcttaagaagtccagagtactaaatattaagtaccatatgtgtaaataatactaatctgaatagaagccacatccttaagatctgagctcaacgactgtgacagtaggatttcttcagaagcagctaaggctcttattttgttcaataaataatgaaaatgaaaattataaagtataccaacctaatgtaactttctcttacactgtataaggtaactttctcttaaccctgtataaaaccctttcttaaagcttctcagagggatgatgaagctttgacaaatactctgttccgttgatgcattttctttaacaacagtaagcactacaagggcaaaaactacattcattcactttgtttccccacacttaccacagtactgagcacgtagcaggctcttagtaaacataacttgaatgaacaaataagtgatttttgttgtatgccaaaggctttatgaacaaggggttaagataatgtgtatgaatgttgtacttctcccctgtattgtaggactggcgggagctgtgggtagacgatgccatctggcgcttgctgttctccangatcctctttgtcatcatggttctctggcgaccatctgcaaacaaccagaggttcttggactcttctgtttactctgctaacatgagatgaccatgtcatcaattaggggtggtgcattgggggacagtatcagggctgtgtcatatagtggaaggaacactgggcctggaatcagaagaactgggttcctatctcagctctcctcttaacttcatgatttttggcatgcggcctctccacctctctggccttagtttcctttctatatactgagggggaattaaacccagcaacatgaagttcctttcagctctgacattttgtgataaatacacaggcatactatggaaataaattgcaagtttggtttcagaccatcacgataaagcagatattgagttacatacatattttgttttttccagtgcatatcaaagttatgtttacactattctgtagtctactatgtgtgcaatagcattatgtctaaaaaatatatgcacgttaatttaaaaacactttgttnccaaaaaaaaaaaaaaaaaaaaaaaaaaaaa

SEQ ID NO: 34 - protein, 441 aa, Homo sapiens
MAAAAWLQVLPVILLLLGAHPSPLSFFSAGPATVAAADRSKWHIPIPSGKNYFSFGKILFRNTTIFLKFDGEPCDLSLNITWYLKSADCYNEIYNFKAEEVELYLEKLKEKRGLSGKYQTSSKLFQNCSELFKTQTFSGDFMHRLPLLGEKQEAKENGTNLTFIGDKTAMHEPLQTWQDAPYIFIVHIGISSSKESSKENSLSNLFTMTVEVKGPYEYLTLEDYPLMIFFMVMCIVYVLFGVLWLAWSACYWRDLLRIQFWIGAVIFLGMLEKAVFYAEFQNIRHKGESVQGALILAELLSAVKRSLARTLVIIVSLGYGIVKPRLGVTLHKVVVAGALYLLFSGMEGVLRVTGAQTDLASLAFIPLAFLDTALCWWIFISLTQTMKLLKLRRNIVKLSLYRHFTNTLILAVAASIVFIIWTTMKFRIVTCQSVSYKHIYE

SEQ ID NO: 35 - DNA, 1670 bp, Homo sapiens
aatcgggctcacccccaagttgggcgggtcattgacaagtcgaagagttgggtccttgtgtatgcatgggtgggatggtaagggaagaagccctggcctggatgtgccgggaaccccggaaagccttctcagccattgttgggcctagcctgggacccgacagcactcctgggtgggggactggggagtgggcaacaggtggagccatccttggcagaccgaccccatgtgcagtccctgggacaggtttctccctcctgagcacttgtagctcccctcgagggccagttccagagacaggccgagggtggcgagtccccaccccatgctctcttccagacctcctacgagatgatgatgcagtgtgtgtcccgcatgttggcccaccccctgcatgtcatctcaatgcgctgcatggtccagtttgtgggacgggaggccaagtacagtggtgtgctgagctccattgggaagattttcaaagaggaagggctgctgggattcttcgttggattaatccctcacctcctgggcgatgtggttttcttgtggggctgtaacctgctggcccacttcatcaatgcctacctggtggatgacagcttcagccaggccctggccatccggagctataccaagttcgtgatggggattgcagtgagcatgctgacctaccccttcctgctagttggcgacctcatggctgtgaacaactgcgggctgcaagctgggctccccccttactccccagtgttcaaatcctggattcactgctggaagtacctgagtgtgcagggccagctcttccgaggctccagcctgcttttccgccgggtgtcatcaggatcatgctttgccctggagtaacctgaatcatctaaaaaacacggtctcaacctggccaccgtgggtgaggcctgaccaccttgggacacctgcaagacgactccaacccaacaacaaccagatgtgctccagcccagccgggcttcagttccatatttgccatgtgtctgtccagatgtggggttgagcgggggtggggctgcacccagtggattgggtcacccggcagacctagggaaggtgaggcgaggtggggagttggcagaatccccatacctcgcagatttgctgagtctgtcttgtgcagagggccagagaatggcttatgggggcccaggttggatggggaaaggctaatggggtcagaccccaccccgtctacccctccagtcagcccagcgcccatcctgcagctcagctgggagcatcattctcctgctttgtacatagggtgtggtcccctggcacgtggccaccatcatgtctaggcctatgctaggaggcaaatggccaggctctgcctgtgtttttctcaacactacttttctgatatgagggcagcacctgcctctgaatgggaaatcatgcaactactcagaatgtgtcctcctcatctaatgctcatctgtttaatggtgatgcctcgcgtacaggatctggttacctgtgcagttgtgaatacccagaggttgggcagatcagtgtctctagtcctacccagttttaaagttcatggtaagatttgacctcatctcccgcaaataaatgtattggtgatttggaaaaaaaaaaaaaaaaaaaaaaaaaaaaaaaaaaaaaaaaaa

SEQ ID NO: 36 - protein, 164 aa, Homo sapiens
MGGMVREEALAWMCREPRKAFSAIVGPSLGPDSTPGWGTGEWATGGAILGRPTPCAVPGTGFSLLSTCSSPRGPVPETGRGWRVPTPCSLPDLLRDDDAVCVPHVGPPPACHLNALHGPVCGTGGQVQWCAELHWEDFQRGRAAGILRWINPSPPGRCGFLVGL

SEQ ID NO: 37 - DNA, 1493 bp, Homo sapiens
  unsure (1415)
ggatggcgcgcgcggggcccgcacgtggaggccggcgcgggggcgcgggcagggccggctgctgagacgcgctgctgccccccgcgcgggcgccgcggcttcaatggcgccatcgcccaggaccggcagccggcaagatgcgaccgccctgcccagcatgtcctcaactttctgggcgttcatgatcctggccagcctgctcatcgcctactgcagtcagctggccgccggcacctgtgagattgtgaccttggaccgggacagcagccagcctcggaggacgatcgcccggcagaccgcccgctgtgcgtgtagaaaggggcagatcgccggcaccacgagagcccggcccgcctgtgtggacgcaagaatcatcaagaccaagcagtggtgtgacatgcttccgtgtctggagggggaaggctgcgacttgttaatcaaccggtcaggctggacgtgcacgcagcccggcgggaggataaagaccaccacggtctcctgacaaacacagcccctgaggggccccgggagtggccttggctccctggagagcccacgtctcagccacagttctccactcgcctcggacttcacccgttctctgccgcccgcccactccgtttccctgtggtccgtgaaggacggcctcaggccttggcatcctgagcttctgtctgtccagccgacccgaggaggccggactcagacacataggcggggggcggcacctggcatcagcaatacgcagtctgtgggagcccggccgcgcccagcccccgccgaccgtggcgttggccctgctgtcctcagaggaggaggaggaggaggcagctccggcagccacagaaggctgcagcccagcccgcctgagacacgacgcctgccccaggggactgtcaggcacagaagcggcctcctcccgtgccccagactgtccgaattggttttattttcttatactttcagtatactccatagaccaaagagcaaaatctatctgaacctggacgcaccctcactgtcagggtccctggggtcgcttgtgcgggcgggagggcaatggtggcagagacatgctgtggccccggcggagcggagagggcggccgtggtggaggcctccaccccaggagcaccccgcgcaccctcggaggacgggtttcggctgcgcggaggccgtggcacacctgcgggaggcagcgacggcccccacgcagacgccgggaacgcaggccgctttattcctctgtacttagatcaacttgaccgtactaaaatccctttctgttttaaccagttaaacatgcctcttctacagctccatttttgatagttggataatccagtatctgccaagagcatgttgggtctcccgtgactgctgcctcatcgataccccatttagctccagaaagnaaagaaaactcgagtaacacttgtttgaaagagatcattaaatgtattttgcaaagcctaaaaaaaaaaaaaaaaaaa

SEQ ID NO: 38 - protein, 132 aa, Homo sapiens
MAPSPRTGSRQDATALPSMSSTFWAFMILASLLIAYCSQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 39 - DNA, 3693 bp, Homo sapiens
  unsure (108)
cgtggccgaaggatgcccgtttgtgtctaaacggaggctcggccacaacgcccctggattggtggtagggcggggcgggccacagtctccaccctgaagcggaagtgnaggaaagatggaggaccatcaccacgtgcccatcgacatccagaccagccagctgctcgattggctggtggacagaaggcactgcagcctgaaatggcagagtctggtgctgacgatccgcgagaagatcaatgctgccatccaggacatgccagagagcgaagagatcgcccagctgctgtctgggtcctgtgagtgcttaggggctgtcactggagtcccctctttgctgaggtagtatgtatcagctgagctactctcttgttatttagccaccagggctggcctttagagagggagcgatttttatttgaggtatagatgctggcttcttccactatgaaatgatttagggaaaagctacacactaaagttactggcagtatattatacatgaatcagccacctgcagtgcccagagtaggtgaagaagatactttagttctcgaaatcccgtctcttgctttctagacattcactactttcactgcctaagaatcctggaccttctcaaaggcacagaggcctccacgaagaatatttttggccgatactcttcacagcggatgaaggattggcaggagattatagctctgtatgagaaggacaacacctacttaggtaaagtggcccggcctgggagccctggtatccatggggaagcccactctcagagttctgagataccaggcttataggaggcacagtctgtgagtgggaagagactggagtgtagatgttgcccatttgtaggtggtaaaatcaattgtttttgatggaattgattttccctgagtggagtgctgggggaaggaggaggtccaggccggtagtggccattcgccgtgcctcagcgagcaggtgtgtgtgggtcctccaccactcacctcttggttagcgggagtgtgctgcccccacccccacccccgtacccccattgtacacaaggcagaagaggcacgggttttcctgggagcgaatatcaagtgcctgagagcaactacaggactaactgtgtttgggttgggtgtagtataaataataataatggctaatatttcctgagcatctactaaatgcaaggaattgtgcttggtgtgtcatgtggattctctcttgcatcttcatgataaatgttattgtcgctgttttaccgatgagggttggattagaggggttaaacaacttgtcttaggctccacagctgggaacaagtggggctgggaagctgacttcgtgctcttcaccaccacaaaggatgtgtgtgcatcctggggcatgcctgcctcatgtgggggtgtcctgggctgaatttcctgggcacttctcagtggaactctctagcctcctggttcggaatgtcaactatgagatcccctcactgaagaagcagattgccaagtgccagcagctgcagcaagaatacagccgcaaggaggaggagtgccaggcaggggctgccgagatgcgggagcagttctaccactcctgcaagcagtatggcatcacggtgagcggcggcagcctcttcgcagccagaggacacctgggcccctgcttgtcttcctctgaccccgtctgacccctcagcctggttgcgccccctttgggccagtgtcttacttttcttcggtctttggatgttttcttcaatctgttggactccacctcttctcccctctctagggcgaaaatgtccgaggagaactgctggccctggtgaaggacctgccgagtcagctggctgagattggggcagcggctcagcagtccctgggggaagccattgacgtgtaccaggcgtctgtggggtttgtgtgtgagaggtagagaggcctcagcttctcctggtgggggtgctttgcctgtgttccccagctcatgacccttctccagttgtcttgttcccatataacatttgaactctttacacacctgaacctgtgggggccttgcccatttgaccatgtggcccaggccaaagcccagtgttggccttacgcatggtcggcaggagagtcagttgtgtgctctgttgaagccccacagagcaggtgttgccaatgctgcggttcgtgcagaagcggggaaactcaacggtgtacgagtggaggacagggacagagccctctgtggtggaacgaccccacctcgaggagcttcctgagcaggtggcagaagatgcgattgactggggcgactttggggtagaggcagtgtctgaggggactgactctggcatctctgccgaggctgctggaatcgactggggcatcttcccggaatcagattcaaaggatcctggaggtgatgggatagactggggagacgatgctgttgctttgcagatcacagtgctggaagcaggaacccaggctccagaaggtgttgccaggggcccagatgccctgacactgcttgaatacactgagacccggaatcagttccttgatgagctcatggagcttgagatcttcttagcccagagagcagtggagttgagtgaggaggcagatgtcctgtctgtgagccagttccagctggctccagccatcctgcagggccagaccaaagagaagatggttaccatggtgtcagtgctggaggatctgattggcaagcttaccagtcttcagctgcaacacctgtttatgatcctggcctcaccaaggtctggcttccccttgatgcaaggctctgccatcttgagcagctctgcctccttgtattcctcctcttgttccatgaccccttaaaccccatccctgcctcctggccattgccatccactggggataggggttctctttgggacaagagggggaggtttcacatatacaggaagaatctgcttgcttcctgagtaggacaggggaactgggagtgggttttccttaaaaggaaagggtttaaggatgtgagggtaagcggccagttgggggtttgtttcccgagcctctcacctccccagcagctgaatgggaatgctcaggatgcacagctaacccagcactcacctgagtgccccgcacaggtatgtggaccgagtgactgaattcctccagcaaaagctgaagcagtcccagctgctggctttgaagaaagagctgatggtgcagaagcagcaggaggcacttgaggagcaggcggctctggagcctaagctggacctgctactggagaagaccaaggagctgcagaagctgattgaagctgacatctccaagaggtacagcgggcgccctgtgaacctgatgggaacctctctgtgacaccctccgtgttcttgcctgcccatcttctccgcttttgggatgaagatgatagccagggctgttgttttggggcccttcaaggcaaaagaccaggctgactggaagatggaaagccacaggaaggaagcggcacctgatggtgatcttggcactctccatgttctctacaagaagctgtggtgattggccctgtggtctaccaggcgaaaaccacagattctccttctagttagtatagcggacttaataaaagaggaaaaaactcttgcttcaaaaaaaaaaaaaaaaaaaaaaaaaaaaaaaaaaaaaa

SEQ ID NO: 40 - protein, 230 aa, Homo sapiens
MLRFVQKRGNSTVYEWRTGTEPSVVERPHLEELPEQVAEDAIDWGDFGVEAVSEGTDSGISAEAAGIDWGIFPESDSKDPGGDGIDWGDDAVALQITVLEAGTQAPEGVARGPDALTLLEYTETRNQFLDELMELEIFLAQRAVELSEEADVLSVSQFQLAPAILQGQTKEKMVTMVSVLEDLIGKLTSLQLQHLFMILASPRSGFPLMQGSAILSSSASLYSSSCSMTP

SEQ ID NO: 41 - DNA, 1701 bp, Homo sapiens
cccttgagatgattttctcttttcaacttcttgaacttggacatgaaggatgtgggcccagaatcatgtggccagcccaccccctgttggccctcaccagccttggagtctgttctagggaaggcctcccagcatctgggactcgagagtgggcagcccctctacctcctggagctgaactggggtggaactgagtgtgttcttagctctaccgggaggacagctgcctgtttcctccccaccagcctcctccccacatccccagctgcctggctgggtcctgaagccctctgtctacctgggagaccagggaccacaggccttagggatacagggggtccccttctgttaccaccccccaccctcctccaggacaccactaggtggtgctggatgcttgttctttggccagccaaggttcacggcgattctccccatgggatcttgagggaccaagctgctgggattgggaaggagtttcaccctgaccgttgccctagccaggttcccaggaggcctcaccatactccctttcagggccagggctccagcaagcccagggcaaggatcctgtgctgctgtctggttgagagcctgccaccgtgtgtcgggagtgtgggccaggctgagtgcataggtgacagggccgtgagcatgggcctgggtgtgtgtgagctcaggcctaggtgcgcagtgtggagacgggtgttgtcggggaagaggtgtggcttcaaagtgtgtgtgtgcagggggtgggtgtgttagcgtgggttaggggaacgtgtgtgcgcgtgctggtgggcatgtgagatgagtgactgccggtgaatgtgtccacagttgagaggttggagcaggatgagggaatcctgtcaccatcaataatcacttgtggagcgccagctctgcccaagacgccacctgggcggacagccaggagctctccatggccaggctgcctgtgtgcatgttccctgtctggtgcccctttgcccgcctcctgcaaacctcacagggtccccacacaacagtgccctccagaagcagcccctcggaggcagaggaaggaaaatggggatggctggggctctctccatcctccttttctccttgccttcgcatggctggccttcccctccaaaacctccattcccctgctgccagcccctttgccatagcctgattttggggaggaggaaggggcgatttgagggagaaggggagaaagcttatggctgggtctggtttcttcccttcccagagggtcttactgttccagggtggccccagggcaggcaggggccacactatgcctgcgccctggtaaaggtgacccctgccatttaccagcagccctggcatgttcctgccccacaggaatagaatggagggagctccagaaactttccatcccaaaggcagtctccgtggttgaagcagactggatttttgctctgcccctgaccccttgtccctctttgagggaggggagctatgctaggactccaacctcagggactcgggtggcctgcgctagcttcttttgatactgaaaacttttaaggtgggagggtggcaagggatgtgcttaataaatcaattccaagcctcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - protein, 240 aa, Homo sapiens [sequence omitted]

SEQ ID NO: 43 - DNA, 1784 bp, Homo sapiens
aggtctagaattcaatcgggaatatcttttaagttttaaaaaaactggaataattatatctatcttttttgccgtttatatttaggggtttttgttgataaaatcaagtcttggttgtggcttgctgaattaaatatttatgagtggtgcatttttaagtatagtgaacaagacaccatattaagtacagtgataaagcatctatattctgtaaaaaaaaaaaaaatctgcctatgcatgttttttaagaaaaaaaaaatggctgtatcggcctgtatgggactgtaatgcgcttagtggtctgacatatactggaaatgtatgtatactggcgtactttatattctctaaaatgcttaatgcctttgaaattttgtaatcaaaaaaaagctttgaaaaatctaaaggggagagtattctttaaagtttttaacataagcttgtcaatgcacatgtagatggttagcatgtttagcaaaccttgtgaaattataataagtttgtagttacatgtgaaactctaaatgcatggcaactgttaatgtcataacagtttagttattttgttctgttctgtcatgtgccacaaaatatgtacttttttcacttttttccctttgtatatcagttacgggttacaactggttcattctgaaaacaacaacaacaaaagtccattcatattttttaacaattgtataagtgcccaagtaattcactacagcctaaagccttgcctttgtaatttgacttctgacatgttggcaatcaaagcatgcacttgtaacaatgaaaaagaaaaagcattttatattactactcaataaaatgtgcatgaacttacagaattctcatccttccactgagtccgctgaagggatttatgtgcacaaccaccatgtgtcttctaggtgctggcccaccaccacacatcacaggctgatttccacaggcttcttcctaggggcctcgtgatctgaggggtggtgcctacttccactgtaagaaagaatcttggtggatttgtgtctcaaatcagataagagaagcctgtttaaagagcagatgccatcttctggcttcctcaaggagccagttaaaaaaccagagcattcctttttattgaaaaataaaattaatttgttatcaggttgtttcagttgtattggatgccctatctatctgctaaagcaaaaagtactaggctactaagtgcattttcatcacagaaaagagttgcatttgtattaacaagaaatttgtatacccacgcttcagctactatctaatcatcacccgaagatttaagatacaccaaatttcagtttgtttgtaacattgttcatctttagtgcactttgttttatataataaagtatgcctgttatattaaataataagaatatggcaattagcgatatagcatacccaaacaaagatgttctcgatacagtctggcaaagactatcccaaggttattttaatgaattcagacattttttcctgtggatatttctccatcctaaaaaaagtggcaaccaaggaaaatatttagatgcaacttactagagtgatgatgtgaaagaaatggtgattctggtatcatggtgtttattttctttcttataactgcagagaaaatatcctgactaaaaaaaattcatttttttggattcctttcttttacaaattgtgctgaggcaactatggcatagaaataaacatttgacattaaaataaaaaaaaaaaaaaaaaaaaaaaaaaaaaaaaaaaaaaaaaaaaaa

SEQ ID NO: 44 - protein, 82 aa, Homo sapiens
MCHKICTFFTFFPLYISYGLQLVHSENNNNKSPFIFFNNCISAQVIHYSLKPCLCNLTSDMLAIKACTCNNEKEKAFYITTQ

SEQ ID NO: 45 - DNA, 1034 bp, Homo sapiens
  unsure (598)
ggaagatggcggcctctggggcggagccgcaggtcctggtacaatacttggtgttacgaaaggatctatcacaagctccgttctcctggccggcgggcgcactggtagcgcaggcttgtcacgcggccaccgcggccttgcacactcaccgcgaccacccgcacacagccgcttacctccaagagctggggcgcatgcgcaaagtggtcctcgaggccccagatgagaccaccctaaaggagctggccgagaccctgcaacagaagaacattgaccacatgctgtggcttgagcaaccagagaatatcgccacttgtattgctctccggccctaccccaaggaagaagtgggccagtatttgaagaagttccgattgttcaagtaactgctgctttgatgtgtttgaatacgcaggccacccattccaaagcatcatgtgttccttgcagtgtcagcttgctcccgtctttcagttgtgacaatttcttgagggttaagcacatgttcatattaaagttgtcattaataactacttcctcttattaataagttcaagtggggaaggtgggagagcagtattgtctggggatcattgcncaaatagaagatttggttagactctcctgtggggctcaaggaaactcccttccagtcactcgggtttgaaactttgcttttgaattccttcttattcacatccagttatcatatttcattgaatccaagataacctcaacttcaagatgcggtagtattttatgtattgttaaaaaatatgccggcaaattaaacacttgtatttcaataacaaagatgttaaaatttggccagtgtggtggctcacatctgttaattccagggttttgggaagccaaggcaggaggatcgcttgagcccatgagttcaaggttacagtcagttctaatcaggccaccgcactccagcgtgggcaacagagtgagacacggtttctataaagattaataacaagttaaaaaaaaaaaaaaaaaaaaaaaaaaaaaaaaaaaaaaaa

SEQ ID NO: 46 - protein, 126 aa, Homo sapiens [sequence omitted]

SEQ ID NO: 47 - DNA, 1626 bp, Homo sapiens
caacttgtgtagctgaaggtttgtttgtgacttattacagagcctgtgacttaaaaatccttcccacaaccacaagctaaagtgggagaagacaaactacctcaccttttcaaccaagagggaggagcaaaaatcagtgaacttttacagaagaacctgccagcctgtgatgatcctaccaaagagaaacctcaatgagttatggaatttcctttttggtgaattgagtgctgtttttgcttttctcagattccaaatgagagtatacatttttctttgtttgatgtgctgggtgagatctgataataaaagaccatgccttgaattctctcagctaagtgtaaaggattccttcagagatttatttattccgagaatagagaccattctgatgatgtatacaaggaacaacctaaactgtgctgagccactgtttgaacaaaataactcacttaatgttaatttcaacacacaaaagaaaacagtctggcttattcacggatacagaccagtaggctccatcccattatggcttcagaacttcgtaaggattttgctgaatgaagaagatatgaatgtaattgtagtagactggagccggggtgctacaacttttatttataatagagcagttaaaaacaccagaaaagttgctgtgagtttgagtgtgcacattaaaaatcttttgaagcatggtgcatctcttgacaattttcatttcataggtgtgagtttaggggctcatatcagtggatttgttggaaagatatttcatggtcaacttggaagaataacaggtcttgaccctgctgggccaaggttctccagaaaaccaccatatagcagattagattacacggatgcaaagtttgtggatgtcatccattctgactccaatggaattcaattcattaaatgcaaccaccagagagcagttcacttgttcatggcatctttagaaacaaactgcaattttatttcatttccttgtcgttcatacaaagattacaagactagcttatgtgtggactgtgactgttttaaggaaaaatcatgtcctcggctgggttatcaagccaagctatttaaaggtgttttaaaagaaaggatggaaggaagacctcttaggaccactgtgtttttggatacaagtgcctattattttgttctcagtataattgttccagataaaactatgatggatggctcgttttcatttaaattattaaatcagcttggaatgattgaagagccaaggctttatgaagaaagataacatatgttaaagaggcacccttactctaaacaactagtgactttaaaagttctaagcgtatcaggagatggagaccatcctggctaacatggtgaaaccctgtctctactaaaaattcagaaaattagctgggcatggtggcacgtgcctgtagtcccagctactcaggaggctgaggcaagagaattgcttgaacccaggaggtggaggttgcagtgagctgagattgcaccgctgccctccagcctgggtgacagagcaagactccatttcaaataaataaataaataaataaataaataaataaataaataaataaaataagttaaagagtaaaaaaaaaaaaaaaa

SEQ ID NO: 48 - protein, 368 aa, Homo sapiens
MILPKRNLNELWNFLFGELSAVFAFLRFQMRVYIFLCLMCWVRSDNKRPCLEFSQLSVKDSFRDLFIPRIETILMMYTRNNLNCAEPLFEQNNSLNVNFNTQKKTVWLIHGYRPVGSIPLWLQNFVRILLNEEDMNVIVVDWSRGATTFIYNRAVKNTRKVAVSLSVHIKNLLKHGASLDNFHFIGVSLGAHISGFVGKIFHGQLGRITGLDPAGPRFSRKPPYSRLDYTDAKFVDVIHSDSNGIQFIKCNHQRAVHLFMASLETNCNFISFPCRSYKDYKTSLCVDCDCFKEKSCPRLGYQAKLFKGVLKERMEGRPLRTTVFLDTSAYYFVLSIIVPDKTMMDGSFSFKLLNQLGMIEEPRLYEER

SEQ ID NO: 49 - DNA, 1221 bp, Homo sapiens
ggaaaagctgagaataatcacctctgataaagatcacagaagctgcccgggaggtgtttgattaaattcatgtattgaaaatattgttcagaccccatgtgacataactggagccagtgcagtgccatgaagaactacgagattagcctggatattaacttgtcttctagagaatagatttcatgttccattcttctgcaatggttaattcacacagaaaaccaatgtttaacattcacagaggattttactgcttaacagccatcttgccccaaatatgcatttgttctcagttctcagtgccatctagttatcacttcactgaggatcctggggctttcccagtagccactaatggggaacgatttccttggcaggagctaaggctccccagtgtggtcattcctctccattatgacctctttgtccaccccaatctcacctctctggactttgttgcatctgagaagatcgaagtcttggtcagcaatgctacccagtttatcatcttgcacagcaaagatcttgaaatcacgaatgccacccttcagtcagaggaagattcaagatacatgaaaccaggaaaagaactgaaagttttgagttaccctgctcatgaacaaattgcactgctggttccagagaaacttacgcctcacctgaaatactatgtggctatggacttccaagccaagttaggtgatggctttgaagggttttataaaagcacatacagaactcttggtggtgaaacaagaattcttgcagtaacagattttgagccaacccaggcacgcatggctttcccttgctttgatgaaccgttgttcaaagccaacttttcaatcaagatacgaagagagagcaggcatattgcactatccaacatgccaaaggtgtccatctatgcatccccagacaaacggaatcaaacacattatgctttgcaggcatcactgaagctacttgatttttatgaaaagtactttgatatctactatccactctccaaactgggtatgttcaaattccacattattgtcttcatttttgctcataaaacttgcttagatctcttccctctttctctttgtatgtgatttaaatgagcactgaggaattcagttagctcaggaaaaaataatttgttcctcagagatgattcttgagtgtagaaaataaaatatttatgacatgccccaaaaaaaaaaaaaaaaaaaa

SEQ ID NO: 50 - protein, 305 aa, Homo sapiens
MFHSSAMVNSHRKPMFNIHRGFYCLTAILPQICICSQFSVPSSYHFTEDPGAFPVATNGERFPWQELRLPSVVIPLHYDLFVHPNLTSLDFVASEKIEVLVSNATQFIILHSKDLEITNATLQSEEDSRYMKPGKELKVLSYPAHEQIALLVPEKLTPHLKYYVAMDFQAKLGDGFEGFYKSTYRTLGGETRILAVTDFEPTQARMAFPCFDEPLFKANFSIKIRRESRHIALSNMPKVSIYASPDKRNQTHYALQASLKLLDFYEKYFDIYYPLSKLGMFKFHIIVFIFAHKTCLDLFPLSLCM

SEQ ID NO: 51 - DNA, 951 bp, Homo sapiens
ggtgggtgcggagtctgcggccgttcccgcggcctcctcctcctccccgttcccttcacccccaccccgcacccctttccccatcccggctccgtcaccctcccgtcccccacactcaggacaagaatgccctgcccggaacaacccagcagcgcctagatggctttggtcacggtccagcggtcacctacccccagcaccacctccagcccctgcgcctcggaggcagacagtggggaggaagaatgccggtcacagcccaggagcatcagcgagagctttctaactgtcaaaggtgctgccctttttctaccacggggaaatggctcatccacaccaagaatcagccacagacggaacaagcatgcaggcgatctccaacagcatctccaagcaatgttcattttactccgcccagaagacaacatcaggctggctgtaagactggaaagtacttaccagaatcgaacacgctatatggtagtggtttcaactaatggtagacaagacactgaagaaagcatcgtcctaggaatggatttctcctctaatgacagcacttgtaccatgggcttagttttgcctctctggagcgacacgctaattcatttggatggtgatggtgggttcagtgtatcgacggataacagagttcacatattcaaacctgtatctgtgcaggcaatgtgggttgacagggattcaaggaacaaacactgtgatgtactattggtggaagaatgaactggagcagcctttctggagagtgatttgccaatatgccttatcattttgcatgatctttgtcctagtaactctatttctatggatttactctaagtttgtaaacatggatgtgtgcaaagattttagctctaagaatgtttgtcagtgttctaataatagcaaaaaataaaaaacaaatgattgaaaaataaaaaaaaaaaaaaa

SEQ ID NO: 52 - protein, 194 aa, Homo sapiens
MALVTVQRSPTPSTTSSPCASEADSGEEECRSQPRSISESFLTVKGAALFLPRGNGSSTPRISHRRNKHAGDLQQHLQAMFILLRPEDNIRLAVRLESTYQNRTRYMVVVSTNGRQDTEESIVLGMDFSSNDSTCTMGLVLPLWSDTLIHLDGDGGFSVSTDNRVHIFKPVSVQAMWVDRDSRNKHCDVLLVEE

SEQ ID NO: 53 - DNA, 1514 bp, Homo sapiens
gcatgatatttttacggttcacccatattgcatgtatcaggaatataatcctttttattattgagtagtgttctattgtatgtatataccacagtttatttctcccttcatcctttgctagattttggggttttttcacattgcgctattcagtataaacctgctctcaacattcatgtgcaagtctttgagtggacatatatttgcgtttctcttgagtgaatgcaccttgttgggtcacgtggcttaacttaaaaaaattttaatcactgtggtgcatatgtagtgattattagtgattatctcataattttattttcttgtttaatgatgttgagtgtatttcatttgtattttagtttgcaaatgtttgttcaaattcttcacctgtttttaatgaagacgtacgacttatttttgtgttctgaacataagttctttgtcacataaaatgtgctatgaatgttgagttttaaatactccaaatgaatggctagagaattactatttgtagaaatatttatatgtcaaagggatgctaacaatttactttattgctctaaaatagaaaagttgccagaatgctgtggagttttagtggaaaacatgatagctggtgttactgagtaaatttgagtgttaaatgtcaatgtaagctaacggccaagatagggaccactgcagggtggttacttgcagctgtgactcaactggtccttcactgccaaacatacctggggttggatcattggcctgacgtttgcaaattgaggaaccttagggcaaatcagtgaacttctgaactgccttcgtcttcagttatatggggatttccccacttttgagatccttgtaaggattatatgagatgaagagatgagacaaggtatataaaagtcctagcacagagcgtgtcatataatatggcttcacaagtaccctcatctcctttccagtcgttttttgtttttgtttttgtttttttgagaccatctcactctgttgcccaggctggagtgcctcttcatttttatttctttattcagcaagtattgatcaaatgtgctttgtaccaggtactgagctcttcgttgggatataatggtgatcaaggagattgtagattctggcagggaaaactgacatcaaacacggcgacccgacatagtgagaccctgtctctactagaagaactttaaaaatcacctaggtgtgggccgggcacggtggctaacgcctgtggtcccagcactttgggatgctgaggcgggtggatcacggggtcaggagatcgagaccatcctggataacacggagaaaccccgtctctactggaaatacaaggaaattggccgggcgtgggggcgggcatctgtggtcccaattactcgggaggctgcagcaggagagtggcatgaacccgggaggcggatcttgcattgagccgagatcacgccactgcactccagcctgggcgacagaatgagactccatctcaaaaaaaaaaaaaaaaa

SEQ ID NO: 54 - protein, 91 aa, Homo sapiens [sequence omitted]

SEQ ID NO: 55 - DNA, 1417 bp, Homo sapiens
gtccaaatcctattgtccacagtcagacttctacaacctcctctgaacaaatgcagcctccaatgtttcactctcaaagtaccattgctgtgttacagggctcttcagttcctcaagaccagcagtcaaccaacatatttctttcccagagtcccatgaataatcttcagactaacacagtagcccaagaagcattttttgcagcaccgaactcaatttctccacttcagtcaacatcaaacagtgaacaacaagctgctttccaacagcaagctccaatatcacacatccagactcctatgctttcccaagaacaggcacaacccccgcagcagggtttatttcagcctcaggtggccctgggctcccttccacctaatccaatgcctcaaagccaacaaggaaccatgttccagtcacagcactcaatagttgccatgcagagtaactctccatcccaggaacagcagcagcagcagcaacagcagcagcaacagcagcagcaacaacaacagagcattttattcagtaatcagaataccatggctacaatggcgtctccaaagcaaccaccaccaaacatgatattcaacccaaatcaaaatccaatggctaatcaggagcaacagaaccagtcaatttttcaccaacaaagtaacatggccccaatgaatcaagagcaacagcccatgcaatttcagagtcagtccacagtttcctcacttcagaacccaggtcctacccagtcggaatcatcacagacccccttgttccatagctctcctcagattcagttggtacaagggtcacctagttctcaagagcagcaagtaactctcttcttatctccagcatccatgtctgccttgcagaccagtataaatcaacaagatatgcaacagtctcctctttattcccctcagaacaacatgcctggaattcaaggagccacattttcgcctcaaccacaggctactttatttcacaacacagcaggaggcacaatgaaccaactgcagaattctcctggctcatctcagcagacatcaggaatgttcttatttggcattcaaaataactgtagtcagcttttaacctctggaccagctacattgcctgatcagttgatggccataagtcagccaggccaaccacaaaacgagggccagccacctgtgacaacacttctttctcagcaaatgccagagaattctccactggcatcctctataaacaccaaccagaacatcgaaaagattgatttgcttgtttcattgcaaaaccaagggaacaacttgactggctccttttaactggatataaattccacgaagaaaatcctgattccaagatgtcctgagatcttgtggttccatgagaattattactttaaaaacaaaacaaaaaaaaaaaaaaa

SEQ ID NO: 56 - protein, 420 aa, Homo sapiens
MQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATFSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 57 - DNA, 2297 bp, Homo sapiens
gaagtgagggttgaatgatccccacttaactaaaaaatgaataagcgtacttgaaatgattttttaaagtgtttggtagtctatacttatgttctttctttgtttccactatagacagtattcgtggctactttggggaaacaattgctctgtactttggatttttggagtatttcacttttgcattaatccccatggctgtcattgggttaccttactacttgtttgtgtgggaagactatgacaagtacgtgatctttgcctcgttcaacctcatctggtccacggtgattctggaactgtggaagcgtggctgtgccaacatgacctacaggtgggggacactgctcatgaagagaaagtttgaggagccccggccaggatttcatggtgtcttgggtatcaattccatcactgggaaggaggagcctctgtaccccagctacaagagacagttgcgcatttacctggtctccctgccattcgtgtgcctctgcctctatttctcactgtatgtcatgatgatttacttcgacatggaggtttgggccttgggtctacatgagaacagcgggtctgagtggaccagtgtcctgttgtatgtgcccagcatcatctatgccattgtgattgagatcatgaatcgtctctatcgatatgctgccgagtttttaacttcatgggagaatcacagattggaatctgcctatcagaaccatctaattctgaaagttttagtgttcaacttcctcaattgctttgcctcactcttctatattgcctttgtcttgaaagatatgaagcttttgcgccagagcttggccactctcctaattacctcccagatcctcaaccaaattatggaatcttttcttccttattggctccaaaggaagcatggtgtgcaggtgaagaggaaggtgcaggctttaaaggcagacattgatgctacattatatgaacaagtcatcctggaaaaagaaatgggaacttatttgggcacctttgatgattacttggagttattcctgcagtttggttatgtgagccttttctcctgtgtttacccattagcagctgcctttgctgtgttaaataacttcactgaagtaaattcagatgccttaaaaatgtgcagggtcttcaaacgtccattctcagaaccttcagccaatattggtgtgtggcagatgatattttgtttggacacaggtgtaaagagagggctgaattgcaaggtcatgaggaatcttttgggggaaatggaaatgtcctgtgtcttgtttgtggtggtggtggtttcacaggtaaatacacctatcaaaaggtaatgaagtatataccttaaatgcatgcagtttattgtatgtaaattataactcaatataggtgattttaaaaaaaacctgaaagtttagttacaaacatattgcaagttcaggaagccaggcactgtcatatgctgctggtggaagcatgagctggtcaacccatggagtgcagcttcgtatttatctatcaaaattacaaatgcatgtcccctttgactcagccatttcactttccagaatttagcctaattccacatttgttaaatgatgtacttataagatcaccaattgtagcactgtttgtaatagcaactaaatgcccaccaataagaaaatggttacataaattctgatacgtccatgtaataaaatgcagaagcagtgtggcaaagaatgagggagctcttttagtattgacacagaaagtccctcaagacactttaaatgactaaagcaaggggcctgacagtatgtagtatgctgaaaagggagtaggaaagagtgtatatattcaatatgcttattttgcatacaaactgtctggaagaatacataagaaattgcaaatagtggttgtcttctagggagaatgggagctgcgaagtggaagaaaaggggacaagggaaaaagacactcttcactatgtacttagaattttttatttttaagccatgagaatataatcaaaagtaaataaatagaaattttaaactaaagtaaagacagtcttagatttcttatgaagaaaactgtggaaaaataatcagactacacaaagatttctaaattgtaagatggcagaagttctccatgggacaaaatgtattcatattaagtttggattcagtcatcttttgttttgcttcttttaaaattaatgtttctaatagtacttgtgtttcttggaattaatgggtaaattattaagtggtgatcaccatatactttgtaaaaaaaaaaaaaaa

SEQ ID NO: 58 - protein, 378 aa, Homo sapiens
MAVIGLPYYLFVWEDYDKYVIFASFNLIWSTVILELWKRGCANMTYRWGTLLMKRKFEEPRPGFHGVLGINSITGKEEPLYPSYKRQLRIYLVSLPFVCLCLYFSLYVMMIYFDMEVWALGLHENSGSEWTSVLLYVPSIIYAIVIEIMNRLYRYAAEFLTSWENHRLESAYQNHLILKVLVFNFLNCFASLFYIAFVLKDMKLLRQSLATLLITSQILNQIMESFLPYWLQRKHGVQVKRKVQALKADIDATLYEQVILEKEMGTYLGTFDDYLELFLQFGYVSLFSCVYPLAAAFAVLNNFTEVNSDALKMCRVFKRPFSEPSANIGVWQMIFCLDTGVKRGLNCKVMRNLLGEMEMSCVLFVVVVVSQVNTPIKR

SEQ ID NO: 59 - DNA, 4145 bp, Homo sapiens
aggtctagaattcaagatgaagtaaagaaggaaagagagggtctggagaatgacttgaaatctgtgaattttgacatgacaagcaagtttttgacagccctggctcaagatggtgtgataaatgaagaagctctttctgttactgaactagatcgagtctatggaggtcttacaactaaagtccaagaatctctaaagaaacaggagggacttcttaaaaatattcaggtctcacatcaggaattttcaaaaatgaaacaatctaataatgaagctaacttaagagaagaagttttgaagaatttagctactgcatatgacaactttgttgaacttgtagctaatttgaaggaaggcacaaagttttacaatgagttgactgaaatcctggtcaggttccagaacaaatgcagtgatatagtttttgcacggaagacagaaagagatgaactcttaaaggacttgcaacaaagcattgccagagaacctagtgctccttcaattcctacacctgcgtatcagtcctcaccagcaggaggacatgcaccaactcctccaactccagcgccaagaaccatgccgcctactaagccccagcccccagccaggcctccaccacctgtgcttccagcaaatcgagctccttctgctactgctccatctccagtgggggctgggactgctgcgccagctccatcacaaacgcctggctcagctcctcctccacaggcgcagggaccaccctatcccacctatccaggatatcctgggtattgccaaatgcccatgcccatgggctataatccttatgcgtatggccagtataatatgccatatccaccagtgtatcaccagagtcctggacaggctccatacccgggaccccagcagccttcataccccttccctcagcccccacagcagtcttactatccacagcagtaatatgtctgctcagcagctcagctgattcagatcagagggaaagaaataccaaccctgcaataagtgtactaaactctacgctctggttaatgtaatgtactctcctggactgaatgcagtgtataatttctgtctacagctagaagctgtgccccagttccacatttgattacacatgtgagatttgctgctgttgcagtataaacactaggtataataggatttgaaattgcattacagttcataaaaattgaaaatgagaaattaaacctgcaagtgaaacatttgaaacgattatacttttctacataagacatggttgggacatcagatacttacaaagatggtttaagtatggatactagagaaaattaagttttctttctctttggtttattgatttggtttaatttccattatgctattttgcataatcaaggcactgtaaatcttataattttaaaataaattacttaagaacagttgtcattgttatgttttgttattgattctcattactgtctaattttttttctggtattagtctcattttgtatgtatataagttaaacagatactgtttttaagtgcatgaatagtacaagttattatcaaggatgttttacagggaaatcaaaagaatattatcatactttatctttcgtatgctgattagtaaacgatttttgacatttattttagaaagtcctataatgtggaagaaacaaacagttgctaccaaagattcttcaaataaacatacaaataaatgtgtatatttaatgttttattgttagcttctccagaaaattgatgcaaattctggtaataattcttgcattttttccccataacctggttaaaataaatacgccattggcaatacttcataatgtaatggaattgtttggggaacacttactgtaccctctcatcctttttccaccttactgtgttaacttagtgacatttaatgcccaatatgtatgaatagatctaagccatttaattttttttccttaaaagattggagtattttataattcaaggagcatacaaaacaatggttgggaacatatgccaattatggaataggctatgtatttaatattaatctctgccattaggatatctactcactgtataaacctcagtaaaaatagtgaagacatgcatcatggaatgagaaaatgagaaaggaatgagttgtctaacatcacagtgggatctgttttttgtgaggttcatttctgaacacattaggcatatgagcagatttccagtgaatctatttatgtttattttctgagtttcaacgctgaccttttcttgcattattgtttcattttaatgatagtgttacttgtcccactgttgttttcattgagtttggatttatattttaatgttcgaatgaaagtatgattgtaaaagggagtgaattggtttaaaaatatatgtatattttaaactttgttgtgtgtaggaaacatgaaggcatgttaattcaatataaatgacctttgatttcatggaatattaaagttggtttaaagtccaatagttaaaccttagcaaaaatagctttttacttcatcagttgctaagatttaatactttggattcatcaaagtgtgacatgggcttgtttgactttctgtaagtggcatttaagttccacattcttattacttgaggtactttatactaacataagacagtgagagttagaggtattacaagttgctagtttataatgtcttactaatgcagaaacaaggaaaaaagcaaaattggcctgaatattctcttggggaaagagggcaccaaagaaaagggtaagtgcatctgagggccaaaagagatgtataagccttttagcccattccccatgctgggcctgctcacagagccacaggaagatcattcagaaactaggaaaggaggcccccacagctgatcctgccacagcacacctgactcactcggctctgttagtgtaaccttttaaatgtagcaacacaaaccctttccctcttgtcagttcactcatcctttggtttctttttaatcacctgtgtctgggcacagacaatcacaataaatgcagccctttattactgttaaggatcatactgttggtttggagttggaagggtactactctgtgattcaggtgtgttgtacccatatttataattaggctttattatcttcctaaatcaaggaaaggaaatcatccccagaccatttatgctgagctttggaatactattttaaactggattgtacttaaataatgaagctctgcatagaggaactagtcagaagtggggaaaacactgtctaatttttatcagtctggtataaagtattgatctaagagaactctccctgtgccccttggtctttattctcaattaagaaaaacagtcacatgtcacgacaaaccaatcaatctttatgagatattcctgtatccataccccagcttgtttgcaatttataaacctccccttcaaaactaaggagttgcagaaaaaaatggatttcacagagccttgtgtccctaaagttctgtcccagtcagcagtctttatagtccaaacagattataaaaaatgttttccatttgaactttacagtttgcaaaagtgcttttatacattttctaatttcagaaacaggataatttgttaagtgggtttcagtttgctaatagggattttttgtgttttgttttttaattttcagcatctcttgaagaatcttgctacagccaaatggcatctcactttttaaagacgtttgcaattattagttgattcacagtacagaacaaggtataaaggaaaaaaccctgctaggtagtgttataattgctagattaaaaatagactagaacaggttcattttaagatttacttggaagagcaaagaaggaaaaattatatttttaaagaaagagaatattcaggctttatttctggtatgaagtttatattttttaaaaaaatcctatattatcacaccagagattttagattcttttctggttagaaacattgctggtagttggattatatttttattgtattcatttatcttagggggaacattgtaaagaaacaaaaaggtccagatgaatgtatgctagaaataaaagttgaaagattcttacttcaaaaaaaaaaaaaaaa

SEQ ID NO: 60 - protein, 289 aa, Homo sapiens [sequence omitted]

SEQ ID NO: 61 - DNA, 1417 bp, Homo sapiens
ggtgcccgacatggcgagtgtagtgctgccgagcggatcccagtgtgcggcggcagcggcggcggcggcgcctcccgggctccggctccggcttctgctgttgctcttctccgccgcggcactgatccccacaggtgatgggcagaatctgtttacgaaagacgtgacagtgatcgagggagaggttgcgaccatcagttgccaagtcaataagagtgacgactctgtgattcagctactgaatcccaacaggcagaccatttatttcagggacttcaggcctttgaaggacagcaggtttcagttgctgaatttttctagcagtgaactcaaagtatcattgacaaacgtctcaatttctgatgaaggaagatacttttgccagctctataccgatcccccacaggaaagttacaccaccatcacagtcctggtcccaccacgtaatctgatgatcgatatccagaaagacactgcggtggaaggtgaggagattgaagtcaactgcactgctatggccagcaagccagccacgactatcaggtggttcaaagggaacacagagctaaaaggcaaatcggaggtggaagagtggtcagacatgtacactgtgaccagtcagctgatgctgaaggtgcacaaggaggacgatggggtcccagtgatctgccaggtggagcaccctgcggtcactggaaacctgcagacccagcggtatctagaagtacagtataagcctcaagtgcacattcagatgacttatcctctacaaggcttaacccgggaaggggacgcgcttgagttaacatgtgaagccatcgggaagccccagcctgtgatggtaacttgggtgagagtcgatgatgaaatgcctcaacacgccgtactgtctgggcccaacctgttcatcaataacctaaacaaaacagataatggtacataccgctgtgaagcttcaaacatagtggggaaagctcactcggattatatgctgtatgtatacgattcccgagcaggtgaagaaggctcgatcagggcagtggatcatgccgtgatcggtggcgtcgtggcggtggtggtgttcgccatgctgtgcttgctcatcattctggggcgctattttgccagacataaaggtacatacttcactcatgaagccaaaggagccgatgacgcagcagacgcagacacagctataatcaatgcagaaggaggacagaacaactccgaagaaaagaaagagtacttcatctagatcagcctttttgtttcaatgaggtgtccaactggccctatttagatgataaagagacagtgatattggaacttgcgagaaattcgtgtgtttttttatgaatgggtggaaaggtgtgagactgggaaggcttgggatttgctgtgtaaaaaaaaaaaaaaa

SEQ ID NO: 62 - protein, 414 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDSRAGEEGSIRAVDHAVIGGVVAVVVFAMLCLLIILGRYFARHKGTYFTHEAKGADDAADADTAIINAEGGQNNSEEKKEYFI

SEQ ID NO: 63 - DNA, 1571 bp, Homo sapiens
ggccgcggagactgcgaccctcttctctcagtctgccttactaccatgccgctctacgagggcctggggagcggcggggagaagacggcggtcgtgatcgacctgggagaggcctttaccaagtgtggatttgctggagaaactggtccaagatgtataattcctagtgtgataaaaagagctgggatgcctaagcctgtcagagttgttcagtataatatcaatacagaagaattatattcctacctaaaggaattcatccacatactatatttcaggcatctattggtgaatcccagagaccgccgagttgtgattatcgaatcggtattatgtccttctcacttcagagagacactcactcgtgttcttttcaaatattttgaggttccatctgtcttgcttgctccaagtcatctaatggctcttctgacgcttggaattaattctgccatggtcctagattgtggatatagggaaagcctggtgttacccatatatgaaggaatcccagttctaaattgttggggagcactacccctaggaggaaaagctcttcacaaagagttggaaactcaactattggaacaatgtactgttgacacaagtgttgctaaagaacagagccttccctcagtgatgggttcagttccggaaggtgtcttagaggacattaaagcgcgtacttgctttgtaagtgatctgaagcgaggactaaaaatccaagcagcaaaatttaatattgatgggaataatgagcgtccctccccacccccaaatgttgactatccattagatggagagaagattttacatatccttggatcaatcagagattcagttgtggaaattctttttgaacaagataatgaagagcaatcagttgccactttaatattggattcccttatacagtgtccgatagacaccaggaagcaactagcagagaatttggtagtcataggtggcacttctatgttgccaggatttctccacagattgcttgcagaaataaggtatttggtagaaaaaccaaaatataaaaaagcacttggcactaagacatttcgaattcatactccacctgcaaaagctaattgtgtggcctggttgggaggggctatttttggagcattacaagatatacttgggagccgttctgtttcaaaggaatattataatcagacgggccgtatacctgattggtgttctctcaataacccacctttggaaatgatgtttgatgtcgggaaaactcaaccacctctgatgaagagagcattttccactgagaaatagaagtttgattaaaaatcaaccttgcttcatatcaaatatttaaccaattataagcaaattgtacaaagtatgtaggatgttttgttatagaggactatagtggaagtgaaagcattctgtgtttactctttgcattaatatataattcttttgactttgtttctcttgtgtagtggtaaaatggtagctggtgcttattgagatttgctgtatttatatcaataaagtatagtaaagcaaaaaaaaaaaaaaaaaaaaaaaaaaaaaaaaaa

SEQ ID NO: 64 - protein, 417 aa, Homo sapiens
MPLYEGLGSGGEKTAVVIDLGEAFTKCGFAGETGPRCIIPSVIKRAGMPKPVRVVQYNINTEELYSYLKEFIHILYFRHLLVNPRDRRVVIIESVLCPSHFRETLTRVLFKYFEVPSVLLAPSHLMALLTLGINSAMVLDCGYRESLVLPIYEGIPVLNCWGALPLGGKALHKELETQLLEQCTVDTSVAKEQSLPSVMGSVPEGVLEDIKARTCFVSDLKRGLKIQAAKFNIDGNNERPSPPPNVDYPLDGEKILHILGSIRDSVVEILFEQDNEEQSVATLILDSLIQCPIDTRKQLAENLVVIGGTSMLPGFLHRLLAEIRYLVEKPKYKKALGTKTFRIHTPPAKANCVAWLGGAIFGALQDILGSRSVSKEYYNQTGRIPDWCSLNNPPLEMMFDVGKTQPPLMKRAFSTEK

SEQ ID NO: 65 - DNA, 1752 bp, Homo sapiens
ggccaatcagagggacggccccagaatggcatggtagatggaacgcagctgagaggtctgacaagatgtaccaggtcccactaccactggatcgggatgggaccctggtacggctccgcttcaccatggtggccctggtcacggtctgctgtccacttgtcgccttcctcttctgcatcctctggtccctgctcttccacttcaaggagacaacggccacacactgtggggtgcccaattacctgccctcggtgagctcagccatcggcggggaggtgccccagcgctacgtgtggcgtttctgcatcggcctgcactcggcgcctcgcttcttggtggccttcgcctactggaaccactacctcagctgcacctccccgtgttcctgctatcgcccgctctgccgcctcaacttcggcctcaatgtcgtggagaacctcgcgttgctagtgctcacttatgtctcctcctccgaggacttcaccatccacgaaaatgctttcattgtgttcattgcctcatccctcgggcacatgctcctcacctgcattctctggcggttgaccaagaagcacacagtaagtcaggaggatcgcaagtcctacagctggaaacagcggctcttcatcatcaacttcatctccttcttctcggcgctggctgtctactttcggcacaacatgtattgtgaggctggagtgtacaccatctttgccatcctggagtacactgttgtcttaaccaacatggcgttccacatgacggcctggtgggacttcgggaacaaggagctgctcataacctctcagcctgaggaaaagcgattctgaacccttcagtcctgcttgggaggacgcagcccactgcccagaaacaagaaacacgataccattctggccttccccaccccacatcctctcttggccttactgaagatgggggaagggtaagaaggaagggtgtaggccaaggctcaccccagtgctgctggcttctcctctccacccctcatatgggcgtggggtcctcaaacatcacctttacctgagaggccccaagaagctgagctggcagagagctccaccatttggtgctaaaaaaaaaaacgtcctgaggttcatgaccaccatccagtttctggcctttacacagtcacctttcactgaggtcaggagcccctgagcagtggctgctccctgacaaccacagccatttctctgcacgggggtcattcataggactaatgtatttcatgatctactgtgcacatccaggcctgtggccacagtcccctgctaaagttgctcaggtgttctagtcctgacttcacctttttgatttggtgtgtgccctagggtatgtacccttccccatctgagcctcggtgtgtccatgtgtctggcgggggatgggtggactgtatgatttccaaggactctaccagtcagtggttctgatgtcatcgggtggaggtggtgttctatacctaaaggatgacctgctccagaaacagcaccagcacagcatgtattttcttctcttctgaaagttctggcttgtagacccctcccctcctttgcaaaggtatgggatagaggggtcagatgcagatctctactgtaaaatgggctccctggtatctcctgtcttccctactgctccaaaccctaaattttggttgtacattttatttgaaaggaaaataaattttttttttgggccaaaaaaaaaaaaaaaaaaaaaaaaaaaaaa

SEQ ID NO: 66 - protein, 254 aa, Homo sapiens
MYQVPLPLDRDGTLVRLRFTMVALVTVCCPLVAFLFCILWSLLFHFKETTATHCGVPNYLPSVSSAIGGEVPQRYVWRFCIGLHSAPRFLVAFAYWNHYLSCTSPCSCYRPLCRLNFGLNVVENLALLVLTYVSSSEDFTIHENAFIVFIASSLGHMLLTCILWRLTKKHTVSQEDRKSYSWKQRLFIINFISFFSALAVYFRHNMYCEAGVYTIFAILEYTVVLTNMAFHMTAWWDFGNKELLITSQPEEKRF

SEQ ID NO: 67 - DNA, 781 bp, Homo sapiens
cactcctgcagacaaggcactgattgccccagaccatgtagttccagctccagaagagtgctatgtgtatagtccattgggctctgcttataaacttcaaagttacactgaaggatacggtaaaaacaccagtttagtaaccatttttatgatttggaataccatgatgggaacatctatactaagcattccttggggcataaaacaggctggatttactactggaatgtgtgtcatcatactgatgggccttttaacactttattgctgctacagagtagtgaaatcacggactatgatgttttcattggataccactacctgggaatatccagatgtctgcagacattatttcggctcctttgggcagtggtcgagtctcctcttctccttggtgtctctcattggagcaatgatagtttattgggtgcttatgtcaaattttctttttaatactggaaagtttatttttagtaagtatctatatcatatgcttttaacacagtactttcaaatactattaccactgtaatgttagttctagccttaaattctaggacttgggataaataaaataagaagtaacatatataattttggaaaatatattttattcagttggctttctgtggttgtgctctcaaatatagtgtatgcttatttccaaacattaatctttgaaggaataatattcctccaaaatctttagttaaaataaaatatgtctataatccaaaaaaaaaaaaaaaaaaaaaaaaaaaaaaaaaaaaaaaaaaaaaaa

SEQ ID NO: 68 - protein, 127 aa, Homo sapiens
MIWNTMMGTSILSIPWGIKQAGFTTGMCVIILMGLLTLYCCYRVVKSRTMMFSLDTTTWEYPDVCRHYFGSFGQWSSLLFSLVSLIGAMIVYWVLMSNFLFNTGKFIFSKYLYHMLLTQYFQILLPL

SEQ ID NO: 69 - DNA, 649 bp, Homo sapiens
gagcaactcccttccccatctctgctcaccatgtggacgctgaaatcgtccctggtcctgcttctgtgcctcacctgcagctatgcctttatgttctcttctctgagacagaaaactagcgaaccccaggggaaggtgcaatacggagagcactttcggattcggcagaatctaccagagcacacccaaggctggcttgggagcaaatggctctggcttctttttgttgttgtgccgtttgtgatactgcagtgtcaaagagacagtgagaagaataaggagcagagtcctcctggccttcgaggcggccaacttcactctccattaaagaaaaaaagaaatgcttcccccaacaaagactgtgcattcaataccttaatggaactcgaggtggagcttatgaaatttgtgtccaaagtgcggaatcttaaacgtgccatggcaacaggtagtggcagtaacctcaggcttcgaaagtcagagatgcctgcagatccataccatgtcacgatctgtgaaatatggggagaagaaagctctagctgaatggatttgtgtgtcaggagagaaaaaagttgagtgttgacaaactgtatgcaaactaataaaactattctgaagaaaagaaaaaaaaaaaaaaaaaaaaaaa

SEQ ID NO: 70 - protein, 171 aa, Homo sapiens
MWTLKSSLVLLLCLTCSYAFMFSSLRQKTSEPQGKVQYGEHFRIRQNLPEHTQGWLGSKWLWLLFVVVPFVILQCQRDSEKNKEQSPPGLRGGQLHSPLKKKRNASPNKDCAFNTLMELEVELMKFVSKVRNLKRAMATGSGSNLRLRKSEMPADPYHVTICEIWGEESSS

SEQ ID NO: 71 - DNA, 1456 bp, Homo sapiens
cacggctgtcttatctgcaagtgcagagaggcctctgcttcagctgggccacccatcctgtcgggcacttgtctcaccgtggatggtcatcatcataaaaatgaggagagctggcacgatgggtgccgggaatgctactgtctcaatggacgggaaatgtgtgccctgatcacctgcccggtgcctgcctgtggcaaccccaccattcaccctggacagtgctgcccatcatgtgcagatgactttgtggtgcagaagccagagctcagtactccctccatttgccacgcccctggaggagaatactttgtggaaggagaaacgtggaacattgactcctgtactcagtgcacctgccacagcggacgggtgctgtgtgagacagaggtgtgcccaccgctgctctgccagaacccctcacgcacccaggattcctgctgcccacagtgtacagatcaaccttttcggccttccttgtcccgcaataacagcgtacctaattattgcaaaaatgatgaaggggatatattcctggcagctgagtcctggaagcctgacgtttgtaccagctgcatctgcattgatagcgtaattagctgtttctctgagtcctgcccttctgtatcctgtgaaagacctgtcttgagaaaaggccagtgttgtccctactgcatagaagacacaattccaaagaaggtggtgtgccacttcagtgggaaggcctatgccgacgaggagcggtgggaccttgacagctgcacccactgctactgcctgcagggccagaccctctgctcgaccgtcagctgcccccctctgccctgtgttgagcccatcaacgtggaaggaagttgctgcccaatgtgtccagaaatgtatgtcccagaaccaaccaatatacccattgagaagacaaaccatcgaggagaggttgacctggaggttcccctgtggcccacgcctagtgaaaatgatatcgtccatctccctagagatatgggtcacctccaggtagattacagagataacaggctgcacccaagtgaagattcttcactggactccattgcctcagttgtggttcccataattatatgcctctctattataatagcattcctattcatcaatcagaagaaacagtggataccactgctttgctggtatcgaacaccaactaagccttcttccttaaataatcagctagtatctgtggactgcaagaaaggaaccagagtccaggtggacagttcccagagaatgctaagaattgcagaaccagatgcaagattcagtggcttctacagcatgcaaaaacagaaccatctacaggcagacaatttctaccaaacagtgtgaagaaaggcaactaggatgaggtttcaaaagacggaagacgactaaatctgctctaaaaagtaaactagaatttgtgcacttaaaaaaaaaaaaaaaa

SEQ ID NO: 72 - protein, 400 aa, Homo sapiens [sequence omitted]

SEQ ID NO: 73 - DNA, 4723 bp, Homo sapiens
ggccttcatggcctatttttttttttttttaaatgatacaacttaattttattaggacaaggctggtgggcactggagtggcaccttcagggccaggagaggcactggggaggggtcacaggatgctactcgggcacctagaagccacagctgccctccacagagcggcactgcaccatgcgcaggaatgtctcgaccttgtccatgtccttcctgaagcagtagagcagcccgtagttcttgagcagtgcgtcatggttgtgcgagtttgtgtcaaacttgctgtaggtctgcttgaggatctgcccagtccggcggctgccgtcttccagcctccccatcagcgtttggatgccttcctctaggtcctttaggaggtgatagtcatcgttgtccgaggtgtcatacaccaggttgttggcgaacatactcctgaggaaccgcacgggctccagccacgactcgatgagcagcagggagatgcggagcagctctagattggatttctgttgcgtttcctccatgttggagggtgtcggaatagagtctgagaagcagaaggaggtctgggagtcatgcaggaatgaatacttctggtcctttgggatataggtttcttcaaactcctggtaggtgtcaatggccagctggtgcgcgcgatgggcttggagcatagcgtggtcaaaaagcctggataacggaacggtttggacggcaccagcctcttgaagccagggcaggcagagcagggcaaaagccaggagcagggacgtccgggagcctggagccattgccactaggtgagctgtccacaggaccctgagtggttcggggagttcggccttcatggcctaggagcggcgcaggagtgaggcgagcggggcgcgcggagcggacgccgcggatcttgtgctgcgccaccgcgcccactcggcagctcgggaggcggggaccggcccggaggctgcgccgctgcggggccggccgactcggaggaggagagggaggaggcgccgccggcccgggctggagccgagcgcagcagccaccgccgccgccgcgccagaagtttgggttgaaccggagctgccgggaggaaacttttttcttttttccccctccctcccgggaggaggaggaggaggaggaggggaagctgccgccggcgccaaggctcgtgggctcggggtcggcgcggcccgcagaaggggcgggggcctcgccccgcgaggggaggcgcgccccgggggccccgagaggggcggtgaggaccgcgggctgctggtgcggcggcggcggcggcgcgtgtgccccgcgcaggggagggcgcccgccccgctcccggcccggctgcgaggaggaggcggcggcggcgcaggaggatgtacttggtggcgggggacagggggttggccggctgcgggcacctcctggtctcgctgctggggctgctgctgctgctggcgcgctccggcacccgggcgctggtctgcctgccctgtgacgagtccaagtgcgaggagcccaggaactgcccggggagcatcgtgcagggcgtctgcggctgctgctacacgtgcgccagccagaggaacgagagctgcggcggcaccttcgggatttacggaacctgcgaccgggggctgcgttgtgtcatccgccccccgctcaatggcgactccctcaccgagtacgaagcgggcgtttgcgaagatgagaactggactgatgaccaactgcttggttttaaaccatgcaatgaaaaccttattgctggctgcaatataatcaatgggaaatgtgaatgtaacaccattcgaacctgcagcaatccctttgagtttccaagtcaggatatgtgcctttcagctttaaagagaattgaagaagagaagccagattgctccaaggcccgctgtgaagtccagttctctccacgttgtcctgaagattctgttctgatcgagggttatgctcctcctggggagtgctgtcccttacccagccgctgcgtgtgcaaccccgcaggctgtctgcgcaaagtctgccagccgggaaacctgaacatactagtgtcaaaagcctcagggaagccgggagagtgctgtgacctctatgagtgcaaaccagttttcggcgtggactgcaggactgtggaatgccctcctgttcagcagaccgcgtgtcccccggacagctatgaaactcaagtcagactaactgcagatggttgctgtactttgccaacaagatgcgagtgtctctctggcttatgtggtttccccgtgtgtgaggtgggatccactccccgcatagtctctcgtggcgatgggacacctggaaagtgctgtgatgtctttgaatgtgttaatgatacaaagccagcctgcgtatttaacaatgtggaatattatgatggagacatgtttcgaatggacaactgtcggttctgtcgatgccaagggggcgttgccatctgcttcactgcccagtgtggtgagataaactgcgagaggtactacgtgcccgaaggagagtgctgcccagtgtgtgaagatccagtgtatccttttaataatcccgctggctgctatgccaatggcctgatccttgcccacggagaccggtggcgggaagacgactgcacattctgccagtgcgtcaacggtgaacgccactgcgttgcgaccgtctgcggacagacctgcacaaaccctgtgaaagtgcctggggagtgttgccctgtgtgcgaagaaccaaccatcatcacagttgatccacctgcatgtggggagttatcaaactgcactctgacagggaaggactgcattaatggtttcaaacgcgatcacaatggttgtcggacctgtcagtgcataaacaccgaggaactatgttcagaacgtaaacaaggctgcaccttgaactgtcccttcggtttccttactgatgcccaaaactgtgagatctgtgagtgccgcccaaggcccaagaagtgcagacccataatctgtgacaagtattgtccacttggattgctgaagaataagcacggctgtgacatctgtcgctgtaagaaatgtccagagctctcatgcagtaagatctgccccttgggtttccagcaggacagtcacggctgtcttatctgcaagtgcagagaggcctctgcttcagctgggccacccatcctgtcgggcacttgtctcaccgtggatggtcatcatcataaaaatgaggagagctggcacgatgggtgccgggaatgctactgtctcaatggacgggaaatgtgtgccctgatcacctgcccggtgcctgcctgtggcaaccccaccattcaccctggacagtgctgcccatcatgtgcagatgactttgtggtgcagaagccagagctcagtactccctccatttgccacgcccctggaggagaatactttgtggaaggagaaacgtggaacattgactcctgtactcagtgcacctgccacagcggacgggtgctgtgtgagacagaggtgtgcccaccgctgctctgccagaacccctcacgcacccaggattcctgctgcccacagtgtacagatcaaccttttcggccttccttgtcccgcaataacagcgtacctaattactgcaaaaatgatgaaggggatatattcctggcagctgagtcctggaagcctgacgtttgtaccagctgcatctgcattgatagcgtaattagctgtttctctgagtcctgcccttctgtatcctgtgaaagacctgtcttgagaaaaggccagtgttgtccctactgcatagaagacacaattccaaagaaggtggtgtgccacttcagtgggaaggcctatgccgacgaggagcggtgggaccttgacagctgcacccactgctactgcctgcagggccagaccctctgctcgaccgtcagctgcccccctctgccctgtgttgagcccatcaacgtggaaggaagttgctgcccaatgtgtccagaaatgtatgtcccagaaccaaccaatatacccattgagaagacaaaccatcgaggagaggttgacctggaggttcccctgtggcccacgcctagtgaaaatgatatcgtccatctccctagagatatgggtcacctccaggtagattacagagataacaggctgcacccaagtgaagattcttcactggactccattgcctcagttgtggttcccataattatatgcctctctattataatagcattcctattcatcaatcagaagaaacagtggataccactgctttgctggtatcgaacaccaactaagccttcttccttaaataatcagttagtatctgtggactgcaagaaaggaaccagagtccaggtggacagttcccagagaatgctaagaattgcagaaccagatgcaagattcagtggcttctacagcatgcaaaaacagaaccatctacaggcagacaatttctaccaaacagtgtgaagaaaggcaactaggatgaggtttcaaaagacggaagacgactaaatctgctctaaaaagtaaactagaatttgtgcacttgcttagtggattgtattggattgtgacttgatgtacagcgctaagaccttactgggatgggctctgtctacagcaatgtgcagaacaagcattccccctcaaacctaaaaaaaaaaaaaaaaaaaaaaaaaaaaaaaaaaaaaaaaaaa

SEQ ID NO: 74 - protein, 1036 aa, Homo sapiens
MYLVAGDRGLAGCGHLLVSLLGLLLLLARSGTRALVCLPCDESKCEEPRNCPGSIVQGVCGCCYTCASQRNESCGGTFGIYGTCDRGLRCVIRPPLNGDSLTEYEAGVCEDENWTDDQLLGFKPCNENLIAGCNIINGKCECNTIRTCSNPFEFPSQDMCLSALKRIEEEKPDCSKARCEVQFSPRCPEDSVLIEGYAPPGECCPLPSRCVCNPAGCLRKVCQPGNLNILVSKASGKPGECCDLYECKPVFGVDCRTVECPPVQQTACPPDSYETQVRLTADGCCTLPTRCECLSGLCGFPVCEVGSTPRIVSRGDGTPGKCCDVFECVNDTKPACVFNNVEYYDGDMFRMDNCRFCRCQGGVAICFTAQCGEINCERYYVPEGECCPVCEDPVYPFNNPAGCYANGLILAHGDRWREDDCTFCQCVNGERHCVATVCGQTCTNPVKVPGECCPVCEEPTIITVDPPACGELSNCTLTGKDCINGFKRDHNGCRTCQCINTEELCSERKQGCTLNCPFGFLTDAQNCEICECRPRPKKCRPIICDKYCPLGLLKNKHGCDICRCKKCPELSCSKICPLGFQQDSHGCLICKCREASASAGPPILSGTCLTVDGHHHKNEESWHDGCRECYCLNGREMCALITCPVPACGNPTIHPGQCCPSCADDFVVQKPELSTPSICHAPGGEYFVEGETWNIDSCTQCTCHSGRVLCETEVCPPLLCQNPSRTQDSCCPQCTDQPFRPSLSRNNSVPNYCKNDEGDIFLAAESWKPDVCTSCICIDSVISCFSESCPSVSCERPVLRKGQCCPYCIEDTIPKKVVCHFSGKAYADEERWDLDSCTHCYCLQGQTLCSTVSCPPLPCVEPINVEGSCCPMCPEMYVPEPTNIPIEKTNHRGEVDLEVPLWPTPSENDIVHLPRDMGHLQVDYRDNRLHPSEDSSLDSIASVVVPIIICLSIIIAFLFINQKKQWIPLLCWYRTPTKPSSLNNQLVSVDCKKGTRVQVDSSQRMLRIAEPDARFSGFYSMQKQNHLQADNFYQTV

SEQ ID NO: 75 - DNA, 3861 bp, Homo sapiens
gtgcacgcgtggcagacggagaaggccagtgcccagcttgaaggttctgtcaccttttgcagtggtccaaatgagaaaaaagtggaaaatgggaggcatgaaatacatcttttcgttgttgttctttcttttgctagaaggaggcaaaacagagcaagtaaaacattcagagacatattgcatgtttcaagacaagaagtacagagtgggtgagagatggcatccttacctggaaccttatgggttggtttactgcgtgaactgcatctgctcagagaatgggaatgtgctttgcagccgagtcagatgtccaaatgttcattgcctttctcctgtgcatattcctcatctgtgctgccctcgctgcccagactccttacccccagtgaacaataaggtgaccagcaagtcttgcgagtacaatgggacaacttaccaacatggagagctgttcgtagctgaagggctctttcagaatcggcaacccaatcaatgcacccagtgcagctgttcggagggaaacgtgtattgtggtctcaagacttgccccaaattaacctgtgccttcccagtctctgttccagattcctgctgccgggtatgcagaggagatggagaactgtcatgggaacattctgatggtgatatcttccggcaacctgccaacagagaagcaagacattcttaccaccgctctcactatgatcctccaccaagccgacaggctggaggtctgtcccgctttcctggggccagaagtcaccggggagctcttatggattcccagcaagcatcaggaaccattgtgcaaattgtcatcaataacaaacacaagcatggacaagtgtgtgtttccaatggaaagacctattctcatggcgagtcctggcacccaaacctccgggcatttggcattgtggagtgtgtgctatgtacttgtaatgtcaccaagcaagagtgtaagaaaatccactgccccaatcgatacccctgcaagtatcctcaaaaaatagacggaaagtgctgcaaggtgtgtccaggtaaaaaagcaaaagaagaacttccaggccaaagctttgacaataaaggctacttctgcggggaagaaacgatgcctgtgtatgagtctgtattcatggaggatggggagacaaccagaaaaatagcactggagactgagagaccacctcaggtagaggtccacgtttggactattcgaaagggcattctccagcacttccatattgagaagatctccaagaggatgtttgaggagcttcctcacttcaagctggtgaccagaacaaccctgagccagtggaagatcttcaccgaaggagaagctcagatcagccagatgtgttcaagtcgtgtatgcagaacagagcttgaagatttagtcaaggttttgtacctggagagatctgaaaagggccactgttaggcaagacagacagtattggatagggtaaagcaagaaaactcaagctgcagctggactgcaggcttattttgcttaagtcaacagtgccctaaaactccaaactcaaatgcagtcaattattcacgccatgcacagcataatttgctcctttgtgtggagtggtgtgtcagcccttgaacatctcctccaaagagactagaagagtcttaaattatatgtgggaggaggagggatagaacatcacaacactgctctagtttcttggagaatcacatttctttacaggttaaagacaaacaagaccccagggtttttatctagaaagttattcaagtgaaagaaagagaagggaattgcttagtaggagttctgcagtatagaacaattacttgtatgaaattatacctttgaattttagaatgtcatgtgttcttttaaaaaaattagctccccatcctccctcctcactccctccctccctccttctctctctctctctctctccctctctcacagacacacacacacacacacacacacacacgcacgtccacactcacattaaactaaagctttatttgaagcaaagctagccaaaattctacgttacttttcccttgactggatcccaagtagcttggaagtttttgtgcccaggagagtaaataactgtgaacaagaggctctgcccttaggtctttgtggctgtttaagtcaccaacaatagagtcagggtaaagaataaaaacactttcatagcctcattcattcacttagaagtggtaataatttttccctaatgataccacttttcttttccccctgtacctatgggacttccagaaagaagttaaattgagtaaaatcatcagaaactgaatccatgtaagaaaaaataattgttgaagaaagaagttgatagaattcaaaaaggccatctttttgctttcacatcaataaaatttaccaagtaatagatcagtactcactaatatttttgagaccatagttgtctggtcagaaaaattatattaaattagtaaattctagaagctctttaaaagggaagttttccttcttctccaattataggagttgatttttactttgcaaagtggctcggtcctcatgagcatctgcatgttgactcttcagttaagaaaattgttgttcatttagggaggtggatattctgatgaagatctttatcctaaaccttcctactatccttgtcttattcatcaagcagatattttagtcaagaattccagagaaggctgctcctaaaatgtctacttgcagcccaataccagagcataaactatccattctggggtctggctttagaaatcatctttgtgggaagacctaattcttcacagcaaggatctcaggcatgccttctagatttgttccctctgaggggcaggaatgaactgtagaaatgttttaaggacccagaaaccccatatgtctcattccatgactataggtgagagaattctttcctaagagggtttgataccaataggggaaaatgtaaaatgttcagtctttatgacaacctggcataaaggagtcaattcttatgaaagagacacaagggccttatggccagggtttcttgggacaagactctcaccagcacatcacacacgttctccttggaagagagaagcagtacatcccggttgagaggtcacaaagcattagtgtgtgtgtgtgtgtgtgtgtgtgtgtgtgtgtgtgtgtgtgtgtgtgtgtgtgtgtgtatgtggtaaaggggggaaggtgttatgcggctgctccctccgtcccagaggtggcagtgattccataatgtggagactagtaactagatcctaaggcaaagaggtgtttctccttttggatgattcatcccaaagccttcccacccaggtgttctctgaaagcttagccttaagagaacacgcagagagtttccctagatatactcctgcctccaggtgctgggacacacctttgcaaaatgctgtgggaagcaggagctggggagctgtgttaagtcaaagtagaaaccctccagtgtttggtgttgtgtagagaataggacatagggtaaagaggccaagctgcctgtagttagtagagaagaatggatgtggttcttcttgtgtatttatttgtatcataaacacttggaacaacaaagaccataagcatcatttagcagttgtagccattttctagttaactcatgtaaacaagtaagagtaacataacagtattaccctttcactgttctcacaggacatgtacctaattatggtacttatttatgtagtcactgtatttctggatttttaaattaataaaaaagttaattttgaaaaatcaaaaaaaaaaaaaaaaaaaaaaaaaaaaaaaaaaaaaaa

SEQ ID NO: 76 - protein, 457 aa, Homo sapiens
MRKKWKMGGMKYIFSLLFFLLLEGGKTEQVKHSETYCMFQDKKYRVGERWHPYLEPYGLVYCVNCICSENGNVLCSRVRCPNVHCLSPVHIPHLCCPRCPDSLPPVNNKVTSKSCEYNGTTYQHGELFVAEGLFQNRQPNQCTQCSCSEGNVYCGLKTCPKLTCAFPVSVPDSCCRVCRGDGELSWEHSDGDIFRQPANREARHSYHRSHYDPPPSRQAGGLSRFPGARSHRGALMDSQQASGTIVQIVINNKHKHGQVCVSNGKTYSHGESWHPNLRAFGIVECVLCTCNVTKQECKKIHCPNRYPCKYPQKIDGKCCKVCPGKKAKEELPGQSFDNKGYFCGEETMPVYESVFMEDGETTRKIALETERPPQVEVHVWTIRKGILQHFHIEKISKRMFEELPHFKLVTRTTLSQWKIFTEGEAQISQMCSSRVCRTELEDLVKVLYLERSEKGHC

SEQ ID NO: 77 - DNA, 2050 bp, Homo sapiens
gtgctctgagaagccggactacgcggcagcggctcttcaaagcggagccgggagtttttgctacagttttcgccaccatgagtcgcagctataatgatgagctgcagttcttggagaagatcaataaaaactgctggaggatcaagaagggcttcgtgcccaacatgcaggttgaaggtgttttctatgtgaatgatgctctggagaaattgatgtttgaggaattaaggaatgcctgtcgaggtggtggtgttggtggcttcctgccagccatgaaacagattggcaatgtggcagccctgcctggaattgttcatcgatctattgggcttcctgatgtccattcaggatatgggtttgctattgggaacatggcagcctttgatatgaatgaccctgaagcagtagtatccccaggtggtgtcgggtttgacatcaactgtggtgtccgcttgctaagaaccaatttagatgaaagtgatgtccagcctgtgaaggagcaacttgcccaagctatgtttgaccacattcctgttggggtggggtcaaaaggtgtcatcccaatgaatgccaaagacttggaggaggccttggagatgggggtggactggtccttaagagaagggtatgcctgggctgaagacaaggagcactgcgaggagtacggaaggatgctgcaggctgaccccaataaagtttctgcaagggcgaagaaaagaggccttcctcagttggggaccctgggagcaggcaaccattatgcagaaatccaggttgtggatgagattttcaatgagtatgctgctaaaaaaatgggcatcgaccataagggacaggtgtgtgtgatgatccacagtggaagcagaggcttgggccaccaagtagccacagatgcgctggtagctatggagaaggccatgaagagagacaagattatagtcaatgatcggcagttggcttgtgctcgaatcgcttccccagagggtcaagactatctgaagggaatggcagctgctgggaactatgcctgggtcaaccgctcttccatgaccttcttaacccgtcaggctttcgccaaggtcttcaacacaacccctgatgacttggacctacatgtgatctatgatgtttctcacaacattgccaaagtggagcagcatgtggtggacggaaaggaacggacactgttagtacacaggaagggatccacccgcgctttccctcctcaccatcccctcattgctgttgattaccaactcactggacagccagtgctcattggtggcaccatgggaacctgtagttatgttcttactggcactgaacagggcatgactgagacctttggaacaacctgtcatggagcgggccgtgcattgtcccgagcaaaatctcgacgtaatttagatttccaggatgtcttagacaaattggcagatatgggaattgcgatccgtgttgcctcacccaaactggttatggaagaggctcctgagtcctataagaatgtgacagatgtggtaaatacctgccatgatgctggaatcagcaagaaagccattaaactgagaccaattgctgtgatcaaaggatagaaccttggacagcagggctgcctgacaccaccaaccctctctgaagtggaagtggactgacatgctcttctgacatcagactcaaggcgggacaagttgcaaagtgtgcagctgtaactgctcacgccaaaatggctgatggggaggctgctgctttcaggggcccgtgcttgtaaaataaccttccaggaagaggcacattgcccacctttggaaagggaggaatatgccttctccttggttgttccacagagttttaggaaaatctgttagggatgggtagatgtcaaactgccttacgcagtcatactgatctttagccatcagattgatcttcttcacaccaagctctgtttacattccgagaggtgtcatgaagaaagttctgttcaataagggaaaaaaaaaaaaaaaaaaaaaaaaaaaaaaaaaaaaaaaaaaaaaaaaaaaaaaaa

SEQ ID NO: 78 - protein, 505 aa, Homo sapiens
MSRSYNDELQFLEKINKNCWRIKKGFVPNMQVEGVFYVNDALEKLMFEELRNACRGGGVGGFLPAMKQIGNVAALPGIVHRSIGLPDVHSGYGFAIGNMAAFDMNDPEAVVSPGGVGFDINCGVRLLRTNLDESDVQPVKEQLAQAMFDHIPVGVGSKGVIPMNAKDLEEALEMGVDWSLREGYAWAEDKEHCEEYGRMLQADPNKVSARAKKRGLPQLGTLGAGNHYAEIQVVDEIFNEYAAKKMGIDHKGQVCVMIHSGSRGLGHQVATDALVAMEKAMKRDKIIVNDRQLACARIASPEGQDYLKGMAAAGNYAWVNRSSMTFLTRQAFAKVFNTTPDDLDLHVIYDVSHNIAKVEQHVVDGKERTLLVHRKGSTRAFPPHHPLIAVDYQLTGQPVLIGGTMGTCSYVLTGTEQGMTETFGTTCHGAGRALSRAKSRRNLDFQDVLDKLADMGIAIRVASPKLVMEEAPESYKNVTDVVNTCHDAGISKKAIKLRPIAVIKG

SEQ ID NO: 79 - DNA, 1178 bp, Homo sapiens
gccaaatgtccggtcaagatgtcacacagctccagtggctcagccagtctgagtcaggtttctccagggaaagaaacagatcaaactgaaaccgtgtcagttcagtcttcggtattggggaagggtgtaaaacatcgacccccaccaatcaaacttccctcaagctcaggaaatagttcctcaggtaactattttacaccacaacagacaagcagctttctcaaatctccaactcctcctccttcttctaagccatcaagtattcctcggaaatcatctgtggatctcaatcaagttagcatgctttctccagctgccctatcacctgccagctcatcacaaagaaccacggccacccaggtcatggcaaactctgctggacttaacttcatcaatgtagtgggctctgtttgtggggcccaggctttgatgagtggttcaaaccccatgctgggctgtaacactggtgccataactcctgcaggaataaacctgagcggccttctaccctcaggaggtctgctaccaaatgcactgcccagtgcaatgcaggcagcttctcaagcaggtgttccatttggtttaaaaaatacttcaagtctcaggcccttaaatctactccagcttccaggtggttcacttatttttaacactctgcagcagcagcaacagcagctctcccagtttacaccacaacaacctcagcagcccacaacttgtagtcctcaacagccaggggagcagggttctgagcaaggttcaaccagtcaagaacaggccttatctgctcagcaagctgctgttattaaccttactggagtaggaagttttatgcagtcacaggcagctgcagttgcgattcttgcagcatcaaatggctatggcagcagcagcagcacaaacagctcagctacatcatcatcggcatacaggcagccagtcaaaaagtaaaatgaagagaggcatgccaaccactccaaaattttgagtcttgcattactttttgttccttttttaaaaacacaagagcactgaatcaaaagaattgagtttctactttttgttttttttaatgtgtcagtattttacattgctagatgtacaaactttatacagaagcacaaccttatcatttttaaataaaaacagggaaatggtttaacaaaaaaaaaaaaaaa

SEQ ID NO: 80 - protein, 310 aa, Homo sapiens
MSHSSSGSASLSQVSPGKETDQTETVSVQSSVLGKGVKHRPPPIKLPSSSGNSSSGNYFTPQQTSSFLKSPTPPPSSKPSSIPRKSSVDLNQVSMLSPAALSPASSSQRTTATQVMANSAGLNFINVVGSVCGAQALMSGSNPMLGCNTGAITPAGINLSGLLPSGGLLPNALPSAMQAASQAGVPFGLKNTSSLRPLNLLQLPGGSLIFNTLQQQQQQLSQFTPQQPQQPTTCSPQQPGEQGSEQGSTSQEQALSAQQAAVINLTGVGSFMQSQAAAVAILAASNGYGSSSSTNSSATSSSAYRQPVKK

SEQ ID NO: 81 - DNA, 641 bp, Homo sapiens
gacgatgtcaccctgtcctccctccttgcttcttgctctgctaactcaactctgccttcctctttttcattcttctactctgccctatatggaggacaaatggacaccaggggtgctaaccttattggtgcctgccccagcctaccccaggtgccagcagactctcgtgcacaggaggctcccacagttatggagccaggaaagaatttctctgcactggatggactgtatattgagattaaaaattatattccttatattcctgcttatatcaatgctctctctgtaaaacctcttcctagcctcatttctctcaactgatcttgtttaggcgttgtattccttttatttactctttgcttgactgcttcctcctaaccctctacccactagcactctacttcctaaagctgttgtgtcattaactctgttggatcaactctctgggaaaagattctgttaatgtaagtgcacttactccctggatgttgtcactagtctagtggcttttgctaaataaacctttcttatttctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 82 - protein, 94 aa, Homo sapiens
MSPCPPSLLLALLTQLCLPLFHSSTLPYMEDKWTPGVLTLLVPAPAYPRCQQTLVHRRLPQLWSQERISLHWMDCILRLKIIFLIFLLISMLSL

SEQ ID NO: 83 - DNA, 832 bp, Homo sapiens
ccttgcattaccttcttctgccctatctgctgctaggtgtaaacctgttttttttcaccctgacttgtggaaccaatcctggcattataacaaaagcaaatgaattattatttcttcatgtttatgaatttgatgaagtgatgtttccaaagaacgtgaggtgctctacttgtgatttaaggaaaccagctcgatccaagcactgcagtgtgtgtaactggtgtgtgcaccgtttcgaccatcactgtgtttgggtgaacaactgcatcggggcctggaacatcaggtacttcctcatctacgtcttgaccttgacggcctcggctgccaccgtcgccattgtgagcaccacttttctggtccacttggtggtgatgtcagatttataccaggagacttacatcgatgaccttggacaccttccatgttatggacacggtctttcttattcagtacctgttcctgacttttccacggattgtcttcatgctgggctttgtcgtggttctgagcttcctcctgggtggctacctgttgtttgtcctgtatctggcggccaccaaccagactactaacgagtggtacagaggtgactgggcctggtgccagcgttgtccccttgtggcctggcctccgtcagcagagccccaagtccaccggaacattcactcccatgggcttcggagcaaccttcaagagatctttctacctgcctttccatgtcatgagaggaagaaacaagaatgacaagtgtatgactgcctttgagctgtagttcccgtttatttacacatgtggatcctcgttttcctcccgattgaattctagacct

SEQ ID NO: 84 - protein, 144 aa, Homo sapiens
MFPKNVRCSTCDLRKPARSKHCSVCNWCVHRFDHHCVWVNNCIGAWNIRYFLIYVLTLTASAATVAIVSTTFLVHLVVMSDLYQETYIDDLGHLPCYGHGLSYSVPVPDFSTDCLHAGLCRGSELPPGWLPVVCPVSGGHQPDY

SEQ ID NO: 85 - DNA, 3790 bp, Homo sapiens
aacggcagtctcaatctggcccccaccttttcttgggcttgtaggaaggtggacatgggctcccggagacaagacaagtgatatgttgaactgttcggtggctggaatcaactgctcctggagtgacctaaggccagtgtttatcagaacttagccagggccagccaagcaggcacagatgctctgctatgaaatgccacgcaggcagagactgacaagcggtaggaactgagctttccccttggactgctgcttcctgctgtgttcaggggagggggtcactttctggcaactctgctgctgctgctgctgctgctgctacttcagcttcctctccactcaaggtaagcaggctaagggagggcaggctgctagggaaagctttgtaccatgaacaggatccgaaagtttttccgaggaagtgggcgagtcttggcatttatctttgtagcttctgtcatctggctcctctttgacatggcagctctccgcctctcattcagtgagatcaacactcgggtcatcaaggaagacattgtgaggagggagcggataggattcagagttcagccagaccaaggaaaaattttttacagcagcataaaagagatgaaacctcccctaaggggacatgggaaaggggcatggggcaaagagaatgttagaaaaactgaggagagtgtgctcaaggttgaggtggacttggaccaaacccagagggaaagaaaaatgcagaatgccctgggaaggggcaaggttgtgccgttgtggcatcctgcacatctgcagaccctccctgtgactcctaacaagcagaagacagacgggagaggcaccaaacctgaagcctcctctcaccaggggacaccaaagcaaacgacagctcagggggctccaaagacctcattcatagcagcaaaaggaactcaggtagtcaaaatatcagtacacatgggacgtgtcagtttaaaacaggagccccggaagagtcatagtcccagcagtgacacatcaaaactagcagctgaaagggacttgaatgtgaccatcagtcttagtactgatagaccaaagcagcgatcacaggcagtagcaaacgagagggcacaccctgccagcacagcagtgccgaagtctggggaagccatggccttaaacaaaactaagactcagagcaaagaagtcaatgcaaataaacacaaagccaatacgagtcttccttttcctaagttcactgtcaattcaaatcgcttaaggaagcaatctattaatgagacacctttgggaagtttgtcaaaggatgatggagctagaggggctcatgggaagaaactcaatttctctgaaagccatcttgtgattataaccaaagaggaagagcaaaaggcagaccccaaagaggtctctaattctaaaaccaaaacaatatttcctaaagtattgggtaaaagccaaagtaaacacatttccaggaatagaagtgagatgtcttcctcttcacttgctccacatagagtgccactgtcccaaactaaccatgctttaactggagggctagagccagcaaaaatcaacataactgccaaagccccctctacagaatacaaccagagtcatataaaagcccttttacctgaagacagtggaacgcaccaggtgttaagaattgatgtgacactttctccaagggaccccaaagctccagggcagtttgggcgtcctgtagttgtcccccatggaaaggagaaggaggcagaaagaagatggaaagaaggaaacttcaatgtctaccttagcgatttgatcccagtggatagagccattgaagacaccagacctgctggatgtgcagagcagctagttcacaataacctcccaaccaccagtgtcatcatgtgctttgtggatgaagtgtggtccactctcctgagatctgttcacagtgtcatcaatcgctctcctccacacctcatcaaggagattctgctggtagatgacttcagcaccaaagactatctaaaagataatttggataaatacatgtcccagtttccaaaagttcggattcttcgcctcaaagagagacatggcttaataagggccaggctggcaggagcacagaatgcaacaggtgatgtgttgacatttttagattctcatgtggaatgtaacgttggttggttggaacctcttctggaaagagtttatttaagtagaaagaaagtggcctgtccagtaatcgaagtcatcaatgataaggatatgagttacatgacagtggataactttcaaagaggcatctttgtgtggcccatgaactttggttggagaacaattcctccagatgtcattgcaaaaaacagaattaaagaaactgatacaataaggtgccctgtcatggctggtggattgttttctattgacaaaagttacttttttgaacttggaacatacgaccctggccttgatgtttggggtggggaaaatatggagctctcattcaaggtgtggatgtgtggtggtgaaattgagatcattccctgctcccgagtgggccatatattcagaaatgacaatccatattccttccccaaagaccggatgaagacagtggagcggaacttggtgcgggttgccgaggtctggctggatgagtataaggagctgttctatggccatggagaccacctcatcgaccaagggctagatgttggcaacctcacccagcaaagggagctgcgaaagaaactgaagtgcaaaagtttcaaatggtacttggagaatgtctttcctgacttaagggctcccattgtgagagctagtggtgtgcttattaatgtggctttgggtaaatgcatttccattgaaaacactacagtcattctggaagactgcgatgggagcaaagagcttcaacaatttaattacacctggttaagacttattaaatgtggagaatggtgtatagcccccatccctgataaaggagccgtaaggctgcacccttgtgataacagaaacaaagggctaaaatggctgcataaatcaacatcagtctttcatccagaactggtgaatcacattgtttttgaaaacaatcagcaattattatgcttggaaggaaatttttctcaaaagatcctgaaagtagctgcctgtgacccagtgaagccatatcaaaagtggaaatttgaaaaatattatgaagcctgaagtgtaactgatgtttttatatagtaaacccattaaatactgtgaaaataacactgaacttggaaactatatttctcagcggtagtttaaattttcaattttaataacatttgaatggaagattttttataaatcacaatatttggaataccaaaagatgactcaggaaaacagtccaacattggactgaagtccttcttcggaactgggtggcctttgaattgcctgctttccaccctatgctagacctcatcatgcaaatttccctgtgaaagctaacaggtaactggaaatgaagacagaaggacttgagaaagcatgaggatattcccaatgactatgtttggtaataatcagctcttctggcccacaagtaggaatgatcaatgagaacttaacttagtcctttatttggggattttttcatcaaacaaaaatttcttgagttcttatggctagaagacctcagatgcccacagctgtcacgtttgtgaaatccctccagactacatgcatgcttacctaacagtttgaaatagtattgatctactgctggtaaaaaaaaaaaaaaaaaaaa

SEQ ID NO: 86 - protein, 940 aa, Homo sapiens
MNRIRKFFRGSGRVLAFIFVASVIWLLFDMAALRLSFSEINTRVIKEDIVRRERIGFRVQPDQGKIFYSSIKEMKPPLRGHGKGAWGKENVRKTEESVLKVEVDLDQTQRERKMQNALGRGKVVPLWHPAHLQTLPVTPNKQKTDGRGTKPEASSHQGTPKQTTAQGAPKTSFIAAKGTQVVKISVHMGRVSLKQEPRKSHSPSSDTSKLAAERDLNVTISLSTDRPKQRSQAVANERAHPASTAVPKSGEAMALNKTKTQSKEVNANKHKANTSLPFPKFTVNSNRLRKQSINETPLGSLSKDDGARGAHGKKLNFSESHLVIITKEEEQKADPKEVSNSKTKTIFPKVLGKSQSKHISRNRSEMSSSSLAPHRVPLSQTNHALTGGLEPAKINITAKAPSTEYNQSHIKALLPEDSGTHQVLRIDVTLSPRDPKAPGQFGRPVVVPHGKEKEAERRWKEGNFNVYLSDLIPVDRAIEDTRPAGCAEQLVHNNLPTTSVIMCFVDEVWSTLLRSVHSVINRSPPHLIKEILLVDDFSTKDYLKDNLDKYMSQFPKVRILRLKERHGLIRARLAGAQNATGDVLTFLDSHVECNVGWLEPLLERVYLSRKKVACPVIEVINDKDMSYMTVDNFQRGIFVWPMNFGWRTIPPDVIAKNRIKETDTIRCPVMAGGLFSIDKSYFFELGTYDPGLDVWGGENMELSFKVWMCGGEIEIIPCSRVGHIFRNDNPYSFPKDRMKTVERNLVRVAEVWLDEYKELFYGHGDHLIDQGLDVGNLTQQRELRKKLKCKSFKWYLENVFPDLRAPIVRASGVLINVALGKCISIENTTVILEDCDGSKELQQFNYTWLRLIKCGEWCIAPIPDKGAVRLHPCDNRNKGLKWLHKSTSVFHPELVNHIVFENNQQLLCLEGNFSQKILKVAACDPVKPYQKWKFEKYYEA

SEQ ID NO: 87 - DNA, 1200 bp, Homo sapiens
ggcttctcggagcggcgctgggcgaccggagcagggtcgagatgtcctacatcccgggccagccggtcaccgccgtggtgcaaagagttgaaattcacaagctgcgtcaaggtgagaacttaatcctgggtttcagcattggaggtggaatcgaccaggatccttcccagaatcccttctatgaagacaagacggacaagggtatttatgtcacacgggtgtctgaaggaggccctgctgaaatcgctgggctgcagattggagacaagatcatgcaggtgaacggctgggacatgaccatggtcacacacgaccaggcccgcaagcggctcaccaagcgctcggaggaggtggtgcgtctgctggtgacgcggcagtcgctgcagaaggcgtgcagcagtcatgctgtcctagcagccaccaccatctgcgactcctgcctgccgcctctctgtacagtaacgccacttccacactctgtccccatctggcttctgctgaccagctttctctcctggacaccgaggattggaaataagggcctggagctgagtagtagccagtctgctgtgaccacaggctcaggtccgaccctgctgcttggccacagcagtggctgggcaagtgggaaccactatctcttgggagcccccaaaagctgggaaatgctggaggaaccaggcctttcccgcttttgcctggctgcagggttgggctccgcccctgccccccagccttggtgtgtccacaccgcagtgcttctgcccctcgggggactggacacacatcctgccagaggcgctacgaagctttgcccagatgaagccaggtgggctccgcgttcactcccactctcccgaggggtgctggcctccccagggtttgccttcttacggatttagacgaggttcgaggctcacctatcagggcagctctcaggattgtcattttcctctttgcctgtgggtttaacttttgtatttttttaatcacaagtttgatacaaaatgtttttatcgtactctttggagatgcccattctacttttgaatttagcttttactaattcgcatctggaagctcagcaagtgcacaagccttactttggttaccgtggaaaccactgccgcccctcccggatgtggtgtgctgaataaaaatgctggaattcaaaaaaaaaaaaaaaaaaaaaaaaaaaaa

SEQ ID NO: 88 - protein, 286 aa, Homo sapiens [sequence omitted]

SEQ ID NO: 89 - DNA, 1023 bp, Homo sapiens
ccaacatggagactttgtaccgtgtcccgttcttagtgctcgaatgtcccaacctgaagctgaagaagccgccctggttgcacatgccgtcggccatgactgtgtatgctctggtggtggtgtcttacttcctcatcaccggaggaataatttatgatgttattgttgaacctccaagtgtcggttctatgactgatgaacatgggcatcagaggccagtagctttcttggcctacagagtaaatggacaatatattatggaaggacttgcatccagcttcctatttacaatgggaggtttaggtttcataatcctggaccgatcgaatgcaccaaatatcccaaaactcaatagattccttcttctgttcattggattcgtctgtgtcctattgagttttttcatggctagagtattcatgagaatgaaactgccgggctatctgatgggttagagtgcctttgagaagaaatcagtggatactggatttgctcctgtcaatgaagttttaaaggctgtaccaatcctctaatatgaaatgtggaaaagaatgaagagcagcagtaaaagaaatatctagtgaaaaaacaggaagcgtattgaagcttggactagaatttcttcttggtattaaagagacaagtttatcacagaattttttttcctgctggcctattgctataccaatgatgttgagtggcattttctttttagtttttcattaaaatatattccatatctacaactataatatcaaataaagtgattattttttacaaccctcttaacattttttggagatgacatttctgattttcagaaattaacataaaatccagaagcaagattccgtaagctgagaactctggacagttgatcagctttacctatggtgctttgcctttaactagagtgtgtgatggtagattatttcagatatgtatgtaaaactgtttcctgaacaataagatgtatgaacggagcagaaataaatactttttctaattaaaaaaaaaaaaaaa

SEQ ID NO: 90 - protein, 149 aa, Homo sapiens
METLYRVPFLVLECPNLKLKKPPWLHMPSAMTVYALVVVSYFLITGGIIYDVIVEPPSVGSMTDEHGHQRPVAFLAYRVNGQYIMEGLASSFLFTMGGLGFIILDRSNAPNIPKLNRFLLLFIGFVCVLLSFFMARVFMRMKLPGYLMG

SEQ ID NO: 91 - DNA, 3901 bp, Homo sapiens
gccatggagggagtgagcgcgctgctggcccgctgccccacggccggcctggccggcggcctgggggtcacggcgtgcgccgcggccggcgtgttgctctaccggatcgcgcggaggatgaagccaacgcacacgatggtcaactgctggttctgcaaccaggatacgctggtgccctatgggaaccgcaactgctgggactgtccccactgcgagcagtacaacggcttccaggagaacggcgactacaacaagccgatccccgcccagtacttggagcacctgaaccacgtggtgagcagcgcgcccagcctgcgcgacccttcgcagccgcagcagtgggtgagcagccaagtcctgctgtgcaagaggtgcaaccaccaccagaccaccaagatcaagcagctggccgccttcgctccccgcgaggagggcaggtatgacgaggaggtcgaggtgtaccggcatcacctggagcagatgtacaagctgtgccggccgtgccaagcggctgtggagtactacatcaagcaccagaaccgccagctgcgcgccctgttgctcagccaccagttcaagcgccgggaggccgaccagacccacgcacagaacttctcctccgccgtgaagtccccggtccaggtcatcctgctccgtgccctcgccttcctggcctgcgccttcctactgaccaccgcgctgtatggggccagcggacacttcgccccaggcaccactgtgcccctggccctgccacctggtggcaatggctcagccacacctgacaatggcaccacccctggggccgagggctggcggcagttgctgggcctactccccgagcacatggcggagaagctgtgtgaggcctgggcctttgggcagagccaccagacgggcgtcgtggcactgggcctactcacctgcctgctggcaatgctgctggctggccgcatcaggctccggaggatcgatgccttctgcacctgcctgtgggccctgctgctggggctgcacctggctgaacagcacctgcaggccgcctcgcctagctggctaaacacgctcaagttcagcaccacatctttgtgctgcctggttggcttcacggcggctgtggccacaaggaaggcaacgggcccacggaggttccggccccgaaggtcagagaagcagccatgactgcggggggaggacacacggatgctcaggcccaggctttgccaggtccgaagcgggcccctctctgtcctgcctcttttcacctgctcacgccctcccacccccaccctacagccccaggtcctggcccagtccctccactgcctcgaagagtcagtctgccctgccttttcctttcgggcaccaccagccatccccgagtgccctgtagccactcaccactgctgccacctctctggccaatggccctttcactggcctggtgactggaatgtgggcagcgcccacacaggctctggcccatggcttcctactggcagctccaggcacccccctctcaccacgccgtttgctggctctgacactgttgggtgagggtcctggtcctgctgtcttcccttctggcctctgcacaggggtggtgacagtggctacaggctgggcccctggcgtgccctgaccgtgcagcagagtgaggctggggcagcagagagccccagcctcacccctgaggagcacctgtggtctgtccccttggtcctgcttatggctggaccggccctgcaggaggtggtggagccgtgaaggaggccgagctgcagctctggctgctgcttggcctcctgctccaagaccctcccgagtccccggaaatggagagtgcagttcttgggcccagcctggccttcgccatgagtttggggagcgagaccccacctgagacaggcagtaggagcctgtgctgaccttggggaatctgagcttttccaagggtaaggggcccagggtatgcaggccttcagtgacatcaggtcgttgtcatcctttccctccctgacctgtcacgagcctctgcaggtgcctgctcaccatggcccagcgccactctgtcctccgactcaggtgagggggcagcccacagacctgctcctcagtagcagggcctggccaggcccctgctgttctcagcctcagtttgccatctatgaaatgaggtggacccctctccatagcccttgggtgccagctcagtgggtgtggggatcacatgaggtggctcatgaggacacactctggaagtcgaggggctgccacgtgcagaggaagttcccggcctgggggctttatccaggggtcccagtcgagagtggcccgaggccgtccctcaccgggcatgttccctctggctgcccactccctcagggcccacatgtcctgccactcgccactctgagcacgagttcaccttccagatgtggccagggtgtgccagctcctctctcctgtgcgttggaaccccgggggaggcaagagcagatcacaggtgcatgagggttacacccgtcacctgggtctgccgggatgggttgggggggcaggtgccaggcctcactgctgtgaatctgccacgcctgggggtcctagaggctgccccaccccagtgattgggtagcagctcacatcccacccagcttcacaagtgaggaacccaggtgcatcgggagaccctcgggggcttctgtggcctctgtgcccgatgacctgcgtggcttcagacaaggccccagcgttactgggctcagcttgttgttctgtgtggagcgtgaggtgagaaaacccctctgaaaagatgtggtcggggccacgcttcccactggttctgcagtgaggagttggggcgggtgagccaaagcggccccccatggtgtctacctgaggggcagggaaccgcctgcctgtgcactcacgccaccccccagcccacaaagagcccatctgagagaaggacgtggtggagccaggacgggaaagcgtcctgtcggctggccatgctgttgcttgcgtctcgaatcttcggttctcgaggaagtgttgacagtgtgatgctaatgtctgcttttcttggcgttgggtagaagcaggacatctgtgtgtatgtgcgtatttaaattagattatttataataaccagagccagccctcgcgctggccaggatcctcctgccgagctgatgtcgctcctgccctctgccggggtccggaagcgacatctcaggaggtagctctcagcagagtgaggattcctgcctttcgtagagttttgtgtgactttttaaattattcatgtgtcccttaaaagtttcactacgtggagaaaattccagcaccaagtgttgtggcaacagctgagagagtgcaggcaccactgtgttgtggcttgttgaccgggaatgtgtcacccctgccagggaactcttctcctcgcgggggacttgggatggccatcagaccttctagggtctggctggggtcatcctaggtatgggtgaccgtccctgagacataagcgaggtagattcagccatcctcaccctcagacttgaggtccccacccaggccaagccggccccccgtaccccttgcctgggagcaaaccgccaggacgcagcctccacgccgcacctgccacattcagccctgcccaggaaggaacacatgacccttctgtctgtgactgttgctgagtctctgtctcatgtcgtagaattgtggataattgtctagtgaccctctcatcactgtaaccatcgcgcctggcctagatgtcgtgttttggatgctgtgttttcaataaatgcctctggggccctgcttttaccaaaaaaaaaaaaaaaaaaaaaaaaaaaaaaaaaaaaaaaaaaaaaaaaaaaaaaaaaaa

SEQ ID NO: 92 - protein, 392 aa, Homo sapiens
MEGVSALLARCPTAGLAGGLGVTACAAAGVLLYRIARRMKPTHTMVNCWFCNQDTLVPYGNRNCWDCPHCEQYNGFQENGDYNKPIPAQYLEHLNHVVSSAPSLRDPSQPQQWVSSQVLLCKRCNHHQTTKIKQLAAFAPREEGRYDEEVEVYRHHLEQMYKLCRPCQAAVEYYIKHQNRQLRALLLSHQFKRREADQTHAQNFSSAVKSPVQVILLRALAFLACAFLLTTALYGASGHFAPGTTVPLALPPGGNGSATPDNGTTPGAEGWRQLLGLLPEHMAEKLCEAWAFGQSHQTGVVALGLLTCLLAMLLAGRIRLRRIDAFCTCLWALLLGLHLAEQHLQAASPSWLNTLKFSTTSLCCLVGFTAAVATRKATGPRRFRPRRSEKQP

SEQ ID NO: 93 - DNA, 2203 bp, Homo sapiens
cagcggtgggaggcggcgaccagccggttgaggccccaggcttggcctcaccacaatgtggcacgaggctcggaagcatgagcggaagcttcgaggcatgatggtcgactacaagaagagggcggagcggagacgggagtattatgaaaagatcaagaaggacccagcccagttcctgcaggtacatggccgagcttgcaaggtgcacctggattctgcagtcgccctggccgctgagagccctgttaatatgatgccctggcagggggacaccaacaacatgattgaccgattcgatgtccgtgcccacctggaccacatccccgactacaccccccctctgctcaccaccatctccccagaacaggagtcggacgaacggaagtgtaactacgagcgctacagaggcctggtgcagaacgactttgccggcatctcagaggagcagtgcctgtaccagatctacattgatgagttgtacggaggcctccagagacccagcgaagatgagaagaagaagctggcagagaagaaggcttccatcggttatacctacgaggacagcacggtggccgaggtagagaaggcggcagaaaagccagaggaggaggagtcagcggccgaggaggagagcaactcggacgaagatgaggtcatccccgacatcgacgtggaggtggacgtggatgaattgaaccaggagcaggtggcagatctcaacaaacaggccacgacttatggcatggccgacggtgacttcgtcaggatgctccggaaagacaaggaggaggcagaggccatcaagcatgccaaggctcttgaggaggagaaggccatgtactcgggacgccgctctcgacgccagcggagagagtttcgggagaagcggctgaggggtcgcaagatcagcccacccagctatgcccgccgagacagccccacctatgacccctataagcggtcaccctcggagtccagctcagagtcccgctcccgctcccgctccccgaccccgggccgcgaggagaagatcacgttcatcaccagttttgggggcagcgatgaggaggcagccgcagccgctgctgccgcagcagcatcaggagtcaccacagggaagccccccgcacctccccagcctggcggccccgccccgggacgtaatgccagcgcccgccgccgctcctcctcctcctcctcctcctcttctgcctcgaggacctccagctcccgctccagctctcgctccagctcccgctctcgccgtggtgggggctactaccgttccggccgccacgcccgctcccggtcccgctcctggtcccgctcccgctcccgctcccggcgctattcccggtcccgtagccgtggccggcggcactcaggtgggggctcccgagacggacaccggtactcccgctcgcccgcccggcgtggtggttacgggccccggcgcagaagcaggagccgctcccactcaggggaccgctacaggcggggcggccggggcctcaggcaccacagcagtagccgcagccgcagcagctggtccctcagcccgtcccgcagtcgcagcctgactcgcagccgcagccatagccccagccccagccagagccgcagccgcagccgcagccgcagccagagcccctcgccatcacccgcaagagagaagctgaccaggccggccgcgtcccctgctgtgggcgagaagctgaaaaagaccgaacctgccgctggtaaagagacaggagctgccaaacccaagctgacgcctcaggagaagctgaaactgaggatgcagaaggcgctgaacaggcagttcaaggcggataagaaggcggcacaagaaaagatgatccagcaggagcatgagcggcaggagcgggaagacgagcttcgagccatggcccgcaagatccgcatgaaggagcgggaacgccgagagaaggagagagaagagtgggaacgccagtacagccggcagagccgctcaccctccccccgatacagtcgagaatacagctcttctcgaaggcgctcaaggtcccgatcccgaagcccccattaccgacattaggcagaagagtggggggtggggaggacaagggggtgggtaaggggctcaagctgtgatgctgctggttttatctctagtgaaataaagtcaaaagttatttaattcccgtcaaaaaaaaaaaaa

SEQ ID NO: 94 - protein, 674 aa, Homo sapiens
MWHEARKHERKLRGMMVDYKKRAERRREYYEKIKKDPAQFLQVHGRACKVHLDSAVALAAESPVNMMPWQGDTNNMIDRFDVRAHLDHIPDYTPPLLTTISPEQESDERKCNYERYRGLVQNDFAGISEEQCLYQIYIDELYGGLQRPSEDEKKKLAEKKASIGYTYEDSTVAEVEKAAEKPEEEESAAEEESNSDEDEVIPDIDVEVDVDELNQEQVADLNKQATTYGMADGDFVRMLRKDKEEAEAIKHAKALEEEKAMYSGRRSRRQRREFREKRLRGRKISPPSYARRDSPTYDPYKRSPSESSSESRSRSRSPTPGREEKITFITSFGGSDEEAAAAAAAAAASGVTTGKPPAPPQPGGPAPGRNASARRRSSSSSSSSSASRTSSSRSSSRSSSRSRRGGGYYRSGRHARSRSRSWSRSRSRSRRYSRSRSRGRRHSGGGSRDGHRYSRSPARRGGYGPRRRSRSRSHSGDRYRRGGRGLRHHSSSRSRSSWSLSPSRSRSLTRSRSHSPSPSQSRSRSRSRSQSPSPSPAREKLTRPAASPAVGEKLKKTEPAAGKETGAAKPKLTPQEKLKLRMQKALNRQFKADKKAAQEKMIQQEHERQEREDELRAMARKIRMKERERREKEREEWERQYSRQSRSPSPRYSREYSSSRRRSRSRSRSPHYRH

SEQ ID NO: 95 - DNA, 1014 bp, Homo sapiens
gggcgccgccgcctcctcctccatggctgtttacccggctgcattgtgggagtttgacctccgccgccgccaaccgccgcctcagcttgcgccgccgccgccgccgccgccgccgcgcacgccatgggagccgtgactgacgacgaagttatacggaagcgtctcctcattgatggagatggtgctggagatgatcggagaattaatctgctagtgaagagtttcattaaatggtgcaactctgggtcccaggaagagggatatagccagtaccaacgtatgctgagcacgctgtctcaatgtgaattttcaatgggcaaaactttactagtatatgatatgaatctcagagaaatggaaaattatgaaaaaatttacaaggaaatagaatgtagcatagctggagcacatgaaaaaattgctgagtgcaaaaagcaaattcttcaagcaaaacgaatacgaaaaaatcgccaagaatatgatgctttggcaaaagtgattcagcaccatccagacaggcatgagacattaaaggaactagaggctctgggaaaagaattagagcatctttcacacattaaagaaagtgttgaagataagctggaattgagacggaaacagtttcatgttcttcttagtaccatccatgaacttcagcaaacattggaaaatgatgaaaaactctcagaggtagaagaagctcaggaagcaagcatggaaacagatcctaagccatagacaggctaattgcccaccactcccaggaatattgaaatagctacatgaccataatgtgtttaaaatgtggtatgctcttgagatatttaaagttttggcagtaaaatactctgtttttaagtatgaatgtatttcattcatatttcctctcacaaaggaaaatgacttcagtatagatttgtttttattaaaatgcattttttattcttaagtggtaggaagcaacatccaaaaatgcttaataaaatgcttttaagctgcaaaaaaaaaaaaaaa

SEQ ID NO: 96 - protein, 204 aa, Homo sapiens
MGAVTDDEVIRKRLLIDGDGAGDDRRINLLVKSFIKWCNSGSQEEGYSQYQRMLSTLSQCEFSMGKTLLVYDMNLREMENYEKIYKEIECSIAGAHEKIAECKKQILQAKRIRKNRQEYDALAKVIQHHPDRHETLKELEALGKELEHLSHIKESVEDKLELRRKQFHVLLSTIHELQQTLENDEKLSEVEEAQEASMETDPKP

SEQ ID NO: 97 - DNA, 955 bp, Homo sapiens
aatcctcaacaaaatagtagcaaaacaaatccaatagtacatcacgaagataatacagtatgatcaaatgggatttatttcaaggatgcatagatgattcaacattcacgatcaataaaatttattctgttaatttttcataaggtatgtttttaaataagaatggtattgtatattagacataaaatgactgttttagttagcattcttagagctagcttcataatccaattaatatacttgcaacttgagtgcaggtgttttaatttttataactgtatcctgtatgctattcaaatgagctaattgtagttattcttatacccattggtattggtttccatagtatacataagttttatttttgtttttcctgttagaccttcaaatatttactttccatagtttctctggcataaaagctcccagtttctatcttcaacagttcaggtcttgggatatctatcgttttatttgtttttataacttttatttagaagagttacatcctttttagcttatttaatgataaaaagttcactttttccacttttgtatttgaatgaattgctgcccctaacatggatctatcttggttttacagagagaagagaagaggaagatattgaagagaagaaatcgattaagaaaaaaattaaagaacttaagtttttagattctaaaattgcccagaacctttgtaagtatcatattccaataccattcaaggacagtggaaatatttctttaaatgatttcattttctttaagaccgattattcattatttgctattttcattttgttattatatgcatgataaattcacagataactctcctttaggtaaattatgggattaaatgcttcaaaagataagtgcatattagaaaatacaaataagaagaggttttaaaatgaaattctacctttcataactgaaaaaaaaaaaaaaa

SEQ ID NO: 98 - protein, 97 aa, Homo sapiens [sequence omitted]

SEQ ID NO: 99 - DNA, 1375 bp, Homo sapiens
gtcttcttttagggagcaggagtgcatctggtaattgagggtggatgttgtgtgtgctggggaggggtccttctgtttggtgctacccttgtctactctgcccctggatggtgcggggtgctttctccacccccacactccctgctcagctcctcgtgctgccctgcatgcccaggcttgtgagccaagctgctttttggggcagggagtagcagcaggtgggaggggttacccatcagcccttgcaagtcccccactcaggcctctggaaggtccagggatgggctctgatgagagggtaaaagatgctcagggaaacacaggcctcagctgcctagaggaccctccccctgccttgcagtgggctcgggtagagcagtatcaggagctagggttgtctgctgcccacactcctgctttttgggatatctaactgctaaggagggagttgacatcccccttctggctcatgtgtctgacaccaacaacatggtctctgtccctctctctttgactctccctttgtcctccccatagagctggggtggggtggatccctatacctggggcaggcagccccaaagtgggggagggggatggcagagactgtaaaggcgccactggactctggcaaggcctttattacctttactcccctccctctcccatcaccagcctcaaggcctgaggggtgcaggggctcctggcagctactgggtgaggtttcctggcacagactcacccttctttctggcaccacctctttcccttttgaagagacagcaacagccgtagcaaaagcagctgctgctcctgctatgagggtgtatatattttttacccaaagctctggaattgtacatttattttttaaaactcaaagagggaaagagccttgtatcatatgtgaacattgtatcataggtaatgttgtacagacccttttatacagtgatctgtcttgttcctgcagcaaaaatcctctatggacataggaggtgctgtgtcccatgccctcttgccctgacagtgtcccatgggcccccttctgctccctgccccctccctgctactgctgatgcactctcctctccctgcagcccctggcttcccagccttcctcctgaccccttccaacagccttggaactccagctgccaccaccctctgggtcggacactgggacccactggcccagtcttggctgctgcttacccctagccttgatgcctgcccagggacccccagccccctcccgttgccctgcagctttaacagagtgaaccatgtgtattgtacaggcgcggttgtcattgcagaaaccgctgggtggagaagaagccgataaagtctatgaatcaaaaaaaaaaaaaaa

SEQ ID NO: 100 - protein, 132 aa, Homo sapiens [sequence omitted]

SEQ ID NO: 101 - DNA, 1213 bp, Homo sapiens
ggcttcaggttgaagtccctggttcttccagttcctcacgggttaggtaggggctcctgcatcaccttcagaatccagttccaacccccactctccttaggccttgtgctctgctctgccctgccaggctgcccttgtccatgtgagtagcatgggcgggtggtggggacggcagtggtgatgaagggggtgcaccacaggcctcatgaagcagttcccacatgggcgtgtggctggggcgtggccaccacagagcacatggctgtgtctaggcgcaagcactttagcagtatctgtttacatgcgcaaggatcaagccgactacctgtgctgtctactgggacagcagtctccgagctactccgtacctccctctgccaggtcgtggagttaggccccagtccctacttgtcactggttcccactgtgctcctaactgtgcagcacctgggagctctggcctggggctggaggccctggtaggagctgcagttggaggccgttctgtgcccagcagcggtgagcggctcccatgggccctgtgtctgcagggagccagggctgcggcacatgtgctgtgaaactggcacccacctggcgtgctgctgccgccacttgcttcctgcagcacctcctaccctgctccgtgtcctccctctccccgcgcctggctcaggagtgctggaaaagctcacgcctcggcctgggagcctggcctcttgatatacctcgagcttcccctgtgctccccagccccaggaccactggccccttggcctgaggggctgggggccccacgacctgcagcgtcgagtccgggagagagcccggagcggcgtgccatctcggctcggccttgctgagagcctccgccctggctttctccctgtctggattcagtggctcacgttggtgctacacagctagaatagatatatttagagagagagatatttttaagacaaagcccacaattagctgtcctttaacaccgcagaaccccctcccagaagaagagcgatccctcggacggtccgggcgggcaccctcagccgggctctttgcagaagcagcaccgctgactgtgggcccggccctcagatgtgtacatatacggctatttcctattttactgttcttcagatttagtacttgtaaataaacacacacattaaggagagattaaacatttttgctaaaaaaaaaaaaaaaa

SEQ ID NO: 102 - protein, 100 aa, Homo sapiens
MKGVHHRPHEAVPTWACGWGVATTEHMAVSRRKHFSSICLHAQGSSRLPVLSTGTAVSELLRTSLCQVVELGPSPYLSLVPTVLLTVQHLGALAWGWRPW

SEQ ID NO: 103 - DNA, 1036 bp, Homo sapiens
cctcaaatgctttctttcttcagatgctttttcgtgtacatgatactagtagacacttttctctttatatttactgatagtgaaaatcatacgcaataaaatattgatgtttgaaggcagtggtcaccaattggttaaaaaactatgaaatgtaaactgaattgttatatctctatcctttttgcttttctctgtgtttttaatgtatggaataaatctcataaatagaaagaaaaataatctagaaatttttcaaagctagtactctttctccttataaatgtacacaattttaatctttttacaaatttatttaactgtacctactgtacttattgtagattcaatgacgcagttaagtcatcacccaaggatttatgaatttgagattactgacctgttttcttcatattgcattcacatcaatatttgtgaatttgttgttcagcttttcattcaaacaaaaaatattccctcaagaaagctccatttttatcataaacatttcaacataaccaacattagaacaagtctgccatgttaaaaataatttaaagacttatctctgaaaacggtatccagaaacgcaggtgttcccagtaatgtagcttcaaaaataaaatgtgctatttatatgacatgaaattcataacttttggaagggtatatttatgacagcataaaaaataaattctgtgctataaagaagatccaacaaattaaccatataagcacagaaaatagagaaacacagttattgaatctactcttgtcattaacattttcaaaaaacaaaatgcatattgtaatatttggtacatgacacttgcatgttgatatgcctatatacttacaaagtattcaatgtgtacttagcggcgcttaaaatatgtcatgtacaactcttataaacatttttacagggttcccatttgcacttcatctttcagtaaagtcttgtcagaaaaaaattgtctgataaatatggaaaaataaaatttgaattttagttaaaaaaaaaaaaaaaaaaaaaaaa

SEQ ID NO: 104 - protein, 87 aa, Homo sapiens
MYTILIFLQIYLTVPTVLIVDSMTQLSHHPRIYEFEITDLFSSYCIHINICEFVVQLFIQTKNIPSRKLHFYHKHFNITNIRTSLPC

SEQ ID NO: 105 - DNA, 2349 bp, Homo sapiens
tttttttttttttttggattcttggtaaaattttatccaaaaaacaggatacatatatatttagagaaggaaatatgaaatcaagagttttggcagcccctgctttttttttttttttagctccctaaagactgtagcaggataaaaggatcactggctccgagtctctttgagataacaagtgatgaaataaaaaagaaagcccataccctcaaataaggtcaggtaaccccattgcccaccctccctacaaggtaaaaaatgagtacttttagtaacagttcagaattcatctttatctcctacctgcctcatcggtggaagtttaaagtcatgattttttttagacattgatacttgtgtctatagacaaataaactcatattagacggccaaagaggcctaccactgctgcaccagcagtatacctcacgcactgcctcaccactgcccctgcgcccagatgctcctgttgaaaagtcacccgaggagacagctacccaggtccccagtctggagagtctgactttaaagctagagcacgaggtggtggccaggagccgaccaaccccacaagactatgagatgcgagtatccccctctgatactacccctctggtttcccggagtgttccaccagtcaaactggaggatgaggatgattcggactctgagctggacttgagcaagctgtcaccatcttcttcttcttcctcatcctcatccagctccagctccagcactgatgagagtgaggatgagaaggaagagaagctaactgaccagtcccgctcaaagctctatgatgaagagagtctcctgtccctcactatgtcccaagatggattcccaaatgaagatggagaacaaatgacccctgagcttctgctactgcaggaaagacaaagagcctctgagtggcccaaggatcgtgtcctgataaaccgtattgacctcgtctgccaggctgtactctcagggaagtggccttctagccgtaggagccaggaaatggtaacaggaggaattttggggccaggcaaccacttgctagacagtccctcattgactcctggagaatatggtgactctccagtccccacaccacgaagtagtagtgcagcttccatggcagaggaggaagcatctgcagtcagcacagcggcagcccagttcaccaaacttcgccgaggcatggatgaaaaggagtttacagttcaaatcaaagatgaggaaggattgaagttaacattccagaagcacaagttgatggcgaatggagtaatgggagatggacatccactgtttcataagaagaaggggaacagaaagaagctagtagagctggaggtggagtgcatggaagagcctaatcaccttgatgtggacctggagacccggatccctgtcatcaataaggtggatggtactttgctggtgggtgaggatgcccctcgccgggctgaactggagatgtggttacagggtcatccagagtttgctgttgatccccgatttctagcgtatatggaggatcgcagaaaacagaagtggcaaagatgtaaaaaaaataataaggcagaattgaactgtttgggaatggaaccagtacagacagctaactctagaaatgggaaaaagggtcatcacactgaaacggtgttcaaccgggttttgccagggcctattgcaccagagagcagcaagaagcgggcccgtaggatgcgaccagacctttctaagatgatggccctcatgcagggtggaagcactgggtctctatctctgcataacacgttccaacacagcagtagtggcctacagtctgtgtcatctttgggtcacagcagtgccacttctgcatctttgccttttatgccatttgtgatgggtggtgcaccatcatcccctcatgtagactccagcaccatgcttcatcaccaccaccaccacccccacccccaccatcaccaccatcaccatccaggcttgagagcccctggctacccctcttcaccagtgactaccgcctctggtactaccttgcggttgccaccactgcaacctgaggaggatgacgatgaggatgaagaagatgatgatgacttatctcagggctatgatagctcagaaagggacttctcactcattgatgatcctatgatgccagctaactcagactccagtgaagatgctgatgactgaagccccagcatgggccccattgcttgggcggctgctgtattttcatttactctggcccttggactatggaaacgtgggaggggcaggggagatgtggggaagtccaggactccaggaggtgaaaaggaaaaaaaaaaaaaaaaa

SEQ ID NO: 106 - protein, 539 aa, Homo sapiens
MRVSPSDTTPLVSRSVPPVKLEDEDDSDSELDLSKLSPSSSSSSSSSSSSSSTDESEDEKEEKLTDQSRSKLYDEESLLSLTMSQDGFPNEDGEQMTPELLLLQERQRASEWPKDRVLINRIDLVCQAVLSGKWPSSRRSQEMVTGGILGPGNHLLDSPSLTPGEYGDSPVPTPRSSSAASMAEEEASAVSTAAAQFTKLRRGMDEKEFTVQIKDEEGLKLTFQKHKLMANGVMGDGHPLFHKKKGNRKKLVELEVECMEEPNHLDVDLETRIPVINKVDGTLLVGEDAPRRAELEMWLQGHPEFAVDPRFLAYMEDRRKQKWQRCKKNNKAELNCLGMEPVQTANSRNGKKGHHTETVFNRVLPGPIAPESSKKRARRMRPDLSKMMALMQGGSTGSLSLHNTFQHSSSGLQSVSSLGHSSATSASLPFMPFVMGGAPSSPHVDSSTMLHHHHHHPHPHHHHHHHPGLRAPGYPSSPVTTASGTTLRLPPLQPEEDDDEDEEDDDDLSQGYDSSERDFSLIDDPMMPANSDSSEDADD

SEQ ID NO: 107 - DNA, 3004 bp, Homo sapiens
ggggcatgagcatctcagggctgccagaatggcttttgctgagtgcatagcaccagcgtgtgtcatgtcttggctgcgtttctggggcccatggcccctccttacgtggcaactattgtctttactagtcaaggaggctcagcctctggtgtgggtcaaggacccgctccagctgacctctaaccccctggggccacctgagccctggtcttcccgctcctcccatctcccatgggaatctccccatgcacctgctcccccagcagccccgggggactttgattacctggggccctctgcttcttcgcagatgtcagccctgcctcaggaaccaactgaaaatttggctccattcctgaaggaattggattcagctggagagctgcccctggggccagagccgttcttggctgcacatcaggacttaaatgacaagcggactccagaagaaaggctcccagaggtggttccgcttctcaaccgggatcagaaccaggccctagttcagcttcctcgcctcaagtgggttcaaactacagatctagatcgggctgcaggtcatcaggcagatgaaatacttgttccactagacagtaaggtttcaagaccaaccaaatttgttgtttcgcccaagaacctgaagaaagatctagctgaacgttggagccttcctgagattgttgggattccacaccaattatccaaacctcagcgtcagaaacagactttgccagatgattatttgagtatggacacactgtatcccggcagcctacctccagaactccgggtgaacgcagatgagcctccagggcctcctgagcaagttggactttctcaattccatctagagcccaaaagtcaaaatccagagacccttgaagacatccagtcctcttcactccaggaagaagccccagcgcagcttctacagctccctcaggaggtagaaccttcaacccagcaggaggccccagctctgcctccagagtcctctatggagagtctagctcaaactccactgaatcatgaagtgacagttcaacctccaggtgaggatcaagctcattataatttgcccaagtttacagtcaaacctgcagatgtggaggttaccatgacttcagagcctaaaaatgagacagaatctacccaagcccagcaggaggccccaattcagcctcccgaggaggcggaaccttcttctacagccctgaggactacagatcctcctccagaacaccctgaggtgacacttccaccttcagacaagggtcaggctcagcattcacacctgactgaagccacagttcaacctctggacctggagcttagcataactacagagcctactacagaggttaaaccgtctccaaccacggaggaaacctcagctcagcctccagacccggggcttgccataactccagaacccactacagagattggacattccacagccctggagaagactagagctcctcatccagaccaggttcagactctgcatcgaagcctgactgaagtcacaggtccacctacaaagttagaatcttcgcaggattcattggtgcagtctgaaactgcaccagaggaacagaaggcctccacaagcaccaacatatgtgagctctgcacctgcggagatgagactctgtcatgtgttggtctcagcccaaagcagaggctccgccaagtgcctgtgccagagcccgacacctacaatggcatcttcaccaccttaaatttccaaggaaactatatttcataccttgatggaaatgtatggaaagcatacagttggaccgagaaactaattctcagtgaaaattatttgactgaattacctaaggattcatttgaaggcctgctatacctccagtatttagatttatcctgcaataaaatacgatatattgaaagacaaacatttgaatcactaccatttttgcagtatataaatctgggctgcaatttaattacaaaactgagccttggaacatttcaggcctggcacggaatgcagtttttacacaacttaattctcaatcgcaatcctctgactactgtcgaagatccatatctctttgaactgccggcattaaaatatctagacatgggaacaacacacatcacacttacaacacttaagaacattctcacgatgactgttgaactggaaaaactgatcttacctagccatatggcctgctgcctctgccaatttaaaaatagcattgaggctgtctgcaagacagtcaagctgcattgcaacactgcatgtctgactaacagcatacattgtcctgaagaagcatctgtagggaatccagaaggagcgttcatgaagatgttacaagcccggaagcagcacatgagcactcagctgactattgagtcggaggcgccctcagacagcagtggcatcaacttgtcaggctttgggggtgatcagcttgaaattcagctaaccgagcagctacggtccctcatccccaacgaggatgtgagaaagttcatgtctcatgttatccggaccttgaaaatggaatgttcagaaacacatgtgcaagggagctgtgccaagctcatgtcgcgaacaggcctcctgatgaagcttctcagcgagcagcaggaagcaaaggcattgaatgtagaatgggatacggaccaacaaaaaacaaattatattaatgagaacatggaacagaatgaacagaaagagcagaagtcaagtgagctcatgaaagaagttccaggagatgactataagaacaaactcatcttcgcaatatctgtgactgtaatactaataattttgattataattttttgtcttatagaggtgaattcacataaaagggcatcagaaaaatacaaagacaacccatcaatatcaggagcctgagcatgagttaaagcatgtggatggcctggagctatgtttttaaaattgttattaaatattggttttttacttaaaaaaaaaaaaaaaaaaaaaaaa

SEQ ID NO: 108 - protein, 959 aa, Homo sapiens
MAFAECIAPACVMSWLRFWGPWPLLTWQLLSLLVKEAQPLVWVKDPLQLTSNPLGPPEPWSSRSSHLPWESPHAPAPPAAPGDFDYLGPSASSQMSALPQEPTENLAPFLKELDSAGELPLGPEPFLAAHQDLNDKRTPEERLPEVVPLLNRDQNQALVQLPRLKWVQTTDLDRAAGHQADEILVPLDSKVSRPTKFVVSPKNLKKDLAERWSLPEIVGIPHQLSKPQRQKQTLPDDYLSMDTLYPGSLPPELRVNADEPPGPPEQVGLSQFHLEPKSQNPETLEDIQSSSLQEEAPAQLLQLPQEVEPSTQQEAPALPPESSMESLAQTPLNHEVTVQPPGEDQAHYNLPKFTVKPADVEVTMTSEPKNETESTQAQQEAPIQPPEEAEPSSTALRTTDPPPEHPEVTLPPSDKGQAQHSHLTEATVQPLDLELSITTEPTTEVKPSPTTEETSAQPPDPGLAITPEPTTEIGHSTALEKTRAPHPDQVQTLHRSLTEVTGPPTKLESSQDSLVQSETAPEEQKASTSTNICELCTCGDETLSCVGLSPKQRLRQVPVPEPDTYNGIFTTLNFQGNYISYLDGNVWKAYSWTEKLILSENYLTELPKDSFEGLLYLQYLDLSCNKIRYIERQTFESLPFLQYINLGCNLITKLSLGTFQAWHGMQFLHNLILNRNPLTTVEDPYLFELPALKYLDMGTTHITLTTLKNILTMTVELEKLILPSHMACCLCQFKNSIEAVCKTVKLHCNTACLTNSIHCPEEASVGNPEGAFMKMLQARKQHMSTQLTIESEAPSDSSGINLSGFGGDQLEIQLTEQLRSLIPNEDVRKFMSHVIRTLKMECSETHVQGSCAKLMSRTGLLMKLLSEQQEAKALNVEWDTDQQKTNYINENMEQNEQKEQKSSELMKEVPGDDYKNKLIFAISVTVILIILIIIFCLIEVNSHKRASEKYKDNPSISGA

SEQ ID NO: 109 - DNA, 1331 bp, Homo sapiens
gttcttttcttttccatgatatcattatatggacagtttagggtggtctcatggattataaccatttggatatttggttcactaacaattttcttactggccagagttcttggtggagaagttgcatatggccaagtccttggagttataggatattcattacttcctctcattgtaatagcccctgtacttttggtggttggatcatttgaagtggtgtctacacttataaaactgtttggtgtgttttgggctgcctacagtgctgcttcattgttagtgggtgaagaattcaagaccaaaaagcctcttctgatttatccaatctttttattatacatttattttttgtcgttatatactggtgtgtgatccaagttatacatgaatagaaaaagatggtgttaaatttgtgtgtaggctgggaattcttgctgaaggaattggagaaaacctgttgctgcaaaattttacatgttccagatggaaagggaagtctaagcgctttttaaaacaatttttttttgtatttaattaagcaattgcagttatctgggatttttgggtcagaattttaaattctgtttgattctccatattccagtgaataaaatacaaaagcattgtgtttttaagattgtgtcgatattcacctaaaaacttgtgccaaaagcacctggattggtaattatatttcacttaaagggtaaatttgacaatatcttgataatcaaaagtgcaatttttttcttcaaaatgttttctccagcatcacagatcctgcagatatatatttatatttatacatatatatttatgaaataattcttactcacaaaatatatttctgataaacattaagatattaaatctgatgcacaaactttttaatttggccattaatcttttttatttaaaaatttaaatttgtttttaaaattgtatatagtttttaaaatctcacacatgcttcgatacttccttgttaagaattcttaataactactaaaactgatttttaatagttgctgatatatatttggtttgtttgggtatacttttcaaaaccatttttgaatgtccaaacatctgatttaaagtttctgtttatctttctgaccaaaggagcaagaggtataatggatatggcattcattaaaatctttactatgtacaaaaacagtaatatttacagcatcagtaaatatttttaagtggtacttctaaatcataaaagttggggaaagagacctttaaaatcttgtggtgttgaacaatgttatatgaagtagaaaaaataaaatacttcccagttgaaaaaaaaaaaaaaa

SEQ ID NO: 110 - protein, 118 aa, Homo sapiens
MISLYGQFRVVSWIITIWIFGSLTIFLLARVLGGEVAYGQVLGVIGYSLLPLIVIAPVLLVVGSFEVVSTLIKLFGVFWAAYSAASLLVGEEFKTKKPLLIYPIFLLYIYFLSLYTGV

SEQ ID NO: 111 - DNA, 2610 bp, Homo sapiens
aattgaccatctctgtgtaattctgattgtgtaccttgagacatacagaggatttctatttctcttttttgcctaagaatgtataattagtgtcattttattggggagaaattttatttttttgtcatttctctgaagttgacatttgatgagtgtattctgaattctaccactcctctggggaaaaaaatctcaatggaagattgtacagtttaagtacttgtttttccctcttgaggtatgctatttttaaggtcattaatttaaaatagaaatatttctttaaaccattggggtaaaattttttaaaatatggtgctttgatataattttgagaaaagtgcctgaatcctaaaactaactgtctataaagttaagtccgtataacaaatgattatatatataacaaagtaaaagtaaatagtgtagaatattaaagttctcttccagggcaatgggtattgtgggggattaaacagaagcccctttgctatgatcaactgcaaaggaaagaaactaaatatggttcagtgataaacaaaaatagcaaagactgatcaggaaaatgagaaagagctatgcaaatacagatgatcaccaggccagagaattttaagtgctaatagctttactcctgcatattccatcagtagaatgagctttttcttttagtctaagtgacactttgaaggtctttgcattcattacgcttttatatactgcttttcttttctcttctttttatttcatccaacacactaagaaatgaagaagtttattgatatattgcccaataaaatacaaatctgttgcaacacaatgcatatatcttattagcattcttgttagttctaaaattcaattcatccatagacacttattcccagccttcaaatggaaagctctctttatgcaagagaaaaaggtatttaaaagtgaccaaattttagaatgaggaattggttatgctgaatttctgttccaaaatcactaggtaaaatttccatatctaataatgtattttaggaacaagccaaggtttgtcattaagacatagtaacattaaactaatatttaaaaatagatttagctatatttaatcatgagaaaaggatttctgtggccaggacaaaacccaccttgatcctaaagagacattagcagtgcccatgtcagagccttctcacattcttggaataaagtaaaccactccaaggaaggtgagggcaccaaatggatcattcttcaaaatgaaaggcagtctcttactttctcttgagacatttcttccctatttggggattttatgactgctgttctggagagcccacatgttttgcttccatgcaggccaaatccttctcttgggtttgggcaagagaagttaatccacctaacagcattttgattagacaagaatatcatctgatcttatccagggacaagaaagtggcatgaccaaaggttctccttatgtctctttctttaaatgatttcctaattttcagaagggtcctggatagactagtttctgataaggagattttgctgtggtgtctgttcttccaggttaaaggcacacaaagccttctgtggtccacttgtgcttgtcttctgaattcactgaaacatggcagaataaggttaagggaaaatgaattttgactttacatagttaaatgagtaggagttacagcaaaaaaaaaaaaaaagacctaaaacttttatttaatagtattcttcacctcaggaaattcagacctttagttgttcaggtcagaaaatgtggatatatcctgtaacactgtcttcacctcactcaagttaaatgtaacagcaaatactttcagctttactttcaaaaggcttccagaactttccacatttcatcactttcacttctacccttctggtcaaaattaccaagatttttagtagtttactgttccctggtttctctgatttgatccttgctaccattctgtttagtcctcaaagaaaaaaaatcaacattttaaaacgtttcaattcttactaatggttctcatctcagaagaaaaaacaacgaaatatcttatgttaatctaaaaaaccttcagtgacctacttgatctcattttctaccattttcctcctctttttctgaaatacatcaacacagagcacttttcctctcctttaatgcacaaagatggcaggacttttgaatgttacatttatttatcttcttctagagtgcctttccttatacacccatgtgacttgttcctcccttccttctagtctttgtttatatatatattattatcacagagggctaggaaagaaaacacccactgctgcgccccacactcatccacctgccctgtaccacttactttgttttgtttttctctgtagaattcatgactttttgaaatataatttttttaatgtgtacatactttatgctttctctcattcatatgtaaagtctggaagacacagactggtttttttgttcactgttgatgcttcagtccctaaaatatgcatagcatgaattgccactttttaaattaataaatctggaacattgttaaaattcaaaaaaaaaaaaaaaaaaaaaaa

SEQ ID NO: 112 - protein, 116 aa, Homo sapiens
MAGLLNVTFIYLLLECLSLYTHVTCSSLPSSLCLYIYYYHRGLGKKTPTAAPHTHPPALYHLLCFVFLCRIHDFLKYNFFNVYILYAFSHSYVKSGRHRLVFLFTVDASVPKICIA

SEQ ID NO: 113 - DNA, 2759 bp, Homo sapiens
tttttttttttttgaaagacacacgttattttattaatatagccatctctccccactgccccagtggtgaaggtgtttgcattgcaacatggaggggcaccaaatgctctgcgggccctagcccgctgccacaggctaggcctgcctgcagccaagaaggctgctcaaactctagatgccatttggaggcatgaggacctgagcccagaggtggcagtgtcctacccagggaagtcaacagatcgtgctccaggtcccagctctgggctgggccaggactaaatcctggctcccctttcttggtactaaggggattagtgcttggttgtctgtagggggtcagagtagggagggttccaggaagggttccagagtgggctcacaggggacctcctcccctggcctcttggagtccaggtcgtcgagggcgcaaagctgcacgccatcctgggcaagctgggcccgcagcgtgggcgcggtgaggacgcgcagctcatgcagccgctcccaagagcaagagaaagcgtcggggccttcaccgcagccgccggtgggaggcacactggggtagccggggtgcgccatcagctcggctgtcagggtgtggcccgctagggtaccttccaggacccgcgccagggccccggacacgcggtgagcggacatgtgccggccgcaagtgctcaggcccacgaaggcgtctgtccaccgcaggccgtggcgggagaagggcccacggcggcccgggcgtcgcgctccacggcgcaggcgaaggcacgcgcgggggcctccagccaagtgcagccacccacaccgcgctccagcggcagtcgcgtaaagcgcaccccataggcctgcagcgcctcggcgaacacctggcacacgcctgggagcacgtgcacgtgctggtgcccgtccgcgtgcgtgggggccctgcccagcagctcccggaagcagcttagttgggcctcgagctcctcccgcacctagagggcgagcgagagacaccttgagcgaccgggagtagctgccgaggataccctcctcgtgcttccgtgtgatggctccagtgtttgaatgcggaagtcatccaccgccagctcctaacggcctcacagtaccctccgggcggagctctgggggtcctcgcgagcatcctcctgtagctgcggctccgcacctgaggcaaatccacgtctccggccgccaccgcctcccggaatcccatcttgccaaggaagaagccttccgggccgagcagcgatgaggcgccacggcgggccggacccacggggcggccctcggacaggttggcgtggaggcccgtggggatgctgtgcctgcgggccagctccgccgcgctctccgtggccgcaccgttgaccagcagggacacgctggtcacggccccggccagaaaggcctccacgataccctcatcgcgtcgcgggcagtaaccaaagtcgtccgcggtgaccaccaggcgcccgcgggagccggcagatccgggtcttgtggctaagagtgacgtggtcactcgaatcaaaacagaggagggggaggaagccggcggccagaaacggcagtggcagcagcgtccggagcagccgcagccttctggaagctccaggcggtctttctgccgagcctcggtcccggcccccatcctccccgccccatcggttgttgtctgggcggatttaaacagtcaagtaaaatcaagctgggtaatcatggcagaaggtggatttgatccctgtgaatgtgtttgctctcatgaacatgcaatgagaagactgatcaatctgttacggcagtcccagtcctactgcacagacacagagtgtcttcaggaattaccgggaccctctggtgataatggcatcagtgttacaatgatcttggtagcctggatggttattgcattgatcttgttcttactgagacctcctaatctaagaggatccagcctacctggaaagccaaccagtcctcataatggacaagatccaccagctcctcctgtggactaactttgtgatatgggaagtgaaaatagttaacaccttgcacgaccaaacgaacgaagatgaccagagtactcttaaccccattagaactgtttttccttttgtatctgcaatatgggatggtattgttttcatgagcttctagaaatttcacttgcaagtttatttttgcttcctgtgttactgccattcctatttacagtatatttgagtgaatgattatatttttaaaaagttacatggggcttttttggttgtcctaaacttacaaacattccactcattctgtttgtaactgtgattataatttttgtgataatttctggcctgattgaaggaaatttgagaggtctgcatttatatattttaaatagatttgataggtttttaaattgctttttttcataaggtatttataaagttatttggggttgtctgggattgtgtgaaagaaaattagaaccacgctgtatttacatttaccttggtagtttatttgtggatggcagttttctgtagttttggggactgtggtagctcttggattgttttgcaaattacagctgaaatctgtgtcatggattaaactggcttatgtggctagaataggaagagaaaaaaaatgaaatggttgtttactaattttatactcccattaaaaatctctaatgttaagaaaaccttaaataaacatgattgatcaatatgaaaaaaaaaaaaaaaaaaaaa

SEQ ID NO: 114 - protein, 99 aa, Homo sapiens
MAEGGFDPCECVCSHEHAMRRLINLLRQSQSYCTDTECLQELPGPSGDNGISVTMILVAWMVIALILFLLRPPNLRGSSLPGKPTSPHNGQDPPAPPVD

SEQ ID NO: 115 - DNA, 1404 bp, Homo sapiens
aatcgggacgggacgaattattggttgggggaaacccacgaggggacgcggccgaggagggtcgctgtccacccgggggcgtgggagtgaggtaccagattcagcccatttggccccgacgcctctgttctcggaatccgggtgctgcggattgaggtcccggttcctaacggtgggatcggtgtcctcgggatgagatttggcgtttcctcggggctttggtgggatcggtgtcctcaggatgagatttagggtttcctcggggctttcgggatcttcacctaatatccggtattattttatgagaggagtggtcttggctgtcagaactggatccctggggtgatatttgggaattagtggagtgatctctgaagacctagggctatgatctggagctgctgtggctgaaatttggggcctctgaagtggcatggagattgaggtccagagagcctgagatcttgagggctgacatttggagagatggggtcgagggttgtctttgggccttgactgctttgggcctttctcactctcattcccgggatgctttgccagaatctctgctggattggccgtaaccctgtccccgagcgggctcacagggtctgaaggccacgcatgaggcaaaggtaaagttctgagccacccggtgcctccttcccaggactgcaagatggaggaaggcgggaacctaggaggcctgattaagatggtccatctactggtcttgtcaggtgcctggggcatgcaaatgtgggtgaccttcgtctcaggtagggaccctcagcttggatgtcatgggtacctggggtggggatggaaataagaggggaaccgggaagtgccctaacacccctgtggtccccataccctgcaggcttcctgcttttccgaagccttccccgacataccttcggactagtgcagagcaaactcttccccttctacttccacatctccatgggctgtgccttcatcaacctctgcatcttggcttcacagcatgcttgggctcagctcacattctgggaggccagccagctttacctgctgttcctgagccttacgctggccactgtcaacgcccgctggctggaaccccgcaccacagctgccatgtgggccctgcaaaccgtggagaaggagcgaggcctgggtggggaggtaccaggcagccaccagggtcccgatccctaccgccagctgcgagagaaggaccccaagtacagtgctctccgccagaatttcttccgctaccatgggctgtcctctctttgcaatctgggctgcgtcctgagcaatgggctctgtctcgctggccttgccctggaaataaggagcctctagcatgggccctgcatgctaataaatgcttcttcagaaaaaaaaaaaaaaaaa

SEQ ID NO: 116 - protein, 184 aa, Homo sapiens
MSWVPGVGMEIRGEPGSALTPLWSPYPAGFLLFRSLPRHTFGLVQSKLFPFYFHISMGCAFINLCILASQHAWAQLTFWEASQLYLLFLSLTLATVNARWLEPRTTAAMWALQTVEKERGLGGEVPGSHQGPDPYRQLREKDPKYSALRQNFFRYHGLSSLCNLGCVLSNGLCLAGLALEIRSL

SEQ ID NO: 117 - DNA, 1801 bp, Homo sapiens
tgaagaaggtgtttactttttttgaaattaccttgagacatttcaaactgtgcagaagatatatgcacaaaagcaaatgtcttgcagtttgctatagccacttatacatcatctggctcttgaatagctttaattcagctgttgaatctcacttgaatttgagcaaaaccttcatctttatatgtatctggacaaattacttcaattgcttgacagtaatgaccaatcaatttatttaaaatagtatcatttagtaggacagtgtttttctctggtttgagcaacgaattcaaccagtcctctgggttgatcatcatcatcatcatcatttggttatcagttcctgagttatttttaccagggagttttatacctttagacaactattttgaattatctcaggaatgtcatatatctctgcctctttagagtcagtcactggcactttgtctgtttggtgacatcatgtttccctgactgttcttcatctttgtagttatacattgatatctgtgcattgaatatgtaggtatttataaacagtctttgcaatctggctttgtctgtgattgtccttgtatagtaggtctgtccagaaattgtaagcatactgtcttttttggtctttaagcccgtgaacgctacagcccgtgtagtgccaaatggtgccctaagcccaggttccctgcagtccactctgtgatttgtttgttgactgctgtgagccccacccccattctttgtttctaatttacacctagcaggctaaccctgctggcacctgcagtgcttccagggggaaaggaccatagtgtgcccctgtgaagagtctcagaatggtgcggaaggtgaatgcccacctcccgctctcttttcccactgtagaaactgattccagagaaattctccaagtgcggtgctatgtgggcttgcgggagaggtgttatgatcaaacagaaccattctctttaccctctgttcatggtttttcttggctctgtggtccagtgagttgtcacagcttcactcccaatttctgggatattcagggcaataatcttgccactgggtatttgctagttgaaattatgtggtagggagagaagccagtaagcttcacttctccgtttggctgatgtcactctcgaattctgtactttcatacggattgtaatagggagggtctaaaaggaagcttcagttttggatttttagagcttcttctaagtacaattgttgaatcaagaggtaaagatggtatgttataactggaaatacgctgagattaaaaaggagataaattagccccattgggacagtgctattgggaatgtgaattgataccactttcctggagtctagtttagtatttattttatatgtaatgcctgaaaagatgtgtgtctcctttgactcaatacttataggaatttacacaaaggttataatcaaagattatagaactgttatactggaaaaacacgaaatattctaattatgaattaattggggattggttaaataaactatggtttgatatgctctaaaaataatgttacagaaaaaagtgtactgatatggcaaaatgtatgacttatagttaaaaaagcaggttagatgttgatagatacagtatgatagaaaaagatcaggaaggtatatgctgacatttaaatctggatatttatgagtgttttttttatttcaatctttgtacatgcatgtattttctagaaattgtattactatctttgtaataaagtaaattatttttaagggactaaaaaaaaaaaaaaaaaaa

SEQ ID NO: 118 - protein, 86 aa, Homo sapiens [sequence omitted]

SEQ ID NO: 119 - DNA, 29 bp, Artificial Sequence
  oligonucleotide
ancgggagcctcctgaccatctcctcttc

SEQ ID NO: 120 - DNA, 29 bp, Artificial Sequence
  oligonucleotide
cncacagaaaattcaataagaccctcgct

SEQ ID NO: 121 - DNA, 29 bp, Artificial Sequence
  oligonucleotide
cncagctcttcgtagggaagttctgactt

SEQ ID NO: 122 - DNA, 29 bp, Artificial Sequence
  oligonucleotide
antcctgcacaccagccagtaacgccacc

SEQ ID NO: 123 - DNA, 29 bp, Artificial Sequence
  oligonucleotide
gnggctggaaagatgtgtggggatcaaga

SEQ ID NO: 124 - DNA, 29 bp, Artificial Sequence
  oligonucleotide
anatgggtctaagccacacaacagggtga

SEQ ID NO: 125 - DNA, 29 bp, Artificial Sequence
  oligonucleotide
ancggcagggaacttacagggacagagct

SEQ ID NO: 126 - DNA, 29 bp, Artificial Sequence
  oligonucleotide
gngttttcggtgtcgatggtgtagaggat

SEQ ID NO: 127 - DNA, 29 bp, Artificial Sequence
  oligonucleotide
cnagaacacatagggatgcgagagcaagc

SEQ ID NO: 128 - DNA, 29 bp, Artificial Sequence
  oligonucleotide
anactgaaaactgagtatgtgcgagtgta

SEQ ID NO: 129 - DNA, 29 bp, Artificial Sequence
  oligonucleotide
gntatccatttcctcttcttcatctgagt

SEQ ID NO: 130 - DNA, 29 bp, Artificial Sequence
  oligonucleotide
tncttgaggcaatggttgaagtccggcgg

SEQ ID NO: 131 - DNA, 29 bp, Artificial Sequence
  oligonucleotide
tnctctgctgtgtccttctctatccgaac

SEQ ID NO: 132 - DNA, 29 bp, Artificial Sequence
  oligonucleotide
gngcatctcactggatgtcatcatcatca

SEQ ID NO: 133 - DNA, 29 bp, Artificial Sequence
  oligonucleotide
tngtccatgtgaagggcatgggccagttg

SEQ ID NO: 134 - DNA, 29 bp, Artificial Sequence
  oligonucleotide
gngcactgtattgagctgattgctgaagc

SEQ ID NO: 135 - DNA, 29 bp, Artificial Sequence
  oligonucleotide
cncagaagcagaagaatgacaggcaacac

SEQ ID NO: 136 - DNA, 29 bp, Artificial Sequence
  oligonucleotide
anacattctgagtagttgcatgatttccc

SEQ ID NO: 137 - DNA, 29 bp, Artificial Sequence
  oligonucleotide
gnccagaaagttgaggacatgctgggcag

SEQ ID NO: 138 - DNA, 29 bp, Artificial Sequence
  oligonucleotide
angggaacaagacaactggagaagggtca

SEQ ID NO: 139 - DNA, 29 bp, Artificial Sequence
  oligonucleotide
tngtctcccaggtagacagagggcttcag

SEQ ID NO: 140 - DNA, 29 bp, Artificial Sequence
  oligonucleotide
anccatctacatgtgcattgacaagctta

SEQ ID NO: 141 - DNA, 29 bp, Artificial Sequence
  oligonucleotide
tngtgatagatcctttcgtaacaccaagt

SEQ ID NO: 142 - DNA, 29 bp, Artificial Sequence
  oligonucleotide
angaccagatctcacccagcacatcaaac

SEQ ID NO: 143 - DNA, 29 bp, Artificial Sequence
  oligonucleotide
tntttggggcaagatggctgttaagcagt

SEQ ID NO: 144 - DNA, 29 bp, Artificial Sequence
  oligonucleotide
tnggttgttccgggcagggcattcttgtc

SEQ ID NO: 145 - DNA, 29 bp, Artificial Sequence
  oligonucleotide
tnacacgctctgtgctaggacttttatat

SEQ ID NO: 146 - DNA, 29 bp, Artificial Sequence
  oligonucleotide
cnggatgtgtgatattggagcttgctgtt

SEQ ID NO: 147 - DNA, 29 bp, Artificial Sequence
  oligonucleotide
ancatcaaaggtgcccaaataagttccca

SEQ ID NO: 148 - DNA, 29 bp, Artificial Sequence
  oligonucleotide
anatcactgcatttgttctggaacctgac

SEQ ID NO: 149 - DNA, 29 bp, Artificial Sequence
  oligonucleotide
gntgacttcaatctcctcaccttccaccg

SEQ ID NO: 150 - DNA, 29 bp, Artificial Sequence
  oligonucleotide
gnagtgccacctatgactaccaaattctc

SEQ ID NO: 151 - DNA, 29 bp, Artificial Sequence
  oligonucleotide
gngggatgaggcaatgaacacaatgaaag

SEQ ID NO: 152 - DNA, 29 bp, Artificial Sequence
  oligonucleotide
cnaaactggtgtttttaccgtatccttca

SEQ ID NO: 153 - DNA, 29 bp, Artificial Sequence
  oligonucleotide
tngattctgccgaatccgaaagtgctctc

SEQ ID NO: 154 - DNA, 29 bp, Artificial Sequence
  oligonucleotide
angttgatgggctcaacacagggcagagg

SEQ ID NO: 155 - DNA, 29 bp, Artificial Sequence
  oligonucleotide
anggatgccatctctcacccactctgtac

SEQ ID NO: 156 - DNA, 29 bp, Artificial Sequence
  oligonucleotide
anaccaccacctcgacaggcattccttaa

SEQ ID NO: 157 - DNA, 29 bp, Artificial Sequence
  oligonucleotide
cncctgagggtagaaggccgctcaggttt

SEQ ID NO: 158 - DNA, 29 bp, Artificial Sequence
  oligonucleotide
tngagttagcagagcaagaagcaaggagg

SEQ ID NO: 159 - DNA, 29 bp, Artificial Sequence
  oligonucleotide
tttgcatgtatagttctctgcagtagcat

SEQ ID NO: 160 - DNA, 29 bp, Artificial Sequence
  oligonucleotide
anctgccatgtcaaagaggagccagatga

SEQ ID NO: 161 - DNA, 29 bp, Artificial Sequence
  oligonucleotide
tnaggcttgtgcacttgctgagcttccag

SEQ ID NO: 162 - DNA, 29 bp, Artificial Sequence
  oligonucleotide
cnttcgagcactaagaacgggacacggta

SEQ ID NO: 163 - DNA, 29 bp, Artificial Sequence
  oligonucleotide
angagaagttctgtgcgtgggtctggtcg

SEQ ID NO: 164 - DNA, 29 bp, Artificial Sequence
  oligonucleotide
angtcgttctgcaccaggcctctgtagcg

SEQ ID NO: 165 - DNA, 29 bp, Artificial Sequence
  oligonucleotide
gntcatctccagcaccatctccatcaatg

SEQ ID NO: 166 - DNA, 20 bp, Artificial Sequence
  oligonucleotide
actgtctttgaatggtattg

SEQ ID NO: 167 - DNA, 29 bp, Artificial Sequence
  oligonucleotide
angttcacatatgatacaaggctctttcc

SEQ ID NO: 168 - DNA, 29 bp, Artificial Sequence
  oligonucleotide
cnagtagacagcacaggtagtcggcttga

SEQ ID NO: 169 - DNA, 29 bp, Artificial Sequence
  oligonucleotide
gngaatgcaatatgaagaaaacaggtcag

SEQ ID NO: 170 - DNA, 29 bp, Artificial Sequence
  oligonucleotide
anatcaaggtgattaggctcttccatgca

SEQ ID NO: 171 - DNA, 29 bp, Artificial Sequence
  oligonucleotide
tntacattcaatgcctttgcttcctgctg

SEQ ID NO: 172 - DNA, 29 bp, Artificial Sequence
  oligonucleotide
antccatgagaccaccctaaactgtccat

SEQ ID NO: 173 - DNA, 29 bp, Artificial Sequence
  oligonucleotide
gngaggaaaagtgctctgtgttgatgtat

SEQ ID NO: 174 - DNA, 29 bp, Artificial Sequence
  oligonucleotide
antccaccttctgccatgattacccagct

SEQ ID NO: 175 - DNA, 29 bp, Artificial Sequence
  oligonucleotide
anccaagatgcagaggttgatgaaggcac

SEQ ID NO: 176 - DNA, 29 bp, Artificial Sequence
  oligonucleotide
tntaatgcctgaaaagatgtgtgtctcct

SEQ ID NO: 177 - protein, 388 aa, Homo sapiens
MHLYKTNKMTSLKEDVRRSAMLCILTVPAAMTSHDLMKFVAPFNEVIEQMKIIRDSTPNQYMVLIKFRAQADADSFYMTCNGRQFNSIEDDVCQLVYVERAEVLKSEDGASLPVMDLTELPKCTVCLERMDESVNGILTTLCNHSFHSQCLQRWDDTTCPVCRYCQTPEPVEENKCFECGVQENLWICLICGHIGCGRYVSRHAYKHFEETQHTYAMQLTNHRVWDYAGDNYVHRLVASKTDGKIVQYECEGDTCQEEKIDALQLEYSYLLTSQLESQRIYWENKIVRIEKDTAEEINNMKTKFKETIEKCDNLEHKLNDLLKEKQSVERKCTQLNTKVAKLTNELKEEQEMNKCLRANQVLLQNKLKEEERVLKETCDQKDLQITEI

SEQ ID NO: 178 - protein, 171 aa, Homo sapiens
MMMQCVSRMLAHPLHVISMRCMVQFVGREAKYSGVLSSIGKIFKEEGLLGFFVGLIPHLLGDVVFLWGCNLLAHFINAYLVDDSFSQALAIRSYTKFVMGIAVSMLTYPFLLVGDLMAVNNCGLQAGLPPYSPVFKSWIHCWKYLSVQGQLFRGSSLLFRRVSSGSCFALE

SEQ ID NO: 179 - protein, 142 aa, Homo sapiens
MHQLLQLQRQEPCRLLSPSPQPGLHHLCFQQIELLLLLLHLQWGLGLLRQLHHKRLAQLLLHRRRDHPIPPIQDILGIAKCPCPWAIILMRMASIICHIHQCITRVLDRLHTRDPSSLHTPSLSPHSSLTIHSSNMSAQQLS

SEQ ID NO: 180 - protein, 82 aa, Homo sapiens [sequence omitted]